(19) United States
(12) Patent Application Publication
(10) Pub. No.: US 2026/0001919 A1
(43) Pub. Date: Jan. 1, 2026

(54) ADENO-ASSOCIATED VIRUS WITH ENGINEERED CAPSID

(71) Applicant: Tenaya Therapeutics, Inc., South San Francisco, CA (US)

(72) Inventor: Ze CHENG, South San Francisco, CA (US)

(21) Appl. No.: 19/251,293

(22) Filed: Jun. 26, 2025

Related U.S. Application Data
(60) Provisional application No. 63/665,003, filed on Jun. 27, 2024.

(51) Int. Cl. C07K 14/005; C12N 15/86

(52) U.S. Cl. CPC C07K 14/005; C12N 15/86; C12N 2750/14122; C12N 2750/14143

(57) ABSTRACT

In some aspects, the present disclosure provides engineered adeno-associated virus (AAV) capsid proteins comprising a non-naturally occurring amino acid motif described herein. In some embodiments, the present disclosure provides an AAV9, AAV5, AAVrh.10 or AAVrh.74-based engineered capsid protein, that when assembled into virions, achieves increased transduction efficiency of the heart, and/or other desirable properties. Also provided herein are recombinant AAV virions comprising any of the engineered capsid proteins described herein and uses thereof.

### CROSS-REFERENCE TO RELATED APPLICATIONS

This application claims the benefit of U.S. Provisional Patent Application No. 63/665,003, filed Jun. 27, 2024, which is incorporated by reference herein in its entirety.

### STATEMENT REGARDING SEQUENCE LISTING

The Sequence Listing XML associated with this application is provided in XML file format and is hereby incorporated by reference into the specification. The name of the XML file containing the Sequence Listing XML is TENA_062_01US_SeqList_ST26.xml. The XML file is 491,618 bytes, and created on Jun. 26, 2025, and is being submitted electronically via USPTO Patent Center.

### TECHNICAL FIELD

In some aspects, the present disclosure relates to engineered adeno-associated virus (AAV) capsid proteins comprising amino acid substitutions, amino acid insertions, and/or non-naturally occurring amino acid motifs. In some aspects, the present disclosure relates to virions containing the same, and uses thereof.

### BACKGROUND

AAV holds promise for gene therapy and other biomedical applications. In particular, AAV can be used to deliver gene products to various tissues and cells, both in vitro and in vivo. The capsid proteins of AAV largely determine the immunogenicity and tropism of AAV vectors.

For cardiac tissues, AAV serotype 9 (AAV9) is a preferred AAV vector due to its ability to transduce the heart following systemic delivery. While AAV9 can achieve moderate transduction of the heart, the majority of vector traffics to the liver. Moreover, in order to achieve therapeutic levels of transduction in the heart, relatively high systemic doses are required, potentially leading to systemic inflammation and in turn, toxicity.

Accordingly, there is a need for developing AAVs with engineered capsid proteins that are modified to achieve improved cardiac tropism, and optionally improved selectivity of cardiac tissues over liver. The present disclosure provides engineered AAV capsid proteins modified to have non-naturally occurring amino acid motifs at various locations including, for example, the VR-VIII site, that form rAAV virions capable of transducing cardiac tissues and/or cell types more efficiently and/or with more selectivity than rAAV virions comprising wild-type capsid proteins, which can be used for safe and efficacious cardiac gene therapy.

### SUMMARY

In some aspects, provided herein is an engineered adeno-associated virus (rAAV) capsid protein, wherein the capsid protein comprises an amino acid substitution at one, two, three, four, or five of the following positions relative to a wild-type AAV9 capsid protein sequence: S586, A587, Q588, A589, and Q590, wherein the amino acid numbering is according to the AAV9 VP1 sequence of SEQ ID NO:1. In some embodiments, the amino acid substitutions are selected from S586E, S586A, A587S, A587N, Q588V, Q588R, Q588T, A589T, A589N, A589S, Q590G, Q590L, and Q590R. In some embodiments, the engineered capsid protein comprises at least four amino acid substitutions relative to a wild-type or a parental AAV capsid protein, wherein the amino acid substitutions are selected from S586E, S586A, A587N, Q588R, and Q588T. In some embodiments, the engineered capsid protein comprises amino acid substitutions selected from: a) S586E, A587N, Q588R, and A589T; b) S586A, A587S, Q588T, and Q590G; c) S586E, A587N, Q588R, A589T, and Q590L; d) S586A, A587S, Q588T, A589T, and Q590L; e) S586E, A587N, Q588R, A589N, Q590R; f) S586A, A587S, Q588T, and A589T; and g) S586A, A587S, Q588T, A589S, and Q590G.

In some embodiments, the engineered capsid protein comprises amino acid substitutions selected from: a) A587S and Q588V; b) S586E, A587N, Q588R, and A589T; c) S586A, A587S, Q588T, and Q590G; d) S586E, A587N, Q588R, A589T, and Q590L; e) S586E, A587N, and Q588R; f) S586A, A587S, and Q588T; g) S586A, A587S, Q588T, A589T, and Q590L; h) S586E, A587N, Q588R, A589N, Q590R; i) S586A, A587S, Q588T, and A589T; and j) S586A, A587S, Q588T, A589S, and Q590G.

In some embodiments, the engineered capsid protein comprises amino acid substitutions S586E, A587N, Q588R, and A589T. In some embodiments, the engineered capsid protein comprises amino acid substitutions S586E, A587N, and Q588R. In some embodiments, the engineered capsid protein comprises amino acid substitutions A587S and Q588V.

In some embodiments, the engineered capsid protein comprises a polypeptide sequence inserted between positions 588 and 589, wherein the polypeptide sequence comprises an amino acid sequence RX₁DX₂X₃X₄X₅, wherein: X₁ is Glycine (G) or Threonine (T); X₂ is Histidine (H), Serine (S), Alanine (A), Leucine (L), Threonine (T), Glycine (G), Valine (V), or Phenylalanine (F); X₃ is Glycine (G), Alanine (A), Valine (V), Lysine (K), Asparagine (N), or Threonine (T); X₄ is Valine (V), Serine (S), Glycine (G), Asparagine (N), or Arginine (R); and X₅ is Leucine (L), Tryptophan (W), Threonine (T), Glycine (G), or Arginine (R).

In some embodiments, the polypeptide sequence is selected from SEQ ID NOs: 215-242. In some embodiments, the polypeptide sequence is SEQ ID NOs: 234. In some embodiments, the polypeptide sequence is SEQ ID NOs: 218. In some embodiments, the polypeptide sequence is SEQ ID NOs: 241.

In some aspects, provided herein is an engineered adeno-associated virus (AAV) capsid protein, comprising a non-naturally occurring amino acid motif comprising an amino acid sequence of X₁X₂X₃RX₄DX₅X₆X₇X₈ X₉X₁₀ in the VR-VIII site, wherein: X₁ is Serine (S), Glutamic acid (E), or Alanine (A); X₂ is Alanine (A), Serine (S), or Asparagine (N); X₃ is Glutamine (Q), Valine (V), Arginine (R), or Threonine (T); X₄ is Glycine (G) or Threonine (T); X₅ is Histidine (H), Serine (S), Alanine (A), Leucine (L), Threonine (T), Glycine (G), Valine (V), or Phenylalanine (F); X₆ is Glycine (G), Alanine (A), Valine (V), Lysine (K), Asparagine (N), or Threonine (T); X₇ is Valine (V), Serine (S), Glycine (G), Asparagine (N), or Arginine (R); X₈ is Leucine (L), Tryptophan (W), Threonine (T), Glycine (G), or Arginine (R); X₉ is Alanine (A), Threonine (T), Asparagine (N), or Serine (S); and X₁₀ is Glutamine (Q), Glycine (G), Leucine (L), or Arginine (R).

In some embodiments, the non-naturally occurring amino acid motif comprises: (a) an amino acid sequence selected from any one of SEQ ID NOs: 78-145, or (b) an amino acid sequence having no more than 1 or 2 amino acid substitutions in the amino acid sequence selected from any one of SEQ ID NOs: 78-145.

In some embodiments, the non-naturally occurring amino acid motif comprises SEQ ID NO: 81. In some embodiments, the non-naturally occurring amino acid motif comprises SEQ ID NO: 119. In some embodiments, the non-naturally occurring amino acid motif comprises SEQ ID NO: 135.

In some embodiments, the 1 or 2 amino acid substitutions are conservative amino acid substitutions.

In some embodiments, the engineered AAV capsid protein is a variant of an AAV5, AAV9, AAVrh.74, or AAVrh.10 capsid protein.

In some embodiments, the non-naturally occurring amino acid motif comprises an amino acid insertion. In some embodiments, the non-naturally occurring amino acid motif comprises an amino acid substitution, wherein the amino acid substitution is generated by one, two, three, four, five, or more amino acid substitutions in the amino acid sequence of the wild-type or parental AAV capsid protein.

In some aspects, provided herein is an engineered an engineered adeno-associated virus (AAV) capsid protein, wherein the engineered capsid protein:
(i) comprises at least 80% amino acid sequence identity to SEQ ID NO: 3, and comprises an amino acid sequence of any one of SEQ ID NOs: 78-145 replacing the natural amino acid sequence at amino acid positions 586 to 590, wherein the amino acid numbering is according to SEQ ID NO: 1;
(ii) comprises at least 80% amino acid sequence identity to SEQ ID NO: 12, and comprises an amino acid sequence of any one of SEQ ID NOs: 78-145 replacing the natural amino acid sequence at amino acid positions 575 to 579, wherein the amino acid numbering is according to SEQ ID NO: 10;
(iii) comprises at least 80% amino acid sequence identity to SEQ ID NO: 21 and comprises an amino acid sequence of any one of SEQ ID NOs: 78-145 replacing the natural amino acid sequence at amino acid positions 588 to 592, wherein the amino acid numbering is according to SEQ ID NO: 19; or
(iv) comprises at least 80% amino acid sequence identity to SEQ ID NO: 30, and comprises an amino acid sequence of any one of SEQ ID NOs: 78-145 replacing the natural amino acid sequence at amino acid positions 588 to 592, wherein the amino acid numbering is according to SEQ ID NO: 28.

In some aspects, provided herein is an engineered an engineered adeno-associated virus (AAV) capsid protein, wherein the engineered capsid protein:
(i) comprises at least 80% amino acid sequence identity to SEQ ID NO: 3, and comprises SEQ ID NO: 81 replacing the natural amino acid sequence at amino acid positions 586 to 590, wherein the amino acid numbering is according to SEQ ID NO: 1;
(ii) comprises at least 80% amino acid sequence identity to SEQ ID NO: 12, and comprises SEQ ID NO: 81 replacing the natural amino acid sequence at amino acid positions 575 to 579, wherein the amino acid numbering is according to SEQ ID NO: 10;
(iii) comprises at least 80% amino acid sequence identity to SEQ ID NO: 21, and comprises SEQ ID NO: 81 replacing the natural amino acid sequence at amino acid positions 588 to 592, wherein the amino acid numbering is according to SEQ ID NO: 19; or
(iv) comprises at least 80% amino acid sequence identity to SEQ ID NO: 30, and comprises SEQ ID NO: 81 replacing the natural amino acid sequence at amino acid positions 588 to 592, wherein the amino acid numbering is according to SEQ ID NO: 28.

In some aspects, provided herein is an engineered an engineered adeno-associated virus (AAV) capsid protein, wherein the engineered capsid protein:
(i) comprises at least 80% amino acid sequence identity to SEQ ID NO: 3, and comprises SEQ ID NO: 119 replacing the natural amino acid sequence at amino acid positions 586 to 590, wherein the amino acid numbering is according to SEQ ID NO: 1;
(ii) comprises at least 80% amino acid sequence identity to SEQ ID NO: 12, and comprises SEQ ID NO: 119 replacing the natural amino acid sequence at amino acid positions 575 to 579, wherein the amino acid numbering is according to SEQ ID NO: 10;
(iii) comprises at least 80% amino acid sequence identity to SEQ ID NO: 21, and comprises SEQ ID NO: 119 replacing the natural amino acid sequence at amino acid positions 588 to 592, wherein the amino acid numbering is according to SEQ ID NO: 19; or
(iv) comprises at least 80% amino acid sequence identity to SEQ ID NO: 30, and comprises SEQ ID NO: 119 replacing the natural amino acid sequence at amino acid positions 588 to 592, wherein the amino acid numbering is according to SEQ ID NO: 28.

In some aspects, provided herein is an engineered an engineered adeno-associated virus (AAV) capsid protein, wherein the engineered capsid protein:
(i) comprises at least 80% amino acid sequence identity to SEQ ID NO: 3, and comprises SEQ ID NO: 135 replacing the natural amino acid sequence at amino acid positions 586 to 590, wherein the amino acid numbering is according to SEQ ID NO: 1;
(ii) comprises at least 80% amino acid sequence identity to SEQ ID NO: 12, and comprises SEQ ID NO: 135 replacing the natural amino acid sequence at amino acid positions 575 to 579, wherein the amino acid numbering is according to SEQ ID NO: 10;
(iii) comprises at least 80% amino acid sequence identity to SEQ ID NO: 21, and comprises SEQ ID NO: 135 replacing the natural amino acid sequence at amino acid positions 588 to 592, wherein the amino acid numbering is according to SEQ ID NO: 19; or
(iv) comprises at least 80% amino acid sequence identity to SEQ ID NO: 30, and comprises SEQ ID NO: 135 replacing the natural amino acid sequence at amino acid positions 588 to 592, wherein the amino acid numbering is according to SEQ ID NO: 28.

In some aspects, provided herein is an engineered adeno-associated virus (AAV) capsid protein, comprising a non-naturally occurring amino acid motif comprising an amino acid sequence RX₁DX₂X₃X₄X₅ in the VR-VIII site, wherein: X₁ is Glycine (G) or Threonine (T); X₂=Histidine (H), Serine (S), Alanine (A), Leucine (L), Threonine (T), Glycine (G), Valine (V), or Phenylalanine (F); X₃=Glycine (G), Alanine (A), Valine (V), Lysine (K), Asparagine (N), or Threonine (T); X₄=Valine (V), Serine (S), Glycine (G), Asparagine (N), or Arginine (R); and X₅=Leucine (L), Tryptophan (W), Threonine (T), Glycine (G), or Arginine (R).

In some embodiments, the non-naturally occurring amino acid motif comprises an amino acid sequence RX₁DX₂X₃X₄X₅ in the VR-VIII site, wherein: X₁ is Glycine (G) or Threonine (T); X₂=Serine (S), Alanine (A), Leucine (L), Threonine (T), Glycine (G), or Valine (V); X₃=Glycine (G), Alanine (A), or Asparagine (N); X₄=Valine (V), Serine (S), or Asparagine (N); and X₅=Leucine (L), Tryptophan (W), or Threonine (T).

In some embodiments, the non-naturally occurring amino acid motif comprises an amino acid sequence selected from any one of SEQ ID NOs: 215-227. In some embodiments, the non-naturally occurring amino acid motif comprises SEQ ID NOs: 234. In some embodiments, the non-naturally occurring amino acid motif comprises SEQ ID NOs: 218. In some embodiments, the non-naturally occurring amino acid motif comprises SEQ ID NOs: 241.

In some aspects, provided herein is an engineered adeno-associated virus (AAV) capsid protein, wherein the engineered capsid protein comprises at least 90% or at least 95% amino acid sequence identity to AAV9 VP3 SEQ ID NO: 3, and comprises the amino acid sequence of any one of SEQ ID NOs: 147-214 replacing the natural amino acid sequence at amino acid positions 581 to 595, wherein the amino acid numbering is according to AAV9 VP1 SEQ ID NO: 1.

In some embodiments, the engineered capsid protein comprises at least 90% or at least 95% amino acid sequence identity to AAV9 VP3 SEQ ID NO: 3, and comprises the amino acid sequence of any one of SEQ ID NO: 150 replacing the natural amino acid sequence at amino acid positions 581 to 595, wherein the amino acid numbering is according to AAV9 VP1 SEQ ID NO: 1. In some embodiments, the engineered capsid protein comprises at least 90% or at least 95% amino acid sequence identity to AAV9 VP3 SEQ ID NO: 3, and comprises the amino acid sequence of any one of SEQ ID NO: 188 replacing the natural amino acid sequence at amino acid positions 581 to 595, wherein the amino acid numbering is according to AAV9 VP1 SEQ ID NO: 1. In some embodiments, the engineered capsid protein comprises at least 90% or at least 95% amino acid sequence identity to AAV9 VP3 SEQ ID NO: 3, and comprises the amino acid sequence of any one of SEQ ID NO: 204 replacing the natural amino acid sequence at amino acid positions 581 to 595, wherein the amino acid numbering is according to AAV9 VP1 SEQ ID NO: 1

In some aspects, provided herein is an engineered adeno-associated virus (AAV) capsid protein, comprising or consisting of an amino acid sequence that shares at least 80%, at least 85%, at least 90%, at least 95%, at least 96% at least 97%, at least 98%, at least 99%, or 100% identity to any one of SEQ ID NOs: 243-310.

In some embodiments, the engineered AAV capsid protein comprises or consists of an amino acid sequence that shares at least 80%, at least 85%, at least 90%, at least 95%, at least 96% at least 97%, at least 98%, at least 99%, or 100% identity to SEQ ID NO: 246. In some embodiments, the engineered AAV capsid protein comprises or consists of an amino acid sequence that shares at least 80%, at least 85%, at least 90%, at least 95%, at least 96% at least 97%, at least 98%, at least 99%, or 100% identity to SEQ ID NO: 284. In some embodiments, the engineered AAV capsid protein comprises or consists of an amino acid sequence that shares at least 80%, at least 85%, at least 90%, at least 95%, at least 96% at least 97%, at least 98%, at least 99%, or 100% identity to SEQ ID NO: 300.

In some aspects, provided herein is a recombinant adeno-associated virus (rAAV) virion, comprising the engineered capsid protein according to various embodiments disclosed herein and a vector genome comprising an expression cassette flanked by inverted terminal repeats (ITRs).

In some embodiments, the rAAV virion transduces heart cells. In some embodiments, the rAAV virion transduces cardiomyocytes.

In some embodiments, the rAAV virion traffics to at least one organ other than the liver. In some embodiments, the rAAV virion traffics to the heart.

In some embodiments, the rAAV virion exhibits a higher heart transduction efficiency than an rAAV virion having an AAV9 VP1 capsid protein according to SEQ ID NO: 1.

In some embodiments, the polynucleotide cassette comprises a polynucleotide sequence encoding MYBPC3, DWORF, PKP2, KCNH2, TRPM4, DSG2, TGFBR2, TGFBR1, EMD, KCNQ1, TAZ, COL3A1, JUP, CASQ2, MLRP44, DNAJC19, LMNA, TNNI3, DSP, DSG2, RAF1, SOS1, FBN1, LAMP2, FXN, RAF1, BAG3, KCNQ1, MYLK3, CRYAB, ALPK3, ACTN2, JPH2, PLN, ATP2A2, CACNA1C, DMD, DMPK, EPG5, EVC, EVC2, FBN1, NF1, SCN5A, SOS1, NPR1, ERBB4, VIP, MYH6, MYH7, Cas9, split Cas9, RBM20, MYOCD, ASCL1, GATA4, MEF2C, TBX5, miR-133, or MESP1, or SYNPO2L.

In some embodiments, the polynucleotide cassette comprises a polynucleotide sequence which encodes a protein selected from the group consisting of: MYBPC3, DWORF, PKP2, LMNA, LAMP2, BAG3, CRYAB, JPH2, PLN, TTNI3, MYOCD, ASCL1, DSP, JUP, DSP, MYH6, MYH7, RBM20, Cas9, and split Cas9.

In some aspects, provided herein is a pharmaceutical composition comprising an rAAV virion according to various embodiments disclosed herein and a pharmaceutically acceptable carrier.

In some aspects, provided herein is a polynucleotide encoding a capsid protein according to various embodiments disclosed herein.

In some aspects, provided herein is a method of transducing a cardiac cell, comprising contacting the cardiac cell with an rAAV virion according to various embodiments disclosed herein, wherein the rAAV virion transduces the cardiac cell. In some embodiments, the cardiac cell is a cardiomyocyte. In some embodiments, the rAAV virion exhibits higher transduction efficiency in the cell than an rAAV virion having an AAV9 VP1 capsid protein according to SEQ ID NO: 1.

In some aspects, provided herein is a method of delivering one or more gene products to a cell, wherein the cell is a cardiac cell or a skeletal muscle cell, comprising contacting the cell with a rAAV virion according to various embodiments disclosed herein. In some embodiments, the cell is a cardiomyocyte.

In some aspects, provided herein is a method of treating a cardiac pathology in a subject in need thereof, comprising administering to the subject a therapeutically effective amount of a rAAV virion or a pharmaceutical composition according to various embodiments disclosed herein, wherein the rAAV virion transduces cardiac tissue.

In some aspects, provided herein is a method of treating a heart disease or condition in a subject in need thereof, comprising administering to the subject a therapeutically effective amount of a rAAV virion or a pharmaceutical composition according to various embodiments disclosed herein.

In some aspects, provided herein is a kit comprising a pharmaceutical composition comprising a rAAV virion according to various embodiments disclosed herein and a pharmaceutically acceptable carrier, and instructions for use.

### BRIEF DESCRIPTION OF THE DRAWINGS

FIG. 1 is a schematic of the design of reconstitution sequence variants of the AAV9 VR-VIII region. Reconstitution variants were generated by reconstitution of various combinations of short sequence seeds, some of which contained insertions and/or substitutions, into full sequences at the AAV9 VR-VIII site (581 to 595, based on VP1 numbering).

FIG. 2 shows heart transduction efficiencies (relative to the weighted average of all capsids in the pooled study) of top AAV9 VR-VIII reconstitution variants, measured in C57BL/6 and CD-1 mouse strains.

### DETAILED DESCRIPTION

In some aspects, the disclosure provides engineered capsid proteins. In some embodiments, the engineered capsid proteins comprise any of the substitutions, insertions, non-naturally occurring amino acid motifs, and/or amino acid sequences described herein.

In some aspects, the disclosure provides recombinant adeno-associated virus (rAAV) virions comprising any of the engineered capsid proteins described herein.

The disclosure also provides methods of making and using engineered capsid proteins and rAAV virions.

In some embodiments, provided herein is any engineered AAV capsid protein as disclosed herein with a variant polypeptide sequence relative to parental sequence. In some embodiments, the variant polypeptide sequence comprises any of the insertion and/or substitution motifs described herein.

In some embodiments, the engineered AAV capsid protein provided herein comprises one or more amino acid substitutions relative to a wild-type AAV capsid protein. In some embodiments, the wild-type capsid protein is an AAV9 capsid protein, and the substitutions occur at one, two, three, four, or five of the following positions: S586, A587, Q588, A589, and Q590. In some embodiments, the engineered AAV capsid protein provided herein comprises a polypeptide sequence inserted between positions 588 and 589 relative to a wild-type AAV9 capsid protein. In some embodiments, the engineered AAV capsid protein provided herein comprises a non-naturally occurring amino acid motif. In some embodiments, the engineered AAV capsid protein is a variant of an AAV5, AAV9, AAVrh.74, or AAVrh.10 capsid protein. In some embodiments, the non-naturally occurring amino acid motif comprises an amino acid insertion. In some embodiments, the non-naturally occurring amino acid motif comprises an amino acid substitution. In some embodiments, the non-naturally occurring amino acid motif is in the VR-VIII site. In some embodiments, the recombinant adeno-associated virus (rAAV) virion comprises a vector genome comprising an expression cassette flanked by inverted terminal repeats (ITRs). In some embodiments, the recombinant AAV virion transduces heart cells and/or cardiomyocytes, traffics to at least one organ other than the liver, traffics to the heart, and/or exhibits a higher transduction efficiency than an rAAV virion having a wild-type AAV9 VP1 capsid protein. In some embodiments, the rAAV virion comprises a polynucleotide cassette comprising a polynucleotide sequence, optionally encoding a protein.

In some embodiments, provided herein is a pharmaceutical composition comprising any rAAV virion described herein and a pharmaceutically acceptable carrier or excipient. In some embodiments, provided herein is a polynucleotide encoding any capsid protein described herein.

In some embodiments, provided herein is a method of transducing a cardiac cell or a method of delivering one or more gene products to a cardiac cell, comprising contacting the cardiac cell with any rAAV virion described herein. In some embodiments, provided herein is a method of treating cardiac pathology, or a heart disease or condition, in a subject in need thereof, comprising administering any rAAV virion described herein.

In some embodiments, provided herein is a kit comprising a pharmaceutical composition or an rAAV described herein, and optionally instructions for use.

### Definitions

Unless the context indicates otherwise, the features of the invention can be used in any combination. Any feature or combination of features set forth can be excluded or omitted. Certain features of the invention, which are described in separate embodiments may also be provided in combination in a single embodiment. Features of the invention, which are described in a single embodiment may also be provided separately or in any suitable sub-combination. All combinations of the embodiments are disclosed herein as if each and every combination were individually disclosed. All sub-combinations of the embodiments and elements are disclosed herein as if every such sub-combination were individually disclosed.

Unless defined otherwise, all technical and scientific terms used herein have the same meaning as commonly understood by one of ordinary skill in the art to which this invention belongs. The detailed description is divided into sections only for the reader's convenience and disclosure found in any section may be combined with that in another section. Although any methods and materials similar or equivalent to those described herein can also be used in the practice or testing of the present invention, the exemplary methods and materials are now described. All publications mentioned herein are incorporated by reference to disclose and describe the methods and/or materials in connection with which the publications are cited. Reference to a publication is not an admission that the publication is prior art.

The singular forms “a,” “an,” and “the” include plural referents unless the context clearly dictates otherwise. For example, reference to “a recombinant AAV virion” includes a plurality of such virions and reference to “the cardiac cell” includes one or more cardiac cells.

The conjunction “and/or” means both “and” and “or,” and lists joined by “and/or” encompasses all possible combinations of one or more of the listed items.

The term “vector” refers to a macromolecule or complex of molecules comprising a polynucleotide or protein to be delivered to a cell.

“AAV” is an abbreviation for adeno-associated virus. The term covers all subtypes of AAV, except where a subtype is indicated, and to both naturally occurring and recombinant forms. The abbreviation “rAAV” refers to recombinant adeno-associated virus. “AAV” includes AAV or any subtype. “AAV5” refers to AAV subtype 5. “AAV9” refers to AAV subtype 9. The genomic sequences of various serotypes of AAV, as well as the sequences of the native inverted terminal repeats (ITRs), Rep proteins, and capsid subunits may be found in the literature or in public databases such as GenBank. See, e.g., GenBank Accession Numbers NC_002077 (AAV1), AF063497 (AAV1), NC_001401 (AAV2), AF043303 (AAV2), NC_001729 (AAV3), NC_001829 (AAV4), U89790 (AAV4), NC_006152 (AAV5), AF513851 (AAV7), AF513852 (AAV8), NC_006261 (AAV8), and AY530579 (AAV9). Publications describing AAV include Srivistava et al. (1983) J. Virol. 45:555; Chiorini et al. (1998) J. Virol. 71:6823; Chiorini et al. (1999) J. Virol. 73:1309; Bantel-Schaal et al. (1999) J. Virol. 73:939; Xiao et al. (1999) J. Virol. 73:3994; Muramatsu et al. (1996) Virol. 221:208; Shade et al. (1986) J. Virol. 58:921; Gao et al. (2002) Proc. Nat. Acad. Sci. USA 99: 11854; Moris et al. (2004) Virology 33:375-383; Int'l Pat. Publ Nos. WO2018/222503A1, WO2012/145601A2, WO2000/028061A2, WO1999/61601A2, and WO1998/11244A2; U.S. patent application Ser. Nos. 15/782,980 and 15/433,322; and U.S. Pat. Nos. 10,036,016, 9,790,472, 9,737,618, 9,434,928, 9,233,131, 8,906,675, 7,790,449, 7,906,111, 7,718,424, 7,259,151, 7,198,951, 7,105,345, 6,962,815, 6,984,517, and 6,156,303.

An “AAV vector” or “rAAV vector” as used in the art to refer either to the DNA packaged into in the rAAV virion or to the rAAV virion itself, depending on context. As used herein, unless otherwise apparent from context, rAAV vector refers to a nucleic acid (typically a plasmid) comprising a polynucleotide sequence capable of being packaged into an rAAV virion, but with the capsid or other proteins of the rAAV virion. Generally an rAAV vector comprises a heterologous polynucleotide sequence (i.e., a polynucleotide not of AAV origin) and one or two AAV inverted terminal repeat sequences (ITRs) flanking the heterologous polynucleotide sequence. Only one of the two ITRs may be packaged into the rAAV and yet infectivity of the resulting rAAV virion may be maintained. See Wu et al. (2010) Mol Ther. 18:80. An rAAV vector may be designed to generate either single-stranded (ssAAV) or self-complementary (scAAV). See McCarty D. (2008) Mo. Ther. 16:1648-1656; WO2001/11034; WO2001/92551; WO2010/129021.

An “rAAV virion” refers to an extracellular viral particle including at least one viral capsid protein (e.g. VP1) and an encapsulated rAAV vector (or fragment thereof), including the capsid proteins.

For brevity and clarity, the disclosure refers to “capsid protein” or “capsid proteins.” Those skilled in the art understand that such references refer to VP1, VP2, or VP3, or combinations of VP1, VP2, and VP3. As in wild-type AAV and most recombinant expression systems VP1, VP2, and VP3 are expressed from the same open reading frame, engineering of the sequence that encodes VP3 inevitably alters the sequences of the C-terminal domain of VP1 and VP2. One may also express the capsid proteins from different open reading frames, in which case the capsid of the resulting rAAV virion could contain a mixture of wild-type and engineered capsid proteins, and mixtures of different engineered capsid proteins.

Positions within a sequence alignment are generally denoted in terms of a reference sequence. Unless otherwise specified, amino acid positions in the engineered capsid proteins disclosed herein are numbered according to the VP1 sequence of AAV9 provided as SEQ ID NO: 1. Positions may be determined using a best fit alignment of a sequence of interest to a reference sequence. An insertion “at” a position means inserting sequence between that amino acid position and the preceding position in the alignment. The term “about” allows for substitutions or insertions in positions near to the reference position. Those of skill in the art can used techniques such as structural modeling to determine suitable nearby positions (e.g., by identifying the residues in the loop region exposed on the surface of the capsid).

The term “inverted terminal repeats” or “ITRs” as used herein refers to AAV viral cis-elements named so because of their symmetry. These elements are essential for efficient multiplication of an AAV genome. Without being bound by theory, it is believed that the minimal elements indispensable for ITR function are a Rep-binding site and a terminal resolution site plus a variable palindromic sequence allowing for hairpin formation. The disclosure contemplates that alternative means of generating an AAV genome may exist or may be prospectively developed to be compatible with the capsid proteins of the disclosure.

“Helper virus functions” refers to functions encoded in a helper virus genome which allow AAV replication and packaging.

“Packaging” refers to a series of intracellular events that result in the assembly of an rAAV virion including encapsidation of the rAAV vector. AAV “rep” and “cap” genes refer to polynucleotide sequences encoding replication and encapsidation proteins of adeno-associated virus. AAV rep and cap are referred to herein as AAV “packaging genes.” Packaging requires either a helper virus itself or, more commonly in recombinant systems, helper virus function supplied by a helper-free system (i.e. one or more helper plasmids).

A “helper virus” for AAV refers to a virus that allows AAV (e.g. wild-type AAV) to be replicated and packaged by a mammalian cell. The helper viruses may be an adenovirus, herpesvirus or poxvirus, such as vaccinia.

An “infectious” virion or viral particle is one that comprises a competently assembled viral capsid and is capable of delivering a polynucleotide component into a cell for which the virion is tropic. The term does not necessarily imply any replication capacity of the virus.

“Infectivity” refers to a measurement of the ability of a virion to inflect a cell. Infectivity can be expressed as the ratio of infectious viral particles to total viral particles. Infectivity is general determined with respect to a particular cell type. It can be measured both in vivo or in vitro. Methods of determining the ratio of infectious viral particle to total viral particle are known in the art. See, e.g., Grainger et al. (2005) Mol. Ther. 11:S337 (describing a TCID₅₀ infectious titer assay); and Zolotukhin et al. (1999) Gene Ther. 6:973.

The terms “parental capsid” or “parental sequence” refer to a reference sequence from which a capsid or sequence is derived. Unless otherwise specified, parental sequence refers to the sequence of the wild-type capsid protein of the same serotype as the engineered capsid protein.

A “replication-competent” virus (e.g. a replication-competent AAV) refers to a virus that is infectious, and is also capable of being replicated in an infected cell (i.e. in the presence of a helper virus or helper virus functions). In some embodiments, the rAAV virion of the disclosure comprises a genome that lacks the rep gene, or both the rep and cap genes, and therefore is replication incompetent.

The practice of the present disclosure will employ, unless otherwise indicated, conventional techniques of tissue culture, immunology, molecular biology, cell biology and recombinant DNA, which are within the skill of the art. See, e.g., Sambrook and Russell eds. (2001) Molecular Cloning: A Laboratory Manual, 3^{rd} edition; Ausubel et al. eds. (2007) Current Protocols in Molecular Biology; Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; IRL Press (1986) Immobilized Cells and Enzymes; Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3^{rd} edition (2002) Cold Spring Harbor Laboratory Press; Sohail (2004) Gene Silencing by RNA Interference: Technology and Application (CRC Press); and Sell (2013) Stem Cells Handbook.

The term “isolated” means separated from constituents, cellular and otherwise, in which the virion, cell, tissue, polynucleotide, peptide, polypeptide, or protein is normally associated in nature. For example, an isolated cell is a cell that is separated form tissue or cells of dissimilar phenotype or genotype.

As used herein, “sequence identity” or “identity” refers to the percentage of number of amino acids that are identical between a sequence of interest and a reference sequence. Generally identity is determined by aligning the sequence of interest to the reference sequence, determining the number of amino acids that are identical between the aligned sequences, dividing that number by the total number of amino acids in the reference sequence, and multiplying the result by 100 to yield a percentage. Sequences can be aligned using various computer programs, such BLAST, available at ncbi.nlm.nih.gov. Other techniques for alignment are described in Methods in Enzymology, vol. 266: Computer Methods for Macromolecular Sequence Analysis (1996); and Meth. Mol. Biol. 70: 173-187 (1997); J. Mol. Biol. 48: 44. Skill artisans are capable of choosing an appropriate alignment method depending on various factors including sequence length, divergence, and the presence of absence of insertions or deletions with respect to the reference sequence.

“Recombinant,” as applied to a polynucleotide means that the polynucleotide is the product of various combinations of cloning, restriction or ligation steps, and other procedures that result in a construct that is distinct from a polynucleotide found in nature, or that the polynucleotide is assembled from synthetic oligonucleotides. A “recombinant” protein is a protein produced from a recombinant polypeptide. A recombinant virion is a virion that comprises a recombinant polynucleotide and/or a recombinant protein, e.g. a recombinant capsid protein.

“Engineered,” as used herein, refers to a sequence that results from deliberate modification of residues within the parental sequence. For example, an engineered polypeptide comprises one or more amino acids that are not found at the corresponding location in the parental polypeptide. An engineered sequence may comprise one or more inserted residues, one or more deleted residues, and/or one or more substituted residues.

A “gene” refers to a polynucleotide containing at least one open reading frame that is capable of encoding a particular protein after being transcribed and translated. A “gene product” is a molecule resulting from expression of a particular gene. Gene products may include, without limitation, a polypeptide, a protein, an aptamer, an interfering RNA, or an mRNA. Gene-editing systems (e.g. a CRISPR/Cas system) may be described as one gene product or as the several gene products required to make the system (e.g. a Cas protein and a guide RNA).

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements include transcriptional regulatory sequences such as promoters and/or enhancers.

A “promoter” is a DNA sequence capable under certain conditions of binding RNA polymerase and initiating transcription of a coding region usually located downstream (in the 3′ direction) from the promoter. The term “tissue-specific promoter” as used herein refers to a promoter that is operable in cells of a particular organ or tissue, such as the cardiac tissue.

“Operatively linked” or “operably linked” refers to a juxtaposition of genetic elements, wherein the elements are in a relationship permitting them to operate in the expected manner. For instance, a promoter is operatively linked to a coding region if the promoter helps initiate transcription of the coding sequence. There may be intervening residues between the promoter and coding region so long as this functional relationship is maintained.

The term “polynucleotide cassette” refers to the portion of a vector genome between the inverted terminal repeats (ITRs). A polynucleotide cassette may comprises polynucleotide sequences encoding any genetic element whose delivery to a target cell is desired, including but not limited to a coding sequence for a gene, a promoter, or a repair template for gene editing. Unless otherwise specified, the expression cassette of an AAV vector includes only the polynucleotide between (and not including) the ITRs.

An “expression vector” is a vector comprising a coding sequence which encodes a gene product of interest used to effect the expression of the gene product in target cells. An expression vector comprises control elements operatively linked to the coding sequence to facilitate expression of the gene product.

The term “expression cassette” refers to a polynucleotide cassette comprising a coding sequence which encodes a gene product of interest used to effect the expression of the gene product in target cells. Unless otherwise specified, the expression cassette of an AAV vector includes only the polynucleotides between (and not including) the ITRs.

The term “gene delivery” or “gene transfer” as used herein refers to methods or systems for reliably inserting foreign nucleic acid sequences, e.g., DNA, into host cells. Such methods can result in transient expression of non-integrated transferred DNA, extra-chromosomal replication and expression of transferred replicons (e.g., episomes), or integration of transferred genetic material into the genomic DNA of host cells.

“Heterologous” means derived from a genotypically distinct entity from that of the rest of the entity to which it is being compared. For example, a polynucleotide introduced by genetic engineering techniques into a plasmid or vector derived from a different species is a heterologous polynucleotide. A promoter removed from its native coding sequence and operatively linked to a coding sequence with which it is not naturally found linked is a heterologous promoter. Thus, for example, an rAAV that includes a heterologous nucleic acid is an rAAV that includes a nucleic acid not normally included in a naturally-occurring AAV.

The terms “genetic alteration” and “genetic modification” (and grammatical variants thereof), are used interchangeably herein to refer to a process wherein a genetic element (e.g., a polynucleotide) is introduced into a cell other than by mitosis or meiosis. The element may be heterologous to the cell, or it may be an additional copy or improved version of an element already present in the cell. Genetic alteration may be effected, for example, by transfecting a cell with a recombinant plasmid or other polynucleotide through any process known in the art, such as electroporation, calcium phosphate precipitation, or contacting with a polynucleotide-liposome complex. Genetic alteration may also be effected, for example, by transduction or infection with a vector.

A cell is said to be “stably” altered, transduced, genetically modified, or transformed with a polynucleotide sequence if the sequence is available to perform its function during extended culture of the cell in vitro. Generally, such a cell is “heritably” altered (genetically modified) in that a genetic alteration is introduced which is also inheritable by progeny of the altered cell.

The term “transfection” is as used herein refers to the uptake of an exogenous nucleic acid molecule by a cell. A cell has been “transfected” when exogenous nucleic acid has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acid molecules into suitable host cells.

The term “transduction” is as used herein refers to the transfer of an exogenous nucleic acid into a cell by a recombinant virion, in contrast to “infection” by a wild-type virion. When infection is used with respect to a recombinant virion, the terms “transduction” and “infectious” are synonymous, and therefore “infectivity” and “transduction efficiency” are equivalent and can be determined using similar methods.

The phrase “assessed in a primate” refers to testing by methods described in the Examples or variations upon them. Assessment may be done using a population of rAAV virions having a common capsid protein screen or pooled testing by re-screening.

Unless otherwise specified, all medical terminology is given the ordinary meaning of the term used by medical professional as, for example, in Harrison's Principles of Internal Medicine, 15ed., which is incorporated by reference in its entirety for all purposes, in particular the chapters on cardiac or cardiovascular diseases, disorders, conditions, and dysfunctions.

“Treatment,” “treating,” and “treat” are defined as acting upon a disease, disorder, or condition with an agent to reduce or ameliorate harmful or any other undesired effects of the disease, disorder, or condition and/or its symptoms.

“Administration,” “administering” and the like, when used in connection with a composition of the invention refer both to direct administration (administration to a subject by a medical professional or by self-administration by the subject) and/or to indirect administration (prescribing a composition to a patient). Typically, an effective amount is administered, which amount can be determined by one of skill in the art. Any method of administration may be used. Administration to a subject can be achieved by, for example, intravenous, intra-arterial, intramuscular, intravascular, or intramyocardial delivery.

As used herein the term “effective amount” and the like in reference to an amount of a composition refers to an amount that is sufficient to induce a desired physiologic outcome (e.g., reprogramming of a cell or treatment of a disease). An effective amount can be administered in one or more administrations, applications or dosages. Such delivery is dependent on a number of variables including the time period which the individual dosage unit is to be used, the bioavailability of the composition, the route of administration, etc. It is understood, however, that specific amounts of the compositions (e.g., rAAV virions) for any particular subject depends upon a variety of factors including the activity of the specific agent employed, the age, body weight, general health, sex, and diet of the subject, the time of administration, the rate of excretion, the composition combination, severity of the particular disease being treated and form of administration.

The terms “individual,” “subject,” and “patient” are used interchangeably herein, and refer to a mammal, including, but not limited to, human and non-human primates (e.g., simians); mammalian sport animals (e.g., horses); mammalian farm animals (e.g., sheep, goats, etc.); mammalian pets (e.g., dogs, cats, etc.); and rodents (e.g., mice, rats, etc.).

The terms “cardiac pathology” or “cardiac dysfunction” are used interchangeably and refer to any impairment in the heart's pumping function. This includes, for example, impairments in contractility, impairments in ability to relax (sometimes referred to as diastolic dysfunction), abnormal or improper functioning of the heart's valves, diseases of the heart muscle (sometimes referred to as cardiomyopathies), diseases such as angina pectoris, myocardial ischemia and/or infarction characterized by inadequate blood supply to the heart muscle, infiltrative diseases such as amyloidosis and hemochromatosis, global or regional hypertrophy (such as may occur in some kinds of cardiomyopathy or systemic hypertension), and abnormal communications between chambers of the heart.

As used herein, the term “cardiomyopathy” refers to any disease or dysfunction that affects myocardium directly. The etiology of the disease or disorder may be, for example, inflammatory, metabolic, toxic, infiltrative, fibroplastic, hematological, genetic, or unknown in origin. Two fundamental forms are recognized (1) a primary type, consisting of heart muscle disease of unknown cause; and (2) a secondary type, consisting of myocardial disease of known cause or associated with a disease involving other organ systems. “Specific cardiomyopathy” refers to heart diseases associated with certain systemic or cardiac disorders; examples include hypertensive and metabolic cardiomyopathy. The cardiomyopathies include dilated cardiomyopathy (DCM), a disorder in which left and/or right ventricular systolic pump function is impaired, leading to progressive cardiac enlargement; hypertrophic cardiomyopathy, characterized by left ventricular hypertrophy without obvious causes such as hypertension or aortic stenosis; and restrictive cardiomyopathy, characterized by abnormal diastolic function and excessively rigid ventricular walls that impede ventricular filling. Cardiomyopathies also include left ventricular non-compaction, arrhythmogenic right ventricular cardiomyopathy, and arrhythmogenic right ventricular dysplasia.

“Heart failure” refers to the pathological state in which an abnormality of cardiac function is responsible for failure of the heart to pump blood at a rate commensurate with the requirements of the metabolizing tissues and/or allows the heart to do so only from an abnormally elevated diastolic volume. Heart failure includes systolic and diastolic failure. Patient with heart failure are classified into those with low cardiac output (typically secondary to ischemic heart disease, hypertension, dilated cardiomyopathy, and/or valvular or pericardial disease) and those with elevated cardiac output (typically due to hyperthyroidism, anemia, pregnancy, arteriovenous fistulas, beriberi, and Paget's disease). Heart failure includes heart failure with reduced ejection fraction (HFrEF) and heart failure with preserved ejection fraction (HFpEF).

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “purified” as used herein refers to material that has been isolated under conditions that reduce or eliminate the presence of unrelated materials, i.e. impurities, including native materials from which the material is obtained. For example, purified rAAV vector DNA is preferably substantially free of cell or culture components, including tissue culture components, contaminants, and the like.

The terms “regenerate,” “regeneration” and the like as used herein in the context of injured cardiac tissue shall be given their ordinary meanings and shall also refer to the process of growing and/or developing new cardiac tissue in a heart or cardiac tissue that has been injured, for example, injured due to ischemia, infarction, reperfusion, or other disease. In some embodiments, cardiac tissue regeneration comprises generation of cardiomyocytes.

The term “therapeutic gene” as used herein refers to a gene that, when expressed, confers a beneficial effect on the cell or tissue in which it is present, or on a mammal in which the gene is expressed. Examples of beneficial effects include amelioration of a sign or symptom of a condition or disease, prevention or inhibition of a condition or disease, or conferral of a desired characteristic. Therapeutic genes include genes that partially or wholly correct a genetic deficiency in a cell or mammal.

As used herein, the term “functional cardiomyocyte” refers to a differentiated cardiomyocyte that is able to send or receive electrical signals. In some embodiments, a cardiomyocyte is said to be a functional cardiomyocyte if it exhibits electrophysiological properties such as action potentials and/or Ca²⁺ transients.

As used herein, a “differentiated non-cardiac cell” can refer to a cell that is not able to differentiate into all cell types of an adult organism (i.e., is not a pluripotent cell), and which is of a cellular lineage other than a cardiac lineage (e.g., a neuronal lineage or a connective tissue lineage). Differentiated cells include, but are not limited to, multipotent cells, oligopotent cells, unipotent cells, progenitor cells, and terminally differentiated cells. In particular embodiments, a less potent cell is considered “differentiated” in reference to a more potent cell.

A “somatic cell” is a cell forming the body of an organism. Somatic cells include cells making up organs, skin, blood, bones and connective tissue in an organism, but not germ cells.

As used herein, the term “totipotent” means the ability of a cell to form all cell lineages of an organism. For example, in mammals, only the zygote and the first cleavage stage blastomeres are totipotent.

As used herein, the term “pluripotent” means the ability of a cell to form all lineages of the body or soma. For example, embryonic stem cells are a type of pluripotent stem cells that are able to form cells from each of the three germs layers, the ectoderm, the mesoderm, and the endoderm. Pluripotent cells can be recognized by their expression of markers such as Nanog and Rex1.

As used herein, the term “multipotent” refers to the ability of an adult stem cell to form multiple cell types of one lineage. For example, hematopoietic stem cells are capable of forming all cells of the blood cell lineage, e.g., lymphoid and myeloid cells.

As used herein, the term “oligopotent” refers to the ability of an adult stem cell to differentiate into only a few different cell types. For example, lymphoid or myeloid stem cells are capable of forming cells of either the lymphoid or myeloid lineages, respectively.

As used herein, the term “unipotent” means the ability of a cell to form a single cell type. For example, spermatogonial stem cells are only capable of forming sperm cells.

As used herein, the term “reprogramming” or “transdifferentiation” refers to the generation of a cell of a certain lineage (e.g., a cardiac cell) from a different type of cell (e.g., a fibroblast cell) without an intermediate process of de-differentiating the cell into a cell exhibiting pluripotent stem cell characteristics.

As used herein the term “cardiac cell” refers to any cell present in the heart that provides a cardiac function, such as heart contraction or blood supply, or otherwise serves to maintain the structure of the heart. Cardiac cells as used herein encompass cells that exist in the epicardium, myocardium or endocardium of the heart. Cardiac cells also include, for example, cardiac muscle cells or cardiomyocytes, and cells of the cardiac vasculatures, such as cells of a coronary artery or vein. Other non-limiting examples of cardiac cells include epithelial cells, endothelial cells, fibroblasts, cardiac stem or progenitor cells, cardiac conducting cells and cardiac pacemaking cells that constitute the cardiac muscle, blood vessels and cardiac cell supporting structure. Cardiac cells may be derived from stem cells, including, for example, embryonic stem cells or induced pluripotent stem cells.

The term “cardiomyocyte” or “cardiomyocytes” as used herein refers to sarcomere-containing striated muscle cells, naturally found in the mammalian heart, as opposed to skeletal muscle cells. Cardiomyocytes are characterized by the expression of specialized molecules, e.g., proteins like myosin heavy chain, myosin light chain, cardiac α-actinin. The term “cardiomyocyte” as used herein is an umbrella term comprising any cardiomyocyte subpopulation or cardiomyocyte subtype, e.g., atrial, ventricular and pacemaker cardiomyocytes.

The term “cardiomyocyte-like cells” is intended to mean cells sharing features with cardiomyocytes, but which may not share all features. For example, a cardiomyocyte-like cell may differ from a cardiomyocyte in expression of certain cardiac genes.

The term “culture” or “cell culture” means the maintenance of cells in an artificial, in vitro environment. A “cell culture system” is used herein to refer to culture conditions in which a population of cells may be grown as monolayers or in suspension. “Culture medium” is used herein to refer to a nutrient solution for the culturing, growth, or proliferation of cells. Culture medium may be characterized by functional properties such as, but not limited to, the ability to maintain cells in a particular state (e.g., a pluripotent state, a quiescent state, etc.), to mature cells—in some instances, specifically, to promote the differentiation of progenitor cells into cells of a particular lineage (e.g., a cardiomyocyte).

As used herein, the term “expression” or “express” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample.

The term “induced cardiomyocyte” or the abbreviation “iCM” refers to a non-cardiomyocyte (and its progeny) that has been transformed into a cardiomyocyte (and/or cardiomyocyte-like cell). The methods of the present disclosure can be used in conjunction with any methods now known or later discovered for generating induced cardiomyocytes, for example, to enhance other techniques.

The term “induced pluripotent stem cell-derived cardiomyocytes” as used herein refers to human induced pluripotent stem cells that have been differentiated into cardiomyocyte-like cells. Exemplary methods for prepared iPS-CM cells are provided by Karakikes et al. Circ Res. 2015 Jun. 19; 117(1): 80-88.

The terms “human cardiac fibroblast” and “mouse cardiac fibroblast” as used herein refer to primary cell isolated from the ventricles of the adult heart of a human or mouse, respectively, and maintain in culture ex vivo.

The term “non-cardiomyocyte” as used herein refers to any cell or population of cells in a cell preparation not fulfilling the criteria of a “cardiomyocyte” as defined and used herein. Non-limiting examples of non-cardiomyocytes include somatic cells, cardiac fibroblasts, non-cardiac fibroblasts, cardiac progenitor cells, and stem cells.

As used herein “reprogramming” includes transdifferentiation, dedifferentiation and the like.

As used herein, the term “reprogramming efficiency” refers to the number of cells in a sample that are successfully reprogrammed to cardiomyocytes relative to the total number of cells in the sample.

The term “reprogramming factor” as used herein includes a factor that is introduced for expression in a cell to assist in the reprogramming of the cell from one cell type into another. For example, a reprogramming factor may include a transcription factor that, in combination with other transcription factors and/or small molecules, is capable of reprogramming a cardiac fibroblast into an induced cardiomyocyte. Unless otherwise clear from context, a reprogramming factor refers to a polypeptide that can be encoded by an AAV-delivered polynucleotide. Reprogramming factors may also include small molecules.

As used herein, the term “equivalents thereof” in reference to a polypeptide or nucleic acid sequence refers to a polypeptide or nucleic acid that differs from a reference polypeptide or nucleic acid sequence, but retains essential properties (e.g., biological activity). A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, deletions, additions, fusions and truncations in the polypeptide encoded by the reference sequence. Generally, differences are limited so that the sequences of the reference polypeptide and the variant are closely similar overall and, in many regions, identical.

As used herein, the term “progenitor cell” refers to a cell that is committed to differentiate into a specific type of cell or to form a specific type of tissue. A progenitor cell, like a stem cell, can further differentiate into one or more kinds of cells, but is more mature than a stem cell such that it has a more limited/restricted differentiation capacity.

The term “genetic modification” refers to a permanent or transient genetic change induced in a cell following introduction of new nucleic acid (i.e., nucleic acid exogenous to the cell). Genetic change can be accomplished by incorporation of the new nucleic acid into the genome of the cardiac cell, or by transient or stable maintenance of the new nucleic acid as an extrachromosomal element. Where the cell is a eukaryotic cell, a permanent genetic change can be achieved by introduction of the nucleic acid into the genome of the cell. Suitable methods of genetic modification include viral infection, transfection, conjugation, protoplast fusion, electroporation, particle gun technology, calcium phosphate precipitation, direct microinjection, and the like.

The term “stem cells” refer to cells that have the capacity to self-renew and to generate differentiated progeny. The term “pluripotent stem cells” refers to stem cells that can give rise to cells of all three germ layers (endoderm, mesoderm and ectoderm), but do not have the capacity to give rise to a complete organism. In some embodiments, the compositions for inducing cardiomyocyte phenotype can be used on a population of cells to induce reprogramming. In other embodiments, the compositions induce a cardiomyocyte phenotype.

The term “induced pluripotent stem cells” shall be given its ordinary meaning and shall also refer to differentiated mammalian somatic cells (e.g., adult somatic cells, such as skin) that have been reprogrammed to exhibit at least one characteristic of pluripotency. See, for example, Takahashi et al. (2007) Cell 131(5):861-872, Kim et al. (2011) Proc. Natl. Acad. Sci. 108(19): 7838-7843, Sell (2013) Stem Cells Handbook.

The term “transduction efficiency” refers to the percentage of cells transduced with at least one AAV genome. For example, if 1×10⁶ cells are exposed to a virus and 0.5×10⁶ cells are determined to contain at least one copy of the AAV genome, then the transduction efficiency is 50%. An illustrative method for determining transduction efficiency is flow cytometry. For example, the percentage of GFP+ cells is a measure of transduction efficiency when the AAV genome comprises a polynucleotide encoding green fluorescence protein (GFP).

The term “selectivity” refers to the ratio of transduction efficiency for one cell type over another, or over all other cells types.

The term “infectivity” refers to the ability of an AAV virion to infect a cell, in particularly an in vivo cell. Infectivity therefore is a function of, at least, biodistribution and neutralizing antibody escape.

Unless stated otherwise, the abbreviations used throughout the specification have the following meanings: AAV, adeno-associated virus, rAAV, recombinant adeno-associated virus; AHCF, adult human cardiac fibroblast; APCF, adult pig cardiac fibroblast, a-MHC-GFP; alpha-myosin heavy chain green fluorescence protein; CF, cardiac fibroblast; cm, centimeter; CO, cardiac output; EF, ejection fraction; FACS, fluorescence activated cell sorting; GFP, green fluorescence protein; GMT, Gata4, Mef2c and Tbx5; GMTc, Gata4, Mef2c, Tbx5, TGF-βi, WNTi; GO, gene ontology; hCF, human cardiac fibroblast; iCM, induced cardiomyocyte; kg, killigram; μg, microgram; μl, microliter; mg, milligram; ml, milliliter; MI, myocardial infarction; msec, millisecond; min, minute; MyAMT, Myocardin, Ascl1, Mef2c and Tbx5; MyA, Myocardin and Ascl1; MyMT, Myocardin, Mef2c and Tbx5; MyMTc, Myocardin, Mef2c, Tbx5, TGF-βi, WNTi; MRI, magnetic resonance imaging; PBS, phosphate buffered saline; PBST, phosphate buffered saline, triton; PFA, paraformaldehyde; qPCR, quantitative polymerase chain reaction; qRT-PCR, quantitative reverse transcriptase polymerase chain reaction; RNA, ribonucleic acid; RNA-seq, RNA sequencing; RT-PCR, reverse transcriptase polymerase chain reaction; sec, second; SV, stroke volume; TGF-β, transforming growth factor beta; TGF-βi, transforming growth factor beta inhibitor; WNT, wingless-Int; WNTi, wingless-Int inhibitor; YFP, yellow fluorescence protein; 4F, Gata4, Mef2c, TBX5, and Myocardin; 4Fc, Gata4, Mef2c, TBX5, and Myocardin+TGF-βi and WNTi; 7F, Gata4, Mef2c, and Tbx5, Essrg, Myocardin, Zfpm2, and Mesp1; 7Fc, Gata4, Mef2c, and Tbx5, Essrg, Myocardin, Zfpm2, and Mesp1+TGF-β and WNTi.

The amino acid abbreviations used herein are abbreviations commonly known and used in the art, and as follows:
Alanine—Ala—A
Arginine—Arg—R
Asparagine—Asn—N
Aspartic acid—Asp—D
Cysteine—Cys—C
Glutamic acid—Glu—E
Glutamine—Gln—Q
Glycine—Gly—G
Histidine—His—H
Isoleucine—Ile—I
Leucine—Leu—L
Lysine—Lys—K
Methionine—Met—M
Phenylalanine—Phe—F
Proline—Pro—P
Serine—Ser—S
Threonine—Thr—T
Tryptophan—Trp—W
Tyrosine—Tyr—Y
Valine—Val—V

Reference to amino acid substitutions are in the format commonly used in the art. E.g., reference to “N452K” substitution, indicates that at position number 452 of the reference sequence, the wild-type amino acid in front of the number (here “N”) has been substituted with the amino acid following the number (here “K”).

The term “conservative amino-acid substitutions” refers to substitutions of amino acid residues that share similar sidechain physical properties with the residues being substituted. Conservative substitutions include polar for polar residues, non-polar for non-polar residues, hydrophobic for hydrophobic residues, small for small residues, and large for large residues. Conservative substitutions further comprise substitutions within the following groups: {S, T}, {A, G}, {F, Y}, {R, H, K, N, E}, {S, T, N, Q}, {C, U, G, P, A}, and {A, V, I, L, M, F, Y, W}.

### Engineered Capsid Proteins

In some aspects, provided are engineered capsid proteins, e.g., AAV capsid proteins, comprising a non-naturally occurring amino acid motif relative to a corresponding wild-type or parental sequence. Capsid proteins are structural proteins that make up the assembled icosahedral packaging of an AAV virion and largely determine the immunogenicity and tropism of the virus. In some embodiments, the engineered capsid protein comprises one or more amino acid substitutions relative to a corresponding wild-type or parental sequence (also referred to as “substitution motifs”). In some embodiments, the engineered capsid protein comprises an insertion peptide sequence (also referred to as “insertion motifs” in the present technology) relative to a corresponding wild-type or parental sequence. In some embodiments, the engineered capsid protein comprises one or more amino acid substitutions relative to a corresponding wild-type or parental capsid sequence and an insertion of one or more peptide sequences. In some embodiments, the reference wild-type or parental capsid protein is of any serotype known in the field or described herein, including, for example, serotypes 1, 2, 3, 4, 5, 6, 7, 8, 9, 10, 11, 12, rh.10, rh.20, rh.74, and any chimeric or mosaic variant derived therefrom. In some embodiments, provided is an engineered AAV9 capsid protein. In some embodiments, provided is an engineered AAV5 capsid protein. In some embodiments, provided is an engineered AAVrh.10 capsid protein. In some embodiments, provided is an engineered AAVrh.74 capsid protein. In some embodiments, provided herein is an engineered AAV5/9 chimeric capsid protein.

### A. Non-Naturally Occurring Amino Acid Motifs

In some embodiments, the engineered capsid proteins described herein comprise one or more non-naturally occurring amino acid motifs relative to a wild-type or parental capsid protein sequence. The non-naturally occurring amino acid motifs described herein comprise (i) one or more substitution motifs; (ii) one or more insertion motifs; or (iii) one or more a substitution motifs and one or more insertion motifs. The wild-type or parental capsid protein can be any wild-type, chimeric, or mosaic capsid protein as described herein or as known in the art, or a variant thereof.

In some embodiments, the non-naturally occurring amino acid motifs described herein comprise insertion of one, two, three, four, five, six, seven, or more amino acids, substitution of one, two, three, four, five, or more amino acids, or any combination of insertion and substitution of amino acids in the wild-type or parental AAV capsid protein. Any non-naturally occurring amino acid motif and any combination of non-naturally occurring amino acid motifs can be made in an engineered AAV capsid protein in accordance with the disclosure.

### Substitution Motifs

In some embodiments, the engineered capsid proteins described herein comprise a non-naturally occurring amino acid motif comprising one or more amino acid substitutions relative to a wild-type or parental capsid protein described herein (referred to herein as a “substitution motif”). In some embodiments, the wild-type or parental capsid protein is an AAV9, AAV5, AAVrh.10, or AAVrh.74 capsid. In some embodiments, the substitution motif does not comprise amino acid insertions.

In some embodiments, the engineered capsid protein comprises a substitution motif comprising an amino acid substitution at one, two, three, four, five, six, seven, eight, nine, or ten positions relative to a wild-type AAV capsid protein sequence. In some embodiments, the engineered capsid protein comprises a substitution motif comprising an amino acid substitution at two positions relative to a wild-type AAV capsid protein sequence. In some embodiments, the engineered capsid protein comprises a substitution motif comprising an amino acid substitution at three positions relative to a wild-type AAV capsid protein sequence. In some embodiments, the engineered capsid protein comprises a substitution motif comprising an amino acid substitution at four positions relative to a wild-type AAV capsid protein sequence. In some embodiments, the engineered capsid protein comprises a substitution motif comprising an amino acid substitution at five positions relative to a wild-type AAV capsid protein sequence. In some embodiments, the engineered capsid protein comprises a substitution motif comprising an amino acid substitution at one, two, three, four, or five of the following positions relative to a wild-type AAV9 capsid protein sequence: S586, A587, Q588, A589, and Q590. In some embodiments, the amino acid numbering is according to the AAV9 VP1 sequence of SEQ ID NO:1.

TABLE 1
Exemplary Substitution Motifs.

| AAV Capsid | Amino Acid Position | | | | |
|---|---|---|---|---|---|
| AAV9 VP1 (SEQ ID NO: 1) | 586 | 587 | 588 | 589 | 590 |
| AAV5 VP1 (SEQ ID NO: 10) | 575 | 576 | 577 | 578 | 579 |
| AAVrh.10 VP1 (SEQ ID NO: 19) | 588 | 589 | 590 | 591 | 592 |
| AAVrh.74 VP1 (SEQ ID NO: 28) | 588 | 589 | 590 | 591 | 592 |
| lib73_variant-1533; 1532; | E | N | R | A | Q |
| 1596; 1595; 1658; 1615; 1553; | | | | | |
| 1637; 1554 | | | | | |
| lib73_variant-357; 419; 356; | S | S | V | A | Q |
| 502; 420; 483; 482; 439; 378; | | | | | |
| 377; 462; 461; 398; 608 | | | | | |
| lib73_variant-1701; 1700; 1764; | A | S | T | A | Q |
| 1846; 1763; 1827; 1783; 1722; | | | | | |
| 1721; 1806; 1805; 1743; 1682; | | | | | |
| 1688; 1703; 1793; 1730 | | | | | |
| ZC739 and lib73_variant-1681; | A | S | T | A | G |
| 1691; 1744; 1754; 1702; 1712; | | | | | |
| 1786; 1733 | | | | | |
| ZC738 | E | N | R | N | R |
| lib73_variant-1769 | A | S | T | S | G |
| ZC736 | E | N | R | T | Q |
| lib73_variant-1602 | E | N | R | T | L |
| lib73_variant-1770 | A | S | T | T | L |
| lib73_variant-1780 | A | S | T | T | Q |

In some embodiments, the substitution motif comprises amino acid substitutions selected from S586E, S586A, A587S, A587N, Q588V, Q588R, Q588T, A589T, A589N, A589S, Q590G, Q590L, and Q590R. In some embodiments, the amino acid substitutions are selected from S586E, S586A, A587N, Q588R, and Q588T.

In some embodiments, the substitution motif comprises amino acid substitutions selected from: a) S586E, A587N, Q588R, and A589T; b) S586A, A587S, Q588T, and Q590G; c) S586E, A587N, Q588R, A589T, and Q590L; d) S586A, A587S, Q588T, A589T, and Q590L; e) S586E, A587N, Q588R, A589N, Q590R; f) S586A, A587S, Q588T, and A589T; g) S586A, A587S, Q588T, A589S, and Q590G; h) S586E, A587N, and Q588R; and i) S586A, A587S, and Q588T.

In some embodiments, the substitution motif comprises amino acid substitutions selected from: a) A587S and Q588V; b) S586E, A587N, Q588R, and A589T; c) S586A, A587S, Q588T, and Q590G; d) S586E, A587N, Q588R, A589T, and Q590L; e) S586E, A587N, and Q588R; f) S586A, A587S, and Q588T; g) S586A, A587S, Q588T, A589T, and Q590L; h) S586E, A587N, Q588R, A589N, Q590R; i) S586A, A587S, Q588T, and A589T; and j) S586A, A587S, Q588T, A589S, and Q590G.

The engineered capsid proteins may comprise any of the substitution motifs described herein. Exemplary substitution motifs are shown above in Table 1 above, in which in bold, underlined text indicates amino acids that differ from those of the wild-type capsid protein sequence at the specified position. In some embodiments, the non-naturally occurring amino acid motif comprises a substitution motif and does not comprise an insertion motif.

### Insertion Motifs

In some embodiments, the engineered capsid proteins described herein comprise a non-naturally occurring amino acid motif comprising a polypeptide sequence inserted between two amino acids in the wild-type or parental AAV capsid protein (referred to herein as an “insertion motif”). In some embodiments, the wild-type or parental capsid protein is an AAV9, AAV5, AAVrh.10, or AAVrh.74 capsid. In some embodiments, the insertion motif does not comprise amino acid substitutions. In some embodiments, the insertion motif is in the VR-VIII site.

In some embodiments, the insertion motif is about 1 to 20 amino acids in length, for example, about 1 to 15 amino acids, about 1 to 10 amino acids, about 1 to 9 amino acids, about 1 to 8 amino acids, about 1 to 7 amino acids, about 1 to 6 amino acids, about 1 to 5 amino acids, about 5 to 15 amino acids, about 5 to 10 amino acids, about 5 to 9 amino acids, about 5 to 8 amino acids, or about 6 to 8 amino acids in length. In some embodiments, the insertion motif is about 1 amino acid, about 2 amino acids, about 3 amino acids, about 4 amino acids, about 5 amino acids, about 6 amino acids, about 7 amino acids, about 8 amino acids, about 9 amino acids, about 10 amino acids, about 11 amino acids, about 12 amino acids, about 13 amino acids, about 14 amino acids, about 15 amino acids, about 16 amino acids, about 17 amino acids, about 18 amino acids, about 19 amino acids, or about 20 amino acids in length.

In some embodiments, the engineered capsid protein comprises an insertion motif between positions 588 and 589 relative to a wild-type AAV9 capsid protein, wherein the amino acid positions are numbered according to SEQ ID NO: 1. In some embodiments, the insertion motif comprises an amino acid sequence RX₁DX₂X₃X₄X₅, wherein: X₁ is Glycine (G) or Threonine (T); X₂ is Histidine (H), Serine (S), Alanine (A), Leucine (L), Threonine (T), Glycine (G), Valine (V), or Phenylalanine (F); X₃ is Glycine (G), Alanine (A), Valine (V), Lysine (K), Asparagine (N), or Threonine (T); X₄ is Valine (V), Serine (S), Glycine (G), Asparagine (N), or Arginine (R); and X₅ is Leucine (L), Tryptophan (W), Threonine (T), Glycine (G), or Arginine (R). In some embodiments, the insertion motif is selected from any one of SEQ ID NOs: 215-242 provided in Table 2 below. In some embodiments, the insertion motif is selected from any one of SEQ ID NOs: 215-227 provided in Table 2 below.

TABLE 2
Exemplary insertion motifs

| Insertion | SEQ ID NO: |
|---|---|
| RGDAASW | 215 |
| RGDAGVL | 216 |
| RGDGASW | 217 |
| RGDGGVL | 218 |
| RGDLNNT | 219 |
| RGDSASW | 220 |
| RGDSGVL | 221 |
| RGDTASW | 222 |
| RGDTGVL | 223 |
| RGDVASW | 224 |
| RGDVGVL | 225 |
| RTDLGVL | 226 |
| RTDVGVL | 227 |
| RGDAARL | 228 |
| RGDAKGL | 229 |
| RGDGARL | 230 |
| RGDGKGL | 231 |
| RGDLGSG | 232 |
| RGDLTGR | 233 |
| RGDLVST | 234 |
| RGDSARL | 235 |
| RGDSKGL | 236 |
| RGDTARL | 237 |
| RGDTKGL | 238 |
| RGDVKGL | 239 |
| RGDFNNT | 240 |
| RGDHASW | 241 |
| RGDHGVL | 242 |

In some embodiments, the insertion motif comprises or consists of SEQ ID NO: 215. In some embodiments, the insertion motif comprises or consists of SEQ ID NO: 216. In some embodiments, the insertion motif comprises or consists of SEQ ID NO: 217. In some embodiments, the insertion motif comprises or consists of SEQ ID NO: 218. In some embodiments, the insertion motif comprises or consists of SEQ ID NO: 219. In some embodiments, the insertion motif comprises or consists of SEQ ID NO: 220. In some embodiments, the insertion motif comprises or consists of SEQ ID NO: 221. In some embodiments, the insertion motif comprises or consists of SEQ ID NO: 222. In some embodiments, the insertion motif comprises or consists of SEQ ID NO: 223. In some embodiments, the insertion motif comprises or consists of SEQ ID NO: 224. In some embodiments, the insertion motif comprises or consists of SEQ ID NO: 225. In some embodiments, the insertion motif comprises or consists of SEQ ID NO: 226. In some embodiments, the insertion motif comprises or consists of SEQ ID NO: 227. In some embodiments, the insertion motif comprises or consists of SEQ ID NO: 228. In some embodiments, the insertion motif comprises or consists of SEQ ID NO: 229. In some embodiments, the insertion motif comprises or consists of SEQ ID NO: 230. In some embodiments, the insertion motif comprises or consists of SEQ ID NO: 231. In some embodiments, the insertion motif comprises or consists of SEQ ID NO: 232. In some embodiments, the insertion motif comprises or consists of SEQ ID NO: 233. In some embodiments, the insertion motif comprises or consists of SEQ ID NO: 234. In some embodiments, the insertion motif comprises or consists of SEQ ID NO: 235. In some embodiments, the insertion motif comprises or consists of SEQ ID NO: 236. In some embodiments, the insertion motif comprises or consists of SEQ ID NO: 237. In some embodiments, the insertion motif comprises or consists of SEQ ID NO: 238. In some embodiments, the insertion motif comprises or consists of SEQ ID NO: 239. In some embodiments, the insertion motif comprises or consists of SEQ ID NO: 240. In some embodiments, the insertion motif comprises or consists of SEQ ID NO: 241. In some embodiments, the insertion motif comprises or consists of SEQ ID NO: 242.

In some embodiments, the insertion motif comprises an amino acid sequence of RX₁DX₂X₃X₄X₅ in the VR-VIII site, wherein: X₁ is Glycine (G) or Threonine (T); X₂=Histidine (H), Serine (S), Alanine (A), Leucine (L), Threonine (T), Glycine (G), Valine (V), or Phenylalanine (F); X₃=Glycine (G), Alanine (A), Valine (V), Lysine (K), Asparagine (N), or Threonine (T); X₄=Valine (V), Serine (S), Glycine (G), Asparagine (N), or Arginine (R); and X₅=Leucine (L), Tryptophan (W), Threonine (T), Glycine (G), or Arginine (R). In some embodiments, the insertion motif comprises an amino acid sequence selected from SEQ ID NOs: 215-242.

In some embodiments, the insertion motif comprises an amino acid sequence of RX₁DX₂X₃X₄X₅ in the VR-VIII site, wherein: X₁ is Glycine (G) or Threonine (T); X₂=Histidine (H), Serine (S), Alanine (A), Leucine (L), Threonine (T), Glycine (G), Valine (V), or Phenylalanine (F); X₃=Glycine (G), Alanine (A), or Asparagine (N); X₄=Valine (V), Serine (S), or Asparagine (N); and X₅=Leucine (L), Tryptophan (W), or Threonine (T). In some embodiments, the insertion motif comprises an amino acid sequence selected from SEQ ID NOs: 215-227 and 240-242.

In some embodiments, the insertion motif comprises an amino acid sequence of RX₁DX₂X₃X₄X₅ in the VR-VIII site, wherein: X₁ is Glycine (G) or Threonine (T); X₂=, Serine (S), Alanine (A), Leucine (L), Threonine (T), Glycine (G), or Valine (V); X₃=Glycine (G), Alanine (A), or Asparagine (N); X₄=Valine (V), Serine (S), or Asparagine (N); and X₅=Leucine (L), Tryptophan (W), or Threonine (T). In some embodiments, the insertion motif comprises an amino acid sequence selected from SEQ ID NOs: 215-227.

The engineered capsid proteins may comprise any of the insertion motifs described herein. In some embodiments, the non-naturally occurring amino acid motif comprises an insertion motif and does not comprise a substitution motif.

### Combined Substitution and Insertion Motifs

In some embodiments, the non-naturally occurring amino acid motif comprises an insertion motif and a substitution motif in any wild-type or parental AAV capsid protein described herein (e.g., AAV9-based, AAV5-based, AAVrh.10-based, or AAVrh.74 based). In some embodiments, the substitution motif is selected from Table 1 and the insertion motif is selected from Table 2.

In some embodiments, the non-naturally occurring amino acid motif comprises an amino acid sequence of X₁X₂X₃RX₄DX₅X₆X₇X₈X₉X₁₀ in the VR-VIII site, wherein X₁ corresponds to S586, X₂ corresponds to A587, X₃ corresponds to Q588, X₉ corresponds to A589, and X₁₀ corresponds to Q590, wherein the amino acid positions are numbered according to SEQ ID NO: 1. In some embodiments, X₁ is Serine (S), Glutamic acid (E), or Alanine (A); X₂ is Alanine (A), Serine (S), or Asparagine (N); X₃ is Glutamine (Q), Valine (V), Arginine (R), or Threonine (T); X₄ is Glycine (G) or Threonine (T); X₅ is Histidine (H), Serine (S), Alanine (A), Leucine (L), Threonine (T), Glycine (G), Valine (V), or Phenylalanine (F); X₆ is Glycine (G), Alanine (A), Valine (V), Lysine (K), Asparagine (N), or Threonine (T); X₇ is Valine (V), Serine (S), Glycine (G), Asparagine (N), or Arginine (R); X₈ is Leucine (L), Tryptophan (W), Threonine (T), Glycine (G), or Arginine (R); X₉ is Alanine (A), Threonine (T), Asparagine (N), or Serine (S); and X₁₀ is Glutamine (Q), Glycine (G), Leucine (L), or Arginine (R). In some embodiments, the non-naturally occurring amino acid motif is selected from any one SEQ ID NOs: 78-145 provided in Table 3B below. In some embodiments, the non-naturally occurring amino acid motif comprises an amino acid sequence having no more than 1 or 2 amino acid substitutions in the amino acid sequence selected from any one of SEQ ID NOs: 78-145. In some embodiments, the 1 or 2 amino acid substitutions are conservative amino acid substitutions.

In some embodiments, the engineered AAV capsid protein is an engineered AAV9 capsid protein comprising a non-naturally occurring amino acid motif as described herein in the wild-type AAV9 VP1 (SEQ ID NO: 1), AAV9 VP2 (SEQ ID NO: 2), or AAV9 VP3 (SEQ ID NO: 3). In some embodiments, the non-naturally occurring amino acid motif as described herein is located anywhere within the VR-VIII site (between amino acids 581 and 595 of the parental sequence of SEQ ID NO: 1). In some embodiments, the non-naturally occurring amino acid motif as described herein is located between amino acids 585 and 591 of the parental sequence of SEQ ID NO: 1. In some embodiments, the non-naturally occurring amino acid motif as described herein replaces the natural amino acid sequence at amino acid positions 586 to 590, wherein the amino acid numbering is according to SEQ ID NO: 1. In some embodiments, the engineered AAV9 capsid protein comprises at least 80% amino acid sequence identity to SEQ ID NO: 3, and comprises an amino acid sequence of any one of SEQ ID NOs: 78-145 replacing the natural amino acid sequence at amino acid positions 586 to 590, wherein the amino acid numbering is according to SEQ ID NO: 1.

In some embodiments, the engineered AAV capsid protein is an engineered AAV5 capsid protein comprising a non-naturally occurring amino acid motif as described herein in the wild-type AAV5 VP1 (SEQ ID NO: 10), AAV5 VP2 (SEQ ID NO: 11), or AAV5 VP3 (SEQ ID NO: 12). In some embodiments, the non-naturally occurring amino acid motif as described herein is located anywhere within the VR-VIII site (between amino acids 570 and 584 of the parental sequence of SEQ ID NO: 10). In some embodiments, the non-naturally occurring amino acid motif as described herein is located between amino acids 574 and 580 of the parental sequence of SEQ ID NO: 10. In some embodiments, the non-naturally occurring amino acid motif as described herein replaces the natural amino acid sequence at amino acid positions 575 to 579, wherein the amino acid numbering is according to SEQ ID NO: 10. In some embodiments, the engineered AAV5 capsid protein comprises at least 80% amino acid sequence identity to SEQ ID NO: 12, and comprises an amino acid sequence of any one of SEQ ID NOs: 78-145 replacing the natural amino acid sequence at amino acid positions 575 to 579, wherein the amino acid numbering is according to SEQ ID NO: 10.

In some embodiments, the engineered AAV capsid protein is an engineered AAVrh.10 capsid protein comprising a non-naturally occurring amino acid motif as described herein in the wild-type AAVrh.10 VP1 (SEQ ID NO: 19), AAVrh.10 VP2 (SEQ ID NO: 20), or AAVrh.10 VP3 (SEQ ID NO: 21). In some embodiments, the non-naturally occurring amino acid motif as described herein is located anywhere within the VR-VIII site (between amino acids 583 and 597 of the parental sequence of SEQ ID NO: 19). In some embodiments, the non-naturally occurring amino acid motif as described herein is located between amino acids 587 and 593 of the parental sequence of SEQ ID NO: 19. In some embodiments, the non-naturally occurring amino acid motif as described herein replaces the natural amino acid sequence at amino acid positions 588 to 592 of the parental sequence of SEQ ID NO: 19. In some embodiments, the engineered AAVrh.10 capsid protein comprises at least 80% amino acid sequence identity to SEQ ID NO: 21 and comprises an amino acid sequence of any one of SEQ ID NOs: 78-145 replacing the natural amino acid sequence at amino acid positions 588 to 592, wherein the amino acid numbering is according to SEQ ID NO: 19.

In some embodiments, the engineered AAV capsid protein is an engineered AAVrh.74 capsid protein comprising a non-naturally occurring amino acid motif as described herein in the wild-type AAVrh.74 VP1 (SEQ ID NO: 28), AAVrh.74 VP2 (SEQ ID NO: 29), or AAVrh.74 VP3 (SEQ ID NO: 30). In some embodiments, the non-naturally occurring amino acid motif as described herein is located anywhere within the VR-VIII site (between amino acids 583 and 597 of the parental sequence of SEQ ID NO: 28). In some embodiments, the non-naturally occurring amino acid motif as described herein is located between amino acids 587 and 594 of the parental sequence of SEQ ID NO: 28. In some embodiments, the non-naturally occurring amino acid motif as described herein replaces the natural amino acid sequence at amino acid positions 588 to 592 of the parental sequence of SEQ ID NO: 28. In some embodiments, the engineered AAVrh.10 capsid protein comprises at least 80% amino acid sequence identity to SEQ ID NO: 30 and comprises an amino acid sequence of any one of SEQ ID NOs: 78-145 replacing the natural amino acid sequence at amino acid positions 588 to 592, wherein the amino acid numbering is according to SEQ ID NO: 28.

### B. Specific Embodiments of Non-Naturally Occurring Motifs and Engineered Capsid Proteins

The specific embodiments discussed in this section are for illustrative purposes only and are not meant to be limiting.

In some embodiments, provided is an engineered AAV9 capsid protein comprising a non-naturally occurring motif comprising one or more polypeptide sequences inserted between two amino acids (i.e., an “insertion”) with respect to the wild-type or parental AAV9 sequence at one or more VR sites. In some embodiments, the non-naturally occurring motif additionally comprises one or more amino acid substitutions with respect to the wild-type or parental AAV9 capsid protein sequence. In some embodiments, the one or more sites of the parental sequence comprise the VR-VIII site.

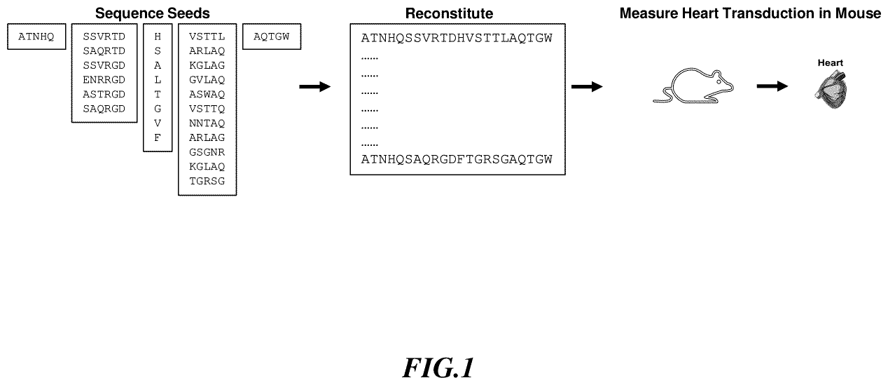

In some embodiments, the engineered AAV9 capsid protein comprises an insertion at the VR-VIII site, e.g., between amino acids 588 (glutamine (Q)) and 589 (alanine (A)) within the VR-VIII site in reference to the wild-type full-length AAV9 capsid protein of SEQ ID NO: 1 (FIG. 1). The insertion can be any described herein, including those provided in Table 3A below. In some embodiments, the engineered AAV9 capsid protein further comprises one or more amino acid substitutions within the VR-VIII site, including, for example, at one or more of amino acid positions 586-590 in reference to the wild-type full-length AAV9 capsid protein of SEQ ID NO: 1 (FIG. 1).

In some embodiments, provided is an engineered AAV capsid protein wherein the engineered capsid protein comprises at least 90% or at least 95% amino acid sequence identity to AAV9 VP3 SEQ ID NO: 3, and comprises the amino acid sequence of any one of SEQ ID NOs: 147-214 provided in Table 3A below replacing the natural amino acid sequence at amino acid positions 581 to 595, wherein the amino acid numbering is according to AAV9 VP1 SEQ ID NO: 1.

Solely for purposes of clarity and without limitation, it is noted that reference to amino acid positions at which modifications (e.g., insertions and/or substitutions) occur is relative to the positions of the corresponding full-length wild-type sequence (e.g., full-length wild-type AAV9 capsid protein sequence of SEQ ID NO:1). In some embodiments, the engineered capsid protein does not comprise the full-length sequence but comprises a shorter variant of the full-length sequence. In such embodiments, the modifications described herein may not occur at the same numerical positions as in the full-length wild-type sequence but occur at the same site or consensus sequence as the full-length wild-type sequence.

TABLE 3A
Exemplary engineered AAV9 capsid protein VR-VIII sequences

| | | Amino acid positions in reference to wild-type AAV9 | | | | | |
|---|---|---|---|---|---|---|---|
| | VR-VIII sequence | VP1 (SEQ ID NO: 1) | | | | | |
| Name | (with insertion) | 586 | 587 | 588 | Insertion | 589 | 590 |
| wild-type | ATNHQSAQAQAQTGW | S | A | Q | — | A | Q |
| AAV9 | (SEQ ID NO: 146) | | | | | | |
| lib73_variant- | ATNHQSSVRGDHGVLAQA | S | S | V | RGDHGVL (SEQ ID | A | Q |
| 482 | QTGW (SEQ ID NO: 147) | | | | NO: 242) | | |
| lib73_variant- | ATNHQSSVRGDSASWAQA | S | S | V | RGDSASW (SEQ ID | A | Q |
| 378 | QTGW (SEQ ID NO: 148) | | | | NO: 220) | | |
| lib73_variant- | ATNHQSSVRGDAGVLAQA | S | S | V | RGDAGVL (SEQ ID | A | Q |
| 356 | QTGW (SEQ ID NO: 149) | | | | NO: 216) | | |
| ZC736 | ATNHQENRRGDLVSTTQA | E | N | R | RGDLVST (SEQ ID | T | Q |
| | QTGW (SEQ ID NO: 150) | | | | NO: 234) | | |
| ZC739 | ATNHQASTRGDTKGLAGA | A | S | T | RGDTKGL (SEQ ID | A | G |
| | QTGW (SEQ ID NO: 151) | | | | NO: 238) | | |
| lib73_variant- | ATNHQENRRGDLVSTTLAQ | E | N | R | RGDLVST (SEQ ID | T | L |
| 1602 | TGW (SEQ ID NO: 152) | | | | NO: 234) | | |
| lib73_variant- | ATNHQENRRGDHGVLAQA | E | N | R | RGDHGVL (SEQ ID | A | Q |
| 1658 | QTGW (SEQ ID NO: 153) | | | | NO: 242) | | |
| lib73_variant- | ATNHQSSVRGDTGVLAQA | S | S | V | RGDTGVL (SEQ ID | A | Q |
| 461 | QTGW (SEQ ID NO: 154) | | | | NO: 223) | | |
| lib73_variant- | ATNHQSSVRGDSGVLAQA | S | S | V | RGDSGVL (SEQ ID | A | Q |
| 377 | QTGW (SEQ ID NO: 155) | | | | NO: 221) | | |
| lib73_variant- | ATNHQASTRGDAKGLAGA | A | S | T | RGDAKGL (SEQ ID | A | G |
| 1691 | QTGW (SEQ ID NO: 156) | | | | NO: 229) | | |
| lib73_variant- | ATNHQSSVRGDGASWAQA | S | S | V | RGDGASW (SEQ ID | A | Q |
| 420 | QTGW (SEQ ID NO: 157) | | | | NO: 217) | | |
| lib73_variant- | ATNHQSAQRGDSASWAQA | S | A | Q | RGDSASW (SEQ ID | A | Q |
| 882 | QTGW (SEQ ID NO: 158) | | | | NO: 220) | | |
| lib73_variant- | ATNHQSSVRGDLNNTAQA | S | S | V | RGDLNNT (SEQ ID | A | Q |
| 439 | QTGW (SEQ ID NO: 159) | | | | NO: 219) | | |
| lib73_variant- | ATNHQSAQRGDHGVLAQA | S | A | Q | RGDHGVL (SEQ ID | A | Q |
| 986 | QTGW (SEQ ID NO: 160) | | | | NO: 242) | | |
| lib73_variant- | ATNHQASTRGDAGVLAQA | A | S | T | RGDAGVL (SEQ ID | A | Q |
| 1700 | QTGW (SEQ ID NO: 161) | | | | NO: 216) | | |
| lib73_variant- | ATNHQSSVRTDLGVLAQA | S | S | V | RTDLGVL (SEQ ID | A | Q |
| 608 | QTGW (SEQ ID NO: 162) | | | | NO: 226) | | |
| lib73_variant- | ATNHQSSVRGDGGVLAQA | S | S | V | RGDGGVL (SEQ ID | A | Q |
| 419 | QTGW (SEQ ID NO: 163) | | | | NO: 218) | | |
| lib73_variant- | ATNHQASTRGDSASWAQA | A | S | T | RGDSASW (SEQ ID | A | Q |
| 1722 | QTGW (SEQ ID NO: 164) | | | | NO: 220) | | |
| lib73_variant- | ATNHQASTRGDVKGLAGA | A | S | T | RGDVKGL (SEQ ID | A | G |
| 1733 | QTGW (SEQ ID NO: 165) | | | | NO: 239) | | |
| lib73_varian-t | ATNHQASTRGDSGVLAQA | A | S | T | RGDSGVL (SEQ ID | A | Q |
| 1721 | QTGW (SEQ ID NO: 166) | | | | NO: 221) | | |
| lib73_variant- | ATNHQSAQRGDGASWAQA | S | A | Q | RGDGASW (SEQ ID | A | Q |
| 924 | QTGW (SEQ ID NO: 167) | | | | NO: 217) | | |
| lib73_variant- | ATNHQSAQRGDAGVLAQA | S | A | Q | RGDAGVL (SEQ ID | A | Q |
| 860 | QTGW (SEQ ID NO: 168) | | | | NO: 216) | | |
| lib73_variant- | ATNHQSSVRGDFNNTAQA | S | S | V | RGDFNNT (SEQ ID | A | Q |
| 502 | QTGW (SEQ ID NO: 169) | | | | NO: 240) | | |
| lib73_variant- | ATNHQENRRGDSGVLAQA | E | N | R | RGDSGVL (SEQ ID | A | Q |
| 1553 | QTGW (SEQ ID NO: 170) | | | | NO: 221) | | |
| lib73_variant- | ATNHQENRRGDAGVLAQA | E | N | R | RGDAGVL (SEQ ID | A | Q |
| 1532 | QTGW (SEQ ID NO: 171) | | | | NO: 216) | | |
| lib73_variant- | ATNHQENRRGDLNNTAQA | E | N | R | RGDLNNT (SEQ ID | A | Q |
| 1615 | QTGW (SEQ ID NO: 172) | | | | NO: 219) | | |
| lib73_variant- | ATNHQASTRGDTGVLAQA | A | S | T | RGDTGVL (SEQ ID | A | Q |
| 1805 | QTGW (SEQ ID NO: 173) | | | | NO: 223) | | |
| lib73_variant- | ATNHQASTRGDAASWAQA | A | S | T | RGDAASW (SEQ ID | A | Q |
| 1701 | QTGW (SEQ ID NO: 174) | | | | NO: 215) | | |
| lib73_variant- | ATNHQSAQRGDAASWAQA | S | A | Q | RGDAASW (SEQ ID | A | Q |
| 861 | QTGW (SEQ ID NO: 175) | | | | NO: 215) | | |
| lib73_variant- | ATNHQASTRGDAARLAGA | A | S | T | RGDAARL (SEQ ID | A | G |
| 1681 | QTGW (SEQ ID NO: 176) | | | | NO: 228) | | |
| lib73_variant- | ATNHQASTRGDGGVLAQA | A | S | T | RGDGGVL (SEQ ID | A | Q |
| 1763 | QTGW (SEQ ID NO: 177) | | | | NO: 218) | | |
| lib73_variant- | ATNHQSAQRGDSGVLAQA | S | A | Q | RGDSGVL (SEQ ID | A | Q |
| 881 | QTGW (SEQ ID NO: 178) | | | | NO: 221) | | |
| lib73_variant- | ATNHQASTRGDLVSTTLAQ | A | S | T | RGDLVST (SEQ ID | T | L |
| 1770 | TGW (SEQ ID NO: 179) | | | | NO: 234) | | |
| ZC738 | ATNHQENRRGDLGSGNRA | E | N | R | RGDLGSG (SEQ ID | N | R |
| | QTGW (SEQ ID NO: 180) | | | | NO: 232) | | |
| lib73_variant- | ATNHQASTRGDLVSTTQAQ | A | S | T | RGDLVST (SEQ ID | T | Q |
| 1780 | TGW (SEQ ID NO: 181) | | | | NO: 234) | | |
| lib73_variant- | ATNHQASTRGDGASWAQA | A | S | T | RGDGASW (SEQ ID | A | Q |
| 1764 | QTGW (SEQ ID NO: 182) | | | | NO: 217) | | |
| lib73_variant- | ATNHQSAQRGDGGVLAQA | S | A | Q | RGDGGVL (SEQ ID | A | Q |
| 923 | QTGW (SEQ ID NO: 183) | | | | NO: 218) | | |
| lib73_variant- | ATNHQASTRGDLTGRSGAQ | A | S | T | RGDLTGR (SEQ ID | S | G |
| 1769 | TGW (SEQ ID NO: 184) | | | | NO: 233) | | |
| lib73_variant- | ATNHQSSVRGDAASWAQA | S | S | V | RGDAASW (SEQ ID | A | Q |
| 357 | QTGW (SEQ ID NO: 185) | | | | NO: 215) | | |
| lib73_variant- | ATNHQSAQRTDLGVLAQA | S | A | Q | RTDLGVL (SEQ ID | A | Q |
| 776 | QTGW (SEQ ID NO: 186) | | | | NO: 226) | | |
| lib73_variant- | ATNHQSAQRGDFNNTAQA | S | A | Q | RGDFNNT (SEQ ID | A | Q |
| 1006 | QTGW (SEQ ID NO: 187) | | | | NO: 240) | | |
| lib73_variant- | ATNHQENRRGDGGVLAQA | E | N | R | RGDGGVL (SEQ ID | A | Q |
| 1595 | QTGW (SEQ ID NO: 188) | | | | NO: 218) | | |
| lib73_variant- | ATNHQASTRGDSARLAGA | A | S | T | RGDSARL (SEQ ID | A | G |
| 1702 | QTGW (SEQ ID NO: 189) | | | | NO: 235) | | |
| lib73_variant- | ATNHQASTRGDTASWAQA | A | S | T | RGDTASW (SEQ ID | A | Q |
| 1806 | QTGW (SEQ ID NO: 190) | | | | NO: 222) | | |
| lib73_variant- | ATNHQSSVRGDTASWAQA | S | S | V | RGDTASW (SEQ ID | A | Q |
| 462 | QTGW (SEQ ID NO: 191) | | | | NO: 222) | | |
| lib73_variant- | ATNHQASTRGDLNNTAQA | A | S | T | RGDLNNT (SEQ ID | A | Q |
| 1783 | QTGW (SEQ ID NO: 192) | | | | NO: 219) | | |
| lib73_variant- | ATNHQASTRGDTARLAGA | A | S | T | RGDTARL (SEQ ID | A | G |
| 1786 | QTGW (SEQ ID NO: 193) | | | | NO: 237) | | |
| lib73_variant- | ATNHQENRRGDSASWAQA | E | N | R | RGDSASW (SEQ ID | A | Q |
| 1554 | QTGW (SEQ ID NO: 194) | | | | NO: 220) | | |
| lib73_variant- | ATNHQASTRGDVKGLAQA | A | S | T | RGDVKGL (SEQ ID | A | Q |
| 1730 | QTGW (SEQ ID NO: 195) | | | | NO: 238) | | |
| lib73_variant- | ATNHQSAQRGDTGVLAQA | S | A | Q | RGDTGVL (SEQ ID | A | Q |
| 965 | QTGW (SEQ ID NO: 196) | | | | NO: 223) | | |
| lib73_variant- | ATNHQASTRGDSKGLAGA | A | S | T | RGDSKGL (SEQ ID | A | G |
| 1712 | QTGW (SEQ ID NO: 197) | | | | NO: 236) | | |
| lib73_variant- | ATNHQASTRGDTKGLAQA | A | S | T | RGDTKGL (SEQ ID | A | Q |
| 1793 | QTGW (SEQ ID NO: 198) | | | | NO: 238) | | |
| lib73_variant- | ATNHQASTRGDSARLAQA | A | S | T | RGDSARL (SEQ ID | A | 0 |
| 1703 | QTGW (SEQ ID NO: 199) | | | | NO: 235) | | |
| lib73_variant- | ATNHQSAQRGDTASWAQA | S | A | Q | RGDTASW (SEQ ID | A | Q |
| 966 | QTGW (SEQ ID NO: 200) | | | | NO: 222) | | |
| lib73_variant- | ATNHQSAQRGDLNNTAQA | S | A | Q | RGDLNNT (SEQ ID | A | Q |
| 943 | QTGW (SEQ ID NO: 201) | | | | NO: 219) | | |
| lib73_variant- | ATNHQASTRGDHASWAQA | A | S | T | RGDHASW (SEQ ID | A | Q |
| 1827 | QTGW (SEQ ID NO: 202) | | | | NO: 241) | | |
| lib73_variant- | ATNHQASTRGDGARLAGA | A | S | T | RGDGARL (SEQ ID | A | G |
| 1744 | QTGW (SEQ ID NO: 203) | | | | NO: 230) | | |
| lib73_variant- | ATNHQSSVRGDHASWAQA | S | S | V | RGDHASW (SEQ ID | A | Q |
| 483 | QTGW (SEQ ID NO: 204) | | | | NO: 241) | | |
| lib73_variant- | ATNHQASTRGDFNNTAQA | A | S | T | RGDFNNT (SEQ ID | A | Q |
| 1846 | QTGW (SEQ ID NO: 205) | | | | NO: 240) | | |
| lib73_variant- | ATNHQSSVRGDVGVLAQA | S | S | V | RGDVGVL (SEQ ID | A | O |
| 398 | QTGW (SEQ ID NO: 206) | | | | NO: 225) | | |
| lib73_variant- | ATNHQENRRGDGASWAQA | E | N | R | RGDGASW (SEQ ID | A | Q |
| 1596 | QTGW (SEQ ID NO: 207) | | | | NO: 217) | | |
| lib73_variant- | ATNHQASTRGDAARLAQA | A | S | T | RGDAARL (SEQ ID | A | Q |
| 1682 | QTGW (SEQ ID NO: 208) | | | | NO: 228) | | |
| lib73_variant- | ATNHQASTRGDGKGLAGA | A | S | T | RGDGKGL (SEQ ID | A | G |
| 1754 | QTGW (SEQ ID NO: 209) | | | | NO: 231) | | |
| lib73_variant- | ATNHQASTRGDAKGLAQA | A | S | T | RGDAKGL (SEQ ID | A | Q |
| 1688 | QTGW (SEQ ID NO: 210) | | | | NO: 229) | | |
| lib73_variant- | ATNHQENRRGDAASWAQA | E | N | R | RGDAASW (SEQ ID | A | Q |
| 1533 | QTGW (SEQ ID NO: 211) | | | | NO: 215) | | |
| lib73_variant- | ATNHQSAQRTDVGVLAQA | S | A | Q | RTDVGVL (SEQ ID | A | Q |
| 734 | QTGW (SEQ ID NO: 212) | | | | NO: 227) | | |
| lib73_variant- | ATNHQENRRGDTGVLAQA | E | N | R | RGDTGVL (SEQ ID | A | Q |
| 1637 | QTGW (SEQ ID NO: 213) | | | | NO: 223) | | |
| lib73_variant- | ATNHQASTRGDVASWAQA | A | S | T | RGDVASW (SEQ ID | A | Q |
| 1743 | QTGW (SEQ ID NO: 214) | | | | NO: 224) | | |

In some embodiments, the engineered AAV9 capsid protein comprises a non-naturally occurring motif comprising (i) an insertion at the VR-VIII site, e.g., between amino acids 588 (glutamine (Q)) and 589 (alanine (A)) within the VR-VIII site in reference to the wild-type full-length AAV9 capsid protein of SEQ ID NO: 1 (FIG. 1), and/or (ii) one or more amino acid substitutions within the VR-VIII site, including, for example, at one or more of amino acid positions 586, 587, 588, and 590, in reference to the wild-type full-length AAV9 capsid protein of SEQ ID NO: 1 (FIG. 1). In some embodiments, the non-naturally occurring motif comprises amino acids 586 to 590, including the insertion motif, within the VR-VIII site in reference to the wild-type full-length AAV9 capsid protein of SEQ ID NO: 1 (FIG. 1). In some embodiments, the non-naturally occurring motif can be any disclosed herein, including those provided in Table 3B below.

TABLE 3B
Exemplary non-naturally occurring amino acid
motifs

| | VR-VIII_ |
|---|---|
| Name | (586-590, plus insertion) |
| wild-type AAV9 | SAQAQ (SEQ ID NO: 77) |
| lib73_variant-482 | SSVRGDHGVLAQ (SEQ ID NO: 78) |
| lib73_variant-378 | SSVRGDSASWAQ (SEQ ID NO: 79) |
| lib73_variant-356 | SSVRGDAGVLAQ (SEQ ID NO: 80) |
| ZC736 | ENRRGDLVSTTQ (SEQ ID NO: 81) |
| ZC739 | ASTRGDTKGLAG (SEQ ID NO: 82) |
| lib73_variant-1602 | ENRRGDLVSTTL (SEQ ID NO: 83) |
| lib73_variant-1658 | ENRRGDHGVLAQ (SEQ ID NO: 84) |
| lib73_variant-461 | SSVRGDTGVLAQ (SEQ ID NO: 85) |
| lib73_variant-377 | SSVRGDSGVLAQ (SEQ ID NO: 86) |
| lib73_variant-1691 | ASTRGDAKGLAG (SEQ ID NO: 87) |
| lib73_variant-420 | SSVRGDGASWAQ (SEQ ID NO: 88) |
| lib73_variant-882 | SAQRGDSASWAQ (SEQ ID NO: 89) |
| lib73_variant-439 | SSVRGDLNNTAQ (SEQ ID NO: 90) |
| lib73_variant-986 | SAQRGDHGVLAQ (SEQ ID NO: 91) |
| lib73_variant-1700 | ASTRGDAGVLAQ (SEQ ID NO: 92) |
| lib73_variant-608 | SSVRTDLGVLAQ (SEQ ID NO: 93) |
| lib73_variant-419 | SSVRGDGGVLAQ (SEQ ID NO: 94) |
| lib73_variant-1722 | ASTRGDSASWAQ (SEQ ID NO: 95) |
| lib73_variant-1733 | ASTRGDVKGLAG (SEQ ID NO: 96) |
| lib73_variant-1721 | ASTRGDSGVLAQ (SEQ ID NO: 97) |
| lib73_variant-924 | SAQRGDGASWAQ (SEQ ID NO: 98) |
| lib73_variant-860 | SAQRGDAGVLAQ (SEQ ID NO: 99) |
| lib73_variant-502 | SSVRGDENNTAQ (SEQ ID NO: 100) |
| lib73_variant-1553 | ENRRGDSGVLAQ (SEQ ID NO: 101) |
| lib73_variant-1532 | ENRRGDAGVLAQ (SEQ ID NO: 102) |
| lib73_variant-1615 | ENRRGDLNNTAQ (SEQ ID NO: 103) |
| lib73_variant-1805 | ASTRGDTGVLAQ (SEQ ID NO: 104) |
| lib73_variant-1701 | ASTRGDAASWAQ (SEQ ID NO: 105) |
| lib73_variant-861 | SAQRGDAASWAQ (SEQ ID NO: 106) |
| lib73_variant-1681 | ASTRGDAARLAG (SEQ ID NO: 107) |
| lib73_variant-1763 | ASTRGDGGVLAQ (SEQ ID NO: 108) |
| lib73_variant-881 | SAQRGDSGVLAQ (SEQ ID NO: 109) |
| lib73_variant-1770 | ASTRGDLVSTTL (SEQ ID NO: 110) |
| ZC738 | ENRRGDLGSGNR (SEQ ID NO: 111) |
| lib73_variant-1780 | ASTRGDLVSTTQ (SEQ ID NO: 112) |
| lib73_variant-1764 | ASTRGDGASWAQ (SEQ ID NO: 113) |
| lib73_variant-923 | SAQRGDGGVLAQ (SEQ ID NO: 114) |
| lib73_variant-1769 | ASTRGDLTGRSG (SEQ ID NO: 115) |
| lib73_variant-357 | SSVRGDAASWAQ (SEQ ID NO: 116) |
| lib73_variant-776 | SAQRTDLGVLAQ (SEQ ID NO: 117) |
| lib73_variant-1006 | SAQRGDENNTAQ (SEQ ID NO: 118) |
| lib73_variant-1595 | ENRRGDGGVLAQ (SEQ ID NO: 119) |
| lib73_variant-1702 | ASTRGDSARLAG (SEQ ID NO: 120) |
| lib73_variant-1806 | ASTRGDTASWAQ (SEQ ID NO: 121) |
| lib73_variant-462 | SSVRGDTASWAQ (SEQ ID NO: 122) |
| lib73_variant-1783 | ASTRGDLNNTAQ (SEQ ID NO: 123) |
| lib73_variant-1786 | ASTRGDTARLAG (SEQ ID NO: 124) |
| lib73_variant-1554 | ENRRGDSASWAQ (SEQ ID NO: 125) |
| lib73_variant-1730 | ASTRGDVKGLAQ (SEQ ID NO: 126) |
| lib73_variant-965 | SAQRGDTGVLAQ (SEQ ID NO: 127) |
| lib73_variant-1712 | ASTRGDSKGLAG (SEQ ID NO: 128) |
| lib73_variant-1793 | ASTRGDTKGLAQ (SEQ ID NO: 129) |
| lib73_variant-1703 | ASTRGDSARLAQ (SEQ ID NO: 130) |
| lib73_variant-966 | SAQRGDTASWAQ (SEQ ID NO: 131) |
| lib73_variant-943 | SAQRGDLNNTAQ (SEQ ID NO: 132) |
| lib73_variant-1827 | ASTRGDHASWAQ (SEQ ID NO: 133) |
| lib73_variant-1744 | ASTRGDGARLAG (SEQ ID NO: 134) |
| lib73_variant-483 | SSVRGDHASWAQ (SEQ ID NO: 135) |
| lib73_variant-1846 | ASTRGDENNTAQ (SEQ ID NO: 136) |
| lib73_variant-398 | SSVRGDVGVLAQ (SEQ ID NO: 137) |
| lib73_variant-1596 | ENRRGDGASWAQ (SEQ ID NO: 138) |
| lib73_variant-1682 | ASTRGDAARLAQ (SEQ ID NO: 139) |
| lib73_variant-1754 | ASTRGDGKGLAG (SEQ ID NO: 140) |
| lib73_variant-1688 | ASTRGDAKGLAQ (SEQ ID NO: 141) |
| lib73_variant-1533 | ENRRGDAASWAQ (SEQ ID NO: 142) |
| lib73_variant-734 | SAQRTDVGVLAQ (SEQ ID NO: 143) |
| lib73_variant-1637 | ENRRGDTGVLAQ (SEQ ID NO: 144) |
| lib73_variant-1743 | ASTRGDVASWAQ (SEQ ID NO: 145) |

In some embodiments, the engineered AAV9 capsid protein comprises, consists essentially of, or consists of an amino acid sequence that shares at least about 70%, at least 75%, at least 80%, at least 85%, at least 90%, at least 95%, at least 96% at least 97%, at least 98%, at least 99%, or 100% identity to any one of SEQ ID NOs: 243-310. In some embodiments, the engineered AAV9 capsid protein comprises, consists essentially of, or consists of an amino acid sequence set forth in any one of SEQ ID NOs: 243-310.

The full-length capsid protein sequences of the above specific embodiments are provided in Table 4 below. In some embodiments, provided is an engineered capsid protein comprising, consisting essentially of, or consisting of an amino acid sequence that shares at least about 70%, at least 75%, at least 80%, at least 85%, at least 90%, at least 95%, at least 96% at least 97%, at least 98%, at least 99%, or 100% identity to any one of SEQ ID NOs: 243-310. In some embodiments, the engineered AAV9 capsid protein comprises, consists essentially of, or consists of an amino acid sequence set forth in any one of SEQ ID NOs: 243-310.

TABLE 4
Exemplary engineered capsid protein sequences

| | | SEQ ID |
|---|---|---|
| Name | Capsid protein sequence | NO: |
| lib73_variant- | MAADGYLPDWLEDNLSEGIREWWALKPGAPQPKANQQHQDNARGL | 243 |
| 482 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQSSVRGDHGVLAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 244 |
| 378 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQSSVRGDSASWAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 245 |
| 356 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQSSVRGDAGVLAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| ZC736 | MAADGYLPDWLEDNLSEGIREWWALKPGAPQPKANQQHQDNARGL | 246 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQENRRGDLVSTTQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| ZC739 | MAADGYLPDWLEDNLSEGIREWWALKPGAPQPKANQQHQDNARGL | 247 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQASTRGDTKGLAGAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 248 |
| 1602 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQENRRGDLVSTTLAQTGWVQN | |
| | QGILPGMVWQDRDVYLQGPIWAKIPHTDGNFHPSPLMGGFGMKHPP | |
| | PQILIKNTPVPADPPTAFNKDKLNSFITQYSTGQVSVEIEWELQKENSK | |
| | RWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 249 |
| 1658 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQENRRGDHGVLAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 250 |
| 461 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQSSVRGDTGVLAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 251 |
| 377 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQSSVRGDSGVLAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 252 |
| 1691 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQASTRGDAKGLAGAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 253 |
| 420 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQSSVRGDGASWAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 254 |
| 882 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQSAQRGDSASWAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 255 |
| 439 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQSSVRGDLNNTAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 256 |
| 986 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQSAQRGDHGVLAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 257 |
| 1700 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQASTRGDAGVLAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 258 |
| 608 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQSSVRTDLGVLAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 259 |
| 419 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQSSVRGDGGVLAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 260 |
| 1722 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQASTRGDSASWAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 261 |
| 1733 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQASTRGDVKGLAGAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 262 |
| 1721 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQASTRGDSGVLAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 263 |
| 924 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQSAQRGDGASWAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 264 |
| 860 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQSAQRGDAGVLAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 265 |
| 502 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQSSVRGDFNNTAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 266 |
| 1553 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQENRRGDSGVLAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 267 |
| 1532 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQENRRGDAGVLAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 268 |
| 1615 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQENRRGDLNNTAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 269 |
| 1805 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQASTRGDTGVLAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 270 |
| 1701 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQASTRGDAASWAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 271 |
| 861 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQSAQRGDAASWAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 272 |
| 1681 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQASTRGDAARLAGAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 273 |
| 1763 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQASTRGDGGVLAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 274 |
| 881 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQSAQRGDSGVLAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 275 |
| 1770 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQASTRGDLVSTTLAQTGWVQN | |
| | QGILPGMVWQDRDVYLQGPIWAKIPHTDGNFHPSPLMGGFGMKHPP | |
| | PQILIKNTPVPADPPTAFNKDKLNSFITQYSTGQVSVEIEWELQKENSK | |
| | RWNPEIQYTSNYYKSNNVEFAVNTEGVYSEPRPIGTRYLTRNL | |
| ZC738 | MAADGYLPDWLEDNLSEGIREWWALKPGAPQPKANQQHQDNARGL | 276 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQENRRGDLGSGNRAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 277 |
| 1780 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQASTRGDLVSTTQAQTGWVQN | |
| | QGILPGMVWQDRDVYLQGPIWAKIPHTDGNFHPSPLMGGFGMKHPP | |
| | PQILIKNTPVPADPPTAFNKDKLNSFITQYSTGQVSVEIEWELQKENSK | |
| | RWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 278 |
| 1764 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQASTRGDGASWAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 279 |
| 923 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQSAQRGDGGVLAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 280 |
| 1769 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQASTRGDLTGRSGAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 281 |
| 357 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQSSVRGDAASWAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 282 |
| 776 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQSAQRTDLGVLAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 283 |
| 1006 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQSAQRGDENNTAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 284 |
| 1595 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQENRRGDGGVLAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 285 |
| 1702 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQASTRGDSARLAGAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 286 |
| 1806 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQASTRGDTASWAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 287 |
| 462 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQSSVRGDTASWAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 288 |
| 1783 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQASTRGDLNNTAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 289 |
| 1786 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQASTRGDTARLAGAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 290 |
| 1554 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQENRRGDSASWAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 291 |
| 1730 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQASTRGDVKGLAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 292 |
| 965 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQSAQRGDTGVLAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 293 |
| 1712 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQASTRGDSKGLAGAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 294 |
| 1793 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQASTRGDTKGLAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 295 |
| 1703 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQASTRGDSARLAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 296 |
| 966 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQSAQRGDTASWAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 297 |
| 943 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQSAQRGDLNNTAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 298 |
| 1827 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQASTRGDHASWAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 299 |
| 1744 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQASTRGDGARLAGAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 300 |
| 483 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQSSVRGDHASWAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 301 |
| 1846 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQASTRGDFNNTAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 302 |
| 398 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQSSVRGDVGVLAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 303 |
| 1596 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQENRRGDGASWAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 304 |
| 1682 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQASTRGDAARLAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 305 |
| 1754 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQASTRGDGKGLAGAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 306 |
| 1688 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQASTRGDAKGLAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 307 |
| 1533 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQENRRGDAASWAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 308 |
| 734 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQSAQRTDVGVLAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 309 |
| 1637 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQENRRGDTGVLAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| lib73_variant- | MAADGYLPDWLEDNLSEGIREWWALKPGAPQPKANQQHQDNARGL | 310 |
| 1743 | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEAA | |
| | KTAPGKKRPVEQSPQEPDSSAGIGKSGAQPAKKRLNFGQTGDTESVP | |
| | DPQPIGEPPAAPSGVGSLTMASGGGAPVADNNEGADGVGSSSGNWH | |
| | CDSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQVK | |
| | EVTDNNGVKTIANNLTSTVQVFTDSDYQLPYVLGSAHEGCLPPFPAD | |
| | VFMIPQYGYLTLNDGSQAVGRSSFYCLEYFPSQMLRTGNNFQFSYEFE | |
| | NVPFHSSYAHSQSLDRLMNPLIDQYLYYLSKTINGSGQNQQTLKFSVA | |
| | GPSNMAVQGRNYIPGPSYRQQRVSTTVTQNNNSEFAWPGASSWALN | |
| | GRNSLMNPGPAMASHKEGEDRFFPLSGSLIFGKQGTGRDNVDADKV | |
| | MITNEEEIKTTNPVATESYGQVATNHQASTRGDVASWAQAQTGWVQ | |
| | NQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |

### C. Additional Substitutions

Additional amino acid substitutions may be incorporated into the engineered capsid proteins described herein, for example, to further improve transduction efficiency or tissue selectivity. Exemplary non-limiting substitutions include, but are not limited to, S651A, T578A or T582A relative to the sequence of AAV5, in either an AAV5 or AAV9-based capsid.

In some embodiments, the engineered capsid protein comprises a mutation selected from S651A, T578A, T582A, K251R, Y709F, Y693F, or S485A relative to the sequence of AAV5, in either an AAV5 or AAV9-based capsid. In some embodiments, the capsid protein comprises a mutation selected from K251R, Y709F, Y693F, or S485A relative to the sequence of AAV5, in either an AAV5 or AAV9-based capsid.

In some of these embodiments, the engineered capsid protein comprises N or K at position 452 relative to reference sequence SEQ ID NO: 1 (in addition to any of the variant polypeptide sequence described herein, such as comprising any of the insertion motifs described herein). In some embodiments, the engineered capsid protein of the present disclosure comprises wild type AAV9 amino acid (which is N) at position 452 of the VR-IV site relative to reference SEQ ID NO: 1.

In some of these embodiments, the engineered capsid protein may further comprise N452K substitution relative to reference sequence SEQ ID NO: 1 (in addition to the variant polypeptide sequence described herein, such as comprising any of the insertion motifs described herein). In some embodiments, the engineered AAV9 capsid protein comprises the amino acid substitution N452K at the VR-IV site of an AAV9-based capsid (wherein the substitution is at position 452 of the wild type AAV9 VP1 capsid protein). In some embodiments, the engineered AAV9 capsid protein comprises any of the variant polypeptide sequences described herein, and an amino acid substitution N452K. In some embodiments, the engineered AAV9 capsid protein comprises any of the VR-VIII modifications (such as insertion motifs) described herein, and an amino acid substitution N452K. In some embodiments, the engineered AAV9 capsid protein comprises any of the VR-IV modifications (such as insertion motifs) described herein, and an amino acid substitution N452K.

In some embodiments, the engineered capsid protein of the present disclosure comprises the amino acid sequence KGSGQNQ or KGSGQNQQT at the VR-IV site relative to reference SEQ ID NO:1. In some embodiments, the VR-IV site of the engineered capsid protein comprises amino acid sequence KGSGQNQQT. In some embodiments, the VR-IV site of the engineered capsid protein comprises, consists essentially of, or consists of a sequence of KGSGQNQQT.

In some embodiments, an engineered AAV9-based capsid protein of the present disclosure (such as an AAV9 capsid protein) comprises amino acid substitution N452K at the VR-IV site in addition to any other substitution or insertion described herein or known in the art. In some embodiments, such substitution is combined with any insertion motif and/or substitution(s) described herein (e.g., any insertion motifs and/or amino acid substitution(s) in the VR-IV and/or VR-VIII site). In some embodiments, the engineered AAV9-based capsid protein of the present disclosure comprises amino acid substitution N452K at the VR-IV site in addition to any insertion motif at the VR-VIII site described herein. In some embodiments, the engineered AAV9-based capsid protein of the present disclosure comprises amino acid substitution N452K, relative to reference sequence SEQ ID NO: 1, in addition to any insertion motif at the VR-VIII site described herein. In some embodiments, the engineered AAV9-based capsid protein of the present disclosure comprises amino acid substitution N452K at the VR-IV site in addition to any insertion motif at the VR-IV site described herein. In some embodiments, the engineered AAV9-based capsid protein of the present disclosure comprises amino acid substitution N452K, relative to reference sequence SEQ ID NO: 1, in addition to any insertion motif at the VR-IV site described herein. In some embodiments, the engineered capsid protein, such as the capsid protein with N452K substitution at the VR-IV site relative to reference SEQ ID NO: 1, increases transduction efficiency (e.g., of any tissue, such as muscle, heart, skeletal muscle, brain, etc.). In some embodiments, the engineered capsid protein of the present disclosure, such as the capsid protein with N452K substitution at the VR-IV site relative to reference SEQ ID NO: 1, increases transduction efficiency of the heart.

In some embodiments, the engineered capsid protein described herein does not comprise N452K substitution (relative to reference sequence SEQ ID NO:1) in the VR-IV site.

### D. AAV9

In some embodiments, the engineered capsid protein is an engineered AAV9 capsid protein comprising a non-naturally occurring amino acid motif (e.g., a substitution motif, an insertion motif, or both) compared to the wild-type AAV9 capsid protein.

The wild-type AAV9 VP1 has the amino acid sequence of SEQ ID NO: 1; the wild-type AAV9 VP2 has the amino acid sequence of SEQ ID NO: 2; the wild-type AAV9 VP3 has the amino acid sequence of SEQ ID NO: 3, as shown below and provided in Table 5. The N-terminal residue of VP1, VP2, and VP3, as well as the variable region (VR) sites (e.g., VR-I, VR-II, VR-IV, VR-V, VR-VII and VR-VIII), are indicated in bold, underlined, and enlarged text in the sequence of full-length VP1 (SEQ ID NO: 1). In some embodiments, the engineered AAV9 capsid protein comprises a sequence that shares at least about 80% (e.g., at least 80%, 85%, 90%, 95%, 96%, 97%, 98%, 99%, or 100%) identity to SEQ ID NO: 1, for example, as compared using a sequence alignment algorism, e.g., BLAST® provided by the National Center for Biotechnology Information (NCBI). In some embodiments, the engineered AAV9 capsid protein comprises a sequence that shares at least about 80% (e.g., at least 80%, 85%, 90%, 95%, 96%, 97%, 98%, 99%, or 100%) identity to SEQ ID NO: 2. In some embodiments, the engineered AAV9 capsid protein comprises a sequence that shares at least about 80% (e.g., at least 80%, 85%, 90%, 95%, 96%, 97%, 98%, 99%, or 100%) identity to SEQ ID NO: 3.

Annotated WT VP1 Sequence
(SEQ ID NO: 1)
MAADGYLPDWLEDNLSEGIREWWALKPGAPQPKANQQHQDNARGLVLPGY
KYLGPGNGLDKGEPVNAADAAALEHDKAYDQQLKAGDNPYLKYNHADAEF
QERLKEDTSFGGNLGRAVEQAKKRLLEPLGLVEEAAKTAPGKKRPVEQSP
QEPDSSAGIGKSGAQPAKKRLNFGQTGDTESVPDPQPIGEPPAAPSGVGS
LTMASGGGAPVADNNEGADGVGSSSGNWHCDSQWLGDRVITTSTRTWALP
TYNNHLYKQISNSTSGGSSNDNAYFGYSTPWGYFDFNRFHCHFSPRDWQR
LINNNWGFRPKRLNFKLFNIQVKEVTDNNGVKTIANNLTSTVQVFTDSDY
QLPYVLGSAHEGCLPPFPADVEMIPQYGYLTLNDGSQAVGRSSFYCLEYF
PSQMLRTGNNFQFSYEFENVPFHSSYAHSQSLDRLMNPLIDQYLYYLSKT
INGSGQNQQTLKFSVAGPSNMAVQGRNYIPGPSYRQQRVSTTVTQNNNSE
FAWPGASSWALNGRNSLMNPGPAMASHKEGEDRFFPLSGSLIFGKQGTGR
DNVDADKVMITNEEEIKTTNPVATESYGQVATNHQSAQAQAQTGWVQNQG
ILPGMVWQDRDVYLQGPIWAKIPHTDGNFHPSPLMGGFGMKHPPPQILIK
NTPVPADPPTAFNKDKLNSFITQYSTGQVSVEIEWELQKENSKRWNPEIQ
YTSNYYKSNNVEFAVNTEGVYSEPRPIGTRYLTRNL

As labeled in AAV9 VP1 (SEQ ID NO: 1) above, the VR-I site is between amino acids 262 and 269 in the parental sequence (“NSTSGGSS”, SEQ ID NO: 4); the VR-II site is between amino acids 328 and 332 in the parental sequence (“NNGVK”, SEQ ID NO: 5); the VR-IV site is between amino acids 448 and 462 in the parental sequence (“SKTINGSGQNQQTLK”, SEQ ID NO: 6); the VR-V site is between amino acids 491 and 504 in the parental sequence (“TTVTQNNNSEFAWP”, SEQ ID NO: 7); the VR-VII site is between amino acids 547 and 557 in the parental sequence (“GTGRDNVDADK”, SEQ ID NO: 8); the VR-VIII site is between amino acids 581 and 595 in the parental sequence (“ATNHQSAQAQAQTGW”, SEQ ID NO: 9). In some embodiments, the engineered AAV9 capsid protein comprises a sequence that shares at least about 80% (e.g., at least 80%, 85%, 90%, 95%, 96%, 97%, 98%, 99%, or 100%) identity to SEQ ID NO: 1, excluding the VR-I, NR-am, VR-V, VR-V, VR-VII, and/or VR-VII site. In some embodiments, the engineered AAV9 capsid protein comprises a sequence that shares at least about 80% (e.g., at least 80%, 85%, 90%, 95%, 96%, 97%, 98%, 99%, or 100%) identity to SEQ ID NO: 1, excluding the VR-IV and/or VR-VIII site. In some embodiments, the engineered AAV9 capsid protein comprises a sequence that shares at least about 80% (e.g., at least 80%, 85%, 90%, 95%, 96%, 97%, 98%, 99%, or 100%) identity to SEQ ID NO: 2, excluding the VR-I, VR-II, VR-IV, VR-V, VR-VII, and/or VR-VIII site. In some embodiments, the engineered AAV9 capsid protein comprises a sequence that shares at least about 80% (e.g., at least 80%, 85%, 90%, 95%, 96%, 97%, 98%, 99%, or 100%) identity to SEQ ID NO: 2, excluding the VR-VI and/or VR-VIII site. In some embodiments, the engineered AAV9 capsid protein comprises a sequence that shares at least about 80% (e.g., at least 80%, 85%, 90%, 95%, 96%, 97%, 98%, 99%, or 100%) identity to SEQ ID NO: 3, excluding the VR-I, VR-II, VR-IV, VR-V, VR-VII, and/or VR-VIII site. In some embodiments, the engineered AAV9 capsid protein comprises a sequence that shares at least about 80% (e.g., at least 80%, 85%, 90%, 95%, 96%, 97%, 98%, 99%, or 100%) identity to SEQ ID NO: 3, excluding the VR-IV and/or VR-VIII site.

TABLE 5
Exemplary wild-type AAV capsid protein sequences

| | | SEQ |
|---|---|---|
| Name | Sequence | ID NO: |
| AAV9 VP1 | MAADGYLPDWLEDNLSEGIREWWALKPGAPQPKAN | 1 |
| (736 amino acids) | QQHQDNARGLVLPGYKYLGPGNGLDKGEPVNAADA | |
| | AALEHDKAYDQQLKAGDNPYLKYNHADAEFQERLK | |
| | EDTSFGGNLGRAVFQAKKRLLEPLGLVEEAAKTAPG | |
| | KKRPVEQSPQEPDSSAGIGKSGAQPAKKRLNFGQTG | |
| | DTESVPDPQPIGEPPAAPSGVGSLTMASGGGAPVADN | |
| | NEGADGVGSSSGNWHCDSQWLGDRVITTSTRTWAL | |
| | PTYNNHLYKQISNSTSGGSSNDNAYFGYSTPWGYFD | |
| | FNRFHCHFSPRDWQRLINNNWGFRPKRLNFKLFNIQ | |
| | VKEVTDNNGVKTIANNLTSTVQVFTDSDYQLPYVLG | |
| | SAHEGCLPPFPADVFMIPQYGYLTLNDGSQAVGRSSF | |
| | YCLEYFPSQMLRTGNNFQFSYEFENVPFHSSYAHSQS | |
| | LDRLMNPLIDQYLYYLSKTINGSGQNQQTLKFSVAGP | |
| | SNMAVQGRNYIPGPSYRQQRVSTTVTQNNNSEFAWP | |
| | GASSWALNGRNSLMNPGPAMASHKEGEDRFFPLSGS | |
| | LIFGKQGTGRDNVDADKVMITNEEEIKTTNPVATESY | |
| | GQVATNHQSAQAQAQTGWVQNQGILPGMVWQDRD | |
| | VYLQGPIWAKIPHTDGNFHPSPLMGGFGMKHPPPQIL | |
| | IKNTPVPADPPTAFNKDKLNSFITQYSTGQVSVEIEWE | |
| | LQKENSKRWNPEIQYTSNYYKSNNVEFAVNTEGVYS | |
| | EPRPIGTRYLTRNL | |
| AAV9 VP2 | TAPGKKRPVEQSPQEPDSSAGIGKSGAQPAKKRLNFG | 2 |
| (Amino acids 138- | QTGDTESVPDPQPIGEPPAAPSGVGSLTMASGGGAPV | |
| 736 of SEQ ID NO: | ADNNEGADGVGSSSGNWHCDSQWLGDRVITTSTRT | |
| 1) | WALPTYNNHLYKQISNSTSGGSSNDNAYFGYSTPWG | |
| | YFDFNRFHCHFSPRDWQRLINNNWGFRPKRLNFKLF | |
| | NIQVKEVTDNNGVKTIANNLTSTVQVFTDSDYQLPY | |
| | VLGSAHEGCLPPFPADVFMIPQYGYLTLNDGSQAVG | |
| | RSSFYCLEYFPSQMLRTGNNFQFSYEFENVPFHSSYA | |
| | HSQSLDRLMNPLIDQYLYYLSKTINGSGQNQQTLKFS | |
| | VAGPSNMAVQGRNYIPGPSYRQQRVSTTVTQNNNSE | |
| | FAWPGASSWALNGRNSLMNPGPAMASHKEGEDRFF | |
| | PLSGSLIFGKQGTGRDNVDADKVMITNEEEIKTTNPV | |
| | ATESYGQVATNHQSAQAQAQTGWVQNQGILPGMV | |
| | WQDRDVYLQGPIWAKIPHTDGNFHPSPLMGGFGMK | |
| | HPPPQILIKNTPVPADPPTAFNKDKLNSFITQYSTGQV | |
| | SVEIEWELQKENSKRWNPEIQYTSNYYKSNNVEFAV | |
| | NTEGVYSEPRPIGTRYLTRNL | |
| AAV9 VP3 | MASGGGAPVADNNEGADGVGSSSGNWHCDSQWLG | 3 |
| (Amino acids 203- | DRVITTSTRTWALPTYNNHLYKQISNSTSGGSSNDNA | |
| 736 of SEQ ID NO: | YFGYSTPWGYFDFNRFHCHFSPRDWQRLINNNWGFR | |
| 1) | PKRLNFKLFNIQVKEVTDNNGVKTIANNLTSTVQVFT | |
| | DSDYQLPYVLGSAHEGCLPPFPADVFMIPQYGYLTLN | |
| | DGSQAVGRSSFYCLEYFPSQMLRTGNNFQFSYEFEN | |
| | VPFHSSYAHSQSLDRLMNPLIDQYLYYLSKTINGSGQ | |
| | NQQTLKFSVAGPSNMAVQGRNYIPGPSYRQQRVSTT | |
| | VTQNNNSEFAWPGASSWALNGRNSLMNPGPAMASH | |
| | KEGEDRFFPLSGSLIFGKQGTGRDNVDADKVMITNEE | |
| | EIKTTNPVATESYGQVATNHQSAQAQAQTGWVQNQ | |
| | GILPGMVWQDRDVYLQGPIWAKIPHTDGNFHPSPLM | |
| | GGFGMKHPPPQILIKNTPVPADPPTAFNKDKLNSFITQ | |
| | YSTGQVSVEIEWELQKENSKRWNPEIQYTSNYYKSN | |
| | NVEFAVNTEGVYSEPRPIGTRYLTRNL | |
| AAV9 VR-I | NSTSGGSS | 4 |
| AAV9 VR-II | NNGVK | 5 |
| AAV9 VR-IV | SKTINGSGQNQQTLK | 6 |
| AAV0 VR-V | TTVTQNNNSEFAWP | 7 |
| AAV9 VR-VII | GTGRDNVDADK | 8 |
| AAV9 VR-VIII | ATNHQSAQAQAQTGW | 9 |
| AAV5 VP1 | MSFVDHPPDWLEEVGEGLREFLGLEAGPPKPKPNQQ | 10 |
| (724 amino acids) | HQDQARGLVLPGYNYLGPGNGLDRGEPVNRADEVA | |
| | REHDISYNEQLEAGDNPYLKYNHADAEFQEKLADDT | |
| | SFGGNLGKAVFQAKKRVLEPFGLVEEGAKTAPTGKR | |
| | IDDHFPKRKKARTEEDSKPSTSSDAEAGPSGSQQLQIP | |
| | AQPASSLGADTMSAGGGGPLGDNNQGADGVGNASG | |
| | DWHCDSTWMGDRVVTKSTRTWVLPSYNNHQYREIK | |
| | SGSVDGSNANAYFGYSTPWGYFDFNRFHSHWSPRD | |
| | WQRLINNYWGFRPRSLRVKIFNIQVKEVTVQDSTTTI | |
| | ANNLTSTVQVFTDDDYQLPYVVGNGTEGCLPAFPPQ | |
| | VFTLPQYGYATLNRDNTENPTERSSFFCLEYFPSKML | |
| | RTGNNFEFTYNFEEVPFHSSFAPSQNLFKLANPLVDQ | |
| | YLYRFVSTNNTGGVQFNKNLAGRYANTYKNWFPGP | |
| | MGRTQGWNLGSGVNRASVSAFATTNRMELEGASYQ | |
| | VPPQPNGMTNNLQGSNTYALENTMIFNSQPANPGTT | |
| | ATYLEGNMLITSESETQPVNRVAYNVGGQMATNNQS | |
| | STTAPATGTYNLQEIVPGSVWMERDVYLQGPIWAKIP | |
| | ETGAHFHPSPAMGGFGLKHPPPMMLIKNTPVPGNITS | |
| | FSDVPVSSFITQYSTGQVTVEMEWELKKENSKRWNP | |
| | EIQYTNNYNDPQFVDFAPDSTGEYRTTRPIGTRYLTR | |
| | PL | |
| AAV5 VP2 | TAPTGKRIDDHFPKRKKARTEEDSKPSTSSDAEAGPS | 11 |
| (Amino acids 137- | GSQQLQIPAQPASSLGADTMSAGGGGPLGDNNQGAD | |
| 724 of SEQ ID NO: | GVGNASGDWHCDSTWMGDRVVTKSTRTWVLPSYN | |
| 10) | NHQYREIKSGSVDGSNANAYFGYSTPWGYFDFNRFH | |
| | SHWSPRDWQRLINNYWGFRPRSLRVKIFNIQVKEVT | |
| | VQDSTTTIANNLTSTVQVFTDDDYQLPYVVGNGTEG | |
| | CLPAFPPQVFTLPQYGYATLNRDNTENPTERSSFFCLE | |
| | YFPSKMLRTGNNFEFTYNFEEVPFHSSFAPSQNLFKL | |
| | ANPLVDQYLYRFVSTNNTGGVQFNKNLAGRYANTY | |
| | KNWFPGPMGRTQGWNLGSGVNRASVSAFATTNRME | |
| | LEGASYQVPPQPNGMTNNLQGSNTYALENTMIFNSQ | |
| | PANPGTTATYLEGNMLITSESETQPVNRVAYNVGGQ | |
| | MATNNQSSTTAPATGTYNLQEIVPGSVWMERDVYL | |
| | QGPIWAKIPETGAHFHPSPAMGGFGLKHPPPMMLIKN | |
| | TPVPGNITSFSDVPVSSFITQYSTGQVTVEMEWELKK | |
| | ENSKRWNPEIQYTNNYNDPQFVDFAPDSTGEYRTTR | |
| | PIGTRYLTRPL | |
| AAV5 VP3 | MSAGGGGPLGDNNQGADGVGNASGDWHCDSTWM | 12 |
| (Amino acids 193- | GDRVVTKSTRTWVLPSYNNHQYREIKSGSVDGSNAN | |
| 724 of SEQ ID NO: | AYFGYSTPWGYFDFNRFHSHWSPRDWQRLINNYWG | |
| 10) | FRPRSLRVKIFNIQVKEVTVQDSTTTIANNLTSTVQVF | |
| | TDDDYQLPYVVGNGTEGCLPAFPPQVFTLPQYGYAT | |
| | LNRDNTENPTERSSFFCLEYFPSKMLRTGNNFEFTYN | |
| | FEEVPFHSSFAPSQNLFKLANPLVDQYLYRFVSTNNT | |
| | GGVQFNKNLAGRYANTYKNWFPGPMGRTQGWNLG | |
| | SGVNRASVSAFATTNRMELEGASYQVPPQPNGMTN | |
| | NLQGSNTYALENTMIFNSQPANPGTTATYLEGNMLIT | |
| | SESETQPVNRVAYNVGGQMATNNQSSTTAPATGTYN | |
| | LQEIVPGSVWMERDVYLQGPIWAKIPETGAHFHPSPA | |
| | MGGFGLKHPPPMMLIKNTPVPGNITSFSDVPVSSFITQ | |
| | YSTGQVTVEMEWELKKENSKRWNPEIQYTNNYNDP | |
| | QFVDFAPDSTGEYRTTRPIGTRYLTRPL | |
| AAV5 VR-I | SGSVD | 13 |
| AAV5 VR-II | QDSTT | 14 |
| AAV5 VR-IV | RFVSTNNTGGVQFNKNLAGRYANTY | 15 |
| AAV5 VR-V | LGSGVNRASVSAFA | 16 |
| AAV5 VR-VII | PANPGTTATYLEGN | 17 |
| AAV5 VR-VIII | ATNNQSSTTAPATGT | 18 |
| AAVrh.10 VP1 | MAADGYLPDWLEDNLSEGIREWWDLKPGAPKPKAN | 19 |
| (738 amino acids) | QQKQDDGRGLVLPGYKYLGPFNGLDKGEPVNAADA | |
| | AALEHDKAYDQQLKAGDNPYLRYNHADAEFQERLQ | |
| | EDTSFGGNLGRAVFQAKKRVLEPLGLVEEGAKTAPG | |
| | KKRPVEPSPQRSPDSSTGIGKKGQQPAKKRLNFGQTG | |
| | DSESVPDPQPIGEPPAGPSGLGSGTMAAGGGAPMAD | |
| | NNEGADGVGSSSGNWHCDSTWLGDRVITTSTRTWA | |
| | LPTYNNHLYKQISNGTSGGSTNDNTYFGYSTPWGYF | |
| | DFNRFHCHFSPRDWQRLINNNWGFRPKRLNFKLFNI | |
| | QVKEVTQNEGTKTIANNLTSTIQVFTDSEYQLPYVLG | |
| | SAHQGCLPPFPADVFMIPQYGYLTLNNGSQAVGRSSF | |
| | YCLEYFPSQMLRTGNNFEFSYQFEDVPFHSSYAHSQS | |
| | LDRLMNPLIDQYLYYLSRTQSTGGTAGTQQLLFSQA | |
| | GPNNMSAQAKNWLPGPCYRQQRVSTTLSQNNNSNF | |
| | AWTGATKYHLNGRDSLVNPGVAMATHKDDEERFFP | |
| | SSGVLMFGKQGAGKDNVDYSSVMLTSEEEIKTTNPV | |
| | ATEQYGVVADNLQQQNAAPIVGAVNSQGALPGMV | |
| | WQNRDVYLQGPIWAKIPHTDGNFHPSPLMGGFGLKH | |
| | PPPQILIKNTPVPADPPTTESQAKLASFITQYSTGQVSV | |
| | EIEWELQKENSKRWNPEIQYTSNYYKSTNVDFAVNT | |
| | DGTYSEPRPIGTRYLTRNL | |
| AAVrh.10 VP2 | TAPGKKRPVEPSPQRSPDSSTGIGKKGQQPAKKRLNF | 20 |
| (Amino acids 138- | GQTGDSESVPDPQPIGEPPAGPSGLGSGTMAAGGGAP | |
| 738 of SEQ ID NO: | MADNNEGADGVGSSSGNWHCDSTWLGDRVITTSTR | |
| 19) | TWALPTYNNHLYKQISNGTSGGSTNDNTYFGYSTPW | |
| | GYFDFNRFHCHFSPRDWQRLINNNWGFRPKRLNFKL | |
| | FNIQVKEVTQNEGTKTIANNLTSTIQVFTDSEYQLPYV | |
| | LGSAHQGCLPPFPADVFMIPQYGYLTLNNGSQAVGR | |
| | SSFYCLEYFPSQMLRTGNNFEFSYQFEDVPFHSSYAH | |
| | SQSLDRLMNPLIDQYLYYLSRTQSTGGTAGTQQLLFS | |
| | QAGPNNMSAQAKNWLPGPCYRQQRVSTTLSQNNNS | |
| | NFAWTGATKYHLNGRDSLVNPGVAMATHKDDEERF | |
| | FPSSGVLMFGKQGAGKDNVDYSSVMLTSEEEIKTTN | |
| | PVATEQYGVVADNLQQQNAAPIVGAVNSQGALPGM | |
| | VWQNRDVYLQGPIWAKIPHTDGNFHPSPLMGGFGLK | |
| | HPPPQILIKNTPVPADPPTTFSQAKLASFITQYSTGQVS | |
| | VEIEWELQKENSKRWNPEIQYTSNYYKSTNVDFAVN | |
| | TDGTYSEPRPIGTRYLTRNL | |
| AAVrh.10 VP3 | MAAGGGAPMADNNEGADGVGSSSGNWHCDSTWLG | 21 |
| (Amino acids 204- | DRVITTSTRTWALPTYNNHLYKQISNGTSGGSTNDNT | |
| 738 of SEQ ID NO: | YFGYSTPWGYFDFNRFHCHFSPRDWQRLINNNWGFR | |
| 19) | PKRLNFKLFNIQVKEVTQNEGTKTIANNLTSTIQVFTD | |
| | SEYQLPYVLGSAHQGCLPPFPADVFMIPQYGYLTLNN | |
| | GSQAVGRSSFYCLEYFPSQMLRTGNNFEFSYQFEDVP | |
| | FHSSYAHSQSLDRLMNPLIDQYLYYLSRTQSTGGTAG | |
| | TQQLLFSQAGPNNMSAQAKNWLPGPCYRQQRVSTT | |
| | LSQNNNSNFAWTGATKYHLNGRDSLVNPGVAMATH | |
| | KDDEERFFPSSGVLMFGKQGAGKDNVDYSSVMLTSE | |
| | EEIKTTNPVATEQYGVVADNLQQQNAAPIVGAVNSQ | |
| | GALPGMVWQNRDVYLQGPIWAKIPHTDGNFHPSPL | |
| | MGGFGLKHPPPQILIKNTPVPADPPTTFSQAKLASFIT | |
| | QYSTGQVSVEIEWELQKENSKRWNPEIQYTSNYYKS | |
| | TNVDFAVNTDGTYSEPRPIGTRYLTRNL | |
| AAVrh.10 VR-I | NGTSG | 22 |
| AAVrh.10 VR-II | NEGTK | 23 |
| AAVrh.10 VR-IV | SRTQSTGGTAGTQQLL | 24 |
| AAVrh.10 VR-V | TTLSQNNNSNFAWT | 25 |
| AAVrh.10 VR-VII | GAGKDNVDYSS | 26 |
| AAVrh.10 VR-VIII | ADNLQQQNAAPIVGA | 27 |
| AAVrh.74 VP1 | MAADGYLPDWLEDNLSEGIREWWDLKPGAPKPKAN | 28 |
| (738 amino acids) | QQKQDNGRGLVLPGYKYLGPFNGLDKGEPVNAADA | |
| | AALEHDKAYDQQLQAGDNPYLRYNHADAEFQERLQ | |
| | EDTSFGGNLGRAVFQAKKRVLEPLGLVESPVKTAPG | |
| | KKRPVEPSPQRSPDSSTGIGKKGQQPAKKRLNFGQTG | |
| | DSESVPDPQPIGEPPAGPSGLGSGTMAAGGGAPMAD | |
| | NNEGADGVGSSSGNWHCDSTWLGDRVITTSTRTWA | |
| | LPTYNNHLYKQISNGTSGGSTNDNTYFGYSTPWGYF | |
| | DFNRFHCHFSPRDWQRLINNNWGFRPKRLNFKLFNI | |
| | QVKEVTQNEGTKTIANNLTSTIQVFTDSEYQLPYVLG | |
| | SAHQGCLPPFPADVFMIPQYGYLTLNNGSQAVGRSSF | |
| | YCLEYFPSQMLRTGNNFEFSYNFEDVPFHSSYAHSQS | |
| | LDRLMNPLIDQYLYYLSRTQSTGGTAGTQQLLFSQA | |
| | GPNNMSAQAKNWLPGPCYRQQRVSTTLSQNNNSNF | |
| | AWTGATKYHLNGRDSLVNPGVAMATHKDDEERFFP | |
| | SSGVLMFGKQGAGKDNVDYSSVMLTSEEEIKTTNPV | |
| | ATEQYGVVADNLQQQNAAPIVGAVNSQGALPGMV | |
| | WQNRDVYLQGPIWAKIPHTDGNFHPSPLMGGFGLKH | |
| | PPPQILIKNTPVPADPPTTFNQAKLASFITQYSTGQVS | |
| | VEIEWELQKENSKRWNPEIQYTSNYYKSTNVDFAVN | |
| | TEGTYSEPRPIGTRYLTRNL | |
| AAVrh.74 VP2 | TAPGKKRPVEPSPQRSPDSSTGIGKKGQQPAKKRLNF | 29 |
| (Amino acids 138- | GQTGDSESVPDPQPIGEPPAGPSGLGSGTMAAGGGAP | |
| 738 of SEQ ID NO: | MADNNEGADGVGSSSGNWHCDSTWLGDRVITTSTR | |
| 19 | TWALPTYNNHLYKQISNGTSGGSTNDNTYFGYSTPW | |
| | GYFDFNRFHCHFSPRDWQRLINNNWGFRPKRLNFKL | |
| | FNIQVKEVTQNEGTKTIANNLTSTIQVFTDSEYQLPYV | |
| | LGSAHQGCLPPFPADVFMIPQYGYLTLNNGSQAVGR | |
| | SSFYCLEYFPSQMLRTGNNFEFSYNFEDVPFHSSYAH | |
| | SQSLDRLMNPLIDQYLYYLSRTQSTGGTAGTQQLLFS | |
| | QAGPNNMSAQAKNWLPGPCYRQQRVSTTLSQNNNS | |
| | NFAWTGATKYHLNGRDSLVNPGVAMATHKDDEERF | |
| | FPSSGVLMFGKQGAGKDNVDYSSVMLTSEEEIKTTN | |
| | PVATEQYGVVADNLQQQNAAPIVGAVNSQGALPGM | |
| | VWQNRDVYLQGPIWAKIPHTDGNFHPSPLMGGFGLK | |
| | HPPPQILIKNTPVPADPPTTFNQAKLASFITQYSTGQV | |
| | SVEIEWELQKENSKRWNPEIQYTSNYYKSTNVDFAV | |
| | NTEGTYSEPRPIGTRYLTRNL | |
| AAVrh.74 VP3 | MAAGGGAPMADNNEGADGVGSSSGNWHCDSTWLG | 30 |
| (Amino acids 204- | DRVITTSTRTWALPTYNNHLYKQISNGTSGGSTNDNT | |
| 738 of SEQ ID NO: | YFGYSTPWGYFDFNRFHCHFSPRDWQRLINNNWGFR | |
| 19) | PKRLNFKLFNIQVKEVTQNEGTKTIANNLTSTIQVFTD | |
| | SEYQLPYVLGSAHQGCLPPFPADVFMIPQYGYLTLNN | |
| | GSQAVGRSSFYCLEYFPSQMLRTGNNFEFSYNFEDVP | |
| | FHSSYAHSQSLDRLMNPLIDQYLYYLSRTQSTGGTAG | |
| | TQQLLFSQAGPNNMSAQAKNWLPGPCYRQQRVSTT | |
| | LSQNNNSNFAWTGATKYHLNGRDSLVNPGVAMATH | |
| | KDDEERFFPSSGVLMFGKQGAGKDNVDYSSVMLTSE | |
| | EEIKTTNPVATEQYGVVADNLQQQNAAPIVGAVNSQ | |
| | GALPGMVWQNRDVYLQGPIWAKIPHTDGNFHPSPL | |
| | MGGFGLKHPPPQILIKNTPVPADPPTTFNQAKLASFIT | |
| | QYSTGQVSVEIEWELQKENSKRWNPEIQYTSNYYKS | |
| | TNVDFAVNTEGTYSEPRPIGTRYLTRNL | |
| AAVrh.74 VR-I | NGTSG | 22 |
| AAVrh.74 VR-II | NEGTK | 23 |
| AAVrh.74 VR-IV | SRTQSTGGTAGTQQLL | 24 |
| AAVrh.74 VR-V | TTLSQNNNSNFAWT | 25 |
| AAVrh.74 VR-VII | GAGKDNVDYSS | 26 |
| AAVrh.74 VR-VIII | ADNLQQQNAAPIVGA | 27 |

### E. AAV5

In some embodiments, the engineered capsid protein is an engineered AAV5 capsid protein comprising a non-naturally occurring amino acid motif (e.g., a substitution motif, an insertion motif, or both) compared to the wild-type AAV5 capsid protein.

The wild-type AAV5 VP1 has the amino acid sequence of SEQ ID NO: 10; the wild-type AAV5 VP2 has the amino acid sequence of SEQ ID NO: 11; the wild-type AAV5 VP3 has the amino acid sequence of SEQ ID NO: 12, as shown below and provided in Table 5. The N-terminal residue of VP1, VP2, and VP3, as well as the variable region (VR) sites (e.g., VR-I, VR-II, VR-IV, VR-V, VR-VII and VR-VIII), are indicated in bold, underlined, and enlarged text in the sequence of full-length VP1 (SEQ ID NO: 10). In some embodiments, the engineered AAV5 capsid protein comprises a sequence that shares at least about 80% (e.g., at least 80%, 85%, 90%, 95%, 96%, 97%, 98%, 99%, or 100%) identity to SEQ ID NO: 10, for example, as compared using a sequence alignment algorism, e.g., BLAST® provided by the National Center for Biotechnology Information (NCBI). In some embodiments, the engineered AAV5 capsid protein comprises a sequence that shares at least about 80% (e.g., at least 80%, 85%, 90%, 95%, 96%, 97%, 98%, 99%, or 100%) identity to SEQ ID NO: 11. In some embodiments, the engineered AAV5 capsid protein comprises a sequence that shares at least about 80% (e.g., at least 80%, 85%, 90%, 95%, 96%, 97%, 98%, 99%, or 100%) identity to SEQ ID NO: 12.

Annotated WT AAV5 VP1 Sequence (SEQ ID NO: 10)

MSFVDHPPDWLEEVGEGLREFLGLEAGPPKPKPNQQHQDQARGLVLPGYNYLGPGNGLDRGEPVNR
ADEVAREHDISYNEQLEAGDNPYLKYNHADAEFQEKLADDTSFGGNLGKAVFQAKKRVLEPFGLVEE
GAKTAPTGKRIDDHFPKRKKARTEEDSKPSTSSDAEAGPSGSQQLQIPAQPASSLGADTMSAGGGG
PLGDNNQGADGVGNASGDWHCDSTWMGDRVVTKSTRTWVLPSYNNHQYREIKSGSVDGSNANAYF
GYSTPWGYFDFNRFHSHWSPRDWQRLINNYWGFRPRSLRVKIFNIQVKEVTVQDSTTTIANNLTS
TVQVFTDDDYQLPYVVGNGTEGCLPAFPPQVFTLPQYGYATLNRDNTENPTERSSFFCLEYFPSKML
RTGNNFEFTYNFEEVPFHSSFAPSQNLFKLANPLVDQYLYRFVSTNNTGGVQFNKNLAGRY
ANTYKNWFPGPMGRTQGWNLGSGVNRASVSAFATTNRMELEGASYQVPPQPNGMINNLQGSN
TYALENTMIFNSQPANPGTTATYLEGNMLITSESETQPVNRVAYNVGGQMATNNQSSTTA
PATGTYNLQEIVPGSVWMERDVYLQGPIWAKIPETGAHFHPSPAMGGFGLKHPPPMMLIKNTPVP
GNITSFSDVPVSSFITQYSTGQVIVEMEWELKKENSKRWNPEIQYTNNYNDPQFVDFAPDSTGEYRT
TRPIGTRYLTRPL

As labeled in AAV5 VP1 (SEQ ID NO: 10) above, the VR-I site is between amino acids 252 and 256 in the parental sequence (“SGSVD”, SEQ ID NO: 13); the VR-II site is between amino acids 317 and 321 in the parental sequence (“QDSTT”, SEQ ID NO: 14); the VR-IV site is between amino acids 437 and 461 in the parental sequence (“RFVSTNNTGGVQFNKNLAGRYANTY”, SEQ ID NO: 15); the VR-V site is between amino acids 477 and 490 in the parental sequence (“LGSGVNRASVSAFA”, SEQ ID NO: 16); the VR-VII site is between amino acids 533 and 546 in the parental sequence (“PANPGTTATYLEGN”, SEQ ID NO: 17); the VR-VIII site is between amino acids 570 and 584 in the parental sequence (“ATNNQSSTTAPATGT”, SEQ ID NO: 18). In some embodiments, the engineered AAV5 capsid protein comprises a sequence that shares at least about 80% (e.g., at least 80%, 85%, 90%, 95%, 96%, 97%, 98%, 99%, or 100%) identity to SEQ ID NO: 10, excluding the VR-I, VR-II, VR-IV, VR-V, VR-VII, and/or VR-VIII site. In some embodiments, the engineered AAV5 capsid protein comprises a sequence that shares at least about 80% (e.g., at least 80%, 85%, 90%, 95%, 96%, 97%, 98%, 99%, or 100%) identity to SEQ ID NO: 10, excluding the VR-IV and/or VR-VIII site. In some embodiments, the engineered AAV5 capsid protein comprises a sequence that shares at least about 80% (e.g., at least 80%, 85%, 90%, 95%, 96%, 97%, 98%, 99%, or 100%) identity to SEQ ID NO: 11, excluding the VR-I, VR-II, VR-IV, VR-V, VR-VII, and/or VR-VIII site. In some embodiments, the engineered AAV5 capsid protein comprises a sequence that shares at least about 80% (e.g., at least 80%, 85%, 90%, 95%, 96%, 97%, 98%, 99%, or 100%) identity to SEQ ID NO: 11, excluding the VR-VI and/or VR-VIII site. In some embodiments, the engineered AAV5 capsid protein comprises a sequence that shares at least about 80% (e.g., at least 80%, 85%, 90%, 95%, 96%, 97%, 98%, 99%, or 100%) identity to SEQ ID NO: 12, excluding the VR-I, VR-II, VR-IV, VR-V, VR-VII, and/or VR-VIII site. In some embodiments, the engineered AAV5 capsid protein comprises a sequence that shares at least about 80% (e.g., at least 80%, 85%, 90%, 95%, 96%, 97%, 98%, 99%, or 100%) identity to SEQ ID NO: 12, excluding the VR-IV and/or VR-VIII site.

### F. AAVrh.10

In some embodiments, the engineered capsid protein is an engineered AAVrh.10 capsid protein comprising a non-naturally occurring amino acid motif (e.g., a substitution motif, an insertion motif, or both) compared to the wild-type AAVrh.10 capsid protein.

The wild-type AAVrh.10 VP1 has the amino acid sequence of SEQ ID NO: 19; the wild-type AAVrh.10 VP2 has the amino acid sequence of SEQ ID NO: 20; the wild-type AAVrh.10 VP3 has the amino acid sequence of SEQ ID NO: 21, as shown below and provided in Table 5. The N-terminal residue of VP1, VP2, and VP3, as well as the variable region (VR) sites (e.g., VR-I, VR-II, VR-IV, VR-V, VR-VII and VR-VIII), are indicated in bold, underlined, and enlarged text in the sequence of full-length VP1 (SEQ ID NO: 19), for example, as compared using a sequence alignment algorism, e.g., BLAST® provided by the National Center for Biotechnology Information (NCBI). In some embodiments, the engineered AAVrh.10 capsid protein comprises a sequence that shares at least about 80% (e.g., at least 80%, 85%, 90%, 95%, 96%, 97%, 98%, 99%, or 100%) identity to SEQ ID NO: 19. In some embodiments, the engineered AAVrh.10 capsid protein comprises a sequence that shares at least about 80% (e.g., at least 80%, 85%, 90%, 95%, 96%, 97%, 98%, 99%, or 100%) identity to SEQ ID NO: 20. In some embodiments, the engineered AAVrh.10 capsid protein comprises a sequence that shares at least about 80% (e.g., at least 80%, 85%, 90%, 95%, 96%, 97%, 98%, 99%, or 100%) identity to SEQ ID NO: 21.

Annotated WT AAVrh.10 VP1 Sequence (SEQ ID NO: 19)

MAADGYLPDWLEDNLSEGIREWWDLKPGAPKPKANQQKQDDGRGLVLPGYKYLGPFNGLDKGEPVN
AADAAALEHDKAYDQQLKAGDNPYLRYNHADAEFQERLQEDTSFGGNLGRAVFQAKKRVLEPLGLVE
EGAKTAPGKKRPVEPSPQRSPDSSTGIGKKGQQPAKKRLNFGQTGDSESVPDPQPIGEPPAGPSGL
GSGTMAAGGGAPMADNNEGADGVGSSSGNWHCDSTWLGDRVITTSTRTWALPTYNNHLYKQISNGT
SGGSTNDNTYFGYSTPWGYFDFNRFHCHESPRDWQRLINNNWGFRPKRLNFKLFNIQVKEVTQNE
GTKTIANNLTSTIQVETDSEYQLPYVLGSAHQGCLPPFPADVFMIPQYGYLTLNNGSQAVGRSSFY
CLEYFPSQMLRTGNNFEFSYQFEDVPFHSSYAHSQSLDRLMNPLIDQYLYYLSRTQSTGGTAG
TQQLLFSQAGPNNMSAQAKNWLPGPCYRQQRVSTTLSQNNNSNFAWTGATKYHLNGRDSLV
NPGVAMATHKDDEERFFPSSGVLMFGKQGAGKDNVDYSSVMLTSEEEIKTTNPVATEQYGVVA
DNLQQQNAAPIVGAVNSQGALPGMVWQNRDVYLQGPIWAKIPHTDGNFHPSPLMGGFGLKHPP
PQILIKNTPVPADPPTTFSQAKLASFITQYSTGQVSVEIEWELQKENSKRWNPEIQYTSNYYKSTNV
DFAVNTDGTYSEPRPIGTRYLTRNL

As labeled in AAVrh.10 VP1 (SEQ ID NO: 19) above, the VR-I site is between amino acids 263 and 267 in the parental sequence (“NGTSG”, SEQ ID NO: 22); the VR-II site is between amino acids 329 and 333 in the parental sequence (“NEGTK”, SEQ ID NO: 23); the VR-IV site is between amino acids 449 and 464 in the parental sequence (“SRTQSTGGTAGTQQLL”, SEQ ID NO: 24); the VR-V site is between amino acids 493 and 506 in the parental sequence (“TTLSQNNNSNFAWT”, SEQ ID NO: 25); the VR-VII site is between amino acids 549 and 559 in the parental sequence (“GAGKDNVDYSS”, SEQ ID NO: 26); the VR-VIII site is between amino acids 583 and 597 in the parental sequence (“ADNLQQQNAAPIVGA”, SEQ ID NO: 27). In some embodiments, the engineered AAVrh.10 capsid protein comprises a sequence that shares at least about 80% (e.g., at least 80%, 85%, 90%, 95%, 96%, 97%, 98%, 99%, or 100%) identity to SEQ ID NO: 19, excluding the VR-I, VR-II, VR-IV, VR-V, VR-VII, and/or VR-VIII site. In some embodiments, the engineered AAVrh.10 capsid protein comprises a sequence that shares at least about 80% (e.g., at least 80%, 85%, 90%, 95%, 96%, 97%, 98%, 99%, or 100%) identity to SEQ ID NO: 19, excluding the VR-IV and/or VR-VIII site. In some embodiments, the engineered AAVrh.10 capsid protein comprises a sequence that shares at least about 80% (e.g., at least 80%, 85%, 90%, 95%, 96%, 97%, 98%, 99%, or 100%) identity to SEQ ID NO: 20, excluding the VR-I, VR-II, VR-IV, VR-V, VR-VII, and/or VR-VIII site. In some embodiments, the engineered AAVrh.10 capsid protein comprises a sequence that shares at least about 80% (e.g., at least 80%, 85%, 90%, 95%, 96%, 97%, 98%, 99%, or 100%) identity to SEQ ID NO: 20, excluding the VR-VI and/or VR-VIII site. In some embodiments, the engineered AAVrh.10 capsid protein comprises a sequence that shares at least about 80% (e.g., at least 80%, 85%, 90%, 95%, 96%, 97%, 98%, 99%, or 100%) identity to SEQ ID NO: 21, excluding the VR-I, VR-II, VR-IV, VR-V, VR-VII, and/or VR-VIII site. In some embodiments, the engineered AAVrh.10 capsid protein comprises a sequence that shares at least about 80% (e.g., at least 80%, 85%, 90%, 95%, 96%, 97%, 98%, 99%, or 100%) identity to SEQ ID NO: 21, excluding the VR-IV and/or VR-VIII site.

### G. AAVrh.74

In some embodiments, the engineered capsid protein is an engineered AAVrh.74 capsid protein comprising a non-naturally occurring amino acid motif (e.g., a substitution motif, an insertion motif, or both) compared to the wild-type AAVrh.74 capsid protein.

The wild-type AAVrh.74 VP1 has the amino acid sequence of SEQ ID NO: 28; the wild-type AAVrh.74 VP2 has the amino acid sequence of SEQ ID NO: 29; the wild-type AAVrh.74 VP3 has the amino acid sequence of SEQ ID NO: 30, as shown below and provided in Table 5. The N-terminal residue of VP1, VP2, and VP3, as well as the variable region (VR) sites (e.g., VR-I, VR-II, VR-IV, VR-V, VR-VII and VR-VIII), are indicated in bold, underlined, and enlarged text in the sequence of full-length VP1 (SEQ ID NO: 28). In some embodiments, the engineered AAVrh.74 capsid protein comprises a sequence that shares at least about 80% (e.g., at least 80%, 85%, 90%, 95%, 96%, 97%, 98%, 99%, or 100%) identity to SEQ ID NO: 28, for example, as compared using a sequence alignment algorism, e.g., BLAST® provided by the National Center for Biotechnology Information (NCBI). In some embodiments, the engineered AAVrh.74 capsid protein comprises a sequence that shares at least about 80% (e.g., at least 80%, 85%, 90%, 95%, 96%, 97%, 98%, 99%, or 100%) identity to SEQ ID NO: 29. In some embodiments, the engineered AAVrh.74 capsid protein comprises a sequence that shares at least about 80% (e.g., at least 80%, 85%, 90%, 95%, 96%, 97%, 98%, 99%, or 100%) identity to SEQ ID NO: 30.

Annotated WT AAVrh.74 VP1 Sequence (SEQ ID NO: 28)

MAADGYLPDWLEDNLSEGIREWWDLKPGAPKPKANQQKQDNGRGLVLPGYKYLGPFNGLDKGEPVN
AADAAALEHDKAYDQQLQAGDNPYLRYNHADAEFQERLQEDTSFGGNLGRAVFQAKKRVLEPLGLVE
SPVKTAPGKKRPVEPSPQRSPDSSTGIGKKGQQPAKKRLNFGQTGDSESVPDPQPIGEPPAGPSGL
GSGTMAAGGGAPMADNNEGADGVGSSSGNWHCDSTWLGDRVITTSTRTWALPTYNNHLYKQISNGT
SGGSTNDNTYFGYSTPWGYFDFNRFHCHFSPRDWQRLINNNWGFRPKRLNFKLFNIQVKEVTQNE
GTKTIANNLISTIQVFTDSEYQLPYVLGSAHQGCLPPFPADVFMIPQYGYLTLNNGSQAVGRSSFY
CLEYFPSQMLRTGNNFEFSYNFEDVPFHSSYAHSQSLDRLMNPLIDQYLYYLSRTQSTGGTAG
TQQLLFSQAGPNNMSAQAKNWLPGPCYRQQRVSTTLSQNNNSNFAWTGATKYHLNGRDSLV
NPGVAMATHKDDEERFFPSSGVLMFGKQGAGKDNVDYSSVMLTSEEEIKTTNPVATEQYGVVA
DNLQQQNAAPIVGAVNSQGALPGMVWQNRDVYLQGPIWAKIPHTDGNFHPSPLMGGFGLKHPP
PQILIKNTPVPADPPTTFNQAKLASFITQYSTGQVSVEIEWELQKENSKRWNPEIQYTSNYYKSTNV
DFAVNTEGTYSEPRPIGTRYLTRNL

As labeled in AAVrh.74 VP1 (SEQ ID NO: 28) above, the VR-I site is between amino acids 263 and 267 in the parental sequence (“NGTSG”, SEQ ID NO: 22); the VR-II site is between amino acids 329 and 333 in the parental sequence (“NEGTK”, SEQ ID NO: 23); the VR-IV site is between amino acids 449 and 464 in the parental sequence (“SRTQSTGGTAGTQQLL”, SEQ ID NO: 24); the VR-V site is between amino acids 493 and 506 in the parental sequence (“TTLSQNNNSNFAWT”, SEQ ID NO: 25); the VR-VII site is between amino acids 549 and 559 in the parental sequence (“GAGKDNVDYSS”, SEQ ID NO: 26); the VR-VIII site is between amino acids 583 and 597 in the parental sequence (“ADNLQQQNAAPIVGA”, SEQ ID NO: 27). In some embodiments, the engineered AAVrh.74 capsid protein comprises a sequence that shares at least about 80% (e.g., at least 80%, 85%, 90%, 95%, 96%, 97%, 98%, 99%, or 100%) identity to SEQ ID NO: 28, excluding the VR-I, VR-II, VR-IV, VR-V, VR-VII, and/or VR-VIII site. In some embodiments, the engineered AAVrh.74 capsid protein comprises a sequence that shares at least about 80% (e.g., at least 80%, 85%, 90%, 95%, 96%, 97%, 98%, 99%, or 100%) identity to SEQ ID NO: 28, excluding the VR-IV and/or VR-VIII site. In some embodiments, the engineered AAVrh.74 capsid protein comprises a sequence that shares at least about 80% (e.g., at least 80%, 85%, 90%, 95%, 96%, 97%, 98%, 99%, or 100%) identity to SEQ ID NO: 29, excluding the VR-I, VR-II, VR-IV, VR-V, VR-VII, and/or VR-VIII site. In some embodiments, the engineered AAVrh.74 capsid protein comprises a sequence that shares at least about 80% (e.g., at least 80%, 85%, 90%, 95%, 96%, 97%, 98%, 99%, or 100%) identity to SEQ ID NO: 29, excluding the VR-VI and/or VR-VIII site. In some embodiments, the engineered AAVrh.74 capsid protein comprises a sequence that shares at least about 80% (e.g., at least 80%, 85%, 90%, 95%, 96%, 97%, 98%, 99%, or 100%) identity to SEQ ID NO: 30, excluding the VR-I, VR-II, VR-IV, VR-V, VR-VII, and/or VR-VIII site. In some embodiments, the engineered AAVrh.74 capsid protein comprises a sequence that shares at least about 80% (e.g., at least 80%, 85%, 90%, 95%, 96%, 97%, 98%, 99%, or 100%) identity to SEQ ID NO: 30, excluding the VR-IV and/or VR-VIII site.

### H. Chimeric AAV Capsid Proteins

In some embodiments, the engineered capsid protein is an engineered chimeric capsid protein comprising a non-naturally occurring amino acid motif (e.g., a substitution motif, an insertion motif, or both) compared to a parental chimeric capsid protein described herein. The parental chimeric capsid can be any chimeric capsid protein described herein or known in the art.

In some embodiments, the parental chimeric capsid protein is a AAV5/9 chimeric capsid protein. In some embodiments, the AAV5/9 chimeric capsid protein sequence is at least about 50%, 55%, 60%, 65%, 70%, 75%, 80%, 85%, 90%, 95%, 96%, 97%, 98%, 99%, or 99.5% identical to the AAV9 capsid protein sequence (SEQ ID NO: 1), for example, as compared using a sequence alignment algorism, e.g., BLAST® provided by the National Center for Biotechnology Information (NCBI). In some embodiments, the C-terminal 500 residues of the AAV5/9 chimeric capsid protein sequence is at least about 80%, 85%, 90%, 95%, 96%, 97%, 98%, 99%, 99.5%, or 100% identical to the C-terminal 500 residues of the AAV9 capsid protein sequence (SEQ ID NO: 1). In some embodiments, the residue at the position equivalent to Q688 of the AAV9 capsid protein sequence (SEQ ID NO: 1) is a lysine (K) in the chimeric capsid protein.

In some embodiments, the AAV5/9 chimeric capsid protein comprises at least 1, 2, 3, 4, 5 or more polypeptide segments that are derived from an AAV5 capsid protein. In some embodiments, the AAV5/9 chimeric capsid protein comprises at least 1, 2, 3, 4, 5 or more polypeptide segments that are derived from an AAV9 capsid protein. In some embodiments, at least one polypeptide segment is derived from the AAV5 capsid protein and at least one polypeptide segment is derived from the AAV9 capsid protein.

In some embodiments, the first 250 residues at the N-terminus of the AAV5/9 chimeric capsid protein comprise one or more AAV5 capsid derived polypeptide segments. In some embodiments, the first 225 residues at the N-terminus of the AAV5/9 chimeric capsid protein comprise one or more AAV5 capsid derived polypeptide segments. In some embodiments, the first 200 residues at the N-terminus of the AAV5/9 chimeric capsid protein comprise one or more AAV5 capsid derived polypeptide segments. In some embodiments, the first 150 residues at the N-terminus of the AAV5/9 chimeric capsid protein comprise one or more AAV5 capsid derived polypeptide segments. In some embodiments, the first 100 residues at the N-terminus of the AAV5/9 chimeric capsid protein comprise one or more AAV5 capsid derived polypeptide segments. In some embodiments, the first 50 residues at the N-terminus of the AAV5/9 chimeric capsid protein comprise one or more AAV5 capsid derived polypeptide segments. In some embodiments, each of the one or more AAV5 capsid derived polypeptide segments has at least about 80%, 85%, 90%, 95%, 96%, 97%, 98%, 99%, 99.5%, or 100% identity to the corresponding AAV5 capsid sequence.

In some embodiments, residues 50-250 of the AAV5/9 chimeric capsid protein comprise one or more AAV5 capsid derived polypeptide segments. In some embodiments, residues 50-200 of the AAV5/9 chimeric capsid protein comprise one or more AAV5 capsid derived polypeptide segments. In some embodiments, residues 50-150 of the AAV5/9 chimeric capsid protein comprise one or more AAV5 capsid derived polypeptide segments. In some embodiments, residues 100-250 of the AAV5/9 chimeric capsid protein comprise one or more AAV5 capsid derived polypeptide segments. In some embodiments, residues 100-200 of the AAV5/9 chimeric capsid protein comprise one or more AAV5 capsid derived polypeptide segments. In some embodiments, residues 150-250 of the AAV5/9 chimeric capsid protein comprise one or more AAV5 capsid derived polypeptide segments. In some embodiments, each of the one or more AAV5 capsid derived polypeptide segments has at least about 80%, 85%, 90%, 95%, 96%, 97%, 98%, 99%, 99.5%, or 100% identity to the corresponding AAV5 capsid sequence.

In some embodiments, the last 100 residues at the C-terminus of the AAV5/9 chimeric capsid protein comprise one or more AAV5 capsid derived polypeptide segments. In some embodiments, the last 50 residues at the C-terminus of the AAV5/9 chimeric capsid protein comprise one or more AAV5 capsid derived polypeptide segments. In some embodiments, each of the one or more AAV5 capsid derived polypeptide segments has at least about 80%, 85%, 90%, 95%, 96%, 97%, 98%, 99%, 99.5%, or 100% identity to the corresponding AAV5 capsid sequence. In some embodiments, the AAV5/9 chimeric capsid protein comprises one or more AAV5 capsid derived polypeptide segments at or near the N-terminus of the chimeric capsid protein, as described above, and one or more AAV5 capsid derived polypeptide segments at or near the C-terminus of the chimeric capsid protein, as described in this paragraph.

In some embodiments, the AAV5/9 chimeric capsid protein comprises, in N-terminal to C-terminal order, a first polypeptide segment having a sequence at least about 80%, 85%, 90%, 95%, 96%, 97%, 98%, 99%, 99.5%, or 100% identical to SEQ ID NO: 31 or at least about 80%, 85%, 90%, 95%, 96%, 97%, 98%, 99%, 99.5%, or 100% identical to SEQ ID NO: 32; a second polypeptide segment having sequence at least about 80%, 85%, 90%, 95%, 96%, 97%, 98%, 99%, 99.5%, or 100% identical to SEQ ID NO: 33 or at least about 80%, 85%, 90%, 95%, 96%, 97%, 98%, 99%, 99.5%, or 100% identical to SEQ ID NO: 34; a third polypeptide segment having sequence at least about 80%, 85%, 90%, 95%, 96%, 97%, 98%, 99%, 99.5%, or 100% identical to SEQ ID NO: 35 or at least about 80%, 85%, 90%, 95%, 96%, 97%, 98%, 99%, 99.5%, or 100% identical to SEQ ID NO: 36; a fourth polypeptide segment having sequence at least about 80%, 85%, 90%, 95%, 96%, 97%, 98%, 99%, 99.5%, or 100% identical to SEQ ID NO: 37 or at least about 80%, 85%, 90%, 95%, 96%, 97%, 98%, 99%, 99.5%, or 100% identical to SEQ ID NO: 38; and/or a fifth polypeptide segment having sequence at least about 80%, 85%, 90%, 95%, 96%, 97%, 98%, 99%, 99.5%, or 100% identical to SEQ ID NO: 39 or at least about 80%, 85%, 90%, 95%, 96%, 97%, 98%, 99%, 99.5%, or 100% identical to SEQ ID NO: 40. In some embodiments, at least one polypeptide segment is derived from the AAV5 capsid protein and at least one polypeptide segment is derived from the AAV9 capsid protein.

TABLE 6
Exemplary AAV5 or AAV9 derived polypeptide sequences

| | | SEQ |
|---|---|---|
| Name | Sequence | ID NO: |
| AAV9 derived | MAADGYLPDWLEDNLSEGIREWWALKPGAPQPK | 31 |
| polypeptide segment 1 | ANQQHQDNARGLVLPGY | |
| AAV5 derived | MSFVDHPPDWLEEVGEGLREFLGLEAGPPKPKPN | 32 |
| polypeptide segment 1 | QQHQDQARGLVLPGY | |
| AAV9 derived | KYLGPGNGLDKGEPVNAADAAALEHDKAYDQQ | 33 |
| polypeptide segment 2 | LK | |
| AAV5 derived | NYLGPGNGLDRGEPVNRADEVAREHDISYNEQLE | 34 |
| polypeptide segment 2 | | |
| AAV9 derived | AGDNPYLKYNHADAEFQERLKEDTSFGGNLGRA | 35 |
| polypeptide segment 3 | VFQAKKRLLEP | |
| AAV5 derived | AGDNPYLKYNHADAEFQEKLADDTSFGGNLGKA | 36 |
| polypeptide segment 3 | VFQAKKRVLEP | |
| AAV9 derived | LGLVEEAAKTAPGKKRPVEQSPQEPDSSAGIGKSG | 37 |
| polypeptide segment 4 | AQPAKKRLNFGQTGDTESVPDPQPIGEPPAAPSGV | |
| | GSLTMASGGGAPVA | |
| AAV5 derived | FGLVEEGAKTAPTGKRIDDHFPKRKKARTEEDSK | 38 |
| polypeptide segment 4 | PSTSSDAEAGPSGSQQLQIPAQPASSLGADTMSAG | |
| | GGGPLG | |
| AAV9 derived | DNNEGADGVGSSSGNWHCDSQWLGDRVITTSTR | 39 |
| polypeptide segment 5 | TWALPTYNNHLYKQISNSTSGGSSNDNAYFGYST | |
| | PWGYFDFNRFHCHFSPRDWQRLINNNWGFRPKR | |
| | LNFKLFNIQVKEVTDNNGVKTIANNLTSTVQVFT | |
| | DSDYQLPYVLGSAHEGCLPPFPADVFMIPQYGYL | |
| | TLNDGSQAVGRSSFYCLEYFPSQMLRTGNNFQFS | |
| | YEFENVPFHSSYAHSQSLDRLMNPLIDQYLYYLS | |
| | KTINGSGQNQQTLKFSVAGPSNMAVQGRNYIPGP | |
| | SYRQQRVSTTVTQNNNSEFAWPGASSWALNGRN | |
| | SLMNPGPAMASHKEGEDRFFPLSGSLIFGKQGTgr | |
| | dnvDADKVMITNEEEIKTTNPVATESYGQVATNHQ | |
| | SAQAQAQTGWVQNQGILPGMVWQDRDVYLQGP | |
| | IWAKIPHTDGNFHPSPLMGGFGMKHPPPQILIKNT | |
| | PVPADPPTAFNKDKLNSFITQYSTGQVSVEIEWEL | |
| | QKENSKRWNPEIQYTSNYYKSNNVEFAVNTEGV | |
| | YSEPRPIGTRYLTRNL | |
| AAV9 derived | DNNEGADGVGSSSGNWHCDSQWLGDRVITTSTR | 40 |
| polypeptide segment 5 | TWALPTYNNHLYKQISNSTSGGSSNDNAYFGYST | |
| with Q688K mutation | PWGYFDFNRFHCHFSPRDWQRLINNNWGFRPKR | |
| | LNFKLFNIQVKEVTDNNGVKTIANNLTSTVQVFT | |
| | DSDYQLPYVLGSAHEGCLPPFPADVFMIPQYGYL | |
| | TLNDGSQAVGRSSFYCLEYFPSQMLRTGNNFQFS | |
| | YEFENVPFHSSYAHSQSLDRLMNPLIDQYLYYLS | |
| | KTINGSGQNQQTLKFSVAGPSNMAVQGRNYIPGP | |
| | SYRQQRVSTTVTQNNNSEFAWPGASSWALNGRN | |
| | SLMNPGPAMASHKEGEDRFFPLSGSLIFGKQGTgr | |
| | dnvDADKVMITNEEEIKTTNPVATESYGQVATNHQ | |
| | SAQAQAQTGWVQNQGILPGMVWQDRDVYLQGP | |
| | IWAKIPHTDGNFHPSPLMGGFGMKHPPPQILIKNT | |
| | PVPADPPTAFNKDKLNSFITQYSTGQVSVEIEWEL | |
| | KKENSKRWNPEIQYTSNYYKSNNVEFAVNTEGV | |
| | YSEPRPIGTRYLTRNL | |

In some embodiments, the parental chimeric capsid protein comprises, consists essentially of, or consists of a polypeptide sequence at least about 80%, 85%, 90%, 95%, 96%, 97%, 98%, 99%, 99.5%, or 100% identical to any one of SEQ ID NOs: 41-64, or a functional fragment thereof. In some embodiments, the engineered capsid protein comprises a sequence that shares at least about 80% (e.g., at least 80%, 85%, 90%, 95%, 96%, 97%, 98%, 99%, or 100%) identity to any one of SEQ ID NOs: 41-64.

TABLE 7
Exemplary chimeric capsid protein sequences

| | | SEQ |
|---|---|---|
| Name | Sequence | ID NO: |
| ZC23 | MSFVDHPPDWLEEVGEGLREFLGLEAGPPKPKPNQQHQD | 41 |
| | QARGLVLPGYNYLGPGNGLDRGEPVNRADEVAREHDIS | |
| | YNEQLEAGDNPYLKYNHADAEFQEKLADDTSFGGNLGK | |
| | AVFQAKKRVLEPFGLVEEGAKTAPTGKRIDDHFPKRKKA | |
| | RTEEDSKPSTSSDAEAGPSGSQQLQIPAQPASSLGADTMS | |
| | AGGGGPLGDNNQGADGVGNASGDWHCDSTWMGDRVV | |
| | TKSTRTWALPTYNNHLYKQISNSTSGGSSNDNAYFGYST | |
| | PWGYFDFNRFHCHFSPRDWQRLINNNWGFRPKRLNFKLF | |
| | NIQVKEVTDNNGVKTIANNLTSTVQVFTDSDYQLPYVLG | |
| | SAHEGCLPPFPADVFMIPQYGYLTLNDGSQAVGRSSFYCL | |
| | EYFPSQMLRTGNNFQFSYEFENVPFHSSYAHSQSLDRLM | |
| | NPLIDQYLYYLSKTINGSGQNQQTLKFSVAGPSNMAVQG | |
| | RNYIPGPSYRQQRVSTTVTQNNNSEFAWPGASSWALNGR | |
| | NSLMNPGPAMASHKEGEDRFFPLSGSLIFGKQGTGRDNV | |
| | DADKVMITNEEEIKTTNPVATESYGQVATNHQSAQAQAQ | |
| | TGWVQNQGILPGMVWQDRDVYLQGPIWAKIPHTDGNFH | |
| | PSPLMGGFGMKHPPPQILIKNTPVPADPPTAFNKDKLNSFI | |
| | TQYSTGQVSVEIEWELQKENSKRWNPEIQYTNNYNDPQF | |
| | VDFAPDSTGEYRTTRPIGTRYLTRPL | |
| ZC24 | MAADGYLPDWLEDNLSEGIREWWALKPGAPQPKANQQ | 42 |
| | HQDNARGLVLPGYNYLGPGNGLDRGEPVNRADEVAREH | |
| | DISYNEQLEAGDNPYLKYNHADAEFQEKLADDTSFGGNL | |
| | GKAVFQAKKRVLEPFGLVEEGAKTAPTGKRIDDHFPKRK | |
| | KARTEEDSKPSTSSDAEAGPSGSQQLQIPAQPASSLGADT | |
| | MSAGGGGPLGDNNQGADGVGNASGDWHCDSTWMGDR | |
| | VVTKSTRTWVLPSYNNHQYREIKSGSVDGSNANAYFGYS | |
| | TPWGYFDFNRFHSHWSPRDWQRLINNYWGFRPRSLRVKI | |
| | FNIQVKEVTVQDSTTTIANNLTSTVQVFTDDDYQLPYVV | |
| | GNGTEGCLPAFPPQVFTLPQYGYATLNRDNTENPTERSSF | |
| | FCLEYFPSKMLRTGNNFEFTYNFEEVPFHSSFAPSQNLFKL | |
| | ANPLVDQYLYRFVSTNNTGGVQFNKNLAGRYANTYKN | |
| | WFPGPMGRTQGWNLGSGVNRASVSAFATTNRMELEGAS | |
| | YQVPPQPNGMTNNLQGSNTYALENTMIFNSQPANPGTTA | |
| | TYLEGNMLITSESETQPVNRVAYNVGGQMATNNQSSTTA | |
| | PATGTYNLQEIVPGSVWMERDVYLQGPIWAKIPETGAHF | |
| | HPSPAMGGFGLKHPPPMMLIKNTPVPGNITSFSDVPVSSFI | |
| | TQYSTGQVTVEMEWELKKENSKRWNPEIQYTSNYYKSN | |
| | NVEFAVNTEGVYSEPRPIGTRYLTRNL | |
| ZC25 | MSFVDHPPDWLEEVGEGLREFLGLEAGPPKPKPNQQHQD | 43 |
| | QARGLVLPGYNYLGPGNGLDRGEPVNRADEVAREHDIS | |
| | YNEQLEAGDNPYLKYNHADAEFQEKLADDTSFGGNLGK | |
| | AVFQAKKRVLEPFGLVEEAAKTAPTGKRIDDHFPKRKKA | |
| | RTEEDSKPSTSSDAEAGPSGSQQLQIPAQPASSLGADTMS | |
| | AGGGGPLGDNNQGADGVGNASGDWHCDSTWMGDRVV | |
| | TKSTRTWVLPSYNNHQYREIKSGSVDGSNANAYFGYSTP | |
| | WGYFDFNRFHSHWSPRDWQRLINNYWGFRPRSLRVKIFN | |
| | IQVKEVTDNNGVKTIANNLTSTVQVFTDSDYQLPYVLGS | |
| | AHEGCLPPFPADVFMIPQYGYLTLNDGSQAVGRSSFYCLE | |
| | YFPSQMLRTGNNFQFSYEFENVPFHSSYAHSQSLDRLMN | |
| | PLIDQYLYYLSKTINGSGQNQQTLKFSVAGPSNMAVQGR | |
| | NYIPGPSYRQQRVSTTVTQNNNSEFAWPGASSWALNGRN | |
| | SLMNPGPAMASHKEGEDRFFPLSGSLIFGKQGTGRDNVD | |
| | ADKVMITNEEEIKTTNPVATESYGQVATNHQSAQAQAQT | |
| | GWVQNQGILPGMVWQDRDVYLQGPIWAKIPHTDGNFHP | |
| | SPLMGGFGMKHPPPQILIKNTPVPADPPTAFNKDKLNSFIT | |
| | QYSTGQVSVEIEWELQKENSKRWNPEIQYTSNYYKSNNV | |
| | EFAVNTEGVYSEPRPIGTRYLTRNL | |
| ZC26 | MAADGYLPDWLEDNLSEGIREWWALKPGAPQPKANQQ | 44 |
| | HQDNARGLVLPGYKYLGPGNGLDKGEPVNAADAAALE | |
| | HDKAYDQQLKAGDNPYLKYNHADAEFQEKLADDTSFG | |
| | GNLGKAVFQAKKRVLEPFGLVEEGAKTAPTGKRIDDHFP | |
| | KRKKARTEEDSKPSTSSDAEAGPSGSQQLQIPAQPASSLG | |
| | ADTMSAGGGGPLGDNNQGADGVGNASGDWHCDSTWL | |
| | GDRVITTSTRTWALPTYNNHLYKQISNSTSGGSSNDNAYF | |
| | GYSTPWGYFDFNRFHCHFSPRDWQRLINNNWGFRPKRL | |
| | NFKLFNIQVKEVTDNNGVKTIANNLTSTVQVFTDSDYQL | |
| | PYVLGSAHEGCLPPFPADVFMIPQYGYLTLNDGSQAVGR | |
| | SSFYCLEYFPSQMLRTGNNFQFSYEFENVPFHSSYAHSQS | |
| | LDRLMNPLIDQYLYYLSKTINGSGQNQQTLKFSVAGPSN | |
| | MAVQGRNYIPGPSYRQQRVSTTVTQNNNSEFAWPGASS | |
| | WALNGRNSLMNPGPAMASHKEGEDRFFPLSGSLIFGKQG | |
| | TGRDNVDADKVMITNEEEIKTTNPVATESYGQVATNHQS | |
| | AQAQAQTGWVQNQGILPGMVWQDRDVYLQGPIWAKIP | |
| | HTDGNFHPSPLMGGFGMKHPPPQILIKNTPVPADPPTAFN | |
| | KDKLNSFITQYSTGQVSVEIEWELQKENSKRWNPEIQYTS | |
| | NYYKSNNVEFAVNTEGVYSEPRPIGTRYLTRNL | |
| ZC27 | MAADGYLPDWLEDNLSEGIREWWALKPGAPQPKANQQ | 45 |
| | HQDNARGLVLPGYKYLGPGNGLDKGEPVNAADAAALE | |
| | HDKAYDQQLKAGDNPYLKYNHADAEFQERLKEDTSFGG | |
| | NLGRAVFQAKKRLLEPLGLVEEAAKTAPGKKRPVEQSPQ | |
| | EPDSSAGIGKSGAQPAKKRLNFGQTGDTESVPDPQPIGEP | |
| | PAAPSGVGSLTMASGGGAPVADNNEGADGVGSSSGNWH | |
| | CDSQWLGDRVITTSTRTWALPTYNNHLYKQISNSTSGGSS | |
| | NDNAYFGYSTPWGYFDFNRFHCHFSPRDWQRLINNNWG | |
| | FRPKRLNFKLFNIQVKEVTDNNGVKTIANNLTSTVQVFTD | |
| | SDYQLPYVLGSAHEGCLPPFPADVFMIPQYGYLTLNDGS | |
| | QAVGRSSFYCLEYFPSQMLRTGNNFQFSYEFENVPFHSSY | |
| | AHSQSLDRLMNPLIDQYLYYLSKTINGSGQNQQTLKFSV | |
| | AGPSNMAVQGRNYIPGPSYRQQRVSTTVTQNNNSEFAWP | |
| | GASSWALNGRNSLMNPGPAMASHKEGEDRFFPLSGSLIF | |
| | GKQGTGRDNVDADKVMITNEEEIKTTNPVATESYGQVAT | |
| | NHQSAQAQAQTGWVQNQGILPGMVWQDRDVYLQGPIW | |
| | AKIPHTDGNFHPSPLMGGFGMKHPPPQILIKNTPVPADPPT | |
| | AFNKDKLNSFITQYSTGQVTVEMEWELKKENSKRWNPEI | |
| | QYTNNYNDPQFVDFAPDSTGEYRTTRPIGTRYLTRPL | |
| ZC28 | MSFVDHPPDWLEEVGEGLREFLGLEAGPPKPKPNQQHQD | 46 |
| | QARGLVLPGYNYLGPGNGLDRGEPVNRADEVAREHDIS | |
| | YNEQLEAGDNPYLKYNHADAEFQEKLADDTSFGGNLGK | |
| | AVFQAKKRVLEPFGLVEEGAKTAPTGKRIDDHFPKRKKA | |
| | RTEEDSKPSTSSDAEAGPSGSQQLQIPAQPASSLGADTMS | |
| | AGGGGPLGDNNQGADGVGNASGDWHCDSTWMGDRVIT | |
| | TSTRTWALPTYNNHLYKQISNSTSGGSSNDNAYFGYSTP | |
| | WGYFDFNRFHCHFSPRDWQRLINNNWGFRPKRLNFKLF | |
| | NIQVKEVTDNNGVKTIANNLTSTVQVFTDSDYQLPYVLG | |
| | SAHEGCLPPFPADVFMIPQYGYLTLNDGSQAVGRSSFYCL | |
| | EYFPSQMLRTGNNFQFSYEFENVPFHSSFAHSQSLDRLMN | |
| | PLIDQYLYYLSKTINGSGQNQQTLKFSVAGPSNMAVQGR | |
| | NYIPGPSYRQQRVSTTVTQNNNSEFAWPGASSWALNGRN | |
| | SLMNPGPAMASHKEGEDRFFPLSGSLIFGKQGTGRDNVD | |
| | ADKVMITNEEEIKTTNPVATESYGQVATNHQSAQAQAQT | |
| | GWVQNQGILPGMVWQDRDVYLQGPIWAKIPHTDGNFHP | |
| | SPLMGGFGMKHPPPQILIKNTPVPADPPTAFNKDKLNSFIT | |
| | QYSTGQVSVEIEWELQKENSKRWNPEIQYTSNYYKSNNV | |
| | EFAVNTEGVYSEPRPIGTRYLTRNL | |
| ZC29 | MSFVDHPPDWLEEVGEGLREFLGLEAGPPKPKPNQQHQD | 47 |
| | QARGLVLPGYNYLGPGNGLDRGEPVNRADEVAREHDIS | |
| | YNEQLEAGDNPYLKYNHADAEFQEKLADDTSFGGNLGK | |
| | AVFQAKKRVLEPFGLVEEGAKTAPTGKRIDDHFPKRKKA | |
| | RTEEDSKPSTSSDAEAGPSGSQQLQIPAQPASSLGADTMS | |
| | AGGGGPLGDNNQGADGVGNASGDWHCDSQWLGDRVIT | |
| | TSTRTWALPTYNNHLYKQISNSTSGGSSNDNAYFGYSTP | |
| | WGYFDFNRFHCHFSPRDWQRLINNNWGFRPKRLNFKLF | |
| | NIQVKEVTDNNGVKTIANNLTSTVQVFTDSDYQLPYVLG | |
| | SAHEGCLPPFPADVFMIPQYGYLTLNDGSQAVGRSSFYCL | |
| | EYFPSQMLRTGNNFQFSYEFENVPFHSSYAHSQSLDRLM | |
| | NPLIDQYLYYLSKTINGSGQNQQTLKFSVAGPSNMAVQG | |
| | RNYIPGPSYRQQRVSTTVTQNNNSEFAWPGASSWALNGR | |
| | NSLMNPGPAMASHKEGEDRFFPLSGSLIFGKQGTGRDNV | |
| | DADKVMITNEEEIKTTNPVATESYGQVATNHQSAQAQAQ | |
| | TGWVQNQGILPGMVWQDRDVYLQGPIWAKIPHTDGNFH | |
| | PSPLMGGFGMKHPPPQILIKNTPVPADPPTAFNKDKLNSFI | |
| | TQYSTGQVSVEIEWELQKENSKRWNPEIQYTSNYYKSNN | |
| | VEFAVNTEGVYSEPRPIGTRYLTRNL | |
| ZC30 | MSFVDHPPDWLEEVGEGLREFLGLEAGPPKPKPNQQHQD | 48 |
| | QARGLVLPGYNYLGPGNGLDRGEPVNRADEVAREHDIS | |
| | YNEQLEAGDNPYLKYNHADAEFQEKLADDTSFGGNLGK | |
| | AVFQAKKRVLEPFGLVEEGAKTAPTGKRIDDHFPKRKKA | |
| | RTEEDSKPSTSSDAEAGPSGSQQLQIPAQPASSLGADTMS | |
| | AGGGGPLGDNNQGADGVGNASGDWHCDSQWLGDRVIT | |
| | TSTRTWALPTYNNHLYKQISNSTSGGSSNDNAYFGYSTP | |
| | WGYFDFNRFHCHFSPRDWQRLINNNWGFRPKRLNFKLF | |
| | NIQVKEVTDNNGVKTIANNLTSTVQVFTDSDYQLPYVLG | |
| | SAHEGCLPPFPADVFMIPQYGYLTLNDGSQAVGRSSFYCL | |
| | EYFPSQMLRTGNNFQFSYEFENVPFHSSYAHSQSLDRLM | |
| | NPLIDQYLYYLSKTINGSGQNQQTLKFSVAGPSNMAVQG | |
| | RNYIPGPSYRQQRVSTTVTQNNNSEFAWPGASSWALNGR | |
| | NSLMNPGPAMASHKEGEDRFFPLSGSLIFGKQGTGRDNV | |
| | DADKVMITNEEEIKTTNPVATESYGQVATNHQSAQAQAQ | |
| | TGWVQNQGILPGMVWQDRDVYLQGPIWAKIPHTDGNFH | |
| | PSPLMGGFGMKHPPPQILIKNTPVPADPPTAFNKDKLNSFI | |
| | TQYSTGQVSVEIEWELQKENSKRWNPEIQYTNNYNDPQF | |
| | VDFAPDSTGEYRTTRPIGTRYLTRPL | |
| ZC31 | MAADGYLPDWLEDNLSEGIREWWALKPGAPQPKANQQ | 49 |
| | HQDNARGLVLPGYKYLGPGNGLDKGEPVNAADAAALE | |
| | HDKAYDQQLKAGDNPYLKYNHADAEFQERLKEDTSFGG | |
| | NLGRAVFQAKKRLLEPLGLVEEGAKTAPTGKRIDDHFPK | |
| | RKKARTEEDSKPSTSSDAEAGPSGSQQLQIPAQPASSLGA | |
| | DTMSAGGGGPLGDNNQGADGVGNASGDWHCDSTWMG | |
| | DRVITTSTRTWALPTYNNHLYKQISNSTSGGSSNDNAYFG | |
| | YSTPWGYFDFNRFHCHFSPRDWQRLINNNWGFRPKRLNF | |
| | KLFNIQVKEVTDNNGVKTIANNLTSTVQVFTDSDYQLPY | |
| | VLGSAHEGCLPPFPADVFMIPQYGYLTLNDGSQAVGRSSF | |
| | YCLEYFPSQMLRTGNNFQFSYEFENVPFHSSYAHSQSLDR | |
| | LMNPLIDQYLYYLSKTINGSGQNQQTLKFSVAGPSNMAV | |
| | QGRNYIPGPSYRQQRVSTTVTQNNNSEFAWPGASSWALN | |
| | GRNSLMNPGPAMASHKEGEDRFFPLSGSLIFGKQGTGRD | |
| | NVDADKVMITNEEEIKTTNPVATESYGQVATNHQSAQAQ | |
| | AQTGWVQNQGILPGMVWQDRDVYLQGPIWAKIPHTDG | |
| | NFHPSPLMGGFGMKHPPPQILIKNTPVPADPPTAFNKDKL | |
| | NSFITQYSTGQVTVEMEWELKKENSKRWNPEIQYTSNYY | |
| | KSNNVEFAVNTEGVYSEPRPIGTRYLTRNL | |
| ZC32 | MAADGYLPDWLEDNLSEGIREWWALKPGAPQPKANQQ | 50 |
| | HQDNARGLVLPGYKYLGPGNGLDKGEPVNAADAAALE | |
| | HDKAYDQQLKAGDNPYLKYNHADAEFQEKLADDTSFG | |
| | GNLGKAVFQAKKRVLEPFGLVEEGAKTAPTGKRIDDHFP | |
| | KRKKARTEEDSKPSTSSDAEAGPSGSQQLQIPAQPASSLG | |
| | ADTMSAGGGGPLGDNNQGADGVGNASGDWHCDSTWM | |
| | GDRVVTKSTRTWVLPSYNNHQYREIKSGSVDGSNANAY | |
| | FGYSTPWGYFDFNRFHCHFSPRDWQRLINNNWGFRPKRL | |
| | NFKLFNIQVKEVTDNNGVKTIANNLTSTVQVFTDSDYQL | |
| | PYVLGSAHEGCLPPFPADVFMIPQYGYLTLNDGSQAVGR | |
| | SSFYCLEYFPSQMLRTGNNFQFSYEFENVPFHSSYAHSQS | |
| | LDRLMNPLIDQYLYYLSKTINGSGQNQQTLKFSVAGPSN | |
| | MAVQGRNYIPGPSYRQQRVSTTVTQNNNSEFAWPGASS | |
| | WALNGRNSLMNPGPAMASHKEGEDRFFPLSGSLIFGKQG | |
| | TGRDNVDADKVMITNEEEIKTTNPVATESYGQVATNHQS | |
| | AQAQAQTGWVQNQGILPGMVWQDRDVYLQGPIWAKIP | |
| | HTDGNFHPSPLMGGFGMKHPPPQILIKNTPVPADPPTAFN | |
| | KDKLNSFITQYSTGQVSVEIEWELQKENSKRWNPEIQYTS | |
| | NYYKSNNVEFAVNTEGVYSEPRPIGTRYLTRNL | |
| ZC33 | MSFVDHPPDWLEEVGEGLREFLGLEAGPPKPKPNQQHQD | 51 |
| | QARGLVLPGYNYLGPGNGLDRGEPVNRADEVAREHDIS | |
| | YNEQLEAGDNPYLKYNHADAEFQEKLADDTSFGGNLGK | |
| | AVFQAKKRVLEPFGLVEEGAKTAPTGKRIDDHFPKRKKA | |
| | RTEEDSKPSTSSDAEAGPSGSQQLQIPAQPASSLGADTMS | |
| | AGGGGPLGDNNQGADGVGNASGDWHCDSTWMGDRVV | |
| | TKSTRTWALPTYNNHLYKQISNSTSGGSSNDNAYFGYST | |
| | PWGYFDFNRFHCHFSPRDWQRLINNNWGFRPKRLNFKLF | |
| | NIQVKEVTDNNGVKTIANNLTSTVQVFTDSDYQLPYVLG | |
| | SAHEGCLPPFPADVFMIPQYGYLTLNDGSQAVGRSSFYCL | |
| | EYFPSQMLRTGNNFQFSYEFENVPFHSSYAHSQSLDRLM | |
| | NPLIDQYLYYLSKTINGSGQNQQTLKFSVAGPSNMAVQG | |
| | RNYIPGPSYRQQRVSTTVTQNNNSEFAWPGASSWALNGR | |
| | NSLMNPGPAMASHKEGEDRFFPLSGSLIFGKQGTGRDNV | |
| | DADKVMITNEEEIKTTNPVATESYGQVATNHQSAQAQAQ | |
| | TGWVQNQGILPGMVWQDRDVYLQGPIWAKIPHTDGNFH | |
| | PSPLMGGFGMKHPPPQILIKNTPVPADPPTAFNKDKLNSFI | |
| | TQYSTGQVSVEIEWELQKENSKRWNPEIQYTSNYYKSNN | |
| | VEFAVNTEGVYSEPRPIGTRYLTRNL | |
| ZC34 | MAADGYLPDWLEDNLSEGIREWWALKPGAPQPKANQQ | 52 |
| | HQDNARGLVLPGYNYLGPGNGLDRGEPVNRADEVAREH | |
| | DISYNEQLEAGDNPYLKYNHADAEFQERLKEDTSFGGNL | |
| | GKAVFQAKKRVLEPLGLVEEAAKTAPGKKRPVEQSPQEP | |
| | DSSAGIGKSGAQPAKKRLNFGQTGDTESVPDPQPIGEPPA | |
| | APSGVGSLTMASGGGAPVADNNEGADGVGNASGDWHC | |
| | DSQWLGDRVITTSTRTWALPTYNNHLYKQISNSTSGGSS | |
| | NDNAYFGYSTPWGYFDFNRFHCHFSPRDWQRLINNNWG | |
| | FRPKRLNFKLFNIQVKEVTDNNGVKTIANNLTSTVQVFTD | |
| | SDYQLPYVLGSAHEGCLPPFPADVFMIPQYGYLTLNDGS | |
| | QAVGRSSFYCLEYFPSKMLRTGNNFQFSYEFENVPFHSSY | |
| | AHSQSLDRLMNPLIDQYLYYLSKTINGSGQNQQTLKFSV | |
| | AGPSNMAVQGRNYIPGPSYRQQRVSTTVTQNNNSEFAWP | |
| | GASSWALNGRNSLMNPGPAMASHKEGEDRFFPLSGSLIF | |
| | GKQGTGRDNVDADKVMITNEEEIKTTNPVATESYGQVAT | |
| | NHQSAQAQAQTGWVQNQGILPGMVWQDRDVYLQGPIW | |
| | AKIPHTDGNFHPSPLMGGFGMKHPPPQILIKNTPVPADPPT | |
| | AFNKDKLNSFITQYSTGQVTVEMEWELKKENSKRWNPEI | |
| | QYTNNYNDPQFVDFAPDSTGEYRTTRPIGTRYLTRNL | |
| ZC35 | MSFVDHPPDWLEEVGEGLREFLGLEAGPPKPKPNQQHQD | 53 |
| | QARGLVLPGYNYLGPGNGLDRGEPVNAADAAALEHDKA | |
| | YDQQLKAGDNPYLKYNHADAEFQERLKEDTSFGGNLGR | |
| | AVFQAKKRLLEPLGLVEEAAKTAPGKKRPVEQSPQEPDS | |
| | SAGIGKSGAQPAKKRLNFGQTGDTESVPDPQPIGEPPAAP | |
| | SGVGSLTMASGGGAPVADNNEGADGVGSSSGNWHCDS | |
| | QWLGDRVITTSTRTWALPTYNNHLYKQISNSTSGGSSND | |
| | NAYFGYSTPWGYFDFNRFHCHFSPRDWQRLINNNWGFR | |
| | PKRLNFKLFNIQVKEVTDNNGVKTIANNLTSTVQVFTDSD | |
| | YQLPYVLGSAHEGCLPPFPADVFMIPQYGYLTLNDGSQA | |
| | VGRSSFYCLEYFPSQMLRTGNNFQFSYEFENVPFHSSYAH | |
| | SQSLDRLMNPLIDQYLYYLSKTINGSGQNQQTLKFSVAGP | |
| | SNMAVQGRNYIPGPSYRQQRVSTTVTQNNNSEFAWPGAS | |
| | SWALNGRNSLMNPGPAMASHKEGEDRFFPLSGSLIFGKQ | |
| | GTGRDNVDADKVMITNEEEIKTTNPVATESYGQVATNHQ | |
| | SAQAQAQTGWVQNQGILPGMVWQDRDVYLQGPIWAKIP | |
| | HTDGNFHPSPLMGGFGMKHPPPQILIKNTPVPADPPTAFN | |
| | KDKLNSFITQYSTGQVSVEIEWELQKENSKRWNPEIQYTN | |
| | NYNDPQFVDFAPDSTGEYRTTRPIGTRYLTRPL | |
| ZC40/TN8 | MSFVDHPPDWLEEVGEGLREFLGLEAGPPKPKPNQQHQD | 54 |
| | QARGLVLPGYNYLGPGNGLDRGEPVNRADEVAREHDIS | |
| | YNEQLEAGDNPYLKYNHADAEFQEKLADDTSFGGNLGK | |
| | AVFQAKKRVLEPLGLVEEAAKTAPGKKRPVEQSPQEPDS | |
| | SAGIGKSGAQPAKKRLNFGQTGDTESVPDPQPIGEPPAAP | |
| | SGVGSLTMASGGGAPVADNNEGADGVGSSSGNWHCDS | |
| | QWLGDRVITTSTRTWALPTYNNHLYKQISNSTSGGSSND | |
| | NAYFGYSTPWGYFDFNRFHCHFSPRDWQRLINNNWGFR | |
| | PKRLNFKLFNIQVKEVTDNNGVKTIANNLTSTVQVFTDSD | |
| | YQLPYVLGSAHEGCLPPFPADVFMIPQYGYLTLNDGSQA | |
| | VGRSSFYCLEYFPSQMLRTGNNFQFSYEFENVPFHSSYAH | |
| | SQSLDRLMNPLIDQYLYYLSKTINGSGQNQQTLKFSVAGP | |
| | SNMAVQGRNYIPGPSYRQQRVSTTVTQNNNSEFAWPGAS | |
| | SWALNGRNSLMNPGPAMASHKEGEDRFFPLSGSLIFGKQ | |
| | GTGRDNVDADKVMITNEEEIKTTNPVATESYGQVATNHQ | |
| | SAQAQAQTGWVQNQGILPGMVWQDRDVYLQGPIWAKIP | |
| | HTDGNFHPSPLMGGFGMKHPPPQILIKNTPVPADPPTAFN | |
| | KDKLNSFITQYSTGQVSVEIEWELKKENSKRWNPEIQYTS | |
| | NYYKSNNVEFAVNTEGVYSEPRPIGTRYLTRNL | |
| ZC41 | MSFVDHPPDWLEEVGEGLREFLGLEAGPPKPKPNQQHQD | 55 |
| | QARGLVLPGYNYLGPGNGLDRGEPVNRADEVAREHDIS | |
| | YNEQLEAGDNPYLKYNHADAEFQEKLADDTSFGGNLGK | |
| | AVFQAKKRVLEPFGLVEEGAKTAPTGKRIDDHFPKRKKA | |
| | RTEEDSKPSTSSDAEAGPSGSQQLQIPAQPASSLGADTMS | |
| | AGGGGPLGDNNQGADGVGNASGDWHCDSTWMGDRVV | |
| | TKSTRTWALPTYNNHLYKQISNSTSGGSSNDNAYFGYST | |
| | PWGYFDFNRFHCHFSPRDWQRLINNNWGFRPKRLNFKLF | |
| | NIQVKEVTDNNGVKTIANNLTSTVQVFTDSDYQLPYVLG | |
| | SAHEGCLPPFPADVFMIPQYGYLTLNDGSQAVGRSSFYCL | |
| | EYFPSQMLRTGNNFQFSYEFENVPFHSSYAHSQSLDRLM | |
| | NPLIDQYLYYLSKTINGSGQNQQTLKFSVAGPSNMAVQG | |
| | RNYIPGPSYRQQRVSTTVTQNNNSEFAWPGASSWALNGR | |
| | NSLMNPGPAMASHKEGEDRFFPLSGSLIFGKQGTGRDNV | |
| | DADKVMITNEEEIKTTNPVATESYGQVATNHQSAQAQAQ | |
| | TGWVQNQGILPGMVWQDRDVYLQGPIWAKIPHTDGNFH | |
| | PSPLMGGFGMKHPPPQILIKNTPVPADPPTAFNKDKLNSFI | |
| | TQYSTGQVSVEIEWELQKENSKRWNPEIQYTSNYYKSNN | |
| | VEFAVNTEGVYSEPRPIGTRYLTRNL | |
| ZC42 | MSFVDHPPDWLEEVGEGLREFLGLEAGPPKPKPNQQHQD | 56 |
| | QARGLVLPGYNYLGPGNGLDRGEPVNRADEVAREHDIS | |
| | YNEQLEAGDNPYLKYNHADAEFQEKLADDTSFGGNLGK | |
| | AVFQAKKRVLEPFGLVEEGAKTAPTGKRIDDHFPKRKKA | |
| | RTEEDSKPSTSSDAEAGPSGSQQLQIPAQPASSLGADTMS | |
| | AGGGAPVADNNEGADGVGSSSGNWHCDSQWLGDRVIT | |
| | TSTRTWALPTYNNHLYKQISNSTSGGSSNDNAYFGYSTP | |
| | WGYFDFNRFHCHFSPRDWQRLINNNWGFRPKRLNFKLF | |
| | NIQVKEVTDNNGVKTIANNLTSTVQVFTDSDYQLPYVLG | |
| | SAHEGCLPPFPADVFMIPQYGYLTLNDGSQAVGRSSFYCL | |
| | EYFPSQMLRTGNNFQFSYEFENVPFHSSYAHSQSLDRLM | |
| | NPLIDQYLYYLSKTINGSGQNQQTLKFSVAGPSNMAVQG | |
| | RNYIPGPSYRQQRVSTTVTQNNNSEFAWPGASSWALNGR | |
| | NSLMNPGPAMASHKEGEDRFFPLSGSLIFGKQGTGRDNV | |
| | DADKVMITNEEEIKTTNPVATESYGQVATNHQSAQAQAQ | |
| | TGWVQNQGILPGMVWQDRDVYLQGPIWAKIPHTDGNFH | |
| | PSPLMGGFGMKHPPPQILIKNTPVPADPPTAFNKDKLNSFI | |
| | TQYSTGQVSVEIEWELQKENSKRWNPEIQYTSNYYKSNN | |
| | VEFAVNTEGVYSEPRPIGTRYLTRNL | |
| ZC43 | MSFVDHPPDWLEEVGEGLREFLGLEAGPPKPKPNQQHQD | 57 |
| | QARGLVLPGYKYLGPGNGLDKGEPVNAADAAALEHDK | |
| | AYDQQLKAGDNPYLKYNHADAEFQEKLADDTSFGGNLG | |
| | KAVFQAKKRVLEPFGLVEEGAKTAPTGKRIDDHFPKRKK | |
| | ARTEEDSKPSTSSDAEAGPSGSQQLQIPAQPASSLGADTM | |
| | SAGGGAPVADNNEGADGVGSSSGNWHCDSQWLGDRVIT | |
| | TSTRTWALPTYNNHLYKQISNSTSGGSSNDNAYFGYSTP | |
| | WGYFDFNRFHCHFSPRDWQRLINNNWGFRPKRLNFKLF | |
| | NIQVKEVTDNNGVKTIANNLTSTVQVFTDSDYQLPYVLG | |
| | SAHEGCLPPFPADVFMIPQYGYLTLNDGSQAVGRSSFYCL | |
| | EYFPSQMLRTGNNFQFSYEFENVPFHSSYAHSQSLDRLM | |
| | NPLIDQYLYYLSKTINGSGQNQQTLKFSVAGPSNMAVQG | |
| | RNYIPGPSYRQQRVSTTVTQNNNSEFAWPGASSWALNGR | |
| | NSLMNPGPAMASHKEGEDRFFPLSGSLIFGKQGTGRDNV | |
| | DADKVMITNEEEIKTTNPVATESYGQVATNHQSAQAQAQ | |
| | TGWVQNQGILPGMVWQDRDVYLQGPIWAKIPHTDGNFH | |
| | PSPLMGGFGMKHPPPQILIKNTPVPADPPTAFNKDKLNSFI | |
| | TQYSTGQVSVEIEWELQKENSKRWNPEIQYTSNYYKSNN | |
| | VEFAVNTEGVYSEPRPIGTRYLTRNL | |
| ZC44/TN10 | MAADGYLPDWLEDNLSEGIREWWALKPGAPQPKANQQ | 58 |
| | HQDNARGLVLPGYKYLGPGNGLDKGEPVNAADAAALE | |
| | HDKAYDQQLKAGDNPYLKYNHADAEFQERLKEDTSFGG | |
| | NLGRAVFQAKKRLLEPFGLVEEGAKTAPTGKRIDDHFPK | |
| | RKKARTEEDSKPSTSSDAEAGPSGSQQLQIPAQPASSLGA | |
| | DTMSAGGGGPLGDNNEGADGVGSSSGNWHCDSQWLGD | |
| | RVITTSTRTWALPTYNNHLYKQISNSTSGGSSNDNAYFGY | |
| | STPWGYFDFNRFHCHFSPRDWQRLINNNWGFRPKRLNFK | |
| | LFNIQVKEVTDNNGVKTIANNLTSTVQVFTDSDYQLPYV | |
| | LGSAHEGCLPPFPADVFMIPQYGYLTLNDGSQAVGRSSFY | |
| | CLEYFPSQMLRTGNNFQFSYEFENVPFHSSYAHSQSLDRL | |
| | MNPLIDQYLYYLSKTINGSGQNQQTLKFSVAGPSNMAVQ | |
| | GRNYIPGPSYRQQRVSTTVTQNNNSEFAWPGASSWALNG | |
| | RNSLMNPGPAMASHKEGEDRFFPLSGSLIFGKQGTGRDN | |
| | VDADKVMITNEEEIKTTNPVATESYGQVATNHQSAQAQA | |
| | QTGWVQNQGILPGMVWQDRDVYLQGPIWAKIPHTDGNF | |
| | HPSPLMGGFGMKHPPPQILIKNTPVPADPPTAFNKDKLNS | |
| | FITQYSTGQVSVEIEWELQKENSKRWNPEIQYTSNYYKSN | |
| | NVEFAVNTEGVYSEPRPIGTRYLTRNL | |
| ZC45 | MAADGYLPDWLEDNLSEGIREWWALKPGAPQPKANQQ | 59 |
| | HQDNARGLVLPGYKYLGPGNGLDKGEPVNAADAAALE | |
| | HDKAYDQQLEAGDNPYLKYNHADAEFQEKLADDTSFGG | |
| | NLGKAVFQAKKRVLEPFGLVEEGAKTAPTGKRIDDHFPK | |
| | RKKARTEEDSKPSTSSDAEAGPSGSQQLQIPAQPASSLGA | |
| | DTMSAGGGGPLGDNNQGADGVGNASGDWHCDSTWMG | |
| | DRVVTTSTRTWALPTYNNHLYKQISNSTSGGSSNDNAYF | |
| | GYSTPWGYFDFNRFHCHFSPRDWQRLINNNWGFRPKRL | |
| | NFKLFNIQVKEVTDNNGVKTIANNLTSTVQVFTDSDYQL | |
| | PYVLGSAHEGCLPPFPADVFMIPQYGYLTLNDGSQAVGR | |
| | SSFYCLEYFPSQMLRTGNNFQFSYEFENVPFHSSYAHSQS | |
| | LDRLMNPLIDQYLYYLSKTINGSGQNQQTLKFSVAGPSN | |
| | MAVQGRNYIPGPSYRQQRVSTTVTQNNNSEFAWPGASS | |
| | WALNGRNSLMNPGPAMASHKEGEDRFFPLSGSLIFGKQG | |
| | TGRDNVDADKVMITNEEEIKTTNPVATESYGQVATNHQS | |
| | AQAQAQTGWVQNQGILPGMVWQDRDVYLQGPIWAKIP | |
| | HTDGNFHPSPLMGGFGMKHPPPQILIKNTPVPADPPTAFN | |
| | KDKLNSFITQYSTGQVSVEIEWELQKENSKRWNPEIQYTS | |
| | NYYKSNNVEFAVNTEGVYSEPRPIGTRYLTRNL | |
| ZC46 | MSFVDHPPDWLEEVGEGLREFLGLEAGPPKPKPNQQHQD | 60 |
| | QARGLVLPGYNYLGPGNGLDRGEPVNRADEVAREHDIS | |
| | YNEQLEAGDNPYLKYNHADAEFQERLKEDTSFGGNLGR | |
| | AVFQAKKRLLEPLGLVEEAAKTAPGKKRPVEQSPQEPDS | |
| | SAGIGKSGAQPAKKRLNFGQTGDTESVPDPQPIGEPPAAP | |
| | SGVGSLTMASGGGAPVADNNEGADGVGSSSGNWHCDS | |
| | QWLGDRVITTSTRTWALPTYNNHLYKQISNSTSGGSSND | |
| | NAYFGYSTPWGYFDFNRFHCHFSPRDWQRLINNNWGFR | |
| | PKRLNFKLFNIQVKEVTDNNGVKTIANNLTSTVQVFTDSD | |
| | YQLPYVLGSAHEGCLPPFPADVFMIPQYGYLTLNDGSQA | |
| | VGRSSFYCLEYFPSQMLRTGNNFQFSYEFENVPFHSSYAH | |
| | SQSLDRLMNPLIDQYLYYLSKTINGSGQNQQTLKFSVAGP | |
| | SNMAVQGRNYIPGPSYRQQRVSTTVTQNNNSEFAWPGAS | |
| | SWALNGRNSLMNPGPAMASHKEGEDRFFPLSGSLIFGKQ | |
| | GTGRDNVDADKVMITNEEEIKTTNPVATESYGQVATNHQ | |
| | SAQAQAQTGWVQNQGILPGMVWQDRDVYLQGPIWAKIP | |
| | HTDGNFHPSPLMGGFGMKHPPPQILIKNTPVPADPPTAFN | |
| | KDKLNSFITQYSTGQVSVEIEWELQKENSKRWNPEIQYTS | |
| | NYYKSNNVEFAVNTEGVYSEPRPIGTRYLTRNL | |
| ZC47/TN14 | MAADGYLPDWLEDNLSEGIREWWALKPGAPQPKANQQ | 61 |
| | HQDNARGLVLPGYNYLGPGNGLDRGEPVNRADEVAREH | |
| | DISYNEQLEAGDNPYLKYNHADAEFQERLKEDTSFGGNL | |
| | GRAVFQAKKRLLEPLGLVEEAAKTAPGKKRPVEQSPQEP | |
| | DSSAGIGKSGAQPAKKRLNFGQTGDTESVPDPQPIGEPPA | |
| | APSGVGSLTMASGGGAPVADNNEGADGVGSSSGNWHC | |
| | DSQWLGDRVITTSTRTWALPTYNNHLYKQISNSTSGGSS | |
| | NDNAYFGYSTPWGYFDFNRFHCHFSPRDWQRLINNNWG | |
| | FRPKRLNFKLFNIQVKEVTDNNGVKTIANNLTSTVQVFTD | |
| | SDYQLPYVLGSAHEGCLPPFPADVFMIPQYGYLTLNDGS | |
| | QAVGRSSFYCLEYFPSQMLRTGNNFQFSYEFENVPFHSSY | |
| | AHSQSLDRLMNPLIDQYLYYLSKTINGSGQNQQTLKFSV | |
| | AGPSNMAVQGRNYIPGPSYRQQRVSTTVTQNNNSEFAWP | |
| | GASSWALNGRNSLMNPGPAMASHKEGEDRFFPLSGSLIF | |
| | GKQGTGRDNVDADKVMITNEEEIKTTNPVATESYGQVAT | |
| | NHQSAQAQAQTGWVQNQGILPGMVWQDRDVYLQGPIW | |
| | AKIPHTDGNFHPSPLMGGFGMKHPPPQILIKNTPVPADPPT | |
| | AFNKDKLNSFITQYSTGQVSVEIEWELQKENSKRWNPEIQ | |
| | YTSNYYKSNNVEFAVNTEGVYSEPRPIGTRYLTRNL | |
| ZC48 | MAADGYLPDWLEDNLSEGIREWWALKPGAPQPKANQQ | 62 |
| | HQDNARGLVLPGYKYLGPGNGLDKGEPVNAADAAALE | |
| | HDKAYDQQLKAGDNPYLKYNHADAEFQEKLADDTSFG | |
| | GNLGKAVFQAKKRLLEPLGLVEEAAKTAPGKKRPVEQSP | |
| | QEPDSSAGIGKSGAQPAKKRLNFGQTGDTESVPDPQPIGE | |
| | PPAAPSGVGSLTMASGGGAPVADNNEGADGVGSSSGNW | |
| | HCDSQWLGDRVITTSTRTWALPTYNNHLYKQISNSTSGG | |
| | SSNDNAYFGYSTPWGYFDFNRFHCHFSPRDWQRLINNN | |
| | WGFRPKRLNFKLFNIQVKEVTDNNGVKTIANNLTSTVQV | |
| | FTDSDYQLPYVLGSAHEGCLPPFPADVFMIPQYGYLTLND | |
| | GSQAVGRSSFYCLEYFPSQMLRTGNNFQFSYEFENVPFHS | |
| | SYAHSQSLDRLMNPLIDQYLYYLSKTINGSGQNQQTLKFS | |
| | VAGPSNMAVQGRNYIPGPSYRQQRVSTTVTQNNNSEFA | |
| | WPGASSWALNGRNSLMNPGPAMASHKEGEDRFFPLSGS | |
| | LIFGKQGTGRDNVDADKVMITNEEEIKTTNPVATESYGQ | |
| | VATNHQSAQAQAQTGWVQNQGILPGMVWQDRDVYLQ | |
| | GPIWAKIPHTDGNFHPSPLMGGFGMKHPPPQILIKNTPVP | |
| | ADPPTAFNKDKLNSFITQYSTGQVSVEIEWELQKENSKR | |
| | WNPEIQYTSNYYKSNNVEFAVNTEGVYSEPRPIGTRYLTR | |
| | NL | |
| ZC49 | MAADGYLPDWLEDNLSEGIREWWALKPGAPQPKANQQ | 63 |
| | HQDNARGLVLPGYKYLGPGNGLDKGEPVNAADAAALE | |
| | HDKAYDQQLKAGDNPYLKYNHADAEFQEKLADDTSFG | |
| | GNLGKAVFQAKKRVLEPLGLVEEAAKTAPGKKRPVEQSP | |
| | QEPDSSAGIGKSGAQPAKKRLNFGQTGDTESVPDPQPIGE | |
| | PPAAPSGVGSLTMASGGGAPVADNNEGADGVGSSSGNW | |
| | HCDSQWLGDRVITTSTRTWALPTYNNHLYKQISNSTSGG | |
| | SSNDNAYFGYSTPWGYFDFNRFHCHFSPRDWQRLINNN | |
| | WGFRPKRLNFKLFNIQVKEVTDNNGVKTIANNLTSTVQV | |
| | FTDSDYQLPYVLGSAHEGCLPPFPADVFMIPQYGYLTLND | |
| | GSQAVGRSSFYCLEYFPSQMLRTGNNFQFSYEFENVPFHS | |
| | SYAHSQSLDRLMNPLIDQYLYYLSKTINGSGQNQQTLKFS | |
| | VAGPSNMAVQGRNYIPGPSYRQQRVSTTVTQNNNSEFA | |
| | WPGASSWALNGRNSLMNPGPAMASHKEGEDRFFPLSGS | |
| | LIFGKQGTGRDNVDADKVMITNEEEIKTTNPVATESYGQ | |
| | VATNHQSAQAQAQTGWVQNQGILPGMVWQDRDVYLQ | |
| | GPIWAKIPHTDGNFHPSPLMGGFGMKHPPPQILIKNTPVP | |
| | ADPPTAFNKDKLNSFITQYSTGQVSVEIEWELQKENSKR | |
| | WNPEIQYTSNYYKSNNVEFAVNTEGVYSEPRPIGTRYLTR | |
| | NL | |
| ZC50 | MSFVDHPPDWLEEVGEGLREFLGLEAGPPKPKPNQQHQD | 64 |
| | QARGLVLPGYNYLGPGNGLDRGEPVNAADAAALEHDKA | |
| | YDQQLKAGDNPYLKYNHADAEFQERLKEDTSFGGNLGR | |
| | AVFQAKKRLLEPLGLVEEAAKTAPGKKRPVEQSPQEPDS | |
| | SAGIGKSGAQPAKKRLNFGQTGDTESVPDPQPIGEPPAAP | |
| | SGVGSLTMASGGGAPVADNNEGADGVGSSSGNWHCDS | |
| | QWLGDRVITTSTRTWALPTYNNHLYKQISNSTSGGSSND | |
| | NAYFGYSTPWGYFDFNRFHCHFSPRDWQRLINNNWGFR | |
| | PKRLNFKLFNIQVKEVTDNNGVKTIANNLTSTVQVFTDSD | |
| | YQLPYVLGSAHEGCLPPFPADVFMIPQYGYLTLNDGSQA | |
| | VGRSSFYCLEYFPSQMLRTGNNFQFSYEFENVPFHSSYAH | |
| | SQSLDRLMNPLIDQYLYYLSKTINGSGQNQQTLKFSVAGP | |
| | SNMAVQGRNYIPGPSYRQQRVSTTVTQNNNSEFAWPGAS | |
| | SWALNGRNSLMNPGPAMASHKEGEDRFFPLSGSLIFGKQ | |
| | GTGRDNVDADKVMITNEEEIKTTNPVATESYGQVATNHQ | |
| | SAQAQAQTGWVQNQGILPGMVWQDRDVYLQGPIWAKIP | |
| | HTDGNFHPSPLMGGFGMKHPPPQILIKNTPVPADPPTAFN | |
| | KDKLNSFITQYSTGQVSVEIEWELQKENSKRWNPEIQYTN | |
| | NYNDPQFVDFAPDSTGEYRTTRPIGTRYLTRPL | |

### I. Other AAV Capsid Proteins

In some embodiments, the engineered capsid protein is derived from any AAV capsid protein known in the art or described herein and additionally comprising a non-naturally occurring amino acid motif (e.g., a substitution motif, an insertion motif, or both) compared to to the wild-type or parental capsid protein derived therefrom.

In some embodiments, the wild-type or parental capsid protein is an AAV-SLB101 capsid protein or a variant thereof as known in the art or described in, e.g., WO 2021/072197, which is incorporated by reference herein in its entirety. In some embodiments, the wild-type or parental capsid protein is an AAVmod capsid protein or a variant thereof as known in the art or described in, e.g., WO 2022/173847 or in Olivieri et al. (2021) 24^{th} Annual Meeting of the American Society of Gene & Cell Therapy available at https://www.affiniatx.com/pdf/asgct_2021_olivieri.pdf, both of which are incorporated by reference herein in their entirety. In some embodiments, the wild-type or parental capsid protein is an AAV^{mut1dec1}, AAV^{deco1}, and/or AAV^{mut1} capsid protein or a variant thereof as known in the art or described in, e.g., WO 2022/173847. In some embodiments, the wild-type or parental capsid protein is an AAVcc.47 capsid protein or a variant thereof as known in the art or described in, e.g., Gonzalez et al. Nature Communications 13:5947 (2022), which is incorporated by reference herein in its entirety. In some embodiments, the wild-type or parental capsid protein is an AAVHSC16 capsid protein or a variant thereof as known in the art or described in, e.g., Smith et al. Molecular Therapy Methods & Clinical Development 26:224-238 (2022), which is incorporated by reference herein in its entirety. In some embodiments, the wild-type or parental capsid protein is a MyoAAV capsid protein or variant thereof as known in the art or described in, e.g., Tabebordbar et al. Cell 184(19):4919-4938. (2021), which is incorporated by reference herein in its entirety. In some embodiments, the wild-type or parental capsid protein is an MyoAAV-4E, MyoAAV-3F, MyoAAV-4A, or MyoAAV-4D capsid protein or variant thereof as known in the art or described in, e.g., Tabebordbar et al. In some embodiments, the wild-type or parental capsid protein is an 4D-C102 or C102 capsid protein or a variant thereof as known in the art or described in, e.g., US2021/0380643. Exemplary sequences of some of these capsid proteins are provided in Table 8 below. In some embodiments, the engineered capsid protein comprises a sequence that shares at least about 80% (e.g., at least 80%, 85%, 90%, 95%, 96%, 97%, 98%, 99%, or 100%) identity to any one of SEQ ID NOs: 65-76, for example, as compared using a sequence alignment algorism, e.g., BLAST® provided by the National Center for Biotechnology Information (NCBI).

TABLE 8
Exemplary AAV capsid protein sequences

| | | SEQ |
|---|---|---|
| Name | Sequence | ID NO: |
| AAV-SLB101 | MAADGYLPDWLEDNLSEGIREWWALKPGAPQPKANQQ | 65 |
| | HQDNARGLVLPGYKYLGPGNGLDKGEPVNAADAAALE | |
| | HDKAYDQQLKAGDNPYLKYNHADAEFQERLKEDTSFGG | |
| | NLGRAVFQAKKRLLEPLGLVEEAAKTAPGKKRPVEQSPQ | |
| | EPDSSAGIGKSGAQPAKKRLNFGQTGDTESVPDPQPIGEP | |
| | PAAPSGVGSLTMASGGGAPVADNNEGADGVGSSSGNWH | |
| | CDSQWLGDRVITTSTRTWALPTYNNHLYKQISNSTSGGSS | |
| | NDNAYFGYSTPWGYFDFNRFHCHFSPRDWQRLINNNWG | |
| | FRPKRLNFKLFNIQVKEVTDNNGVKTIANNLTSTVQVFTD | |
| | SDYQLPYVLGSAHEGCLPPFPADVFMIPQYGYLTLNDGS | |
| | QAVGRSSFYCLEYFPSQMLRTGNNFQFSYEFENVPFHSSY | |
| | AHSQSLDRLMNPLIDQYLYYLSKTINGSGQNQQTLKFSV | |
| | AGPSNMAVQGRNYIPGPSYRQQRVSTTVTQNNNSEFA WP | |
| | GASSWALNGRNSLMNPGPAMASHKEGEDRFFPLSGSLIF | |
| | GKQGTGRDNVDADKVMITNEEEIKTTNPVATESYGQVAT | |
| | NHQSAQRGDLGLSAQAQTGWVQNQGILPGMVWQDRDV | |
| | YLQGPIWAKIPHTDGNFHPSPLMGGFGMKHPPPQILIKNT | |
| | PVPADPPTAFNKDKLNSFITQYSTGQVSVEIEWELQKENS | |
| | KRWNPEIQYTSNYYKSNNVEFAVNTEGVYSEPRPIGTRY | |
| | LTRNL | |
| AAVmut1dec1 | MAADGYLPDWLEDNLSEGIREWWALKPGAPQPKANQQ | 66 |
| | HQDNARGLVLPGYKYLGPGNGLDKGEPVNAADAAALE | |
| | HDKAYDQQLKAGDNPYLKYNHADAEFQERLKEDTSFGG | |
| | NLGRAVFQAKKRLLEPLGLVEEAAKTAPGKKRPVEQSPQ | |
| | EPDSSAGIGKSGAQPAKKRLNFGQTGDTESVPDPQPIGEP | |
| | PAAPSGVGSLTMASGGGAPVADNNEGADGVGSSSGNWH | |
| | CDSQWLGDRVITTSTRTWALPTYNNHLYKQISNSTSGAS | |
| | TNDNAYFGYSTPWGYFDFNRFHCHFSPRDWQRLINNNW | |
| | GFRPKRLNFKLFNIQVKEVTDNNGVKTIANNLTSTVQVFT | |
| | DSDYQLPYVLGSAHEGCLPPFPADVFMIPQYGYLTLNDG | |
| | SQAVGRSSFYCLEYFPSQMLRTGNNFQFSYEFENVPFHSS | |
| | YAHSQSLDRLMNPLIDQYLYYLSKTINGSGQNQQTLKFS | |
| | VAGPSNMAVQGRNYIPGPSYRQQRVSTTVTQNNNSEFA | |
| | WPGASSWALNGRNSLMNPGPAMASHKEGEDRFFPLSGS | |
| | LIFGKQGTGRDNVDADKVMITNEEEIKTTNPVATESYGQ | |
| | VATNHQSAQRGDLLLSAQAQTGWVQNQGILPGMVWQD | |
| | RDVYLQGPIWAKIPHTDGNFHPSPLMGGFGMKHPPPQILI | |
| | KNTPVPADPPTAFNKDKLNSFITQYSTGQVSVEIEWELQK | |
| | ENSKRWNPEIQYTSNYYKSNNVEFAVNTEGVYSEPRPIGT | |
| | RYLTRNL | |
| AAVdeco1 | MAADGYLPDWLEDNLSEGIREWWALKPGAPQPKANQQ | 67 |
| | HQDNARGLVLPGYKYLGPGNGLDKGEPVNAADAAALE | |
| | HDKAYDQQLKAGDNPYLKYNHADAEFQERLKEDTSFGG | |
| | NLGRAVFQAKKRLLEPLGLVEEAAKTAPGKKRPVEQSPQ | |
| | EPDSSAGIGKSGAQPAKKRLNFGQTGDTESVPDPQPIGEP | |
| | PAAPSGVGSLTMASGGGAPVADNNEGADGVGSSSGNWH | |
| | CDSQWLGDRVITTSTRTWALPTYNNHLYKQISNSTSGGSS | |
| | NDNAYFGYSTPWGYFDFNRFHCHFSPRDWQRLINNNWG | |
| | FRPKRLNFKLFNIQVKEVTDNNGVKTIANNLTSTVQVFTD | |
| | SDYQLPYVLGSAHEGCLPPFPADVFMIPQYGYLTLNDGS | |
| | QAVGRSSFYCLEYFPSQMLRTGNNFQFSYEFENVPFHSSY | |
| | AHSQSLDRLMNPLIDQYLYYLSKTINGSGQNQQTLKFSV | |
| | AGPSNMAVQGRNYIPGPSYRQQRVSTTVTQNNNSEFAWP | |
| | GASSWALNGRNSLMNPGPAMASHKEGEDRFFPLSGSLIF | |
| | GKQGTGRDNVDADKVMITNEEEIKTTNPVATESYGQVAT | |
| | NHQSAQRGDLLLSAQAQTGWVQNQGILPGMVWQDRDV | |
| | YLQGPIWAKIPHTDGNFHPSPLMGGFGMKHPPPQILIKNT | |
| | PVPADPPTAFNKDKLNSFITQYSTGQVSVEIEWELQKENS | |
| | KRWNPEIQYTSNYYKSNNVEFAVNTEGVYSEPRPIGTRY | |
| | LTRNL | |
| AAVmut1 | MAADGYLPDWLEDNLSEGIREWWALKPGAPQPKANQQ | 68 |
| | HQDNARGLVLPGYKYLGPGNGLDKGEPVNAADAAALE | |
| | HDKAYDQQLKAGDNPYLKYNHADAEFQERLKEDTSFGG | |
| | NLGRAVFQAKKRLLEPLGLVEEAAKTAPGKKRPVEQSPQ | |
| | EPDSSAGIGKSGAQPAKKRLNFGQTGDTESVPDPQPIGEP | |
| | PAAPSGVGSLTMASGGGAPVADNNEGADGVGSSSGNWH | |
| | CDSQWLGDRVITTSTRTWALPTYNNHLYKQISNSTSGAS | |
| | TNDNAYFGYSTPWGYFDFNRFHCHFSPRDWQRLINNNW | |
| | GFRPKRLNFKLFNIQVKEVTDNNGVKTIANNLTSTVQVFT | |
| | DSDYQLPYVLGSAHEGCLPPFPADVFMIPQYGYLTLNDG | |
| | SQAVGRSSFYCLEYFPSQMLRTGNNFQFSYEFENVPFHSS | |
| | YAHSQSLDRLMNPLIDQYLYYLSKTINGSGQNQQTLKFS | |
| | VAGPSNMAVQGRNYIPGPSYRQQRVSTTVTQNNNSEFA | |
| | WPGASSWALNGRNSLMNPGPAMASHKEGEDRFFPLSGS | |
| | LIFGKQGTGRDNVDADKVMITNEEEIKTTNPVATESYGQ | |
| | VATNHQSAQAQAQTGWVQNQGILPGMVWQDRDVYLQ | |
| | GPIWAKIPHTDGNFHPSPLMGGFGMKHPPPQILIKNTPVP | |
| | ADPPTAFNKDKLNSFITQYSTGQVSVEIEWELQKENSKR | |
| | WNPEIQYTSNYYKSNNVEFAVNTEGVYSEPRPIGTRYLTR | |
| | NL | |
| AAVcc.47 | MAADGYLPDWLEDNLSEGIREWWALKPGAPQPKANQQ | 69 |
| | HQDNARGLVLPGYKYLGPGNGLDKGEPVNAADAAALE | |
| | HDKAYDQQLKAGDNPYLKYNHADAEFQERLKEDTSFGG | |
| | NLGRAVFQAKKRLLEPLGLVEEAAKTAPGKKRPVEQSPQ | |
| | EPDSSAGIGKSGAQPAKKRLNFGQTGDTESVPDPQPIGEP | |
| | PAAPSGVGSLTMASGGGAPVADNNEGADGVGSSSGNWH | |
| | CDSQWLGDRVITTSTRTWALPTYNNHLYKQISNSTSGGSS | |
| | NDNAYFGYSTPWGYFDFNRFHCHFSPRDWQRLINNNWG | |
| | FRPKRLNFKLFNIQVKEVTDNNGVKTIANNLTSTVQVFTD | |
| | SDYQLPYVLGSAHEGCLPPFPADVFMIPQYGYLTLNDGS | |
| | QAVGRSSFYCLEYFPSQMLRTGNNFQFSYEFENVPFHSSY | |
| | AHSQSLDRLMNPLIDQYLYYLSKTIGVSLGGGQTLKFSVA | |
| | GPSNMAVQGRNYIPGPSYRQQRVSTTVTQNNNSEFAWPG | |
| | ASSWALNGRNSLMNPGPAMASHKEGEDRFFPLSGSLIFG | |
| | KQGTGRDNVDADKVMITNEEEIKTTNPVATESYGQVATN | |
| | HQSAQAQAQTGWVQNQGILPGMVWQDRDVYLQGPIWA | |
| | KIPHTDGNFHPSPLMGGFGMKHPPPQILIKNTPVPADPPTA | |
| | FNKDKLNSFITQYSTGQVSVEIEWELQKENSKRWNPEIQY | |
| | TSNYYKSNNVEFAVNTEGVYSEPRPIGTRYLTRNL | |
| AAVHSC16 | MAADGYLPDWLEDNLSEGIREWWALKPGAPQPKANQQ | 70 |
| | HQDNARGLVLPGYKYLGPGNGLDKGEPVNAADAAALE | |
| | HDKAYDQQLKAGDNPYLKYNHADAEFQERLKEDTSFGG | |
| | NLGRAVFQAKKRLLEPLGLVEEAAKTAPGKKRPVEQSPQ | |
| | EPDSSAGIGKSGAQPAKKRLNFGQTGDTESVPDPQPIGEP | |
| | PAAPSGVGSLTMASGGGAPVADNNEGADGVGSSSGNWH | |
| | CDSQWLGDRVITTSTRTWALPTYNNHLYKQISNSTSGGSS | |
| | NDNAYFGYSTPWGYFDFNRFHCHFSPRDWQRLINNNWG | |
| | FRPKRLNFKLFNIQVKEVTDNNGVKTIANNLTSTVQVFTD | |
| | SDYQLPYVLGSAHEGCLPPFPADVFMIPQYGYLTLNDGS | |
| | QAVGRSSFYCLEYFPSQMLRTGNNFQFSYEFENVPFHSSY | |
| | AHSQSLDRLMNPLIDQYLYYLSKTINGSGQNQQTLKFSV | |
| | AGPSNMAVQGRNYIPGPSYRQQRVSTTVTQNNNSEIAWP | |
| | RASSWALNGRNSLMNPGPAMASHKEGEDRFFPLSGSLIF | |
| | GKQGTGRDNVDADKVMITNEEEIKTTNPVATESYGQVAT | |
| | NHQSAQAQAQTGWVQNQGILPGMVWQDRDVYLQGPIW | |
| | AKIPHTDGNFHPSPLMGGFGMKHPPPQILIKNTPVPADPPT | |
| | AFNKDKLNSFITQYSTGQVSVEIEWELQKENSKRWNPEIQ | |
| | YTSNYCKSNNVEFAVNTEGVYSEPRPIGTRYLTRNL | |
| MyoAAV-4E | MAADGYLPDWLEDNLSEGIREWWALKPGAPQPKANQQ | 71 |
| | HQDNARGLVLPGYKYLGPGNGLDKGEPVNAADAAALE | |
| | HDKAYDQQLKAGDNPYLKYNHADAEFQERLKEDTSFGG | |
| | NLGRAVFQAKKRLLEPLGLVEEAAKTAPGKKRPVEQSPQ | |
| | EPDSSAGIGKSGAQPAKKRLNFGQTGDTESVPDPQPIGEP | |
| | PAAPSGVGSLTMASGGGAPVADNNEGADGVGSSSGNWH | |
| | CDSQWLGDRVITTSTRTWALPTYNNHLYKQISNSTSGGSS | |
| | NDNAYFGYSTPWGYFDFNRFHCHFSPRDWQRLINNNWG | |
| | FRPKRLNFKLFNIQVKEVTDNNGVKTIANNLTSTVQVFTD | |
| | SDYQLPYVLGSAHEGCLPPFPADVFMIPQYGYLTLNDGS | |
| | QAVGRSSFYCLEYFPSQMLRTGNNFQFSYEFENVPFHSSY | |
| | AHSQSLDRLMNPLIDQYLYYLSKTINGSGQNQQTLKFSV | |
| | AGPSNMAVQGRNYIPGPSYRQQRVSTTVTQNNNSEFAWP | |
| | GASSWALNGRNSLMNPGPAMASHKEGEDRFFPLSGSLIF | |
| | GKQGTGRDNVDADKVMITNEEEIKTTNPVATESYGQVAT | |
| | NHQENRRGDFNNTAQAQTGWVQNQGILPGMVWQDRDV | |
| | YLQGPIWAKIPHTDGNFHPSPLMGGFGMKHPPPQILIKNT | |
| | PVPADPPTAFNKDKLNSFITQYSTGQVSVEIEWELQKENS | |
| | KRWNPEIQYTSNYYKSNNVEFAVNTEGVYSEPRPIGTRY | |
| | LTRNL | |
| MyoAAV-3F | MAADGYLPDWLEDNLSEGIREWWALKPGAPQPKANQQ | 72 |
| | HQDNARGLVLPGYKYLGPGNGLDKGEPVNAADAAALE | |
| | HDKAYDQQLKAGDNPYLKYNHADAEFQERLKEDTSFGG | |
| | NLGRAVFQAKKRLLEPLGLVEEAAKTAPGKKRPVEQSPQ | |
| | EPDSSAGIGKSGAQPAKKRLNFGQTGDTESVPDPQPIGEP | |
| | PAAPSGVGSLTMASGGGAPVADNNEGADGVGSSSGNWH | |
| | CDSQWLGDRVITTSTRTWALPTYNNHLYKQISNSTSGGSS | |
| | NDNAYFGYSTPWGYFDFNRFHCHFSPRDWQRLINNNWG | |
| | FRPKRLNFKLFNIQVKEVTDNNGVKTIANNLTSTVQVFTD | |
| | SDYQLPYVLGSAHEGCLPPFPADVFMIPQYGYLTLNDGS | |
| | QAVGRSSFYCLEYFPSQMLRTGNNFQFSYEFENVPFHSSY | |
| | AHSQSLDRLMNPLIDQYLYYLSKTINGSGQNQQTLKFSV | |
| | AGPSNMAVQGRNYIPGPSYRQQRVSTTVTQNNNSEFAWP | |
| | GASSWALNGRNSLMNPGPAMASHKEGEDRFFPLSGSLIF | |
| | GKQGTGRDNVDADKVMITNEEEIKTTNPVATESYGQVAT | |
| | NHQSAQRGDHASWAQAQTGWVQNQGILPGMVWQDRD | |
| | VYLQGPIWAKIPHTDGNFHPSPLMGGFGMKHPPPQILIKN | |
| | TPVPADPPTAFNKDKLNSFITQYSTGQVSVEIEWELQKEN | |
| | SKRWNPEIQYTSNYYKSNNVEFAVNTEGVYSEPRPIGTRY | |
| | LTRNL | |
| MyoAAV-4A | MAADGYLPDWLEDNLSEGIREWWALKPGAPQPKANQQ | 73 |
| | HQDNARGLVLPGYKYLGPGNGLDKGEPVNAADAAALE | |
| | HDKAYDQQLKAGDNPYLKYNHADAEFQERLKEDTSFGG | |
| | NLGRAVFQAKKRLLEPLGLVEEAAKTAPGKKRPVEQSPQ | |
| | EPDSSAGIGKSGAQPAKKRLNFGQTGDTESVPDPQPIGEP | |
| | PAAPSGVGSLTMASGGGAPVADNNEGADGVGSSSGNWH | |
| | CDSQWLGDRVITTSTRTWALPTYNNHLYKQISNSTSGGSS | |
| | NDNAYFGYSTPWGYFDFNRFHCHFSPRDWQRLINNNWG | |
| | FRPKRLNFKLFNIQVKEVTDNNGVKTIANNLTSTVQVFTD | |
| | SDYQLPYVLGSAHEGCLPPFPADVFMIPQYGYLTLNDGS | |
| | QAVGRSSFYCLEYFPSQMLRTGNNFQFSYEFENVPFHSSY | |
| | AHSQSLDRLMNPLIDQYLYYLSKTINGSGQNQQTLKFSV | |
| | AGPSNMAVQGRNYIPGPSYRQQRVSTTVTQNNNSEFAWP | |
| | GASSWALNGRNSLMNPGPAMASHKEGEDRFFPLSGSLIF | |
| | GKQGTGRDNVDADKVMITNEEEIKTTNPVATESYGQVAT | |
| | NHQSNSRGDYNSLAQAQTGWVQNQGILPGMVWQDRDV | |
| | YLQGPIWAKIPHTDGNFHPSPLMGGFGMKHPPPQILIKNT | |
| | PVPADPPTAFNKDKLNSFITQYSTGQVSVEIEWELQKENS | |
| | KRWNPEIQYTSNYYKSNNVEFAVNTEGVYSEPRPIGTRY | |
| | LTRNL | |
| MyoAAV-4D | MAADGYLPDWLEDNLSEGIREWWALKPGAPQPKANQQ | 74 |
| | HQDNARGLVLPGYKYLGPGNGLDKGEPVNAADAAALE | |
| | HDKAYDQQLKAGDNPYLKYNHADAEFQERLKEDTSFGG | |
| | NLGRAVFQAKKRLLEPLGLVEEAAKTAPGKKRPVEQSPQ | |
| | EPDSSAGIGKSGAQPAKKRLNFGQTGDTESVPDPQPIGEP | |
| | PAAPSGVGSLTMASGGGAPVADNNEGADGVGSSSGNWH | |
| | CDSQWLGDRVITTSTRTWALPTYNNHLYKQISNSTSGGSS | |
| | NDNAYFGYSTPWGYFDFNRFHCHFSPRDWQRLINNNWG | |
| | FRPKRLNFKLFNIQVKEVTDNNGVKTIANNLTSTVQVFTD | |
| | SDYQLPYVLGSAHEGCLPPFPADVFMIPQYGYLTLNDGS | |
| | QAVGRSSFYCLEYFPSQMLRTGNNFQFSYEFENVPFHSSY | |
| | AHSQSLDRLMNPLIDQYLYYLSKTINGSGQNQQTLKFSV | |
| | AGPSNMAVQGRNYIPGPSYRQQRVSTTVTQNNNSEFAWP | |
| | GASSWALNGRNSLMNPGPAMASHKEGEDRFFPLSGSLIF | |
| | GKQGTGRDNVDADKVMITNEEEIKTTNPVATESYGQVAT | |
| | NHQASTRGDHGVLAQAQTGWVQNQGILPGMVWQDRDV | |
| | YLQGPIWAKIPHTDGNFHPSPLMGGFGMKHPPPQILIKNT | |
| | PVPADPPTAFNKDKLNSFITQYSTGQVSVEIEWELQKENS | |
| | KRWNPEIQYTSNYYKSNNVEFAVNTEGVYSEPRPIGTRY | |
| | LTRNL | |
| 4D-C102 | MAADGYLPDWLEDTLSEGIRQWWKLKPGPPPPKPAERH | 75 |
| (variant | KDDSRGLVLPGYKYLGPFNGLDKGEPVNEADAAALEHD | |
| sequence-1) | KAYDRQLDSGDNPYLKYNHADAEFQERLKEDTSFGGNL | |
| | GRAVFQAKKRVLEPLGLVEEPVKTAPGKKRPVEHSPVEP | |
| | DSSSGTGKAGQQPARKRLNFGQTGDADSVPDPQPLGQPP | |
| | AAPSGLGTNTMATGSGAPMADNNEGADGVGNSSGNWH | |
| | CDSTWMGDRVITTSTRTWALPTYNNHLYKQISSQSGASN | |
| | DNHYFGYSTPWGYFDFNRFHCHFSPRDWQRLINNNWGF | |
| | RPKRLNFKLFNIQVKEVTQNDGTTTIANNLTSTVQVFTDS | |
| | EYQLPYVLGSAHQGCLPPFPADVFMVPQYGYLTLNNGSQ | |
| | AVGRSSFYCLEYFPSQMLRTGNNFTFSYTFEDVPFHSSYA | |
| | HSQSLDRLMNPLIDQYLYYLSRTNTPSGTTTQSRLQFSQA | |
| | GASDIRDQSRNWLPGPCYRQQRVSKTSADNNNSEYSWT | |
| | GATKYHLNGRDSLVNPGPAMASHKDDEEKFFPQSGVLIF | |
| | GKQGSEKTNVDIEKVMITDEEEIRTTNPVATEQYGSVSTN | |
| | LQRGNLANKTTNKDARQAATADVNTQGVLPGMVWQDR | |
| | DVYLQGPIWAKIPHTDGHFHPSPLMGGFGLKHPPPQILIK | |
| | NTPVPANPSTTFSAAKFASFITQYSTGQVSVEIEWELQKE | |
| | NSKRWNPEIQYTSNYNKSINVDFTVDTNGVYSEPRPIGTR | |
| | YLTRNL | |
| 4D-C102 | MAADGYLPDWLEDTLSEGIRQWWKLKPGPPPPKPAERH | 76 |
| (variant_ | KDDSRGLVLPGYKYLGPFNGLDKGEPVNEADAAALEHD | |
| sequence-2) | KAYDRQLDSGDNPYLKYNHADAEFQERLKEDTSFGGNL | |
| | GRAVFQAKKRVLEPLGLVEEPVKTAPGKKRPVEHSPVEP | |
| | DSSSGTGKAGQQPARKRLNFGQTGDADSVPDPQPLGQPP | |
| | AAPSGLGTNTMATGSGAPMADNNEGADGVGNSSGNWH | |
| | CDSTWMGDRVITTSTRTWALPTYNNHLYKQISSQSGASN | |
| | DNHYFGYSTPWGYFDFNRFHCHFSPRDWQRLINNNWGF | |
| | RPKRLNFKLFNIQVKEVTQNDGTTTIANNLTSTVQVFTDS | |
| | EYQLPYVLGSAHQGCLPPFPADVFMVPQYGYLTLNNGSQ | |
| | AVGRSSFYCLEYFPSQMLRTGNNFTFSYTFEDVPFHSSYA | |
| | HSQSLDRLMNPLIDQYLYYLSRTNTPSGTTTQSRLQFSQA | |
| | GASDIRDQSRNWLPGPCYRQQRVSKTSADNNNSEYSWT | |
| | GATKYHLNGRDSLVNPGPAMASHKDDEEKFFPQSGVLIF | |
| | GKQGSEKTNVDIEKVMITDEEEIRTTNPVATEQYGSVSTN | |
| | LQRGNLANKIQRTDARQAATADVNTQGVLPGMVWQDR | |
| | DVYLQGPIWAKIPHTDGHFHPSPLMGGFGLKHPPPQILIK | |
| | NTPVPANPSTTFSAAKFASFITQYSTGQVSVEIEWELQKE | |
| | NSKRWNPEIQYTSNYNKSINVDFTVDTNGVYSEPRPIGTR | |
| | YLTRNL | |

Recombinant Adeno-Associated Virus (rAAV) Vectors or Virions, Kits, and Pharmaceutical Compositions Thereof

In some embodiments, provided are viral vectors or virions comprising an engineered capsid protein according to various embodiments described herein. In some embodiments, the viral vector or virion is an AAV vector or virion.

In some embodiments, provided are rAAV vectors or rAAV virions comprising:
(a) an engineered capsid protein according to various embodiments described herein; and
(b) a vector genome comprising an expression cassette flanked by inverted terminal repeats (ITRs), wherein the expression cassette comprises one or more nucleotide sequences encoding one or more gene products operatively linked to one or more promoters.

In some embodiments, the rAAV virion specifically transduces muscle cells.

In some embodiments, the rAAV virion specifically transduces cardiac cells.

In some embodiments, the rAAV virion specifically transduces skeletal muscle cells.

In some embodiments, the rAAV virion specifically transduces heart cells.

In some embodiments, the rAAV virion specifically transduces cardiomyocytes.

In some embodiments, the rAAV virion traffics to at least one organ other than the liver.

In some embodiments, the rAAV virion traffics to the heart.

In some embodiments, the rAAV virion exhibits a higher heart transduction efficiency than an rAAV virion having an AAV9 VP1 capsid protein according to SEQ ID NO: 1. In some embodiments, the rAAV virion exhibits a higher (e.g., at least 2, 3, 4, 5, 6, 7, 8, 9 or 10 times higher) heart-to-liver transduction ratio than an rAAV virion having an AAV9 VP1 capsid protein according to SEQ ID NO: 1. In some embodiments, administration of the rAAV virion to a subject leads to a lower (e.g., at least 2, 3, 4, 5, 6, 7, 8, 9 or 10 times lower) liver viral load than administration of an rAAV virion having an AAV9 VP1 capsid protein according to SEQ ID NO: 1. In some embodiments, the rAAV virion exhibits a higher transduction efficiency, optionally higher heart transduction efficiency, than an rAAV virion having an AAV9 VP1 capsid protein according to SEQ ID NO: 1, assessed in a primate. In some embodiments, the rAAV virion exhibits a higher (e.g., at least 2, 3, 4, 5, 6, 7, 8, 9 or 10 times higher) heart-to-liver transduction ratio than an rAAV virion having an AAV9 VP1 capsid protein according to SEQ ID NO: 1, assessed in a primate. In some embodiments, administration of the rAAV virion to a subject leads to a lower (e.g., at least 2, 3, 4, 5, 6, 7, 8, 9 or 10 times lower) liver viral load than administration of an rAAV virion having an AAV9 VP1 capsid protein according to SEQ ID NO: 1, as assessed in a primate.

In some embodiments, the rAAV virion exhibits a higher heart transduction efficiency than an rAAV virion having an AAV5 VP1 capsid protein according to SEQ ID NO: 10. In some embodiments, the rAAV virion exhibits a higher (e.g., at least 2, 3, 4, 5, 6, 7, 8, 9 or 10 times higher) heart-to-liver transduction ratio than an rAAV virion having an AAV5 VP1 capsid protein according to SEQ ID NO: 10. In some embodiments, administration of the rAAV virion to a subject leads to a lower (e.g., at least 2, 3, 4, 5, 6, 7, 8, 9 or 10 times lower) liver viral load than administration of an rAAV virion having an AAV5 VP1 capsid protein according to SEQ ID NO: 10. In some embodiments, the rAAV virion exhibits a higher transduction efficiency, optionally higher heart transduction efficiency, than an rAAV virion having an AAV5 VP1 capsid protein according to SEQ ID NO: 10, assessed in a primate. In some embodiments, the rAAV virion exhibits a higher (e.g., at least 2, 3, 4, 5, 6, 7, 8, 9 or 10 times higher) heart-to-liver transduction ratio than an rAAV virion having an AAV5 VP1 capsid protein according to SEQ ID NO: 10, assessed in a primate. In some embodiments, administration of the rAAV virion to a subject leads to a lower (e.g., at least 2, 3, 4, 5, 6, 7, 8, 9 or 10 times lower) liver viral load than administration of an rAAV virion having an AAV5 VP1 capsid protein according to SEQ ID NO: 10, as assessed in a primate.

In some embodiments, the rAAV virion exhibits a higher heart transduction efficiency than an rAAV virion having an AAVrh.10 VP1 capsid protein according to SEQ ID NO: 19. In some embodiments, the rAAV virion exhibits a higher (e.g., at least 2, 3, 4, 5, 6, 7, 8, 9 or 10 times higher) heart-to-liver transduction ratio than an rAAV virion having an AAVrh.10 VP1 capsid protein according to SEQ ID NO: 19. In some embodiments, administration of the rAAV virion to a subject leads to a lower (e.g., at least 2, 3, 4, 5, 6, 7, 8, 9 or 10 times lower) liver viral load than administration of an rAAV virion having an AAVrh.10 VP1 capsid protein according to SEQ ID NO: 19. In some embodiments, the rAAV virion exhibits a higher transduction efficiency, optionally higher heart transduction efficiency, than an rAAV virion having an AAVrh.10 VP1 capsid protein according to SEQ ID NO: 19, assessed in a primate. In some embodiments, the rAAV virion exhibits a higher (e.g., at least 2, 3, 4, 5, 6, 7, 8, 9 or 10 times higher) heart-to-liver transduction ratio than an rAAV virion having an AAVrh.10 VP1 capsid protein according to SEQ ID NO: 19, assessed in a primate. In some embodiments, administration of the rAAV virion to a subject leads to a lower (e.g., at least 2, 3, 4, 5, 6, 7, 8, 9 or 10 times lower) liver viral load than administration of an rAAV virion having an AAVrh.10 VP1 capsid protein according to SEQ ID NO: 19, as assessed in a primate.

In some embodiments, the rAAV virion exhibits a higher heart transduction efficiency than an rAAV virion having an AAVrh.74 VP1 capsid protein according to SEQ ID NO: 28. In some embodiments, the rAAV virion exhibits a higher (e.g., at least 2, 3, 4, 5, 6, 7, 8, 9 or 10 times higher) heart-to-liver transduction ratio than an rAAV virion having an AAVrh.74 VP1 capsid protein according to SEQ ID NO: 28. In some embodiments, administration of the rAAV virion to a subject leads to a lower (e.g., at least 2, 3, 4, 5, 6, 7, 8, 9 or 10 times lower) liver viral load than administration of an rAAV virion having an AAVrh.74 VP1 capsid protein according to SEQ ID NO: 28. In some embodiments, the rAAV virion exhibits a higher transduction efficiency, optionally higher heart transduction efficiency, than an rAAV virion having an AAVrh.74 VP1 capsid protein according to SEQ ID NO: 28, assessed in a primate. In some embodiments, the rAAV virion exhibits a higher (e.g., at least 2, 3, 4, 5, 6, 7, 8, 9 or 10 times higher) heart-to-liver transduction ratio than an rAAV virion having an AAVrh.74 VP1 capsid protein according to SEQ ID NO: 28, assessed in a primate. In some embodiments, administration of the rAAV virion to a subject leads to a lower (e.g., at least 2, 3, 4, 5, 6, 7, 8, 9 or 10 times lower) liver viral load than administration of an rAAV virion having an AAVrh.74 VP1 capsid protein according to SEQ ID NO: 28, as assessed in a primate.

Transduction efficiency can be determined using methods known in the art. In some embodiments, the rAAV virion with engineered capsid protein exhibits increased transduction efficiency in cardiac cells compared to an AAV virion comprising the parental sequence. The rAAV virion referenced in this section is any rAAV virion with modified or engineered capsid protein described herein.

In some embodiments, the rAAV virion exhibits increased transduction efficiency in induced pluripotent stem cell-derived cardiomyocyte (iPS-CM) cells compared to an AAV virion comprising the parental sequence. Accordingly, the fold improvement discussed in this section is as compared to an AAV virion comprising the parental sequence (e.g., AAV9).

In some embodiments, the rAAV virion exhibits at least 2-, 3-, 4-, 5-, 6, 7-, 8-, 9-, 10-, 11-, 12-, 13-, 14, or 15-fold increased transduction efficiency in iPS-CM cells at a multiplicity of infection (MOI) of 100,000. In some embodiments, the rAAV virion exhibits about 2- to about 16-fold, about 2- to about 14-fold, about 2- to about 12-fold, about 2- to about 10-fold, about 2- to about 8-fold, about 2- to about 6-fold, about 2- to about 4-fold, or about 2- to about 3-fold increased transduction efficiency in iPS-CM cells at a multiplicity of infection (MOI) of 100,000. In some embodiments, the rAAV virion exhibits about 20% to 30%, about 30% to 40%, about 40% to 50%, about 50% to 80%, about 80% to 100%, about 100% to 125%, about 125% to 150%, about 150% to 175%, or about 175% to 200% increased transduction efficiency in iPS-CM cells at a multiplicity of infection (MOI) of 100,000.

In some embodiments, the rAAV virion exhibits at least 2-, 3-, 4-, 5-, 6, 7-, 8-, 9-, 10-, 11-, 12-, 13-, 14, or 15-fold increased transduction efficiency in iPS-CM cells at a multiplicity of infection (MOI) of 75,000. In some embodiments, the rAAV virion exhibits about 2- to about 16-fold, about 2- to about 14-fold, about 2- to about 12-fold, about 2- to about 10-fold, about 2- to about 8-fold, about 2- to about 6-fold, about 2- to about 4-fold, or about 2- to about 3-fold increased transduction efficiency in iPS-CM cells at a multiplicity of infection (MOI) of 75,000. In some embodiments, the rAAV virion exhibits about 20% to 30%, about 30% to 40%, about 40% to 50%, about 50% to 80%, about 80% to 100%, about 100% to 125%, about 125% to 150%, about 150% to 175%, or about 175% to 200% increased transduction efficiency in iPS-CM cells at a multiplicity of infection (MOI) of 75,000.

In some embodiments, the rAAV virion exhibits at least 2-, 3-, 4-, 5-, 6, 7-, 8-, 9-, 10-, 11-, 12-, 13-, 14, or 15-fold increased transduction efficiency in iPS-CM cells at a multiplicity of infection (MOI) of 1,000. In some embodiments, the rAAV virion exhibits about 2- to about 16-fold, about 2- to about 14-fold, about 2- to about 12-fold, about 2- to about 10-fold, about 2- to about 8-fold, about 2- to about 6-fold, about 2- to about 4-fold, or about 2- to about 3-fold increased transduction efficiency in iPS-CM cells at a multiplicity of infection (MOI) of 1,000. In some embodiments, the rAAV virion exhibits about 20% to 30%, about 30% to 40%, about 40% to 50%, about 50% to 80%, about 80% to 100%, about 100% to 125%, about 125% to 150%, about 150% to 175%, or about 175% to 200% increased transduction efficiency in iPS-CM cells at a multiplicity of infection (MOI) of 1,000.

In some embodiments, the rAAV virion comprising the engineered capsid protein of the present disclosure exhibits increased transduction efficiency in heart compared to an AAV virion comprising the parental sequence. In some embodiments, transduction efficiency in heart is measured in mice. In some embodiments, transduction efficiency in heart is measured in non-human primates (NHPs). In some embodiments, the rAAV virion exhibits at least 2-, 3-, 4-, 5-, 6, 7-, 8-, 9-, 10-, 11-, 12-, 13-, 14, or 15-fold increased transduction efficiency in heart. In some embodiments, the rAAV virion exhibits at least 2-, 3-, 4-, 5-, 6, 7-, 8-, 9-, 10-, 11-, 12-13-, 14, or 15-fold increased transduction efficiency in heart relative to wild-type AAV9. In some embodiments, the rAAV virion exhibits about 2- to about 16-fold, about 2- to about 14-fold, about 2- to about 12-fold, about 2- to about 10-fold, about 2- to about 8-fold, about 2- to about 6-fold, about 2- to about 4-fold, or about 2- to about 3-fold increased transduction efficiency in heart. In some embodiments, the rAAV virion exhibits about 2- to about 16-fold, about 2- to about 14-fold, about 2- to about 12-fold, about 2- to about 10-fold, about 2- to about 8-fold, about 2- to about 6-fold, about 2- to about 4-fold, or about 2- to about 3-fold increased transduction efficiency in heart relative to wild-type AAV9. In some embodiments, the rAAV virion exhibits about 20% to 30%, about 30% to 40%, about 40% to 50%, about 50% to 80%, about 80% to 100%, about 100% to 125%, about 125% to 150%, about 150% to 175%, or about 175% to 200% increased transduction efficiency in heart. In some embodiments, the rAAV virion exhibits about 20% to 30%, about 30% to 40%, about 40% to 50%, about 50% to 80%, about 80% to 100%, about 100% to 125%, about 125% to 150%, about 150% to 175%, or about 175% to 200% increased transduction efficiency in heart relative to wild-type AAV9.

In some embodiments, provided are pharmaceutical compositions comprising an rAAV virion according to various embodiments disclosed herein and a pharmaceutically acceptable carrier or excipient.

In some embodiments, provided are kits comprising a pharmaceutical composition or an rAAV virion according to various embodiments disclosed herein, and optionally instructions for use.

In some embodiments, provided are polynucleotides encoding an engineered capsid protein according to various embodiments disclosed herein.

In some embodiments, provided herein is a method of transducing a cardiac cell, comprising contacting the cardiac cell with any rAAV virion described herein.

In some embodiments, provided are methods of delivering one or more gene products to a cardiac cell, the method comprising contacting the cardiac cell with an rAAV virion according to various embodiments disclosed herein.

In some embodiments, provided are methods of treating cardiac pathology, or a heart disease or condition, in a subject in need thereof, comprising administering to the subject an rAAV virion according to various embodiments disclosed herein. In some embodiments, the subject is a human.

In some embodiments, provided herein is an rAAV virion according to various embodiments disclosed herein for use in treating a cardiac pathology, a heart disease, or a heart condition, in a subject in need thereof.

### Viral and Non-Viral Vectors, Kits, and Pharmaceutical Compositions Thereof

In some embodiments, provided are pharmaceutical compositions comprising an rAAV virion according to various embodiments disclosed herein and a pharmaceutically acceptable carrier or excipient.

In some embodiments, provided are kits comprising an rAAV virion according to various embodiments disclosed herein, and optionally instructions for use. In some embodiments, provided are kits comprising a pharmaceutical composition comprising an rAAV virion according to various embodiments disclosed herein, and optionally instructions for use.

In some embodiments, provided herein is a method of transducing a cardiac cell, comprising contacting the cardiac cell with an rAAV virion according to various embodiments disclosed herein.

In some embodiments, provided are methods of delivering one or more gene products to a cardiac cell, the method comprising contacting the cardiac cell with an rAAV virion according to various embodiments disclosed herein.

In some embodiments, provided are methods of treating cardiac pathology, or a heart disease or condition, in a subject in need thereof, comprising administering to the subject a therapeutically effective amount of an rAAV virion according to various embodiments disclosed herein. In some embodiments, the subject is a mammal. In some embodiments, the subject is a human.

### Gene Products/Transgenes

In some embodiments, an rAAV virion according to various embodiments described herein is used to deliver one or more gene products encoded by one or more transgenes, to cells or tissues such as cardiac cells or tissues.

The transgenes and gene products described herein are non-limiting. Any transgene encoding any gene product may be used in association with the engineered capsid proteins described herein.

A transgene can be a gene or nucleotide sequence that encodes a product, or a functional fragment thereof. A product can be, for example, a polypeptide or a non-coding nucleotide. By non-coding nucleotide, it is meant that the sequence transcribed from the transgene or nucleotide sequence is not translated into a polypeptide. In some embodiments, the product encoded by the transgene or nucleotide operably linked to an enhancer described herein is a non-coding polynucleotide. A non-coding polynucleotide can be an RNA, such as for example a microRNA (miRNA or mIR), short hairpin RNA (shRNA), long non-coding RNA (lnRNA), and/or a short interfering RNA (siRNA). In some embodiments, the transgene encodes a product natively expressed by a cardiac cell, e.g., a cardiomyocyte.

In some embodiments, the transgene encodes a polypeptide. In some embodiments, the transgene encodes a non-coding polynucleotide such as, for example, a microRNA (miRNA or mIR).

In some embodiments, the transgene comprises a nucleotide sequence encoding a human protein. In some embodiments, the transgene comprises a human nucleotide sequence (a human DNA sequence). In some embodiments, the transgene comprises a DNA sequence that has been codon-optimized. In some embodiments, the transgene comprises a nucleotide sequence encoding a wild-type protein, or a functionally active fragment thereof. In some embodiments, the transgene comprises a nucleotide sequence encoding a variant of a wild-type protein, such as a functionally active variant thereof.

In some embodiments, the transgene comprises a sequence encoding a product selected from vascular endothelial growth factor (VEGF), a VEGF isoform, VEGF-A, VEGF-B, VEGF-C, VEGF-D, VEGF-D^{dNdC}, VEGF-A_{116A}, VEGF-A₁₆₅, VEGF-A₁₂₁, VEGF-2, placenta growth factor (PIGF), fibroblast growth factor 4 (FGF-4), human growth factor (HGF), human granulocyte colony-stimulating factor (hGCSF), and hypoxia inducible factor 1α (HIF-1α).

In some embodiments, the transgene comprises a sequence encoding a product selected from SERCA2a, stromal cell-derived factor-1 (SDF-1), adenylyl cyclase type 6, S100A1, miRNA-17-92, miR-302-367, anti-miR-29a, anti-miR-30a, antimiR-141, cyclin A2, cyclin-dependent kinase 2, Tbx20, miRNA-590, miRNA-199, anti-sense oligonucleotide against Lp(a), interfering RNA against PCSK9, anti-sense oligonucleotide against apolipoprotein C-III, lipoprotein lipase^{S447X}, anti-sense oligonucleotide against apolipoprotein B, anti-sense oligonucleotide against c-myc, and E2F oligonucleotide decoy.

In some embodiments, the transgene encodes a gene product whose expression complements a defect in a gene responsible for a genetic disorder. In some embodiments, the disclosure provides, without limitation, polynucleotides encoding one or more of the following—e.g., for use, without limitation, in the disorder indicated in parentheses, or for other disorders caused by each: TAZ (Barth syndrome); FXN (Freidrich's Ataxia); CASQ2 (CPVT); FBN1 (Marfan); RAF1 and SOSIs (Noonan); SCN5A (Brugada); KCNQ1 and KCNH2s (Long QT Syndrome); DMPK (Myotonic Dystrophy 1); LMNA (Limb Girdle Dystrophy Type 1B); JUP (Naxos); TGFBR2 (Loeys-Dietz); EMD (X-Linked EDMD); and ELN (SV Aortic Stenosis). In some embodiments, a polynucleotide encodes one or more of: cardiac troponin T (TNNT2); BAG family molecular chaperone regulator 3 (BAG3); myosin heavy chain (MYH7); tropomyosin 1 (TPM1); myosin binding protein C (MYBPC3); 5′-AMP-activated protein kinase subunit gamma-2 (PRKAG2); troponin I type 3 (TNNI3); titin (TTN); myosin, light chain 2 (MYL2); actin, alpha cardiac muscle 1 (ACTC1); potassium voltage-gated channel, KQT-like subfamily, member 1 (KCNQ1); myocyte enhancer factor 2c (MEF2C); and cardiac LIM protein (CSRP3).

In some embodiments, the transgene comprises a nucleotide sequence encoding a protein selected from DWORF, junctophilin (e.g., JPH2), BAG family molecular chaperone regulator 3 (BAG3), phospholamban (PLN), alpha-crystallin B chain (CRYAB), LMNA (such as Lamin A and Lamin C isoforms), troponin I type 3 (TNNI3), lysosomal-associated membrane protein 2 (LAMP2, such as LAMP2a, LAMP2b and LAMP2c isoforms), desmoplakin (DSP, such as DPI and DPII isoforms), desmoglein 2 (DSG2), junction plakoglobin (JUP), and plakophilin-2 (PKP2). In some embodiments, the transgene comprises a nucleotide sequence encoding a matrix metallopeptidase 11 (MMP11) protein, a synaptopodin 2 like (SYNPO2L) protein (e.g., SYNPO2LA or SYNPO2LA), or an RNA binding motif protein 20 (RBM20). In some embodiments, the transgene comprises a nucleotide sequence encoding an inhibitory oligonucleotide targeting metastasis suppressor protein 1 (MTSS1).

In some embodiments, the transgene in the viral vector (such as that in the rAAV virion of the present disclosure) is selected from DWORF, JPH2, BAG3, CRYAB, LMNA (e.g., Lamin A isoform of LMNA, or Lamin C isoform of LMNA), TNNI3, PLN, LAMP2 (e.g., LAMP2a, LAMP2b, or LAMP2c), DSP (e.g., DPI isoform of DSP or DPII isoform of DSP), DSG2 and JUP.

In some embodiments, the transgene comprises a polynucleotide sequence encoding a MYBPC3 polypeptide. In some embodiments, the transgene comprises a polynucleotide sequence encoding a human MYBPC3 polypeptide. In some embodiments, the transgene comprises, essentially consists of, or consists of SEQ ID NO: 311. In some embodiments, a polynucleotide sequence is a codon-optimized sequence encoding MYBPC3, e.g., human MYBPC3. In some embodiments, the transgene comprises a polynucleotide sequence that has at least 70%, 75%, 80%, 85%, 90%, 95%, 98%, 99% or 100% sequence identity to SEQ ID NO: 311. In some embodiments, the MYBPC3 polypeptide comprises, essentially consists of, or consists of SEQ ID NO: 312. In some embodiments, the MYBPC3 polypeptide has least 75%, 80%, 85%, 90%, 95%, 98%, 99% or 100% sequence identity to SEQ ID NO: 312.

In some embodiments, the transgene comprises a polynucleotide sequence encoding a MYBPC3-delC3 variant polypeptide. In some embodiments, the transgene comprises, essentially consists of, or consists of SEQ ID NO: 313. In some embodiments, a polynucleotide sequence is a codon-optimized sequence encoding MYBPC3-delC3. In some embodiments, the transgene comprises a polynucleotide sequence that has at least 70%, 75%, 80%, 85%, 90%, 95%, 98%, 99% or 100% sequence identity to SEQ ID NO: 313. In some embodiments, the MYBPC3-delC3 variant polypeptide comprises, essentially consists of, or consists of SEQ ID NO: 314. In some embodiments, the MYBPC3-delC3 variant polypeptide has least 75%, 80%, 85%, 90%, 95%, 98%, 99% or 100% sequence identity to SEQ ID NO: 314.

In some embodiments, the transgene comprises a polynucleotide sequence encoding a MYBPC3-delC4 variant polypeptide. In some embodiments, the transgene comprises, essentially consists of, or consists of SEQ ID NO: 315. In some embodiments, a polynucleotide sequence is a codon-optimized sequence encoding MYBPC3-delC4. In some embodiments, the transgene comprises a polynucleotide sequence that has at least 70%, 75%, 80%, 85%, 90%, 95%, 98%, 99% or 100% sequence identity to SEQ ID NO: 315. In some embodiments, the MYBPC3-delC4 variant polypeptide comprises, essentially consists of, or consists of SEQ ID NO: 316. In some embodiments, the MYBPC3-delC4 variant polypeptide has least 75%, 80%, 85%, 90%, 95%, 98%, 99% or 100% sequence identity to SEQ ID NO: 316.

In some embodiments, the transgene comprises a polynucleotide sequence encoding a MYBPC3-delC4b variant polypeptide. In some embodiments, the transgene comprises, essentially consists of, or consists of SEQ ID NO: 317. In some embodiments, a polynucleotide sequence is a codon-optimized sequence encoding MYBPC3-delC4b. In some embodiments, the transgene comprises a polynucleotide sequence that has at least 70%, 75%, 80%, 85%, 90%, 95%, 98%, 99% or 100% sequence identity to SEQ ID NO: 317. In some embodiments, the MYBPC3-delC4b variant polypeptide comprises, essentially consists of, or consists of SEQ ID NO: 318. In some embodiments, the MYBPC3-delC4b variant polypeptide has least 75%, 80%, 85%, 90%, 95%, 98%, 99% or 100% sequence identity to SEQ ID NO: 318.

In some embodiments, the transgene comprises a polynucleotide sequence encoding a DWORF polypeptide. In some embodiments, the transgene comprises a polynucleotide sequence encoding a human DWORF polypeptide. In some embodiments, the transgene comprises, essentially consists of, or consists of SEQ ID NO: 319 or SEQ ID NO: 320. In some embodiments, a polynucleotide sequence is a codon-optimized sequence encoding DWORF, e.g., human DWORF. In some embodiments, the transgene comprises a polynucleotide sequence that has at least 70%, 75%, 80%, 85%, 90%, 95%, 98%, 99% or 100% sequence identity to SEQ ID NO: 319 or SEQ ID NO: 320. In some embodiments, the DWORF polypeptide comprises, essentially consists of, or consists of SEQ ID NO: 321. In some embodiments, the DWORF polypeptide has least 75%, 80%, 85%, 90%, 95%, 98%, 99% or 100% sequence identity to SEQ ID NO: 321.

In some embodiments, the transgene comprises a polynucleotide sequence encoding a junctophilin 2 (JPH2) polypeptide. In some embodiments, the transgene comprises a polynucleotide sequence encoding a full-length JPH2 polypeptide. In some embodiments, the transgene comprises a polynucleotide sequence encoding a human JPH2 polypeptide. In some embodiments, the transgene comprises, essentially consists of, or consists of SEQ ID NO: 322. In some embodiments, a polynucleotide sequence is a codon-optimized sequence encoding JPH2, e.g., human JPH2. In some embodiments, the transgene comprises a polynucleotide sequence that has at least 70%, 75%, 80%, 85%, 90%, 95%, 98%, 99% or 100% sequence identity to SEQ ID NO: 322. In some embodiments, the JPH2 polypeptide comprises, essentially consists of, or consists of SEQ ID NO: 323. In some embodiments, the JPH2 polypeptide has least 75%, 80%, 85%, 90%, 95%, 98%, 99% or 100% sequence identity to SEQ ID NO: 323.

In some embodiments, the transgene comprises a polynucleotide sequence encoding an N-terminal fragment of the JPH2 polypeptide. In some embodiments, the transgene comprises a polynucleotide sequence encoding an N-terminal fragment of the JPH2 polypeptide, which retains the JPH2 activity. In some embodiments, the transgene comprises, essentially consists of, or consists of SEQ ID NO: 324. In some embodiments, a polynucleotide sequence is a codon-optimized sequence encoding N-terminal fragment of JPH2. In some embodiments, the transgene comprises a polynucleotide sequence that has at least 70%, 75%, 80%, 85%, 90%, 95%, 98%, 99% or 100% sequence identity to SEQ ID NO: 324. In some embodiments, the N-terminal fragment of JPH2 polypeptide comprises, essentially consists of, or consists of SEQ ID NO: 325. In some embodiments, the N-terminal fragment of JPH2 polypeptide has least 75%, 80%, 85%, 90%, 95%, 98%, 99% or 100% sequence identity to SEQ ID NO: 325

In some embodiments, the transgene comprises a polynucleotide sequence encoding a BAG3 polypeptide. In some embodiments, the transgene comprises a polynucleotide sequence encoding a human BAG3 polypeptide. In some embodiments, the transgene comprises, essentially consists of, or consists of SEQ ID NO: 326. In some embodiments, a polynucleotide sequence is a codon-optimized sequence encoding BAG3, e.g., human BAG3. In some embodiments, the transgene comprises a polynucleotide sequence that has at least 70%, 75%, 80%, 85%, 90%, 95%, 98%, 99% or 100% sequence identity to SEQ ID NO: 326. In some embodiments, the BAG3 polypeptide comprises, essentially consists of, or consists of SEQ ID NO: 327. In some embodiments, the BAG3 polypeptide has least 75%, 80%, 85%, 90%, 95%, 98%, 99% or 100% sequence identity to SEQ ID NO: 327.

In some embodiments, the transgene comprises a polynucleotide sequence encoding a C151R mutant form of BAG3 polypeptide. In some embodiments, a polynucleotide sequence is a codon-optimized sequence encoding a C151R mutant form of BAG3 polypeptide. In some embodiments, a C151R mutant form of BAG3 polypeptide comprises, essentially consists of, or consists of SEQ ID NO: 328. In some embodiments, a C151R mutant form of BAG3 polypeptide has least 75%, 80%, 85%, 90%, 95%, 98%, 99% or 100% sequence identity to SEQ ID NO: 328.

In some embodiments, the transgene comprises a polynucleotide sequence encoding a CRYAB polypeptide. In some embodiments, the transgene comprises a polynucleotide sequence encoding a human CRYAB polypeptide. In some embodiments, the transgene comprises, essentially consists of, or consists of SEQ ID NO: 329. In some embodiments, a polynucleotide sequence is a codon-optimized sequence encoding CRYAB, e.g., human CRYAB. In some embodiments, the transgene comprises a polynucleotide sequence that has at least 70%, 75%, 80%, 85%, 90%, 95%, 98%, 99% or 100% sequence identity to SEQ ID NO: 329. In some embodiments, the CRYAB polypeptide comprises, essentially consists of, or consists of SEQ ID NO: 330. In some embodiments, the CRYAB polypeptide has least 75%, 80%, 85%, 90%, 95%, 98%, 99% or 100% sequence identity to SEQ ID NO: 330.

In some embodiments, the transgene comprises a polynucleotide sequence encoding a LMNA polypeptide. In some embodiments, the transgene comprises a polynucleotide sequence encoding a human LMNA polypeptide. In some embodiments, the transgene comprises a polynucleotide sequence encoding the LaminA isoform of LMNA. In some embodiments, the transgene comprises, essentially consists of, or consists of SEQ ID NO: 331. In some embodiments, a polynucleotide sequence is a codon-optimized sequence encoding LaminA isoform of LMNA, e.g., human. In some embodiments, the transgene comprises a polynucleotide sequence that has at least 70%, 75%, 80%, 85%, 90%, 95%, 98%, 99% or 100% sequence identity to SEQ ID NO: 331. In some embodiments, the LaminA isoform of LMNA polypeptide comprises, essentially consists of, or consists of SEQ ID NO: 332. In some embodiments, the LMNA polypeptide has least 75%, 80%, 85%, 90%, 95%, 98%, 99% or 100% sequence identity to SEQ ID NO: 332.

In some embodiments, the transgene comprises a polynucleotide sequence encoding the LaminC isoform of LMNA. In some embodiments, the transgene comprises, essentially consists of, or consists of SEQ ID NO: 333. In some embodiments, a polynucleotide sequence is a codon-optimized sequence encoding LaminC isoform of LMNA, e.g., human. In some embodiments, the transgene comprises a polynucleotide sequence that has at least 70%, 75%, 80%, 85%, 90%, 95%, 98%, 99% or 100% sequence identity to SEQ ID NO: 333. In some embodiments, the LaminC isoform of LMNA polypeptide comprises, essentially consists of, or consists of SEQ ID NO: 334. In some embodiments, the LMNA polypeptide has least 75%, 80%, 85%, 90%, 95%, 98%, 99% or 100% sequence identity to SEQ ID NO: 334.

In some embodiments, the transgene comprises a polynucleotide sequence encoding a TNNI3 polypeptide. In some embodiments, the transgene comprises a polynucleotide sequence encoding a human TNNI3 polypeptide. In some embodiments, the transgene comprises, essentially consists of, or consists of SEQ ID NO: 335. In some embodiments, a polynucleotide sequence is a codon-optimized sequence encoding TNNI3, e.g., human TNNI3. In some embodiments, the transgene comprises a polynucleotide sequence that has at least 70%, 75%, 80%, 85%, 90%, 95%, 98%, 99% or 100% sequence identity to SEQ ID NO: 335. In some embodiments, the TNNI3 polypeptide comprises, essentially consists of, or consists of SEQ ID NO: 336. In some embodiments, the TNNI3 polypeptide has least 75%, 80%, 85%, 90%, 95%, 98%, 99% or 100% sequence identity to SEQ ID NO: 336.

In some embodiments, the transgene comprises a polynucleotide sequence encoding a PLN polypeptide. In some embodiments, the transgene comprises a polynucleotide sequence encoding a human PLN polypeptide. In some embodiments, the transgene comprises, essentially consists of, or consists of SEQ ID NO: 337. In some embodiments, a polynucleotide sequence is a codon-optimized sequence encoding PLN, e.g., human PLN. In some embodiments, the transgene comprises a polynucleotide sequence that has at least 70%, 75%, 80%, 85%, 90%, 95%, 98%, 99% or 100% sequence identity to SEQ ID NO: 337. In some embodiments, the PLN polypeptide comprises, essentially consists of, or consists of SEQ ID NO: 338. In some embodiments, the PLN polypeptide has least 75%, 80%, 85%, 90%, 95%, 98%, 99% or 100% sequence identity to SEQ ID NO: 338.

In some embodiments, the transgene comprises a polynucleotide sequence encoding a guide RNA targeting a mutant PLN gene (such as a deletious mutant of PLN, e.g., PLN-R14Del).

In some embodiments, the transgene comprises a polynucleotide sequence encoding a LAMP2 polypeptide. In some embodiments, the transgene comprises a polynucleotide sequence encoding a human LAMP2 polypeptide. In some embodiments, the transgene comprises a polynucleotide sequence encoding the LAMP2a isoform. In some embodiments, the transgene comprises, essentially consists of, or consists of SEQ ID NO: 339. In some embodiments, a polynucleotide sequence is a codon-optimized sequence encoding LAMP2a, e.g., human LAMP2a. In some embodiments, the transgene comprises a polynucleotide sequence that has at least 70%, 75%, 80%, 85%, 90%, 95%, 98%, 99% or 100% sequence identity to SEQ ID NO: 339. In some embodiments, the LAMP2a polypeptide comprises, essentially consists of, or consists of SEQ ID NO: 340. In some embodiments, the LAMP2a polypeptide has least 75%, 80%, 85%, 90%, 95%, 98%, 99% or 100% sequence identity to SEQ ID NO: 340.

In some embodiments, the transgene comprises a polynucleotide sequence encoding the LAMP2b isoform. In some embodiments, the transgene comprises, essentially consists of, or consists of SEQ ID NO: 341. In some embodiments, a polynucleotide sequence is a codon-optimized sequence encoding LAMP2b, e.g., human LAMP2b. In some embodiments, the transgene comprises a polynucleotide sequence that has at least 70%, 75%, 80%, 85%, 90%, 95%, 98%, 99% or 100% sequence identity to SEQ ID NO: 341. In some embodiments, the LAMP2b polypeptide comprises, essentially consists of, or consists of SEQ ID NO: 342. In some embodiments, the LAMP2b polypeptide has least 75%, 80%, 85%, 90%, 95%, 98%, 99% or 100% sequence identity to SEQ ID NO: 342.

In some embodiments, the transgene comprises a polynucleotide sequence encoding the LAMP2c isoform. In some embodiments, the transgene comprises, essentially consists of, or consists of SEQ ID NO: 343. In some embodiments, a polynucleotide sequence is a codon-optimized sequence encoding LAMP2c, e.g., human LAMP2c. In some embodiments, the transgene comprises a polynucleotide sequence that has at least 70%, 75%, 80%, 85%, 90%, 95%, 98%, 99% or 100% sequence identity to SEQ ID NO: 343. In some embodiments, the LAMP2c polypeptide comprises, essentially consists of, or consists of SEQ ID NO: 344. In some embodiments, the LAMP2c polypeptide has least 75%, 80%, 85%, 90%, 95%, 98%, 99% or 100% sequence identity to SEQ ID NO: 344.

In some embodiments, the transgene comprises a polynucleotide sequence encoding a DSP polypeptide. In some embodiments, the transgene comprises a polynucleotide sequence encoding a human DSP polypeptide. In some embodiments, the transgene comprises a polynucleotide sequence encoding the DPI isoform of DSP. In some embodiments, the transgene comprises, essentially consists of, or consists of SEQ ID NO: 345. In some embodiments, a polynucleotide sequence is a codon-optimized sequence encoding DPI isoform of DSP, e.g., human. In some embodiments, the transgene comprises a polynucleotide sequence that has at least 70%, 75%, 80%, 85%, 90%, 95%, 98%, 99% or 100% sequence identity to SEQ ID NO: 345. In some embodiments, the DPI isoform of DSP polypeptide comprises, essentially consists of, or consists of SEQ ID NO: 346. In some embodiments, the DPI isoform of DSP polypeptide has least 75%, 80%, 85%, 90%, 95%, 98%, 99% or 100% sequence identity to SEQ ID NO: 346.

In some embodiments, the transgene comprises a polynucleotide sequence encoding the DPII isoform of DSP. In some embodiments, the transgene comprises, essentially consists of, or consists of SEQ ID NO: 347. In some embodiments, a polynucleotide sequence is a codon-optimized sequence encoding DPII isoform of DSP, e.g., human. In some embodiments, the transgene comprises a polynucleotide sequence that has at least 70%, 75%, 80%, 85%, 90%, 95%, 98%, 99% or 100% sequence identity to SEQ ID NO: 347. In some embodiments, the DPII isoform of DSP polypeptide comprises, essentially consists of, or consists of SEQ ID NO: 348. In some embodiments, the DPII isoform of DSP polypeptide has least 75%, 80%, 85%, 90%, 95%, 98%, 99% or 100% sequence identity to SEQ ID NO: 348.

In some embodiments, the transgene comprises a polynucleotide sequence encoding a DSG2 polypeptide. In some embodiments, the transgene comprises a polynucleotide sequence encoding a human DSG2 polypeptide. In some embodiments, the transgene comprises, essentially consists of, or consists of SEQ ID NO: 349. In some embodiments, a polynucleotide sequence is a codon-optimized sequence encoding DSG2, e.g., human DSG2. In some embodiments, the transgene comprises a polynucleotide sequence that has at least 70%, 75%, 80%, 85%, 90%, 95%, 98%, 99% or 100% sequence identity to SEQ ID NO: 349. In some embodiments, the DSG2 polypeptide comprises, essentially consists of, or consists of SEQ ID NO: 350. In some embodiments, the DSG2 polypeptide has least 75%, 80%, 85%, 90%, 95%, 98%, 99% or 100% sequence identity to SEQ ID NO: 350.

In some embodiments, the transgene comprises a polynucleotide sequence encoding a JUP polypeptide. In some embodiments, the transgene comprises a polynucleotide sequence encoding a human JUP polypeptide. In some embodiments, the transgene comprises, essentially consists of, or consists of SEQ ID NO: 351. In some embodiments, a polynucleotide sequence is a codon-optimized sequence encoding JUP, e.g., human JUP. In some embodiments, the transgene comprises a polynucleotide sequence that has at least 70%, 75%, 80%, 85%, 90%, 95%, 98%, 99% or 100% sequence identity to SEQ ID NO: 351. In some embodiments, the JUP polypeptide comprises, essentially consists of, or consists of SEQ ID NO: 352. In some embodiments, the JUP polypeptide has least 75%, 80%, 85%, 90%, 95%, 98%, 99% or 100% sequence identity to SEQ ID NO: 352

In some embodiments, the transgene comprises a polynucleotide sequence encoding MMP11. In some embodiments, the transgene comprises a polynucleotide sequence encoding a human MMP11 polypeptide. In some embodiments, the transgene comprises, essentially consists of, or consists of SEQ ID NO: 353. In some embodiments, a polynucleotide sequence is a codon-optimized sequence encoding MMP11, e.g., human MMP11. In some embodiments, the transgene comprises a polynucleotide sequence that has at least 70%, 75%, 80%, 85%, 90%, 95%, 98%, 99% or 100% sequence identity to SEQ ID NO: 353. In some embodiments, the MMP11 polypeptide comprises, essentially consists of, or consists of SEQ ID NO: 354. In some embodiments, the MMP11 polypeptide has least 75%, 80%, 85%, 90%, 95%, 98%, 99% or 100% sequence identity to SEQ ID NO: 354.

In some embodiments, the transgene comprises a polynucleotide sequence encoding SYNPO2L (e.g., SYNPO2LA or SYNPO2LA). In some embodiments, the transgene comprises a polynucleotide sequence encoding a human SYNPO2L (e.g., SYNPO2LA or SYNPO2LA). In some embodiments, a polynucleotide sequence is a codon-optimized sequence encoding SYNPO2LA, e.g., human. In some embodiments, the transgene comprises, essentially consists of, or consists of SEQ ID NO: 355. In some embodiments, the transgene comprises a polynucleotide sequence that has at least 70%, 75%, 80%, 85%, 90%, 95%, 98%, 99% or 100% sequence identity to SEQ ID NO: 355. In some embodiments, the SYNPO2LA polypeptide comprises, essentially consists of, or consists of SEQ ID NO: 356. In some embodiments, the SYNPO2LA polypeptide has least 75%, 80%, 85%, 90%, 95%, 98%, 99% or 100% sequence identity to SEQ ID NO: 356. In some embodiments, a polynucleotide sequence is a codon-optimized sequence encoding SYNPO2LB, e.g., human. In some embodiments, the transgene comprises, essentially consists of, or consists of SEQ ID NO: 357. In some embodiments, the transgene comprises a polynucleotide sequence that has at least 70%, 75%, 80%, 85%, 90%, 95%, 98%, 99% or 100% sequence identity to SEQ ID NO: 357. In some embodiments, the SYNPO2LB polypeptide comprises, essentially consists of, or consists of SEQ ID NO: 358. In some embodiments, the SYNPO2LB polypeptide has least 75%, 80%, 85%, 90%, 95%, 98%, 99% or 100% sequence identity to SEQ ID NO: 358.

In some embodiments, the transgene comprises a polynucleotide sequence encoding an inhibitory oligonucleotide (e.g., siRNA) targeting MTSS1. In some embodiments, the transgene comprises a polynucleotide sequence encoding an inhibitory oligonucleotide (e.g., siRNA) targeting SEQ ID NO: 359.

In some embodiments, the transgene comprises a polynucleotide sequence encoding saCas9. In some embodiments, the transgene comprises, essentially consists of, or consists of SEQ ID NO: 360. In some embodiments, a polynucleotide sequence is a codon-optimized sequence encoding saCas9. In some embodiments, the transgene comprises a polynucleotide sequence that has at least 70%, 75%, 80%, 85%, 90%, 95%, 98%, 99% or 100% sequence identity to SEQ ID NO: 360. In some embodiments, the saCas9 polypeptide comprises, essentially consists of, or consists of SEQ ID NO: 361. In some embodiments, the saCas9 polypeptide has least 75%, 80%, 85%, 90%, 95%, 98%, 99% or 100% sequence identity to SEQ ID NO: 361.

In some embodiments, the transgene comprises a polynucleotide sequence encoding spCas9. In some embodiments, the spCas9 is a split spCas9 comprising a first polynucleotide sequence that encodes an N-terminal spCas9 fragment polypeptide and a second polynucleotide sequence that encodes a C-terminal spCas9 fragment polypeptide. In some embodiments, the split spCas9 comprises a H840A substitution, wherein the amino acid numbering is with respect to a wild-type spCas9 sequence. In some embodiments, the first polynucleotide sequence comprises, essentially consists of, or consists of SEQ ID NO: 362. In some embodiments, the second polynucleotide sequence comprises, essentially consists of, or consists of SEQ ID NO: 364. In some embodiments, the first and/or second polynucleotide sequence is a codon-optimized sequence encoding a fragment of spCas9. In some embodiments, the transgene comprises a first polynucleotide sequence that has at least 70%, 75%, 80%, 85%, 90%, 95%, 98%, 99% or 100% sequence identity to SEQ ID NO: 362. In some embodiments, the N-terminal spCas9 fragment polypeptide and a second polynucleotide sequence that encodes a C-terminal spCas9 fragment polypeptide comprises a second polynucleotide sequence that has at least 70%, 75%, 80%, 85%, 90%, 95%, 98%, 99% or 100% sequence identity to SEQ ID NO: 364. In some embodiments, the N-terminal spCas9 fragment polypeptide comprises, essentially consists of, or consists of SEQ ID NO: 363. In some embodiments, the N-terminal spCas9 fragment polypeptide has least 75%, 80%, 85%, 90%, 95%, 98%, 99% or 100% sequence identity to SEQ ID NO: 363. In some embodiments, the C-terminal spCas9 fragment polypeptide comprises, essentially consists of, or consists of SEQ ID NO: 365. In some embodiments, the C-terminal spCas9 fragment polypeptide has least 75%, 80%, 85%, 90%, 95%, 98%, 99% or 100% sequence identity to SEQ ID NO: 365.

Exemplary polynucleotide and amino acid sequences of the transgenes and gene products as described are provided in Table 9 below.

TABLE 9
Exemplary transgene and gene product sequences

| | | SEQ |
|---|---|---|
| | | ID |
| Name | Sequence | NO: |
| Human | atgcctgagccggggaagaagccagtctcagcttttagcaagaagccacggtcagtggaagtggccgcaggcagccctg | 311 |
| MYBPC | ccgtgttcgaggccgagacagagcgggcaggagtgaaggtgcgctggcagcgcggaggcagtgacatcagcgccagc | |
| 3 DNA | aacaagtacggcctggccacagagggcacacggcatacgctgacagtgcgggaagtgggccctgccgaccagggatctt | |
| | acgcagtcattgctggctcctccaaggtcaagttcgacctcaaggtcatagaggcagagaaggcagagcccatgctggccc | |
| | ctgcccctgcccctgctgaggccactggagcccctggagaagccccggccccagccgctgagctgggagaaagtgccc | |
| | caagtcccaaagggtcaagctcagcagctctcaatggtcctacccctggagcccccgatgaccccattggcctcttcgtgat | |
| | gcggccacaggatggcgaggtgaccgtgggtggcagcatcaccttctcagcccgcgtggccggcgccagcctcctgaag | |
| | ccgcctgtggtcaagtggttcaagggcaaatgggtggacctgagcagcaaggtgggccagcacctgcagctgcacgaca | |
| | gctacgaccgcgccagcaaggtctatctgttcgagctgcacatcaccgatgcccagcctgccttcactggcagctaccgctg | |
| | tgaggtgtccaccaaggacaaatttgactgctccaacttcaatctcactgtccacgaggccatgggcaccggagacctggac | |
| | ctcctatcagccttccgccgcacgagcctggctggaggtggtcggcggatcagtgatagccatgaggacactgggattctg | |
| | gacttcagctcactgctgaaaaagagagacagtttccggaccccgagggactcgaagctggaggcaccagcagaggagg | |
| | acgtgtgggagatcctacggcaggcacccccatctgagtacgagcgcatcgccttccagtacggcgtcactgacctgcgc | |
| | ggcatgctaaagaggctcaagggcatgaggcgcgatgagaagaagagcacagcctttcagaagaagctggagccggcc | |
| | taccaggtgagcaaaggccacaagatccggctgaccgtggaactggctgaccatgacgctgaggtcaaatggctcaagaa | |
| | tggccaggagatccagatgagcggcagcaagtacatctttgagtccatcggtgccaagcgtaccctgaccatcagccagtg | |
| | ctcattggcggacgacgcagcctaccagtgcgtggtgggggcgagaagtgtagcacggagctctttgtgaaagagcccc | |
| | ctgtgctcatcacgcgccccttggaggaccagctggtgatggtggggcagcgggtggagtttgagtgtgaagtatcggagg | |
| | agggggcgcaagtcaaatggctgaaggacggggtggagctgacccgggaggagaccttcaaataccggttcaagaagg | |
| | acgggcagagacaccacctgatcatcaacgaggccatgctggaggacgcggggcactatgcactgtgcactagcgggg | |
| | gccaggcgctggctgagctcattgtgcaggaaaagaagctggaggtgtaccagagcatcgcagacctgatggtgggcgc | |
| | aaaggaccaggcggtgttcaaatgtgaggtctcagatgagaatgttcggggtgtgtggctgaagaatgggaaggagctggt | |
| | gcccgacagccgcataaaggtgtcccacatcgggcgggtccacaaactgaccattgacgacgtcacacctgccgacgag | |
| | gctgactacagctttgtgcccgagggcttcgcctgcaacctgtcagccaagctccacttcatggaggtcaagattgacttcg | |
| | tacccaggcaggaacctcccaagatccacctggactgcccaggccgcataccagacaccattgtggttgtagctggaaataa | |
| | gctacgtctggacgtccctatctctggggaccccgctcccactgtgatctggcagaaggctatcacgcaggggaataaggc | |
| | cccagccaggccagccccagatgccccagaggacacaggtgacagcgatgagtgggtgtttgacaagaagctgctgtgt | |
| | gagaccgagggccgggtccgcgtggagaccaccaaggaccgcagcatcttcacggtcgagggggcagagaaggaag | |
| | atgagggcgtctacacggtcacagtgaagaaccctgtgggcgaggaccaggtcaacctcacagtcaaggtcatcgacgtg | |
| | ccagacgcacctgcggcccccaagatcagcaacgtgggagaggactcctgcacagtacagtgggagccgcctgcctacg | |
| | atggcgggcagcccatcctgggctacatcctggagcgcaagaagaagaagagctaccggtggatgcggctgaacttcga | |
| | cctgattcaggagctgagtcatgaagcgcggcgcatgatcgagggcgtggtgtacgagatgcgcgtctacgcggtcaacg | |
| | ccatcggcatgtccaggcccagccctgcctcccagcccttcatgcctatcggtccccccagcgaacccacccacctggcag | |
| | tagaggacgtctctgacaccacggtctccctcaagtggcggcccccagagcgcgtgggagcaggaggcctggatggcta | |
| | cagcgtggagtactgcccagagggctgctcagagtgggtggctgccctgcaggggctgacagagcacacatcgatactg | |
| | gtgaaggacctgcccacgggggcccggctgcttttccgagtgcgggcacacaatatggcagggcctggagcccctgttac | |
| | caccacggagccggtgacagtgcaggagatcctgcaacggccacggcttcagctgcccaggcacctgcgccagaccatt | |
| | cagaagaaggtcggggagcctgtgaaccttctcatccctttccagggcaagccccggcctcaggtgacctggaccaaaga | |
| | ggggcagcccctggcaggcgaggaggtgagcatccgcaacagccccacagacaccatcctgttcatccgggccgctcg | |
| | ccgcgtgcattcaggcacttaccaggtgacggtgcgcattgagaacatggaggacaaggccacgctggtgctgcaggttgt | |
| | tgacaagccaagtcctccccaggatctccgggtgactgacgcctggggtcttaatgtggctctggagtggaagccacccca | |
| | ggatgtcggcaacacggaactctgggggtacacagtgcagaaagccgacaagaagaccatggagtggttcaccgtcttgg | |
| | agcattaccgccgcacccactgcgtggtgccagagctcatcattggcaatggctactacttccgcgtcttcagccagaatat | |
| | ggttggctttagtgacagagcggccaccaccaaggagcccgtctttatccccagaccaggcatcacctatgagccacccaac | |
| | tataaggccctggacttctccgaggccccaagcttcacccagcccctggtgaaccgctcggtcatcgcgggctacactgcta | |
| | tgctctgctgtgctgtccggggtagccccaagcccaagatttcctggttcaagaatggcctggacctgggagaagacgcccg | |
| | cttccgcatgttcagcaagcagggagtgttgactctggagattagaaagccctgcccctttgacgggggcatctatgtctgc | |
| | agggccaccaacttacagggcgaggcacggtgtgagtgccgcctggaggtgcgagtgcctcagtaa | |
| Human | MPEPGKKPVSAFSKKPRSVEVAAGSPAVFEAETERAGVKVRWQRGGSDISAS | 312 |
| MYBPC | NKYGLATEGTRHTLTVREVGPADQGSYAVIAGSSKVKFDLKVIEAEKAEPML | |
| 3 | APAPAPAEATGAPGEAPAPAAELGESAPSPKGSSSAALNGPTPGAPDDPIGLFV | |
| protein | MRPQDGEVTVGGSITFSARVAGASLLKPPVVKWFKGKWVDLSSKVGQHLQL | |
| | HDSYDRASKVYLFELHITDAQPAFTGSYRCEVSTKDKFDCSNFNLTVHEAMG | |
| | TGDLDLLSAFRRTSLAGGGRRISDSHEDTGILDFSSLLKKRDSFRTPRDSKLEAP | |
| | AEEDVWEILRQAPPSEYERIAFQYGVTDLRGMLKRLKGMRRDEKKSTAFQKK | |
| | LEPAYQVSKGHKIRLTVELADHDAEVKWLKNGQEIQMSGSKYIFESIGAKRTL | |
| | TISQCSLADDAAYQCVVGGEKCSTELFVKEPPVLITRPLEDQLVMVGQRVEFE | |
| | CEVSEEGAQVKWLKDGVELTREETFKYRFKKDGQRHHLIINEAMLEDAGHYA | |
| | LCTSGGQALAELIVQEKKLEVYQSIADLMVGAKDQAVFKCEVSDENVRGVW | |
| | LKNGKELVPDSRIKVSHIGRVHKLTIDDVTPADEADYSFVPEGFACNLSAKLHF | |
| | MEVKIDFVPRQEPPKIHLDCPGRIPDTIVVVAGNKLRLDVPISGDPAPTVIWQK | |
| | AITQGNKAPARPAPDAPEDTGDSDEWVFDKKLLCETEGRVRVETTKDRSIFTV | |
| | EGAEKEDEGVYTVTVKNPVGEDQVNLTVKVIDVPDAPAAPKISNVGEDSCTV | |
| | QWEPPAYDGGQPILGYILERKKKKSYRWMRLNFDLIQELSHEARRMIEGVVY | |
| | EMRVYAVNAIGMSRPSPASQPFMPIGPPSEPTHLAVEDVSDTTVSLKWRPPER | |
| | VGAGGLDGYSVEYCPEGCSEWVAALQGLTEHTSILVKDLPTGARLLFRVRAH | |
| | NMAGPGAPVTTTEPVTVQEILQRPRLQLPRHLRQTIQKKVGEPVNLLIPFQGKP | |
| | RPQVTWTKEGQPLAGEEVSIRNSPTDTILFIRAARRVHSGTYQVTVRIENMEDK | |
| | ATLVLQVVDKPSPPQDLRVTDAWGLNVALEWKPPQDVGNTELWGYTVQKA | |
| | DKKTMEWFTVLEHYRRTHCVVPELIIGNGYYFRVFSQNMVGFSDRAATTKEP | |
| | VFIPRPGITYEPPNYKALDFSEAPSFTQPLVNRSVIAGYTAMLCCAVRGSPKPKI | |
| | SWFKNGLDLGEDARFRMFSKQGVLTLEIRKPCPFDGGIYVCRATNLQGEARCE | |
| | CRLEVRVPQ | |
| MYBPC | atgcctgagccggggaagaagccagtctcagcttttagcaagaagccacggtcagtggaagtggccgcaggcagccctg | 313 |
| 3-delC3 | ccgtgttcgaggccgagacagagcgggcaggagtgaaggtgcgctggcagcgcggaggcagtgacatcagcgccagc | |
| DNA | aacaagtacggcctggccacagagggcacacggcatacgctgacagtgcgggaagtgggccctgccgaccagggatctt | |
| | acgcagtcattgctggctcctccaaggtcaagttcgacctcaaggtcatagaggcagagaaggcagagcccatgctggccc | |
| | ctgcccctgcccctgctgaggccactggagcccctggagaagccccggccccagccgctgagctgggagaaagtgccc | |
| | caagtcccaaagggtcaagctcagcagctctcaatggtcctacccctggagcccccgatgaccccattggcctcttcgtgat | |
| | gcggccacaggatggcgaggtgaccgtgggtggcagcatcaccttctcagcccgcgtggccggcgccagcctcctgaag | |
| | ccgcctgtggtcaagtggttcaagggcaaatgggtggacctgagcagcaaggtgggccagcacctgcagctgcacgaca | |
| | gctacgaccgcgccagcaaggtctatctgttcgagctgcacatcaccgatgcccagcctgccttcactggcagctaccgctg | |
| | tgaggtgtccaccaaggacaaatttgactgctccaacttcaatctcactgtccacgaggccatgggcaccggagacctggac | |
| | ctcctatcagccttccgccgcacgagcctggctggaggtggtcggcggatcagtgatagccatgaggacactgggattctg | |
| | gacttcagctcactgctgaaaaagagagacagtttccggaccccgagggactcgaagctggaggcaccagcagaggagg | |
| | acgtgtgggagatcctacggcaggcacccccatctgagtacgagcgcatcgccttccagtacggcgtcactgacctgcgc | |
| | ggcatgctaaagaggctcaagggcatgaggcgcgatgagaagaagagcacagcctttcagaagaagctggagccggcc | |
| | taccaggtgagcaaaggccacaagatccggctgaccgtggaactggctgaccatgacgctgaggtcaaatggctcaagaa | |
| | tggccaggagatccagatgagcggcagcaagtacatctttgagtccatcggtgccaagcgtaccctgaccatcagccagtg | |
| | ctcattggcggacgacgcagcctaccagtgcgtggtgggtggcgagaagtgtagcacggagctctttgtgaaagagcccc | |
| | ctgtgtaccagagcatcgcagacctgatggtgggcgcaaaggaccaggcggtgttcaaatgtgaggtctcagatgagaatg | |
| | ttcggggtgtgtggctgaagaatgggaaggagctggtgcccgacagccgcataaaggtgtcccacatcgggcgggtcca | |
| | caaactgaccattgacgacgtcacacctgccgacgaggctgactacagctttgtgcccgagggcttcgcctgcaacctgtca | |
| | gccaagctccacttcatggaggtcaagattgacttcgtacccaggcaggaacctcccaagatccacctggactgcccaggc | |
| | cgcataccagacaccattgtggttgtagctggaaataagctacgtctggacgtccctatctctggggaccccgctcccactg | |
| | tgatctggcagaaggctatcacgcaggggaataaggccccagccaggccagccccagatgccccagaggacacaggtga | |
| | cagcgatgagtgggtgtttgacaagaagctgctgtgtgagaccgagggccgggtccgcgtggagaccaccaaggaccgc | |
| | agcatcttcacggtcgagggggcagagaaggaagatgagggcgtctacacggtcacagtgaagaaccctgtgggcgag | |
| | gaccaggtcaacctcacagtcaaggtcatcgacgtgccagacgcacctgcggcccccaagatcagcaacgtgggagagg | |
| | actcctgcacagtacagtgggagccgcctgcctacgatggcgggcagcccatcctgggctacatcctggagcgcaagaag | |
| | aagaagagctaccggtggatgcggctgaacttcgacctgattcaggagctgagtcatgaagcgcggcgcatgatcgaggg | |
| | cgtggtgtacgagatgcgcgtctacgcggtcaacgccatcggcatgtccaggcccagccctgcctcccagcccttcatgcc | |
| | tatcggtccccccagcgaacccacccacctggcagtagaggacgtctctgacaccacggtctccctcaagtggcggcccc | |
| | cagagcgcgtgggagcaggaggcctggatggctacagcgtggagtactgcccagagggctgctcagagtgggtggctg | |
| | ccctgcaggggctgacagagcacacatcgatactggtgaaggacctgcccacgggggcccggctgcttttccgagtgcg | |
| | ggcacacaatatggcagggcctggagcccctgttaccaccacggagccggtgacagtgcaggagatcctgcaacggcca | |
| | cggcttcagctgcccaggcacctgcgccagaccattcagaagaaggtcggggagcctgtgaaccttctcatccctttccag | |
| | ggcaagccccggcctcaggtgacctggaccaaagaggggcagcccctggcaggcgaggaggtgagcatccgcaacag | |
| | ccccacagacaccatcctgttcatccgggccgctcgccgcgtgcattcaggcacttaccaggtgacggtgcgcattgagaa | |
| | catggaggacaaggccacgctggtgctgcaggttgttgacaagccaagtcctccccaggatctccgggtgactgacgcct | |
| | ggggtcttaatgtggctctggagtggaagccaccccaggatgtcggcaacacggaactctgggggtacacagtgcagaaa | |
| | gccgacaagaagaccatggagtggttcaccgtcttggagcattaccgccgcacccactgcgtggtgccagagctcatcatt | |
| | ggcaatggctactacttccgcgtcttcagccagaatatggttggctttagtgacagagcggccaccaccaaggagcccgtct | |
| | ttatccccagaccaggcatcacctatgagccacccaactataaggccctggacttctccgaggccccaagcttcacccagcc | |
| | cctggtgaaccgctcggtcatcgcgggctacactgctatgctctgctgtgctgtccggggtagccccaagcccaagatttcc | |
| | tggttcaagaatggcctggacctgggagaagacgcccgcttccgcatgttcagcaagcagggagtgttgactctggagatta | |
| | gaaagccctgcccctttgacgggggcatctatgtctgcagggccaccaacttacagggcgaggcacggtgtgagtgccgc | |
| | ctggaggtgcgagtgcctcagtaa | |
| MYBPC | MPEPGKKPVSAFSKKPRSVEVAAGSPAVFEAETERAGVKVRWQRGGSDISAS | 314 |
| 3-delC3 | NKYGLATEGTRHTLTVREVGPADQGSYAVIAGSSKVKFDLKVIEAEKAEPML | |
| protein | APAPAPAEATGAPGEAPAPAAELGESAPSPKGSSSAALNGPTPGAPDDPIGLFV | |
| | MRPQDGEVTVGGSITFSARVAGASLLKPPVVKWFKGKWVDLSSKVGQHLQL | |
| | HDSYDRASKVYLFELHITDAQPAFTGSYRCEVSTKDKFDCSNFNLTVHEAMG | |
| | TGDLDLLSAFRRTSLAGGGRRISDSHEDTGILDFSSLLKKRDSFRTPRDSKLEAP | |
| | AEEDVWEILRQAPPSEYERIAFQYGVTDLRGMLKRLKGMRRDEKKSTAFQKK | |
| | LEPAYQVSKGHKIRLTVELADHDAEVKWLKNGQEIQMSGSKYIFESIGAKRTL | |
| | TISQCSLADDAAYQCVVGGEKCSTELFVKEPPVYQSIADLMVGAKDQAVFKC | |
| | EVSDENVRGVWLKNGKELVPDSRIKVSHIGRVHKLTIDDVTPADEADYSFVPE | |
| | GFACNLSAKLHFMEVKIDFVPRQEPPKIHLDCPGRIPDTIVVVAGNKLRLDVPI | |
| | SGDPAPTVIWQKAITQGNKAPARPAPDAPEDTGDSDEWVFDKKLLCETEGRV | |
| | RVETTKDRSIFTVEGAEKEDEGVYTVTVKNPVGEDQVNLTVKVIDVPDAPAA | |
| | PKISNVGEDSCTVQWEPPAYDGGQPILGYILERKKKKSYRWMRLNFDLIQELS | |
| | HEARRMIEGVVYEMRVYAVNAIGMSRPSPASQPFMPIGPPSEPTHLAVEDVSD | |
| | TTVSLKWRPPERVGAGGLDGYSVEYCPEGCSEWVAALQGLTEHTSILVKDLP | |
| | TGARLLFRVRAHNMAGPGAPVTTTEPVTVQEILQRPRLQLPRHLRQTIQKKVG | |
| | EPVNLLIPFQGKPRPQVTWTKEGQPLAGEEVSIRNSPTDTILFIRAARRVHSGTY | |
| | QVTVRIENMEDKATLVLQVVDKPSPPQDLRVTDAWGLNVALEWKPPQDVGN | |
| | TELWGYTVQKADKKTMEWFTVLEHYRRTHCVVPELIIGNGYYFRVFSQNMV | |
| | GFSDRAATTKEPVFIPRPGITYEPPNYKALDFSEAPSFTQPLVNRSVIAGYTAML | |
| | CCAVRGSPKPKISWFKNGLDLGEDARFRMFSKQGVLTLEIRKPCPFDGGIYVC | |
| | RATNLQGEARCECRLEVRVPQ | |
| MYBPC | atgcctgagccggggaagaagccagtctcagcttttagcaagaagccacggtcagtggaagtggccgcaggcagccctg | 315 |
| 3-delC4 | ccgtgttcgaggccgagacagagcgggcaggagtgaaggtgcgctggcagcgcggaggcagtgacatcagcgccagc | |
| DNA | aacaagtacggcctggccacagagggcacacggcatacgctgacagtgcgggaagtgggccctgccgaccagggatctt | |
| | acgcagtcattgctggctcctccaaggtcaagttcgacctcaaggtcatagaggcagagaaggcagagcccatgctggccc | |
| | ctgcccctgcccctgctgaggccactggagcccctggagaagccccggccccagccgctgagctgggagaaagtgccc | |
| | caagtcccaaagggtcaagctcagcagctctcaatggtcctacccctggagcccccgatgaccccattggcctcttcgtgat | |
| | gcggccacaggatggcgaggtgaccgtgggtggcagcatcaccttctcagcccgcgtggccggcgccagcctcctgaag | |
| | ccgcctgtggtcaagtggttcaagggcaaatgggtggacctgagcagcaaggtgggccagcacctgcagctgcacgaca | |
| | gctacgaccgcgccagcaaggtctatctgttcgagctgcacatcaccgatgcccagcctgccttcactggcagctaccgctg | |
| | tgaggtgtccaccaaggacaaatttgactgctccaacttcaatctcactgtccacgaggccatgggcaccggagacctggac | |
| | ctcctatcagccttccgccgcacgagcctggctggaggtggtcggcggatcagtgatagccatgaggacactgggattctg | |
| | gacttcagctcactgctgaaaaagagagacagtttccggaccccgagggactcgaagctggaggcaccagcagaggagg | |
| | acgtgtgggagatcctacggcaggcacccccatctgagtacgagcgcatcgccttccagtacggcgtcactgacctgcgc | |
| | ggcatgctaaagaggctcaagggcatgaggcgcgatgagaagaagagcacagcctttcagaagaagctggagccggcc | |
| | taccaggtgagcaaaggccacaagatccggctgaccgtggaactggctgaccatgacgctgaggtcaaatggctcaagaa | |
| | tggccaggagatccagatgagcggcagcaagtacatctttgagtccatcggtgccaagcgtaccctgaccatcagccagtg | |
| | ctcattggcggacgacgcagcctaccagtgcgtggtgggtggcgagaagtgtagcacggagctctttgtgaaagagcccc | |
| | ctgtgctcatcacgcgccccttggaggaccagctggtgatggtggggcagcgggtggagtttgagtgtgaagtatcggagg | |
| | agggggcgcaagtcaaatggctgaaggacggggtggagctgacccgggaggagaccttcaaataccggttcaagaagg | |
| | acgggcagagacaccacctgatcatcaacgaggccatgctggaggacgcggggcactatgcactgtgcactagcgggg | |
| | gccaggcgctggctgagctcattgtgcaggaaaagaagctggagcctcccaagatccacctggactgcccaggccgcata | |
| | ccagacaccattgtggttgtagctggaaataagctacgtctggacgtccctatctctggggaccccgctcccactgtgatct | |
| | ggcagaaggctatcacgcaggggaataaggccccagccaggccagccccagatgccccagaggacacaggtgacagcg | |
| | atgagtgggtgtttgacaagaagctgctgtgtgagaccgagggccgggtccgcgtggagaccaccaaggaccgcagcat | |
| | cttcacggtcgagggggcagagaaggaagatgagggcgtctacacggtcacagtgaagaaccctgtgggcgaggacca | |
| | ggtcaacctcacagtcaaggtcatcgacgtgccagacgcacctgcggcccccaagatcagcaacgtgggagaggactcct | |
| | gcacagtacagtgggagccgcctgcctacgatggcgggcagcccatcctgggctacatcctggagcgcaagaagaagaa | |
| | gagctaccggtggatgcggctgaacttcgacctgattcaggagctgagtcatgaagcgcggcgcatgatcgagggcgtgg | |
| | tgtacgagatgcgcgtctacgcggtcaacgccatcggcatgtccaggcccagccctgcctcccagcccttcatgcctatcgg | |
| | tccccccagcgaacccacccacctggcagtagaggacgtctctgacaccacggtctccctcaagtggcggcccccagagc | |
| | gcgtgggagcaggaggcctggatggctacagcgtggagtactgcccagagggctgctcagagtgggggctgccctgca | |
| | ggggctgacagagcacacatcgatactggtgaaggacctgcccacgggggcccggctgcttttccgagtgcgggcacac | |
| | aatatggcagggcctggagcccctgttaccaccacggagccggtgacagtgcaggagatcctgcaacggccacggcttca | |
| | gctgcccaggcacctgcgccagaccattcagaagaaggtcggggagcctgtgaaccttctcatccctttccagggcaagcc | |
| | ccggcctcaggtgacctggaccaaagaggggcagcccctggcaggcgaggaggtgagcatccgcaacagccccacag | |
| | acaccatcctgttcatccgggccgctcgccgcgtgcattcaggcacttaccaggtgacggtgcgcattgagaacatggagg | |
| | acaaggccacgctggtgctgcaggttgttgacaagccaagtcctccccaggatctccgggtgactgacgcctggggtctta | |
| | atgtggctctggagtggaagccaccccaggatgtcggcaacacggaactctgggggtacacagtgcagaaagccgacaa | |
| | gaagaccatggagtggttcaccgtcttggagcattaccgccgcacccactgcgtggtgccagagctcatcattggcaatgg | |
| | ctactacttccgcgtcttcagccagaatatggttggctttagtgacagagcggccaccaccaaggagcccgtctttatcccc | |
| | agaccaggcatcacctatgagccacccaactataaggccctggacttctccgaggccccaagcttcacccagcccctggtga | |
| | accgctcggtcatcgcgggctacactgctatgctctgctgtgctgtccggggtagccccaagcccaagatttcctggttcaa | |
| | gaatggcctggacctgggagaagacgcccgcttccgcatgttcagcaagcagggagtgttgactctggagattagaaagcc | |
| | ctgcccctttgacgggggcatctatgtctgcagggccaccaacttacagggcgaggcacggtgtgagtgccgcctggagg | |
| | tgcgagtgcctcagtaa | |
| MYBPC | MPEPGKKPVSAFSKKPRSVEVAAGSPAVFEAETERAGVKVRWQRGGSDISAS | 316 |
| 3-delC4 | NKYGLATEGTRHTLTVREVGPADQGSYAVIAGSSKVKFDLKVIEAEKAEPML | |
| protein | APAPAPAEATGAPGEAPAPAAELGESAPSPKGSSSAALNGPTPGAPDDPIGLFV | |
| | MRPQDGEVTVGGSITFSARVAGASLLKPPVVKWFKGKWVDLSSKVGQHLQL | |
| | HDSYDRASKVYLFELHITDAQPAFTGSYRCEVSTKDKFDCSNFNLTVHEAMG | |
| | TGDLDLLSAFRRTSLAGGGRRISDSHEDTGILDFSSLLKKRDSFRTPRDSKLEAP | |
| | AEEDVWEILRQAPPSEYERIAFQYGVTDLRGMLKRLKGMRRDEKKSTAFQKK | |
| | LEPAYQVSKGHKIRLTVELADHDAEVKWLKNGQEIQMSGSKYIFESIGAKRTL | |
| | TISQCSLADDAAYQCVVGGEKCSTELFVKEPPVLITRPLEDQLVMVGQRVEFE | |
| | CEVSEEGAQVKWLKDGVELTREETFKYRFKKDGQRHHLIINEAMLEDAGHYA | |
| | LCTSGGQALAELIVQEKKLEPPKIHLDCPGRIPDTIVVVAGNKLRLDVPISGDPA | |
| | PTVIWQKAITQGNKAPARPAPDAPEDTGDSDEWVFDKKLLCETEGRVRVETT | |
| | KDRSIFTVEGAEKEDEGVYTVTVKNPVGEDQVNLTVKVIDVPDAPAAPKISNV | |
| | GEDSCTVQWEPPAYDGGQPILGYILERKKKKSYRWMRLNFDLIQELSHEARR | |
| | MIEGVVYEMRVYAVNAIGMSRPSPASQPFMPIGPPSEPTHLAVEDVSDTTVSL | |
| | KWRPPERVGAGGLDGYSVEYCPEGCSEWVAALQGLTEHTSILVKDLPTGARL | |
| | LFRVRAHNMAGPGAPVTTTEPVTVQEILQRPRLQLPRHLRQTIQKKVGEPVNL | |
| | LIPFQGKPRPQVTWTKEGQPLAGEEVSIRNSPTDTILFIRAARRVHSGTYQVTV | |
| | RIENMEDKATLVLQVVDKPSPPQDLRVTDAWGLNVALEWKPPQDVGNTELW | |
| | GYTVQKADKKTMEWFTVLEHYRRTHCVVPELIIGNGYYFRVFSQNMVGFSDR | |
| | AATTKEPVFIPRPGITYEPPNYKALDFSEAPSFTQPLVNRSVIAGYTAMLCCAV | |
| | RGSPKPKISWFKNGLDLGEDARFRMFSKQGVLTLEIRKPCPFDGGIYVCRATN | |
| | LQGEARCECRLEVRVPQ | |
| MYBPC | atgcctgagccggggaagaagccagtctcagcttttagcaagaagccacggtcagtggaagtggccgcaggcagccctg | 317 |
| 3- | ccgtgttcgaggccgagacagagcgggcaggagtgaaggtgcgctggcagcgcggaggcagtgacatcagcgccagc | |
| delC4b | aacaagtacggcctggccacagagggcacacggcatacgctgacagtgcgggaagtgggccctgccgaccagggatctt | |
| DNA | acgcagtcattgctggctcctccaaggtcaagttcgacctcaaggtcatagaggcagagaaggcagagcccatgctggccc | |
| | ctgcccctgcccctgctgaggccactggagcccctggagaagccccggccccagccgctgagctgggagaaagtgccc | |
| | caagtcccaaagggtcaagctcagcagctctcaatggtcctacccctggagcccccgatgaccccattggcctcttcgtgat | |
| | gcggccacaggatggcgaggtgaccgtgggtggcagcatcaccttctcagcccgcgtggccggcgccagcctcctgaag | |
| | ccgcctgtggtcaagtggttcaagggcaaatgggtggacctgagcagcaaggtgggccagcacctgcagctgcacgaca | |
| | gctacgaccgcgccagcaaggtctatctgttcgagctgcacatcaccgatgcccagcctgccttcactggcagctaccgctg | |
| | tgaggtgtccaccaaggacaaatttgactgctccaacttcaatctcactgtccacgaggccatgggcaccggagacctggac | |
| | ctcctatcagccttccgccgcacgagcctggctggaggtggtcggcggatcagtgatagccatgaggacactgggattctg | |
| | gacttcagctcactgctgaaaaagagagacagtttccggaccccgagggactcgaagctggaggcaccagcagaggagg | |
| | acgtgtgggagatcctacggcaggcacccccatctgagtacgagcgcatcgccttccagtacggcgtcactgacctgcgc | |
| | ggcatgctaaagaggctcaagggcatgaggcgcgatgagaagaagagcacagcctttcagaagaagctggagccggcc | |
| | taccaggtgagcaaaggccacaagatccggctgaccgtggaactggctgaccatgacgctgaggtcaaatggctcaagaa | |
| | tggccaggagatccagatgagcggcagcaagtacatctttgagtccatcggtgccaagcgtaccctgaccatcagccagtg | |
| | ctcattggcggacgacgcagcctaccagtgcgtggtgggtggcgagaagtgtagcacggagctctttgtgaaagagcccc | |
| | ctgtgctcatcacgcgccccttggaggaccagctggtgatggtggggcagcgggtggagtttgagtgtgaagtatcggagg | |
| | agggggcgcaagtcaaatggctgaaggacggggtggagctgacccgggaggagaccttcaaataccggttcaagaagg | |
| | acgggcagagacaccacctgatcatcaacgaggccatgctggaggacgcggggcactatgcactgtgcactagcgggg | |
| | gccaggcgctggctgagctcattgtgcaggaaaagaagctggagcccaggcaggaacctcccaagatccacctggactg | |
| | cccaggccgcataccagacaccattgtggttgtagctggaaataagctacgtctggacgtccctatctctggggaccccgct | |
| | cccactgtgatctggcagaaggctatcacgcaggggaataaggccccagccaggccagccccagatgccccagaggac | |
| | acaggtgacagcgatgagtgggtgtttgacaagaagctgctgtgtgagaccgagggccgggtccgcgtggagaccacca | |
| | aggaccgcagcatcttcacggtcgagggggcagagaaggaagatgagggcgtctacacggtcacagtgaagaaccctgt | |
| | gggcgaggaccaggtcaacctcacagtcaaggtcatcgacgtgccagacgcacctgcggcccccaagatcagcaacgtg | |
| | ggagaggactcctgcacagtacagtgggagccgcctgcctacgatggcgggcagcccatcctgggctacatcctggagc | |
| | gcaagaagaagaagagctaccggtggatgcggctgaacttcgacctgattcaggagctgagtcatgaagcgcggcgcat | |
| | gatcgagggcgtggtgtacgagatgcgcgtctacgcggtcaacgccatcggcatgtccaggcccagccctgcctcccagc | |
| | ccttcatgcctatcggtccccccagcgaacccacccacctggcagtagaggacgtctctgacaccacggtctccctcaagtg | |
| | gcggcccccagagcgcgtgggagcaggaggcctggatggctacagcgtggagtactgcccagagggctgctcagagtg | |
| | ggtggctgccctgcaggggctgacagagcacacatcgatactggtgaaggacctgcccacgggggcccggctgcttttcc | |
| | gagtgcgggcacacaatatggcagggcctggagcccctgttaccaccacggagccggtgacagtgcaggagatcctgca | |
| | acggccacggcttcagctgcccaggcacctgcgccagaccattcagaagaaggtcggggagcctgtgaaccttctcatcc | |
| | ctttccagggcaagccccggcctcaggtgacctggaccaaagaggggcagcccctggcaggcgaggaggtgagcatcc | |
| | gcaacagccccacagacaccatcctgttcatccgggccgctcgccgcgtgcattcaggcacttaccaggtgacggtgcgc | |
| | attgagaacatggaggacaaggccacgctggtgctgcaggttgttgacaagccaagtcctccccaggatctccgggtgact | |
| | gacgcctggggtcttaatgtggctctggagtggaagccaccccaggatgtcggcaacacggaactctgggggtacacagt | |
| | gcagaaagccgacaagaagaccatggagtggttcaccgtcttggagcattaccgccgcacccactgcgtggtgccagagc | |
| | tcatcattggcaatggctactacttccgcgtcttcagccagaatatggttggctttagtgacagagcggccaccaccaagga | |
| | gcccgtctttatccccagaccaggcatcacctatgagccacccaactataaggccctggacttctccgaggccccaagcttc | |
| | acccagcccctggtgaaccgctcggtcatcgcgggctacactgctatgctctgctgtgctgtccggggtagccccaagccca | |
| | agatttcctggttcaagaatggcctggacctgggagaagacgcccgcttccgcatgttcagcaagcagggagtgttgactct | |
| | ggagattagaaagccctgcccctttgacgggggcatctatgtctgcagggccaccaacttacagggcgaggcacggtgtg | |
| | agtgccgcctggaggtgcgagtgcctcagtaa | |
| MYBPC | MPEPGKKPVSAFSKKPRSVEVAAGSPAVFEAETERAGVKVRWQRGGSDISAS | 318 |
| 3- | NKYGLATEGTRHTLTVREVGPADQGSYAVIAGSSKVKFDLKVIEAEKAEPML | |
| delC4b | APAPAPAEATGAPGEAPAPAAELGESAPSPKGSSSAALNGPTPGAPDDPIGLFV | |
| protein | MRPQDGEVTVGGSITFSARVAGASLLKPPVVKWFKGKWVDLSSKVGQHLQL | |
| | HDSYDRASKVYLFELHITDAQPAFTGSYRCEVSTKDKFDCSNFNLTVHEAMG | |
| | TGDLDLLSAFRRTSLAGGGRRISDSHEDTGILDFSSLLKKRDSFRTPRDSKLEAP | |
| | AEEDVWEILRQAPPSEYERIAFQYGVTDLRGMLKRLKGMRRDEKKSTAFQKK | |
| | LEPAYQVSKGHKIRLTVELADHDAEVKWLKNGQEIQMSGSKYIFESIGAKRTL | |
| | TISQCSLADDAAYQCVVGGEKCSTELFVKEPPVLITRPLEDQLVMVGQRVEFE | |
| | CEVSEEGAQVKWLKDGVELTREETFKYRFKKDGQRHHLIINEAMLEDAGHYA | |
| | LCTSGGQALAELIVQEKKLEPRQEPPKIHLDCPGRIPDTIVVVAGNKLRLDVPIS | |
| | GDPAPTVIWQKAITQGNKAPARPAPDAPEDTGDSDEWVFDKKLLCETEGRVR | |
| | VETTKDRSIFTVEGAEKEDEGVYTVTVKNPVGEDQVNLTVKVIDVPDAPAAP | |
| | KISNVGEDSCTVQWEPPAYDGGQPILGYILERKKKKSYRWMRLNFDLIQELSH | |
| | EARRMIEGVVYEMRVYAVNAIGMSRPSPASQPFMPIGPPSEPTHLAVEDVSDT | |
| | TVSLKWRPPERVGAGGLDGYSVEYCPEGCSEWVAALQGLTEHTSILVKDLPT | |
| | GARLLFRVRAHNMAGPGAPVTTTEPVTVQEILQRPRLQLPRHLRQTIQKKVGE | |
| | PVNLLIPFQGKPRPQVTWTKEGQPLAGEEVSIRNSPTDTILFIRAARRVHSGTY | |
| | QVTVRIENMEDKATLVLQVVDKPSPPQDLRVTDAWGLNVALEWKPPQDVGN | |
| | TELWGYTVQKADKKTMEWFTVLEHYRRTHCVVPELIIGNGYYFRVFSQNMV | |
| | GFSDRAATTKEPVFIPRPGITYEPPNYKALDFSEAPSFTQPLVNRSVIAGYTAML | |
| | CCAVRGSPKPKISWFKNGLDLGEDARFRMFSKQGVLTLEIRKPCPFDGGIYVC | |
| | RATNLQGEARCECRLEVRVPQ | |
| Human | atggctgaaaaagcggggtctacattttcacaccttctggttcctattcttctcctgattggctggattgtgggctgcatc | 319 |
| DWORF | ataatgatttatgttgtcttctcttag | |
| DNA | | |
| Human | atggccgagaaggccggatctaccttcagccacctgctggtccctattctgctgctgatcggctggatcgtgggctgcatc | 320 |
| DWORF | atcatgatctacgtggtgttcagctga | |
| DNA | | |
| codon- | | |
| opti- | | |
| mized | | |
| Human | MAEKAGSTFSHLLVPILLLIGWIVGCIIMIYVVFS | 321 |
| DWORF | | |
| protein | | |
| Human | atgagtgggggccgcttcgactttgatgatggaggggcgtactgcgggggctgggaggggggaaaggcccatgggcat | 322 |
| JPH2 | ggactgtgcacaggccccaagggccagggcgaatactctggctcctggaactttggctttgaggtggcaggtgtctacacc | |
| DNA | tggcccagcggaaacacctttgagggatactggagccagggcaaacggcatgggctgggcatagagaccaaggggcgc | |
| | tggctctacaagggcgagtggacacatggcttcaagggacgctacggaatccggcagagctcaagcagcggtgccaagt | |
| | atgagggcacctggaacaatggcctgcaagacggctatggcaccgagacctatgctgatggagggacgtaccaaggcca | |
| | gttcaccaacggcatgcgccatggctacggagtacgccagagcgtgccctacgggatggccgtggtggtgcgctcgccg | |
| | ctgcgcacgtcgctgtcgtccctgcgcagcgagcacagcaacggcacggtggccccggactctcccgcctcgccggcct | |
| | ccgacggccccgcgctgccctcgcccgccatcccgcgtggcggcttcgcgctcagcctcctggccaatgccgaggcggc | |
| | cgcgcgggcgcccaagggcggcggcctcttccagcggggcgcgctgctgggcaagctgcggcgcgcagagtcgcgc | |
| | acgtccgtgggtagccagcgcagccgtgtcagcttccttaagagcgacctcagctcgggcgccagcgacgccgcgtccac | |
| | cgccagcctgggagaggccgccgagggcgccgacgaggccgcacccttcgaggccgatatcgacgccaccaccaccg | |
| | agacctacatgggcgagtggaagaacgacaaacgctcgggcttcggcgtgagcgaacgctccagtggcctccgctacga | |
| | gggcgagtggctggacaacctgcgccacggctatggctgcaccacgctgcccgacggccaccgcgaggagggcaagt | |
| | accgccacaacgtgctggtcaaggacaccaagcgccgcatgctgcagctcaagagcaacaaggtccgccagaaagtgg | |
| | agcacagtgtggagggtgcccagcgcgccgctgctatcgcgcgccagaaggccgagattgccgcctccaggacaagcc | |
| | acgccaaggccaaagctgaggcagcggaacaggccgccctggctgccaaccaggagtccaacattgctcgcactttggc | |
| | cagggagctggctccggacttctaccagccaggtccggaatatcagaagcgccggctgctgcaggagatcctggagaact | |
| | cggagagcctgctggagccccccgaccggggcgccggcgcagcgggcctcccacagccgccccgcgagagcccgca | |
| | gctgcacgagcgtgagacccctcggcccgagggtggctccccgtcaccggccgggacgcccccgcagcccaagcggc | |
| | ccaggcccggggtgtccaaggacggcctgctgagcccaggcgcctggaacggcgagcccagcggtgagggcagccg | |
| | gtcagtcactccgtccgagggcgcgggccgccgcagccccgcgcgtccagccaccgagcgcatggccatcgaggctct | |
| | gcaggcaccgcctgcgccgtcgcgggagccggaggtggcgctttaccagggctaccacagctatgctgtgcgcaccacg | |
| | ccgcccgagcccccaccctttgaggaccagcccgagcccgaggtctccgggtccgagtccgcgccctcgtccccggcca | |
| | ccgccccgctgcaggcccccacgctccgaggccccgagcctgcacgcgagacccccgccaagctggagcccaagccc | |
| | atcatccccaaagccgagcccagggccaaggcccgcaagactgaggctcgagggctgaccaaggcgggggccaagaa | |
| | gaaggcgcggaaggaggccgcactggcggcagaggcggaggtggaggtggaagaggtccccaacaccatcctcatct | |
| | gcatggtgatcctgctgaacatcggcctggccatcctctttgttcacctcctgacctga | |
| Human | MSGGRFDFDDGGAYCGGWEGGKAHGHGLCTGPKGQGEYSGSWNFGFEVAG | 323 |
| JPH2 | VYTWPSGNTFEGYWSQGKRHGLGIETKGRWLYKGEWTHGFKGRYGIRQSSS | |
| protein | SGAKYEGTWNNGLQDGYGTETYADGGTYQGQFTNGMRHGYGVRQSVPYG | |
| | MAVVVRSPLRTSLSSLRSEHSNGTVAPDSPASPASDGPALPSPAIPRGGFALSLL | |
| | ANAEAAARAPKGGGLFQRGALLGKLRRAESRTSVGSQRSRVSFLKSDLSSGAS | |
| | DAASTASLGEAAEGADEAAPFEADIDATTTETYMGEWKNDKRSGFGVSERSS | |
| | GLRYEGEWLDNLRHGYGCTTLPDGHREEGKYRHNVLVKDTKRRMLQLKSN | |
| | KVRQKVEHSVEGAQRAAAIARQKAEIAASRTSHAKAKAEAAEQAALAANQE | |
| | SNIARTLARELAPDFYQPGPEYQKRRLLQEILENSESLLEPPDRGAGAAGLPQP | |
| | PRESPQLHERETPRPEGGSPSPAGTPPQPKRPRPGVSKDGLLSPGAWNGEPSGE | |
| | GSRSVTPSEGAGRRSPARPATERMAIEALQAPPAPSREPEVALYQGYHSYAVR | |
| | TTPPEPPPFEDQPEPEVSGSESAPSSPATAPLQAPTLRGPEPARETPAKLEPKPIIP | |
| | KAEPRAKARKTEARGLTKAGAKKKARKEAALAAEAEVEVEEVPNTILICMVI | |
| | LLNIGLAILFVHLLT | |
| Human | atgagtgggggccgcttcgactttgatgatggaggggcgtactgcgggggctgggaggggggaaaggcccatgggcat | 324 |
| JPH2 N- | ggactgtgcacaggccccaagggccagggcgaatactctggctcctggaactttggctttgaggtggcaggtgtctacacc | |
| term- | tggcccagcggaaacacctttgagggatactggagccagggcaaacggcatgggctgggcatagagaccaaggggcgc | |
| inal | tggctctacaagggcgagtggacacatggcttcaagggacgctacggaatccggcagagctcaagcagcggtgccaagt | |
| frag- | atgagggcacctggaacaatggcctgcaagacggctatggcaccgagacctatgctgatggagggacgtaccaaggcca | |
| ment | gttcaccaacggcatgcgccatggctacggagtacgccagagcgtgccctacgggatggccgtggtggtgcgctcgccg | |
| DNA | ctgcgcacgtcgctgtcgtccctgcgcagcgagcacagcaacggcacggtggccccggactctcccgcctcgccggcct | |
| | ccgacggccccgcgctgccctcgcccgccatcccgcgtggcggcttcgcgctcagcctcctggccaatgccgaggcggc | |
| | cgcgcgggcgcccaagggcggcggcctcttccagcggggcgcgctgctgggcaagctgcggcgcgcagagtcgcgc | |
| | acgtccgtgggtagccagcgcagccgtgtcagcttccttaagagcgacctcagctcgggcgccagcgacgccgcgtccac | |
| | cgccagcctgggagaggccgccgagggcgccgacgaggccgcacccttcgaggccgatatcgacgccaccaccaccg | |
| | agacctacatgggcgagtggaagaacgacaaacgctcgggcttcggcgtgagcgaacgctccagtggcctccgctacga | |
| | gggcgagtggctggacaacctgcgccacggctatggctgcaccacgctgcccgacggccaccgcgaggagggcaagt | |
| | accgccacaacgtgctggtcaaggacaccaagcgccgcatgctgcagctcaagagcaacaaggtccgccagaaagtgg | |
| | agcacagtgtggagggtgcccagcgcgccgctgctatcgcgcgccagaaggccgagattgccgcctccaggacaagcc | |
| | acgccaaggccaaagctgaggcagcggaacaggccgccctggctgccaaccaggagtccaacattgctcgcactttggc | |
| | cagggagctggctccggacttctaccagccaggtccggaatatcagaagcgccggctgctgcaggagatcctggagaact | |
| | cggagagcctgctggagccccccgaccggggcgccggcgcagcgggcctcccacagccgccccgcgagagcccgca | |
| | gctgcacgagcgtgagacccctcggcccgagggtggctccccgtcaccggccgggacgcccccgcagcccaagcggc | |
| | ccaggcccggggtgtccaaggacggcctgctgagcccaggcgcctggaacggcgagcccagcggtgagggcagccg | |
| | gtcagtcactccgtccgagggcgcgggccgccgcagccccgcgcgtccagccaccgagcgcatggccatcgaggctct | |
| | gcaggcaccgcctgcgccgtcgcgggagccggaggtggcgctttaccagggctaccacagctatgctgtgcgc | |
| Human | MSGGRFDFDDGGAYCGGWEGGKAHGHGLCTGPKGQGEYSGSWNFGFEVAG | 325 |
| JPH2 N- | VYTWPSGNTFEGYWSQGKRHGLGIETKGRWLYKGEWTHGFKGRYGIRQSSS | |
| term- | SGAKYEGTWNNGLQDGYGTETYADGGTYQGQFTNGMRHGYGVRQSVPYG | |
| inal | MAVVVRSPLRTSLSSLRSEHSNGTVAPDSPASPASDGPALPSPAIPRGGFALSLL | |
| frag- | ANAEAAARAPKGGGLFQRGALLGKLRRAESRTSVGSQRSRVSFLKSDLSSGAS | |
| ment | DAASTASLGEAAEGADEAAPFEADIDATTTETYMGEWKNDKRSGFGVSERSS | |
| protein | GLRYEGEWLDNLRHGYGCTTLPDGHREEGKYRHNVLVKDTKRRMLQLKSN | |
| | KVRQKVEHSVEGAQRAAAIARQKAEIAASRTSHAKAKAEAAEQAALAANQE | |
| | SNIARTLARELAPDFYQPGPEYQKRRLLQEILENSESLLEPPDRGAGAAGLPQP | |
| | PRESPQLHERETPRPEGGSPSPAGTPPQPKRPRPGVSKDGLLSPGAWNGEPSGE | |
| | GSRSVTPSEGAGRRSPARPATERMAIEALQAPPAPSREPEVALYQGYHSYAVR | |
| Human | atgagcgccgccacccactcgcccatgatgcaggtggcgtccggcaacggtgaccgcgaccctttgccccccggatggg | 326 |
| BAG3 | agatcaagatcgacccgcagaccggctggcccttcttcgtggaccacaacagccgcaccactacgtggaacgacccgcg | |
| DNA | cgtgccctctgagggccccaaggagactccatcctctgccaatggcccttcccgggagggctctaggctgccgcctgctag | |
| | ggaaggccaccctgtgtacccccagctccgaccaggctacattcccattcctgtgctccatgaaggcgctgagaaccggca | |
| | ggtgcaccctttccatgtctatccccagcctgggatgcagcgattccgaactgaggcggcagcagcggctcctcagaggtc | |
| | ccagtcacctctgcggggcatgccagaaaccactcagccagataaacagtgtggacaggtggcagcggcggcggcagc | |
| | ccagcccccagcctcccacggacctgagcggtcccagtctccagctgcctctgactgctcatcctcatcctcctcggccagc | |
| | ctgccttcctccggcaggagcagcctgggcagtcaccagctcccgcgggggtacatctccattccggtgatacacgagca | |
| | gaacgttacccggccagcagcccagccctccttccaccaagcccagaagacgcactacccagcgcagcagggggagta | |
| | ccagacccaccagcctgtgtaccacaagatccagggggatgactgggagccccggcccctgcgggggcatccccgttc | |
| | aggtcatctgtccagggtgcatcgagccgggagggctcaccagccaggagcagcacgccactccactccccctcgcccat | |
| | ccgtgtgcacaccgtggtcgacaggcctcagcagcccatgacccatcgagaaactgcacctgtttcccagcctgaaaacaa | |
| | accagaaagtaagccaggcccagttggaccagaactccctcctggacacatcccaattcaagtgatccgcaaagaggtgg | |
| | attctaaacctgtttcccagaagcccccacctccctctgagaaggtagaggtgaaagttccccctgctccagttccttgtcc | |
| | tcctcccagccctggcccttctgctgtcccctcttcccccaagagtgtggctacagaagagagggcagcccccagcactgcc | |
| | cctgcagaagctacacctccaaaaccaggagaagccgaggctcccccaaaacatccaggagtgctgaaagtggaagccatc | |
| | ctggagaaggtacaggggctggagcaggctgtagacaactttgaaggcaagaagactgacaaaaagtacctgatgatcga | |
| | agagtatttgaccaaagagctgctggccctggattcagtggaccccgagggacgagccgatgtgcgtcaggccaggaga | |
| | gacggtgtcaggaaggttcagaccatcttggaaaaacttgaacagaaagccattgatgtcccaggtcaagtccaggtctatg | |
| | aactccagcccagcaaccttgaagcagatcagccactgcaggcaatcatggagatgggtgccgtggcagcagacaaggg | |
| | caagaaaaatgctggaaatgcagaagatccccacacagaaacccagcagccagaagccacagcagcagcgacttcaaa | |
| | ccccagcagcatgacagacacccctggtaacccagcagcaccgtag | |
| Human | MSAATHSPMMQVASGNGDRDPLPPGWEIKIDPQTGWPFFVDHNSRTTTWNDP | 327 |
| BAG3 | RVPSEGPKETPSSANGPSREGSRLPPAREGHPVYPQLRPGYIPIPVLHEGAENRQ | |
| protein | VHPFHVYPQPGMQRFRTEAAAAAPQRSQSPLRGMPETTQPDKQCGQVAAAA | |
| | AAQPPASHGPERSQSPAASDCSSSSSSASLPSSGRSSLGSHQLPRGYISIPVIHEQ | |
| | NVTRPAAQPSFHQAQKTHYPAQQGEYQTHQPVYHKIQGDDWEPRPLRAASPF | |
| | RSSVQGASSREGSPARSSTPLHSPSPIRVHTVVDRPQQPMTHRETAPVSQPENK | |
| | PESKPGPVGPELPPGHIPIQVIRKEVDSKPVSQKPPPPSEKVEVKVPPAPVPCPPP | |
| | SPGPSAVPSSPKSVATEERAAPSTAPAEATPPKPGEAEAPPKHPGVLKVEAILE | |
| | KVQGLEQAVDNFEGKKTDKKYLMIEEYLTKELLALDSVDPEGRADVRQARR | |
| | DGVRKVQTILEKLEQKAIDVPGQVQVYELQPSNLEADQPLQAIMEMGAVAAD | |
| | KGKKNAGNAEDPHTETQQPEATAAATSNPSSMTDTPGNPAAP | |
| Human | MSAATHSPMMQVASGNGDRDPLPPGWEIKIDPQTGWPFFVDHNSRTTTWNDP | 328 |
| BAG3 | RVPSEGPKETPSSANGPSREGSRLPPAREGHPVYPQLRPGYIPIPVLHEGAENRQ | |
| C151R | VHPFHVYPQPGMQRFRTEAAAAAPQRSQSPLRGMPETTQPDKQRGQVAAAA | |
| mutant | AAQPPASHGPERSQSPAASDCSSSSSSASLPSSGRSSLGSHQLPRGYISIPVIHEQ | |
| protein | NVTRPAAQPSFHQAQKTHYPAQQGEYQTHQPVYHKIQGDDWEPRPLRAASPF | |
| | RSSVQGASSREGSPARSSTPLHSPSPIRVHTVVDRPQQPMTHRETAPVSQPENK | |
| | PESKPGPVGPELPPGHIPIQVIRKEVDSKPVSQKPPPPSEKVEVKVPPAPVPCPPP | |
| | SPGPSAVPSSPKSVATEERAAPSTAPAEATPPKPGEAEAPPKHPGVLKVEAILE | |
| | KVQGLEQAVDNFEGKKTDKKYLMIEEYLTKELLALDSVDPEGRADVRQARR | |
| | DGVRKVQTILEKLEQKAIDVPGQVQVYELQPSNLEADQPLQAIMEMGAVAAD | |
| | KGKKNAGNAEDPHTETQQPEATAAATSNPSSMTDTPGNPAAP | |
| Human | atggacatcgccatccaccacccctggatccgccgccccttctttcctttccactcccccagccgcctctttgaccagttct | 329 |
| CRYAB | tcggagagcacctgttggagtctgatcttttcccgacgtctacttccctgagtcccttctaccttcggccaccctccttcct | |
| DNA | gcgggcacccagctggtttgacactggactctcagagatgcgcctggagaaggacaggttctctgtcaacctggatgtgaag | |
| | cacttctccccagaggaactcaaagttaaggtgttgggagatgtgattgaggtgcatggaaaacatgaagagcgccaggatg | |
| | aacatggtttcatctccagggagttccacaggaaataccggatcccagctgatgtagaccctctcaccattacttcatccct | |
| | gtcatctgatggggtcctcactgtgaatggaccaaggaaacaggtctctggccctgagcgcaccattcccatcacccgtgaa | |
| | gagaagcctgctgtcaccgcagcccccaagaaatag | |
| Human | MDIAIHHPWIRRPFFPFHSPSRLFDQFFGEHLLESDLFPTSTSLSPFYLRPPSFLRA | 330 |
| CRYAB | PSWFDTGLSEMRLEKDRFSVNLDVKHFSPEELKVKVLGDVIEVHGKHEERQD | |
| protein | EHGFISREFHRKYRIPADVDPLTITSSLSSDGVLTVNGPRKQVSGPERTIPITREE | |
| | KPAVTAAPKK | |
| Human | atggagaccccgtcccagcggcgcgccacccgcagcggggcgcaggccagctccactccgctgtcgcccacccgcatc | 331 |
| LMNA | acccggctgcaggagaaggaggacctgcaggagctcaatgatcgcttggcggtctacatcgaccgtgtgcgctcgctgga | |
| LaminA | aacggagaacgcagggctgcgccttcgcatcaccgagtctgaagaggtggtcagccgcgaggtgtccggcatcaaggcc | |
| DNA | gcctacgaggccgagctcggggatgcccgcaagacccttgactcagtagccaaggagcgcgcccgcctgcagctggag | |
| | ctgagcaaagtgcgtgaggagtttaaggagctgaaagcgcgcaataccaagaaggagggtgacctgatagctgctcagg | |
| | ctcggctgaaggacctggaggctctgctgaactccaaggaggccgcactgagcactgctctcagtgagaagcgcacgctg | |
| | gagggcgagctgcatgatctgcggggccaggtggccaagcttgaggcagccctaggtgaggccaagaagcaacttcag | |
| | gatgagatgctgcggcgggtggatgctgagaacaggctgcagaccatgaaggaggaactggacttccagaagaacatct | |
| | acagtgaggagctgcgtgagaccaagcgccgtcatgagacccgactggtggagattgacaatgggaagcagcgtgagttt | |
| | gagagccggctggcggatgcgctgcaggaactgcgggcccagcatgaggaccaggtggagcagtataagaaggagct | |
| | ggagaagacttattctgccaagctggacaatgccaggcagtctgctgagaggaacagcaacctggtgggggctgcccacg | |
| | aggagctgcagcagtcgcgcatccgcatcgacagcctctctgcccagctcagccagctccagaagcagctggcagccaa | |
| | ggaggcgaagcttcgagacctggaggactcactggcccgtgagcgggacaccagccggcggctgctggcggaaaagg | |
| | agcgggagatggccgagatgcgggcaaggatgcagcagcagctggacgagtaccaggagcttctggacatcaagctgg | |
| | ccctggacatggagatccacgcctaccgcaagctcttggagggcgaggaggagaggctacgcctgtcccccagccctac | |
| | ctcgcagcgcagccgtggccgtgcttcctctcactcatcccagacacaggggggggcagcgtcaccaaaaagcgcaaac | |
| | tggagtccactgagagccgcagcagcttctcacagcacgcacgcactagcgggcgcgtggccgtggaggaggtggatg | |
| | aggagggcaagtttgtccggctgcgcaacaagtccaatgaggaccagtccatgggcaattggcagatcaagcgccagaat | |
| | ggagatgatcccttgctgacttaccggttcc | |
| Human | METPSQRRATRSGAQASSTPLSPTRITRLQEKEDLQELNDRLAVYIDRVRSLET | 332 |
| LMNA | ENAGLRLRITESEEVVSREVSGIKAAYEAELGDARKTLDSVAKERARLQLELS | |
| LaminA | KVREEFKELKARNTKKEGDLIAAQARLKDLEALLNSKEAALSTALSEKRTLEG | |
| protein | ELHDLRGQVAKLEAALGEAKKQLQDEMLRRVDAENRLQTMKEELDFQKNIY | |
| | SEELRETKRRHETRLVEIDNGKQREFESRLADALQELRAQHEDQVEQYKKELE | |
| | KTYSAKLDNARQSAERNSNLVGAAHEELQQSRIRIDSLSAQLSQLQKQLAAKE | |
| | AKLRDLEDSLARERDTSRRLLAEKEREMAEMRARMQQQLDEYQELLDIKLAL | |
| | DMEIHAYRKLLEGEEERLRLSPSPTSQRSRGRASSHSSQTQGGGSVTKKRKLES | |
| | TESRSSFSQHARTSGRVAVEEVDEEGKFVRLRNKSNEDQSMGNWQIKRQNGD | |
| | DPLLTYRFPPKFTLKAGQVVTIWAAGAGATHSPPTDLVWKAQNTWGCGNSL | |
| | RTALINSTGEEVAMRKLVRSVTVVEDDEDEDGDDLLHHHHGSHCSSSGDPAE | |
| | YNLRSRTVLCGTCGQPADKASASGSGAQVGGPISSGSSASSVTVTRSYRSVGG | |
| | SGGGSFGDNLVTRSYLLGNSSPRTQSPQNCSIM | |
| Human | atggagaccccgtcccagcggcgcgccacccgcagcggggcgcaggccagctccactccgctgtcgcccacccgcatc | 333 |
| LMNA | acccggctgcaggagaaggaggacctgcaggagctcaatgatcgcttggcggtctacatcgaccgtgtgcgctcgctgga | |
| LaminC | aacggagaacgcagggctgcgccttcgcatcaccgagtctgaagaggtggtcagccgcgaggtgtccggcatcaaggcc | |
| DNA | gcctacgaggccgagctcggggatgcccgcaagacccttgactcagtagccaaggagcgcgcccgcctgcagctggag | |
| | ctgagcaaagtgcgtgaggagtttaaggagctgaaagcgcgcaataccaagaaggagggtgacctgatagctgctcagg | |
| | ctcggctgaaggacctggaggctctgctgaactccaaggaggccgcactgagcactgctctcagtgagaagcgcacgctg | |
| | gagggcgagctgcatgatctgcggggccaggtggccaagcttgaggcagccctaggtgaggccaagaagcaacttcag | |
| | gatgagatgctgcggcgggtggatgctgagaacaggctgcagaccatgaaggaggaactggacttccagaagaacatct | |
| | acagtgaggagctgcgtgagaccaagcgccgtcatgagacccgactggtggagattgacaatgggaagcagcgtgagttt | |
| | gagagccggctggcggatgcgctgcaggaactgcgggcccagcatgaggaccaggtggagcagtataagaaggagct | |
| | ggagaagacttattctgccaagctggacaatgccaggcagtctgctgagaggaacagcaacctggtgggggctgcccacg | |
| | aggagctgcagcagtcgcgcatccgcatcgacagcctctctgcccagctcagccagctccagaagcagctggcagccaa | |
| | ggaggcgaagcttcgagacctggaggactcactggcccgtgagcgggacaccagccggcggctgctggcggaaaagg | |
| | agcgggagatggccgagatgcgggcaaggatgcagcagcagctggacgagtaccaggagcttctggacatcaagctgg | |
| | ccctggacatggagatccacgcctaccgcaagctcttggagggcgaggaggagaggctacgcctgtcccccagccctac | |
| | ctcgcagcgcagccgtggccgtgcttcctctcactcatcccagacacagggtgggggcagcgtcaccaaaaagcgcaaac | |
| | tggagtccactgagagccgcagcagcttctcacagcacgcacgcactagcgggcgcgtggccgtggaggaggtggatg | |
| | aggagggcaagtttgtccggctgcgcaacaagtccaatgaggaccagtccatgggcaattggcagatcaagcgccagaat | |
| | ggagatgatcccttgctgacttaccggttcc | |
| Human | METPSQRRATRSGAQASSTPLSPTRITRLQEKEDLQELNDRLAVYIDRVRSLET | 334 |
| LMNA | ENAGLRLRITESEEVVSREVSGIKAAYEAELGDARKTLDSVAKERARLQLELS | |
| LaminC | KVREEFKELKARNTKKEGDLIAAQARLKDLEALLNSKEAALSTALSEKRTLEG | |
| protein | ELHDLRGQVAKLEAALGEAKKQLQDEMLRRVDAENRLQTMKEELDFQKNIY | |
| | SEELRETKRRHETRLVEIDNGKQREFESRLADALQELRAQHEDQVEQYKKELE | |
| | KTYSAKLDNARQSAERNSNLVGAAHEELQQSRIRIDSLSAQLSQLQKQLAAKE | |
| | AKLRDLEDSLARERDTSRRLLAEKEREMAEMRARMQQQLDEYQELLDIKLAL | |
| | DMEIHAYRKLLEGEEERLRLSPSPTSQRSRGRASSHSSQTQGGGSVTKKRKLES | |
| | TESRSSFSQHARTSGRVAVEEVDEEGKFVRLRNKSNEDQSMGNWQIKRQNGD | |
| | DPLLTYRFPPKFTLKAGQVVTIWAAGAGATHSPPTDLVWKAQNTWGCGNSL | |
| | RTALINSTGEEVAMRKLVRSVTVVEDDEDEDGDDLLHHHHVSGSRR | |
| Human | atggcggatgggagcagcgatgcggctagggaacctcgccctgcaccagccccaatcagacgccgctcctccaactacc | 335 |
| TNNI3 | gcgcttatgccacggagccgcacgccaagaaaaaatctaagatctccgcctcgagaaaattgcagctgaagactctgctgc | |
| DNA | tgcagattgcaaagcaagagctggagcgagaggcggaggagcggcgcggagagaaggggcgcgctctgagcacccg | |
| | ctgccagccgctggagttggccgggctgggcttcgcggagctgcaggacttgtgccgacagctccacgcccgtgtggaca | |
| | aggtggatgaagagagatacgacatagaggcaaaagtcaccaagaacatcacggagattgcagatctgactcagaagatc | |
| | tttgaccttcgaggcaagtttaagcggcccaccctgcggagagtgaggatctctgcagatgccatgatgcaggcgctgctg | |
| | ggggcccgggctaaggagtccctggacctgcgggcccacctcaagcaggtgaagaaggaggacaccgagaaggaaaa | |
| | ccgggaggtgggagactggcgcaagaacatcgatgcactgagtggaatggagggccgcaagaaaaagtttgagagctg | |
| | a | |
| Human | MADGSSDAAREPRPAPAPIRRRSSNYRAYATEPHAKKKSKISASRKLQLKTLL | 336 |
| TNNI3 | LQIAKQELEREAEERRGEKGRALSTRCQPLELAGLGFAELQDLCRQLHARVDK | |
| protein | VDEERYDIEAKVTKNITEIADLTQKIFDLRGKFKRPTLRRVRISADAMMQALL | |
| | GARAKESLDLRAHLKQVKKEDTEKENREVGDWRKNIDALSGMEGRKKKFES | |
| Human | atggagaaagtccaatacctcactcgctcagctataagaagagcctcaaccattgaaatgcctcaacaagcacgtcaaaagc | 337 |
| PLN | tacagaatctatttatcaatttctgtctcatcttaatatgtctcttgctgatctgtatcatcgtgatgcttctctga | |
| DNA | | |
| Human | MEKVQYLTRSAIRRASTIEMPQQARQKLQNLFINFCLILICLLLICIIVMLL | 338 |
| PLN | | |
| protein | | |
| Human | atggtgtgcttccgcctcttcccggttccgggctcagggctcgttctggtctgcctagtcctgggagctgtgcggtcttat | 339 |
| LAMP2a | tgcatggaacttaatttgacagattcagaaaatgccacttgcctttatgcaaaatggcagatgaatttcacagtacgctat | |
| DNA | gaaactacaaataaaacttataaaactgtaaccatttcagaccatggcactgtgacatataatggaagcatttgtggggat | |
| | cgatcagaatggtccaaaatagcagtgcagttcggacctggcttttcctggattgcgaattttaccaaggcagcatctact | |
| | tattcaattgacagcgtctcattttcctacaacactggtgataacacaacatttcctgatgctgaagataaaggaattctt | |
| | actgttgatgaacttttggccatcagaattccattgaatgacctttttagatgcaatagtttatcaactttggaaaagaat | |
| | gatgttgtccaacactactgggatgttcttgtacaagcttttgtccaaaatggcacagtgagcacaaatgagttcctgtgt | |
| | gataaagacaaaacttcaacagtggcacccaccatacacaccactgtgccatctcctactacaacacctactccaaaggaa | |
| | aaaccagaagctggaacctattcagttaataatggcaatgatacttgtctgctggctaccatggggctgcagctgaacatc | |
| | actcaggataaggttgcttcagttattaacatcaaccccaatacaactcactccacaggcagctgccgttctcacactgct | |
| | ctacttagactcaatagcagcaccattaagtatctagactttgtctttgctgtgaaaaatgaaaaccgattttatctgaag | |
| | gaagtgaacatcagcatgtatttggttaatggctccgttttcagcattgcaaataacaatctcagctactgggatgccccc | |
| | ctgggaagttcttatatgtgcaacaaagagcagactgtttcagtgtctggagcatttcagataaatacctttgatctaagg | |
| | ggttcagcctttcaatgtgacacaaggaaagtattctacagctcaagactgcagtcagatgacgacaacttccttgtgccc | |
| | tatagcggtgggagctgccttggcaggagtacttattctagtgttgctggcttattttatggtctcaagcaccatcatgct | |
| | ggatatgagcaattttag | |
| Human | MVCFRLFPVPGSGLVLVCLVLGAVRSYALELNLTDSENATCLYAKWQMNFT | 340 |
| LAMP2a | VRYETTNKTYKTVTISDHGTVTYNGSICGDDQNGPKIAVQFGPGFSWIANFTK | |
| protein | AASTYSIDSVSFSYNTGDNTTFPDAEDKGILTVDELLAIRIPLNDLFRCNSLSTL | |
| | EKNDVVQHYWDVLVQAFVQNGTVSTNEFLCDKDKTSTVAPTIHTTVPSPTTT | |
| | PTPKEKPEAGTYSVNNGNDTCLLATMGLQLNITQDKVASVININPNTTHSTGS | |
| | CRSHTALLRLNSSTIKYLDFVFAVKNENRFYLKEVNISMYLVNGSVFSIANNNL | |
| | SYWDAPLGSSYMCNKEQTVSVSGAFQINTFDLRVQPFNVTQGKYSTAQDCSA | |
| | DDDNFLVPIAVGAALAGVLILVLLAYFIGLKHHHAGYEQF | |
| Human | atggtgtgcttccgcctcttcccggttccgggctcagggctcgttctggtctgcctagtcctgggagctgtgcggtcttat | 341 |
| LAMP2b | gcattggaacttaatttgacagattcagaaaatgccacttgcctttatgcaaaatggcagatgaatttcacagtacgctat | |
| DNA | gaaactacaaataaaacttataaaactgtaaccatttcagaccatggcactgtgacatataatggaagcatttgtggggat | |
| | gatcagaatggtcccaaaatagcagtgcagttcggacctggcttttcctggattgcgaattttaccaaggcagcatctact | |
| | tattcaattgacagcgtctcattttcctacaacactggtgataacacaacatttcctgatgctgaagataaaggaattctt | |
| | actgttgatgaacttttggccatcagaattccattgaatgacctttttagatgcaatagtttatcaactttggaaaagaat | |
| | ggatgttgtccaacactactgggatgttctttacaagcttttgtccaaaatggcacagtgagcacaaatgagttcctgtgt | |
| | gataaagacaaaacttcaacagtggcacccaccatacacaccactgtgccatctcctactacaacacctactccaaaggaa | |
| | aaaccagaagctggaacctattcagttaataatggcaatgatacttgtctgctggctaccatggggctgcagctgaacatc | |
| | actcaggataaggttgcttcagttattaacatcaaccccaatacaactcactccacaggcagctgccgttctcacactgct | |
| | tgtctttgctgtgaaaaatgaaaaccgattttatctgaaggaagtgaacatcagcatgtatttggttaatggctccgtttt | |
| | cactacttagactcaatagcagcaccattaagtatctagacttgcattgcaaataacaatctcagctactgggatgccccc | |
| | ctgggaagttcttatatgtgcaacaaagagcagactgtttcagtgtctggagcatttcagataaatacctttgatctaagg | |
| | gttcagcctttcaatgtgacacaaggaaagtattctacagcccaagagtgttcgctggatgatgacaccattctaatccca | |
| | attatagttggtgctggtctttcaggcttgattatcgttatagtgattgcttacgtaattggcagaagaaaaagttatgct | |
| | ggatatcagactctgtaa | |
| Human | MVCFRLFPVPGSGLVLVCLVLGAVRSYALELNLTDSENATCLYAKWQMNFT | 342 |
| LAMP2b | VRYETTNKTYKTVTISDHGTVTYNGSICGDDQNGPKIAVQFGPGFSWIANFTK | |
| protein | AASTYSIDSVSFSYNTGDNTTFPDAEDKGILTVDELLAIRIPLNDLFRCNSLSTL | |
| | EKNDVVQHYWDVLVQAFVQNGTVSTNEFLCDKDKTSTVAPTIHTTVPSPTTT | |
| | PTPKEKPEAGTYSVNNGNDTCLLATMGLQLNITQDKVASVININPNTTHSTGS | |
| | CRSHTALLRLNSSTIKYLDFVFAVKNENRFYLKEVNISMYLVNGSVFSIANNNL | |
| | SYWDAPLGSSYMCNKEQTVSVSGAFQINTFDLRVQPFNVTQGKYSTAQECSL | |
| | DDDTILIPIIVGAGLSGLIIVIVIAYVIGRRKSYAGYQTL | |
| Human | atggtgtgcttccgcctcttcccggttccgggctcagggctcgttctggtctgcctagtcctgggagctgtgcggtcttat | 343 |
| LAMP2c | gcattggaacttaatttgacagattcagaaaatgccacttgcctttatgcaaaatggcagatgaatttcacagtacgctat | |
| DNA | gaaactacaaataaaacttataaaactgtaaccatttcagaccatggcactgtgacatataatggaagcatttgtggggat | |
| | gatcagaatggtcccaaaatagcagtgcagttcggacctggcttttcctggattgcgaattttaccaaggcagcatctact | |
| | tattcaattgacagcgtctcattttcctacaacactggtgataacacaacatttcctgatgctgaagataaaggaattctt | |
| | actgttgatgaacttttggccatcagaattccattgaatgacctttttagatgcaatagtttatcaactttggaaaagaat | |
| | ggatgttgtccaacactactgggatgttctttacaagcttttgtccaaaatggcacagtgagcacaaatgagttcctgtgt | |
| | gataaagacaaaacttcaacagtggcacccaccatacacaccactgtgccatctcctactacaacacctactccaaaggaa | |
| | gaaaccagaagctggaacctattcagttaataatgcaatgatacttgtctgctggctaccatggggctgcagctgaacatc | |
| | actcaggataaggttgcttcagttattaacatcaaccccaatacaactcactccacaggcagctgccgttctcacactgct | |
| | ctacttagactcaatagcagcaccattaagtatctagactttgtctttgctgtgaaaaatgaaaaccgattttatctgaag | |
| | gaagtgaacatcagcatgtatttggttaatggctccgttttcagcattgcaaataacaatctcagctactgggatgccccc | |
| | ctgggaagttcttatatgtgcaacaaagagcagactgtttcagtgtctggagcatttcagataaatacctttgatctaagg | |
| | gttcagcctttcaatgtgacacaaggaaagtattctacagctgaagaatgttctgctgactctgacctcaactttcttatt | |
| | cctgttgcagtgggtgtggccttgggcttccttataattgttgtctttatctcttatatgattggaagaaggaaaagtcgt | |
| | actggttatcagtctgtgtaa | |
| Human | MVCFRLFPVPGSGLVLVCLVLGAVRSYALELNLTDSENATCLYAKWQMNFT | 344 |
| LAMP2c | VRYETTNKTYKTVTISDHGTVTYNGSICGDDQNGPKIAVQFGPGFSWIANFTK | |
| protein | AASTYSIDSVSFSYNTGDNTTFPDAEDKGILTVDELLAIRIPLNDLFRCNSLSTL | |
| | EKNDVVQHYWDVLVQAFVQNGTVSTNEFLCDKDKTSTVAPTIHTTVPSPTTT | |
| | PTPKEKPEAGTYSVNNGNDTCLLATMGLQLNITQDKVASVININPNTTHSTGS | |
| | CRSHTALLRLNSSTIKYLDFVFAVKNENRFYLKEVNISMYLVNGSVFSIANNNL | |
| | SYWDAPLGSSYMCNKEQTVSVSGAFQINTFDLRVQPFNVTQGKYSTAEECSA | |
| | DSDLNFLIPVAVGVALGFLIIVVFISYMIGRRKSRTGYQSV | |
| Human | atgagctgcaacggaggctcccacccgcggatcaacactctgggccgcatgatccgcgccgagtctggcccggacctgc | 345 |
| DSP DPI | gctacgaggtgaccagcggcggcgggggcaccagcaggatgtactattctcggcgcggcgtgatcaccgaccagaactc | |
| DNA | ggacggctactgtcaaaccggcacgatgtccaggcaccagaaccagaacaccatccaggagctgctgcagaactgctcc | |
| | gactgcttgatgcgagcagagctcatcgtgcagcctgaattgaagtatggagatggaatacaactgactcggagtcgagaat | |
| | tggatgagtgttttgcccaggccaatgaccaaatggaaatcctcgacagcttgatcagagagatgcggcagatgggccagc | |
| | cctgtgatgcttaccagaaaaggcttcttcagctccaagagcaaatgcgagccctttataaagccatcagtgtccctcgagt | |
| | ccgcagggccagctccaagggtggtggaggctacacttgtcagagtggctctggctgggatgagttcaccaaacatgtcacc | |
| | agtgaatgtttggggtggatgaggcagcaaagggcggagatggacatggtggcctggggtgtggacctggcctcagtgga | |
| | gcagcacattaacagccaccggggcatccacaactccatcggcgactatcgctggcagctggacaaaatcaaagccgacc | |
| | tgcgcgagaaatctgcgatctaccagttggaggaggagtatgaaaacctgctgaaagcgtcctttgagaggatggatcacct | |
| | gcgacagctgcagaacatcattcaggccacgtccagggagatcatgtggatcaatgactgcgaggaggaggagctgctgt | |
| | acgactggagcgacaagaacaccaacatcgctcagaaacaggaggccttctccatacgcatgagtcaactggaagttaaa | |
| | gaaaaagagctcaataagctgaaacaagaaagtgaccaacttgtcctcaatcagcatccagcttcagacaaaattgaggcct | |
| | atatggacactctgcagacgcagtggagttggattcttcagatcaccaagtgcattgatgttcatctgaaagaaaatgctgc | |
| | ctactttcagttttttgaagaggcgcagtctactgaagcatacctgaaggggctccaggactccatcaggaagaagtacccc | |
| | tgcgacaagaacatgcccctgcagcacctgctggaacagatcaaggagctggagaaagaacgagagaaaatccttgaataca | |
| | agcgtcaggtgcagaacttggtaaacaagtctaagaagattgtacagctgaagcctcgtaacccagactacagaagcaata | |
| | aacccattattctcagagctctctgtgactacaaacaagatcagaaaatcgtgcataagggggatgagtgtatcctgaagga | |
| | caacaacgagcgcagcaagtggtacgtgacgggcccgggaggcgttgacatgcttgttccctctgtggggctgatcatccct | |
| | cctccgaacccactggccgtggacctctcttgcaagattgagcagtactacgaagccatcttggctctgtggaaccagctct | |
| | acatcaacatgaagagcctggtgtcctggcactactgcatgattgacatagagaagatcagggccatgacaatcgccaagct | |
| | gaaaacaatgcggcaggaagattacatgaagacgatagccgaccttgagttacattaccaagagttcatcagaaatagccaa | |
| | ggctcagagatgtttggagatgatgacaagcggaaaatacagtctcagttcaccgatgcccagaagcattaccagaccctgg | |
| | tcattcagctccctggctatccccagcaccagacagtgaccacaactgaaatcactcatcatggaacctgccaagatgtcaa | |
| | ccataataaagtaattgaaaccaacagagaaaatgacaagcaagaaacatggatgctgatggagctgcagaagattcgcagg | |
| | cagatagagcactgcgagggcaggatgactctcaaaaacctccctctagcagaccagggatcttctcaccacatcacagtg | |
| | aaaattaacgagcttaagagtgtgcagaatgattcacaagcaattgctgaggttctcaaccagcttaaagatatgcttgcca | |
| | acttcagaggttctgaaaagtactgctatttacagaatgaagtatttggactatttcagaaactggaaaatatcaatggtgt | |
| | tacagatggctacttaaatagcttatgcacagtaagggcactgctccaggctattctccaaacagaagacatgttaaaggtt | |
| | tatgaagccaggctcactgaggaggaaactgtctgcctggacctggataaagtggaagcttaccgctgtggactgaagaaaa | |
| | aaaaaatgacttgaacttgaagaagtcgttgttggccactatgaagacagaactacagaaagcccagcagatccactctcag | |
| | acttcacagcagtatccactttatgatctggacttgggcaagttcggtgaaaaagtcacacagctgacagaccgctggcaaa | |
| | ggatagataaacagatcgactttaggttatgggacctggagaaacaaatcaagcaattgaggaattatcgtgataactatca | |
| | ggctttctgcaagtggctctatgatgctaaacgccgccaggattccttagaatccatgaaatttggagattccaacacagtc | |
| | atgcggtttttgaatgagcagaagaacttgcacagtgaaatatctggcaaacgagacaaatcagaggaagtacaaaaaattg | |
| | ctgaactttgcgccaattcaattaaggattatgagctccagctggcctcatacacctcaggactggaaactctgctgaacat | |
| | gacctatcaagagaccatgattcagtccccttctggggtgattctgcaagaggctgcagatgttcatgctcggtacattgaa | |
| | ctacttacaagatctggagactattacaggttcttaagtgagatgctgaagagtttggaagatctgaagctgaaaaatacca | |
| | agatcgaagttttggaagaggagctcagactggcccgagatgccaactcggaaaactgtaataagaacaaattcctggatca | |
| | gaacctgcagaaataccaggcagagtgttcccagttcaaagcgaagcttgcgagcctggaggagctgaagagacaggctgag | |
| | ctggatgggaagtcggctaagcaaaatctagacaagtgctacggccaaataaaagaactcaatgagaagatcacccgactga | |
| | cttatgagattgaagatgaaaagagaagaagaaaatctgtggaagacagatttgaccaacagaagaatgactatgaccaact | |
| | gcagaaagcaaggcaatgtgaaaaggagaaccttggttggcagaaattagagtctgagaaagccatcaaggagaaggagtac | |
| | gagattgaaaggttgagggttctactgcaggaagaaggcacccggaagagagaatatgaaaatgagctggcaaaggtaagaa | |
| | accactataatgaggagatgagtaatttaaggaacaagtatgaaacagagattaacattacgaagaccaccatcaaggagat | |
| | atccatgcaaaaagaggatgattccaaaaatcttagaaaccagcttgatagactttcaagggaaaatcgagatctgaaggat | |
| | gaaattgtcaggctcaatgacagcatcttgcaggccactgagcagcgaaggcgagctgaagaaaacgcccttcagcaaaagg | |
| | cctgtggctctgagataatgcagaagaagcagcatctggagatagaactgaagcaggtcatgcagcagcgctctgaggacaa | |
| | tgcccggcacaagcagtccctggaggaggctgccaagaccattcaggacaaaaataaggagatcgagagactcaaagctgag | |
| | tttcaggaggaggccaagcgccgctgggaatatgaaaatgaactgagtaaggtaagaaacaattatgatgaggagatcatt | |
| | agcttaaaaaatcagtttgagaccgagatcaacatcaccaagaccaccatccaccagctcaccatgcagaaggaagaggat | |
| | accagtggctaccgggctcagatagacaatctcacccgagaaaacaggagcttatctgaagaaataaagaggctgaagaa | |
| | cactctaacccagaccacagagaatctcaggagggtggaagaagacatccaacagcaaaaggccactggctctgaggtgt | |
| | ctcagaggaaacagcagctggaggttgagctgagacaagtcactcagatgcgaacagaggagagcgtaagatataagca | |
| | atctcttgatgatgctgccaaaaccatccaggataaaaacaaggagatagaaaggttaaaacaactgatcgacaaagaaaca | |
| | aatgaccggaaatgcctggaagatgaaaacgcgagattacaaagggtccagtatgacctgcagaaagcaaacagtagtgc | |
| | gacggagacaataaacaaactgaaggttcaggagcaagaactgacacgcctgaggatcgactatgaaagggtttcccagg | |
| | agaggactgtgaaggaccaggatatcacgcggttccagaactctctgaaagagctgcagctgcagaagcagaaggtgga | |
| | agaggagctgaatcggctgaagaggaccgcgtcagaagactcctgcaagaggaagaagctggaggaagagctggaag | |
| | gcatgaggaggtcgctgaaggagcaagccatcaaaatcaccaacctgacccagcagctggagcaggcatccattgttaag | |
| | aagaggagtgaggatgacctccggcagcagagggacgtgctggatggccacctgagggaaaagcagaggacccagga | |
| | agagctgaggaggctctcttctgaggtcgaggccctgaggcggcagttactccaggaacaggaaagtgtcaaacaagctc | |
| | acttgaggaatgagcatttccagaaggcgatagaagataaaagcagaagcttaaatgaaagcaaaatagaaattgagaggc | |
| | tgcagtctctcacagagaacctgaccaaggagcacttgatgttagaagaagaactgcggaacctgaggctggagtacgatg | |
| | acctgaggagaggacgaagcgaagcggacagtgataaaaatgcaaccatcttggaactaaggagccagctgcagatcag | |
| | caacaaccggaccctggaactgcaggggctgattaatgatttacagagagagagggaaaatttgagacaggaaattgaga | |
| | aattccaaaagcaggctttagaggcatctaataggattcaggaatcaaagaatcagtgtactcaggtggtacaggaaagaga | |
| | gagccttctggtgaaaatcaaagtcctggagcaagacaaggcaaggctgcagaggctggaggatgagctgaatcgtgcaa | |
| | aatcaactctagaggcagaaaccagggtgaaacagcgcctggagtgtgagaaacagcaaattcagaatgacctgaatcag | |
| | tggaagactcaatattcccgcaaggaggaggctattaggaagatagaatcggaaagagaaaagagtgagagagagaaga | |
| | acagtcttaggagtgagatcgaaagactccaagcagagatcaagagaattgaagagaggtgcaggcgtaagctggaggat | |
| | tctaccagggagacacagtcacagttagaaacagaacgctcccgatatcagagggagattgataaactcagacagcgccc | |
| | atatgggtcccatcgagagacccagactgagtgtgagtggaccgttgacacctccaagctggtgtttgatgggctgaggaa | |
| | gaaggtgacagcaatgcagctctatgagtgtcagctgatcgacaaaacaaccttggacaaactattgaaggggaagaagtc | |
| | agtggaagaagttgcttctgaaatccagccattccttcggggtgcaggatctatcgctggagcatctgcttctcctaaggaa | |
| | aaatactctttggtagaggccaagagaaagaaattaatcagcccagaatccacagtcatgcttctggaggcccaggcagcta | |
| | caggtggtataattgatccccatcggaatgagaagctgactgtcgacagtgccatagctcgggacctcattgacttcgatga | |
| | ccgtcagcagatatatgcagcagaaaaagctatcactggttttgatgatccattttcaggcaagacagtatctgtttcagaa | |
| | gccatcaagaaaaatttgattgatagagaaaccggaatgcgcctgctggaagcccagattgcttcagggggtgtagtagacc | |
| | ctgtgaacagtgtctttttgccaaaagatgtcgccttggcccgggggctgattgatagagatttgtatcgatccctgaatg | |
| | atccccgagatagtcagaaaaactttgtggatccagtcaccaaaaagaaggtcagttacgtgcagctgaaggaacggtgcag | |
| | aatcgaaccacatactggtctgctcttgctttcagtacagaagagaagcatgtccttccaaggaatcagacaacctgtgacc | |
| | gtcactgagctagtagattctggtatattgagaccgtccactgtcaatgaactggaatctggtcagatttcttatgacgagg | |
| | ttggtgagagaattaaggacttcctccagggttcaagctgcatagcaggcatatacaatgagaccacaaaacagaagcttgg | |
| | catttatgaggccatgaaaattggcttagtccgacctggtactgctctggagttgctggaagcccaagcagctactggcttt | |
| | atagtggatcctgttagcaacttgaggttaccagtggaggaagcctacaagagaggtctggtgggcattgagttcaaagag | |
| | aagctcctgtctgcagaacgagctgtcactgggtataatgatcctgaaacaggaaacatcatctctttgttccaagccatga | |
| | agataaggaactcatcgaaaggccacggtattcgcttattagaagcacagatcgcaaccggggggatcattgacccaaagga | |
| | gagccatcgtttaccagttgacatagcatataagaggggctatttcaatgaggaactcagtgagattctctcagatccaagt | |
| | gatgataccaaaggattttttgaccccaacactgaagaaaatcttacctatctgcaactaaaagaaagatgcattaaggat | |
| | gaggaaacagggctctgtcttctgcctctgaaagaaaagaagaaacaggtgcagacatcacaaaagaataccctcaggaag | |
| | ccgtagagtggtcatagttgaccagaaaccaataaagaaatgtctgttcaggaggcctacaagaagggcctaattgattat | |
| | gaaaccttcaaagaactgtgtgagcaggaatgtgaatgggaagaaataaccatcacgggatcagatggctccaccagggtg | |
| | gtcctggtagatagaaagacaggcagtcagtatgatattcaagatgctattgacaagggccttgttgacaggaagttcttt | |
| | gatcagtaccgatccggcagcctcagcctcactcaatttgctgacatgatctccttgaaaaatggtgtcggcaccagcagc | |
| | agcatgggcagtggtgtcagcgatgatgtttttagcagctcccgacatgaatcagtaagtaagatttccaccatatccagc | |
| | tgtcaggaatttaaccataaggagcagctcttttcagacaccctggaagaatcgagccccattgcagccatctttgacaca | |
| | gaaaacctggagaaaatctccattacagaaggtatagagcggggcatcgttgacagcatcacgggtcagaggcttctggag | |
| | gctcaggcctgcacaggtggcatcatccacccaaccacgggccagaagctgtcacttcaggacgcagtctcccagggtgtg | |
| | attgaccaagacatggccaccaggctgaagcctgctcagaaagccttcataggcttcgagggtgtgaagggaaagaagaag | |
| | atgtcagcagcagaggcagtgaaagaaaaatggctcccgtatgaggctggccagcgcttcctggagttccagtacctcacg | |
| | ggaggtcttgttgacccggaagtgcatgggaggataagcaccgaagaagccatccggaaggggttcatagatggccgcgcc | |
| | gcacagaggctgcaagacaccagcagctatgccaaaatcctgacctgccccaaaaccaaattaaaaatatcctataaggat | |
| | gccataaatcgctccatggtagaagatatcactgggctgcgccttctggaagccgcctccgtgtcgtccaagggcttaccc | |
| | agcccttacaacatgtcttcggctccggggtcccgctccggctcccgctcgggatctcgctccggatctcgctccgggtc | |
| | ccgcagtgggtcccggagaggaagctttgacgccacagggaattcttcctactcttattcctactcatttagcagtagttc | |
| | tattgggcactag | |
| Human | MSCNGGSHPRINTLGRMIRAESGPDLRYEVTSGGGGTSRMYYSRRGVITDQNS | 346 |
| DSP DPI | DGYCQTGTMSRHQNQNTIQELLQNCSDCLMRAELIVQPELKYGDGIQLTRSRE | |
| protein | LDECFAQANDQMEILDSLIREMRQMGQPCDAYQKRLLQLQEQMRALYKAISV | |
| | PRVRRASSKGGGGYTCQSGSGWDEFTKHVTSECLGWMRQQRAEMDMVAW | |
| | GVDLASVEQHINSHRGIHNSIGDYRWQLDKIKADLREKSAIYQLEEEYENLLK | |
| | ASFERMDHLRQLQNIIQATSREIMWINDCEEEELLYDWSDKNTNIAQKQEAFSI | |
| | RMSQLEVKEKELNKLKQESDQLVLNQHPASDKIEAYMDTLQTQWSWILQITK | |
| | CIDVHLKENAAYFQFFEEAQSTEAYLKGLQDSIRKKYPCDKNMPLQHLLEQIK | |
| | ELEKEREKILEYKRQVQNLVNKSKKIVQLKPRNPDYRSNKPIILRALCDYKQD | |
| | QKIVHKGDECILKDNNERSKWYVTGPGGVDMLVPSVGLIIPPPNPLAVDLSCKI | |
| | EQYYEAILALWNQLYINMKSLVSWHYCMIDIEKIRAMTIAKLKTMRQEDYMK | |
| | TIADLELHYQEFIRNSQGSEMFGDDDKRKIQSQFTDAQKHYQTLVIQLPGYPQ | |
| | HQTVTTTEITHHGTCQDVNHNKVIETNRENDKQETWMLMELQKIRRQIEHCE | |
| | GRMTLKNLPLADQGSSHHITVKINELKSVQNDSQAIAEVLNQLKDMLANFRG | |
| | SEKYCYLQNEVFGLFQKLENINGVTDGYLNSLCTVRALLQAILQTEDMLKVY | |
| | EARLTEEETVCLDLDKVEAYRCGLKKIKNDLNLKKSLLATMKTELQKAQQIH | |
| | SQTSQQYPLYDLDLGKFGEKVTQLTDRWQRIDKQIDFRLWDLEKQIKQLRNY | |
| | RDNYQAFCKWLYDAKRRQDSLESMKFGDSNTVMRFLNEQKNLHSEISGKRD | |
| | KSEEVQKIAELCANSIKDYELQLASYTSGLETLLNIPIKRTMIQSPSGVILQEAA | |
| | DVHARYIELLTRSGDYYRFLSEMLKSLEDLKLKNTKIEVLEEELRLARDANSE | |
| | NCNKNKFLDQNLQKYQAECSQFKAKLASLEELKRQAELDGKSAKQNLDKCY | |
| | GQIKELNEKITRLTYEIEDEKRRRKSVEDRFDQQKNDYDQLQKARQCEKENLG | |
| | WQKLESEKAIKEKEYEIERLRVLLQEEGTRKREYENELAKVRNHYNEEMSNL | |
| | RNKYETEINITKTTIKEISMQKEDDSKNLRNQLDRLSRENRDLKDEIVRLNDSIL | |
| | QATEQRRRAEENALQQKACGSEIMQKKQHLEIELKQVMQQRSEDNARHKQS | |
| | LEEAAKTIQDKNKEIERLKAEFQEEAKRRWEYENELSKVRNNYDEEIISLKNQF | |
| | ETEINITKTTIHQLTMQKEEDTSGYRAQIDNLTRENRSLSEEIKRLKNTLTQTTE | |
| | NLRRVEEDIQQQKATGSEVSQRKQQLEVELRQVTQMRTEESVRYKQSLDDAA | |
| | KTIQDKNKEIERLKQLIDKETNDRKCLEDENARLQRVQYDLQKANSSATETIN | |
| | KLKVQEQELTRLRIDYERVSQERTVKDQDITRFQNSLKELQLQKQKVEEELNR | |
| | LKRTASEDSCKRKKLEEELEGMRRSLKEQAIKITNLTQQLEQASIVKKRSEDDL | |
| | RQQRDVLDGHLREKQRTQEELRRLSSEVEALRRQLLQEQESVKQAHLRNEHF | |
| | QKAIEDKSRSLNESKIEIERLQSLTENLTKEHLMLEEELRNLRLEYDDLRRGRS | |
| | EADSDKNATILELRSQLQISNNRTLELQGLINDLQRERENLRQEIEKFQKQALE | |
| | ASNRIQESKNQCTQVVQERESLLVKIKVLEQDKARLQRLEDELNRAKSTLEAE | |
| | TRVKQRLECEKQQIQNDLNQWKTQYSRKEEAIRKIESEREKSEREKNSLRSEIE | |
| | RLQAEIKRIEERCRRKLEDSTRETQSQLETERSRYQREIDKLRQRPYGSHRETQ | |
| | TECEWTVDTSKLVFDGLRKKVTAMQLYECQLIDKTTLDKLLKGKKSVEEVAS | |
| | EIQPFLRGAGSIAGASASPKEKYSLVEAKRKKLISPESTVMLLEAQAATGGIIDP | |
| | HRNEKLTVDSAIARDLIDFDDRQQIYAAEKAITGFDDPFSGKTVSVSEAIKKNLI | |
| | DRETGMRLLEAQIASGGVVDPVNSVFLPKDVALARGLIDRDLYRSLNDPRDSQ | |
| | KNFVDPVTKKKVSYVQLKERCRIEPHTGLLLLSVQKRSMSFQGIRQPVTVTEL | |
| | VDSGILRPSTVNELESGQISYDEVGERIKDFLQGSSCIAGIYNETTKQKLGIYEA | |
| | MKIGLVRPGTALELLEAQAATGFIVDPVSNLRLPVEEAYKRGLVGIEFKEKLLS | |
| | AERAVTGYNDPETGNIISLFQAMNKELIEKGHGIRLLEAQIATGGIIDPKESHRL | |
| | PVDIAYKRGYFNEELSEILSDPSDDTKGFFDPNTEENLTYLQLKERCIKDEETGL | |
| | CLLPLKEKKKQVQTSQKNTLRKRRVVIVDPETNKEMSVQEAYKKGLIDYETF | |
| | KELCEQECEWEEITITGSDGSTRVVLVDRKTGSQYDIQDAIDKGLVDRKFFDQ | |
| | YRSGSLSLTQFADMISLKNGVGTSSSMGSGVSDDVFSSSRHESVSKISTISSVRN | |
| | LTIRSSSFSDTLEESSPIAAIFDTENLEKISITEGIERGIVDSITGQRLLEAQACTGG | |
| | IIHPTTGQKLSLQDAVSQGVIDQDMATRLKPAQKAFIGFEGVKGKKKMSAAE | |
| | AVKEKWLPYEAGQRFLEFQYLTGGLVDPEVHGRISTEEAIRKGFIDGRAAQRL | |
| | QDTSSYAKILTCPKTKLKISYKDAINRSMVEDITGLRLLEAASVSSKGLPSPYN | |
| | MSSAPGSRSGSRSGSRSGSRSGSRSGSRRGSFDATGNSSYSYSYSFSSSSIGH | |
| Human | atgagctgcaacggaggctcccacccgcggatcaacactctgggccgcatgatccgcgccgagtctggcccggacctgc | 347 |
| DSP | gctacgaggtgaccagcggcggcgggggcaccagcaggatgtactattctcggcgcggcgtgatcaccgaccagaactc | |
| DPII | ggacggctactgtcaaaccggcacgatgtccaggcaccagaaccagaacaccatccaggagctgctgcagaactgctcc | |
| DNA | gactgcttgatgcgagcagagctcatcgtgcagcctgaattgaagtatggagatggaatacaactgactcggagtcgagaat | |
| | tggatgagtgttttgcccaggccaatgaccaaatggaaatcctcgacagcttgatcagagagatgcggcagatgggccagc | |
| | cctgtgatgcttaccagaaaaggcttcttcagctccaagagcaaatgcgagccctttataaagccatcagtgtccctcgagt | |
| | ccgcagggccagctccaagggtggtggaggctacacttgtcagagtggctctggctgggatgagttcaccaaacatgtcacc | |
| | agtgaatgtttggggtggatgaggcagcaaagggcggagatggacatggtggcctggggtgtggacctggcctcagtgga | |
| | gcagcacattaacagccaccggggcatccacaactccatcggcgactatcgctggcagctggacaaaatcaaagccgacc | |
| | tgcgcgagaaatctgcgatctaccagttggaggaggagtatgaaaacctgctgaaagcgtcctttgagaggatggatcacct | |
| | gcgacagctgcagaacatcattcaggccacgtccagggagatcatgtggatcaatgactgcgaggaggaggagctgctgt | |
| | acgactggagcgacaagaacaccaacatcgctcagaaacaggaggccttctccatacgcatgagtcaactggaagttaaa | |
| | gaaaaagagctcaataagctgaaacaagaaagtgaccaacttgtcctcaatcagcatccagcttcagacaaaattgaggcct | |
| | atatggacactctgcagacgcagtggagttggattcttcagatcaccaagtgcattgatgttcatctgaaagaaaatgctgc | |
| | actctttcagttttttgaagaggcgcagtctactgaagcatacctgaaggggctccaggactccatcaggaagaagtacccc | |
| | tgcgacaagaacatgcccctgcagcacctgctggaacagatcaaggagctggagaaagaacgagagaaaatccttgaataca | |
| | agcgtcaggtgcagaacttggtaaacaagtctaagaagattgtacagctgaagcctcgtaacccagactacagaagcaata | |
| | aacccattattctcagagctctctgtgactacaaacaagatcagaaaatcgtgcataagggggatgagtgtatcctgaagga | |
| | caacaacgagcgcagcaagtggtacgtgacgggcccgggaggcgttgacatgcttgttccctctgtggggctgatcatccct | |
| | cctccgaacccactggccgtggacctctcttgcaagattgagcagtactacgaagccatcttggctctgtggaaccagctct | |
| | acatcaacatgaagagcctggtgtcctggcactactgcatgattgacatagagaagatcagggccatgacaatcgccaagct | |
| | gaaaacaatgcggcaggaagattacatgaagacgatagccgaccttgagttacattaccaagagttcatcagaaatagccaa | |
| | ggctcagagatgtttggagatgatgacaagcggaaaatacagtctcagttcaccgatgcccagaagcattaccagaccctgg | |
| | tcattcagctccctggctatccccagcaccagacagtgaccacaactgaaatcactcatcatggaacctgccaagatgtcaa | |
| | ccataataaagtaattgaaaccaacagagaaaatgacaagcaagaaacatggatgctgatggagctgcagaagattcgcagg | |
| | cagatagagcactgcgagggcaggatgactctcaaaaacctccctctagcagaccagggatcttctcaccacatcacagtg | |
| | aaaattaacgagcttaagagtgtgcagaatgattcacaagcaattgctgaggttctcaaccagcttaaagatatgcttgcca | |
| | acttcagaggttctgaaaagtactgctatttacagaatgaagtatttggactatttcagaaactggaaaatatcaatggtgt | |
| | tacagatggctacttaaatagcttatgcacagtaagggcactgctccaggctattctccaaacagaagacatgttaaaggtt | |
| | tatgaagccaggctcactgaggaggaaactgtctgcctggacctggataaagtggaagcttaccgctgtggactgaagaaaa | |
| | aaaaaatgacttgaacttgaagaagtcgttgttggccactatgaagacagaactacagaaagcccagcagatccactctcag | |
| | acttcacagcagtatccactttatgatctggacttgggcaagttcggtgaaaaagtcacacagctgacagaccgctggcaaa | |
| | ggatagataaacagatcgactttaggttatgggacctggagaaacaaatcaagcaattgaggaattatcgtgataactatca | |
| | ggctttctgcaagtggctctatgatgctaaacgccgccaggattccttagaatccatgaaatttggagattccaacacagtc | |
| | atgcggtttttgaatgagcagaagaacttgcacagtgaaatatctggcaaacgagacaaatcagaggaagtacaaaaaattg | |
| | ctgaactttgcgccaattcaattaaggattatgagctccagctggcctcatacacctcaggactggaaactctgctgaacat | |
| | acctatcaagaggaccatgattcagtccccttctggggtgattctgcaagaggctgcagatgttcatgctcggtacattgaa | |
| | ctacttacaagatctggagactattacaggttcttaagtgagatgctgaagagtttggaagatctgaagctgaaaaatacca | |
| | agatcgaagttttggaagaggagctcagactggcccgagatgccaactcggaaaactgtaataagaacaaattcctggatca | |
| | gaacctgcagaaataccaggcagagtgttcccagttcaaagcgaagcttgcgagcctggaggagctgaagagacaggctgag | |
| | tctggatgggaagcggctaagcaaaatctagacaagtgctacggccaaataaaagaactcaatgagaagatcacccgactga | |
| | cttatgagattgaagatgaaaagagaagaagaaaatctgtggaagacagatttgaccaacagaagaatgactatgaccaact | |
| | gcagaaagcaaggcaatgtgaaaaggagaaccttggttggcagaaattagagtctgagaaagccatcaaggagaaggagtac | |
| | gagattgaaaggttgagggttctactgcaggaagaaggcacccggaagagagaatatgaaaatgagctggcaaaggcatcta | |
| | ataggattcaggaatcaaagaatcagtgtactcaggtggtacaggaaagagagagccttctggtgaaaatcaaagtcctgga | |
| | gcaagacaaggcaaggctgcagaggctggaggatgagctgaatcgtgcaaaatcaactctagaggcagaaaccagggtgaaa | |
| | cagcgcctggagtgtgagaaacagcaaattcagaatgacctgaatcagtggaagactcaatattcccgcaaggaggaggcta | |
| | ttaggaagatagaatcggaaagagaaaagagtgagagagagaagaacagtcttaggagtgagatcgaaagactccaagca | |
| | gagatcaagagaattgaagagaggtgcaggcgtaagctggaggattctaccagggagacacagtcacagttagaaacag | |
| | aacgctcccgatatcagagggagattgataaactcagacagcgcccatatgggtcccatcgagagacccagactgagtgtg | |
| | agtggaccgttgacacctccaagctggtgtttgatgggctgaggaagaaggtgacagcaatgcagctctatgagtgtcagct | |
| | gatcgacaaaacaaccttggacaaactattgaaggggaagaagtcagtggaagaagttgcttctgaaatccagccattcctt | |
| | cggggtgcaggatctatcgctggagcatctgcttctcctaaggaaaaatactctttggtagaggccaagagaaagaaattaa | |
| | tcagcccagaatccacagtcatgcttctggaggcccaggcagctacaggtggtataattgatccccatcggaatgagaagct | |
| | gactgtcgacagtgccatagctcgggacctcattgacttcgatgaccgtcagcagatatatgcagcagaaaaagctatcact | |
| | ggttttgatgatccattttcaggcaagacagtatctgtttcagaagccatcaagaaaaatttgattgatagagaaaccggaa | |
| | tgcgcctgctggaagcccagattgcttcagggggtgtagtagaccctgtgaacagtgtctttttgccaaaagatgtcgcctt | |
| | ggcccgggggctgattgatagagatttgtatcgatccctgaatgatccccgagatagtcagaaaaactttgtggatccagtc | |
| | accaaaaagaaggtcagttacgtgcagctgaaggaacggtgcagaatcgaaccacatactggtctgctcttgctttcagtac | |
| | agaagagaagcatgtccttccaaggaatcagacaacctgtgaccgtcactgagctagtagattctggtatattgagaccgtc | |
| | cactgtcaatgaactggaatctggtcagatttcttatgacgaggttggtgagagaattaaggacttcctccagggttcaagc | |
| | tgcatagcaggcatatacaatgagaccacaaaacagaagcttggcatttatgaggccatgaaaattggcttagtccgacctg | |
| | gtactgctctggagttgctggaagcccaagcagctactggctttatagtggatcctgttagcaacttgaggttaccagtgga | |
| | ggaagcctacaagagaggtctggtgggcattgagttcaaagagaagctcctgtctgcagaacgagctgtcactgggtataat | |
| | gatcctgaaacaggaaacatcatctctttgttccaagccatgaataaggaactcatcgaaaagggccacggtattcgcttat | |
| | tagaagcacagatcgcaaccggggggatcattgacccaaaggagagccatcgtttaccagttgacatagcatataagagggg | |
| | ctatttcaatgaggaactcagtgagattctctcagatccaagtgatgataccaaaggattttttgaccccaacactgaaga | |
| | aaatcttacctatctgcaactaaaagaaagatgcattaaggatgaggaaacagggctctgtcttctgcctctgaaagaaaa | |
| | gaagaaacaggtgcagacatcacaaaagaataccctcaggaagcgtagagtggtcatagttgacccagaaaccaataaag | |
| | aaatgtctgttcaggaggcctacaagaagggcctaattgattatgaaaccttcaaagaactgtgtgagcaggaatgtgaat | |
| | gggaagaaataaccatcacgggatcagatggctccaccagggtggtcctggtagatagaaagacaggcagtcagtatgata | |
| | ttcaagatgctattgacaagggccttgttgacaggaagttctttgatcagtaccgatccggcagcctcagcctcactcaat | |
| | ttgctgacatgatctccttgaaaaatggtgtcggcaccagcagcagcatgggcagtggtgtcagcgatgatgtttttagca | |
| | gctcccgacatgaatcagtaagtaagatttccaccatatccagcgtcaggaatttaaccataaggagcagctctttttcaga | |
| | caccctggaagaatcgagccccattgcagccatctttgacacagaaaacctggagaaaatctccattacagaaggtataga | |
| | gcggggcatcgttgacagcatcacgggtcagaggcttctggaggctcaggcctgcacaggtggcatcatccacccaaccac | |
| | gggccagaagctgtcacttcaggacgcagtctcccagggtgtgattgaccaagacatggccaccaggctgaagcctgctca | |
| | gaaagccttcataggcttcgagggtgtgaagggaaagaagaagatgtcagcagcagaggcagtgaaagaaaaatggctcccg | |
| | tatgaggctggccagcgcttcctggagttccagtacctcacgggaggtcttgttgacccggaagtgcatgggaggataagca | |
| | ccgaagaagccatccggaaggggttcatagatggccgcgccgcacagaggctgcaagacaccagcagctatgccaaaatcct | |
| | gacctgccccaaaaccaaattaaaaatatcctataaggatgccataaatcgctccatggtagaagatatcactgggctgcgc | |
| | cttctggaagccgcctccgtgtcgtccaagggcttacccagcccttacaacatgtcttcggctccggggtcccgctccggct | |
| | cccgctcgggatctcgctccggatctcgctccgggtcccgcagtgggtcccggagaggaagctttgacgccacagggaattc | |
| | ttcctactcttattcctactcatttagcagtagttctattgggcactag | |
| Human | MSCNGGSHPRINTLGRMIRAESGPDLRYEVTSGGGGTSRMYYSRRGVITDQNS | 348 |
| DSP | DGYCQTGTMSRHQNQNTIQELLQNCSDCLMRAELIVQPELKYGDGIQLTRSRE | |
| DPII | LDECFAQANDQMEILDSLIREMRQMGQPCDAYQKRLLQLQEQMRALYKAISV | |
| protein | PRVRRASSKGGGGYTCQSGSGWDEFTKHVTSECLGWMRQQRAEMDMVAW | |
| | GVDLASVEQHINSHRGIHNSIGDYRWQLDKIKADLREKSAIYQLEEEYENLLK | |
| | ASFERMDHLRQLQNIIQATSREIMWINDCEEEELLYDWSDKNTNIAQKQEAFSI | |
| | RMSQLEVKEKELNKLKQESDQLVLNQHPASDKIEAYMDTLQTQWSWILQITK | |
| | CIDVHLKENAAYFQFFEEAQSTEAYLKGLQDSIRKKYPCDKNMPLQHLLEQIK | |
| | ELEKEREKILEYKRQVQNLVNKSKKIVQLKPRNPDYRSNKPIILRALCDYKQD | |
| | QKIVHKGDECILKDNNERSKWYVTGPGGVDMLVPSVGLIIPPPNPLAVDLSCKI | |
| | EQYYEAILALWNQLYINMKSLVSWHYCMIDIEKIRAMTIAKLKTMRQEDYMK | |
| | TIADLELHYQEFIRNSQGSEMFGDDDKRKIQSQFTDAQKHYQTLVIQLPGYPQ | |
| | HQTVTTTEITHHGTCQDVNHNKVIETNRENDKQETWMLMELQKIRRQIEHCE | |
| | GRMTLKNLPLADQGSSHHITVKINELKSVQNDSQAIAEVLNQLKDMLANFRG | |
| | SEKYCYLQNEVFGLFQKLENINGVTDGYLNSLCTVRALLQAILQTEDMLKVY | |
| | EARLTEEETVCLDLDKVEAYRCGLKKIKNDLNLKKSLLATMKTELQKAQQIH | |
| | SQTSQQYPLYDLDLGKFGEKVTQLTDRWQRIDKQIDFRLWDLEKQIKQLRNY | |
| | RDNYQAFCKWLYDAKRRQDSLESMKFGDSNTVMRFLNEQKNLHSEISGKRD | |
| | KSEEVQKIAELCANSIKDYELQLASYTSGLETLLNIPIKRTMIQSPSGVILQEAA | |
| | DVHARYIELLTRSGDYYRFLSEMLKSLEDLKLKNTKIEVLEEELRLARDANSE | |
| | NCNKNKFLDQNLQKYQAECSQFKAKLASLEELKRQAELDGKSAKQNLDKCY | |
| | GQIKELNEKITRLTYEIEDEKRRRKSVEDRFDQQKNDYDQLQKARQCEKENLG | |
| | WQKLESEKAIKEKEYEIERLRVLLQEEGTRKREYENELAKASNRIQESKNQCT | |
| | QVVQERESLLVKIKVLEQDKARLQRLEDELNRAKSTLEAETRVKQRLECEKQ | |
| | QIQNDLNQWKTQYSRKEEAIRKIESEREKSEREKNSLRSEIERLQAEIKRIEERC | |
| | RRKLEDSTRETQSQLETERSRYQREIDKLRQRPYGSHRETQTECEWTVDTSKL | |
| | VFDGLRKKVTAMQLYECQLIDKTTLDKLLKGKKSVEEVASEIQPFLRGAGSIA | |
| | GASASPKEKYSLVEAKRKKLISPESTVMLLEAQAATGGIIDPHRNEKLTVDSAI | |
| | ARDLIDFDDRQQIYAAEKAITGFDDPFSGKTVSVSEAIKKNLIDRETGMRLLEA | |
| | QIASGGVVDPVNSVFLPKDVALARGLIDRDLYRSLNDPRDSQKNFVDPVTKK | |
| | KVSYVQLKERCRIEPHTGLLLLSVQKRSMSFQGIRQPVTVTELVDSGILRPSTV | |
| | NELESGQISYDEVGERIKDFLQGSSCIAGIYNETTKQKLGIYEAMKIGLVRPGT | |
| | ALELLEAQAATGFIVDPVSNLRLPVEEAYKRGLVGIEFKEKLLSAERAVTGYN | |
| | DPETGNIISLFQAMNKELIEKGHGIRLLEAQIATGGIIDPKESHRLPVDIAYKRG | |
| | YFNEELSEILSDPSDDTKGFFDPNTEENLTYLQLKERCIKDEETGLCLLPLKEKK | |
| | KQVQTSQKNTLRKRRVVIVDPETNKEMSVQEAYKKGLIDYETFKELCEQECE | |
| | WEEITITGSDGSTRVVLVDRKTGSQYDIQDAIDKGLVDRKFFDQYRSGSLSLTQ | |
| | FADMISLKNGVGTSSSMGSGVSDDVFSSSRHESVSKISTISSVRNLTIRSSSFSDT | |
| | LEESSPIAAIFDTENLEKISITEGIERGIVDSITGQRLLEAQACTGGIIHPTTGQKLS | |
| | LQDAVSQGVIDQDMATRLKPAQKAFIGFEGVKGKKKMSAAEAVKEKWLPYE | |
| | AGQRFLEFQYLTGGLVDPEVHGRISTEEAIRKGFIDGRAAQRLQDTSSYAKILT | |
| | CPKTKLKISYKDAINRSMVEDITGLRLLEAASVSSKGLPSPYNMSSAPGSRSGS | |
| | RSGSRSGSRSGSRSGSRRGSFDATGNSSYSYSYSFSSSSIGH | |
| Human | atggcgcggagcccgggacgcgcgtacgccctgctgcttctcctgatctgctttaacgttggaagtggacttcacttacagg | 349 |
| DSG2 | tcttaagcacaagaaatgaaaataagctgcttcctaaacatcctcatttagtgcggcaaaagcgcgcctggatcaccgcccc | |
| DNA | cgtggctcttcgggagggagaggatctgtccaagaagaatccaattgccaagatacattctgatcttgcagaagaaagagga | |
| | ctcaaaattacttacaaatacactggaaaagggattacagagccaccttttggtatatttgtctttaacaaagatactggag | |
| | aactgaatgttaccagcattcttgatcgagaagaaacaccattttttctgctaacaggttacgctttggatgcaagaggaaa | |
| | caatgtagagaaacccttagagctacgcattaaggttcttgatatcaatgacaacgaaccagtgttcacacaggatgtcttt | |
| | gttgggtctgttgaagagttgagtgcagcacatactcttgtgatgaaaatcaatgcaacagatgcagatgagcccaataccc | |
| | tgaattcgaaaatttcctatagaatcgtatctctggagcctgcttatcctccagtgttctacctaaataaagatacaggaga | |
| | gatttatacaaccagtgttaccttggacagagaggaacacagcagctacactttgacagtagaagcaagagatggcaatgga | |
| | gaagttacagacaaacctgtaaaacaagctcaagttcagattcgtattttggatgtcaatgacaatatacctgtagtagaaa | |
| | ataaagtgcttgaagggatggttgaagaaaatcaagtcaacgtagaagttacgcgcataaaagtgttcgatgcagatgaaat | |
| | aggttctgataattggctggcaaattttacatttgcatcaggaaatgaaggaggttatttccacatagaaacagatgctcaa | |
| | actaacgaaggaattgtgacccttattaaggaagtagattatgaagaaatgaagaatcttgacttcagtgttattgtcgcta | |
| | ataaagcagcttttcacaagtcgattaggagtaaatacaagcctacacccattcccatcaaggtcaaagtgaaaaatgtga | |
| | aagaaggcattcattttaaaagcagcgtcatctcaatttatgttagcgagagcatggatagatcaagcaaaggccaaataa | |
| | ttggaaattttcaagcttttgatgaggacactggactaccagcccatgcaagatatgtaaaattagaagatagagataattg | |
| | gatctctgtggattctgtcacatctgaaattaaacttgcaaaacttcctgattttgaatctagatatgttcaaaatggcaca | |
| | tacactgtaaagattgtggccatatcagaagattatcctagaaaaaccatcactggcacagtccttatcaatgttgaagaca | |
| | tcaacgacaactgtcccacactgatagagcctgtgcagacaatctgtcacgatgcagagtatgtgaatgttactgcagagga | |
| | cctggatggacacccaaacagtggccctttcagtttctccgtcattgacaaaccacctggcatggcagaaaaatggaaaata | |
| | gcacgccaagaaagtaccagtgtgctgctgcaacaaagtgagaaaaagcttgggagaagtgaaattcagttcctgatttcag | |
| | acaatcagggttttagttgtcctgaaaagcaggtccttacactcacagtttgtgagtgtctgcatggcagcggctgcaggg | |
| | aagcacagcatgactcctatgtgggcctgggacccgcagcaattgcgctcatgattttggcctttctgctcctgctattgg | |
| | taccacttttactgctga | |
| Human | MARSPGRAYALLLLLICFNVGSGLHLQVLSTRNENKLLPKHPHLVRQKRAWIT | 350 |
| DSG2 | APVALREGEDLSKKNPIAKIHSDLAEERGLKITYKYTGKGITEPPFGIFVENKDT | |
| protein | GELNVTSILDREETPFFLLTGYALDARGNNVEKPLELRIKVLDINDNEPVFTQD | |
| | VFVGSVEELSAAHTLVMKINATDADEPNTLNSKISYRIVSLEPAYPPVFYLNKD | |
| | TGEIYTTSVTLDREEHSSYTLTVEARDGNGEVTDKPVKQAQVQIRILDVNDNIP | |
| | VVENKVLEGMVEENQVNVEVTRIKVFDADEIGSDNWLANFTFASGNEGGYFH | |
| | IETDAQTNEGIVTLIKEVDYEEMKNLDFSVIVANKAAFHKSIRSKYKPTPIPIKV | |
| | KVKNVKEGIHFKSSVISIYVSESMDRSSKGQIIGNFQAFDEDTGLPAHARYVKL | |
| | EDRDNWISVDSVTSEIKLAKLPDFESRYVQNGTYTVKIVAISEDYPRKTITGTV | |
| | LINVEDINDNCPTLIEPVQTICHDAEYVNVTAEDLDGHPNSGPFSFSVIDKPPGM | |
| | AEKWKIARQESTSVLLQQSEKKLGRSEIQFLISDNQGFSCPEKQVLTLTVCECL | |
| | HGSGCREAQHDSYVGLGPAAIALMILAFLLLLLVPLLLLMCHCGKGAKGFTPI | |
| | PGTIEMLHPWNNEGAPPEDKVVPSFLPVDQGGSLVGRNGVGGMAKEATMKG | |
| | SSSASIVKGQHEMSEMDGRWEEHRSLLSGRATQFTGATGAIMTTETTKTARAT | |
| | GASRDMAGAQAAAVALNEEFLRNYFTDKAASYTEEDENHTAKDCLLVYSQE | |
| | ETESLNASIGCCSFIEGELDDRFLDDLGLKFKTLAEVCLGQKIDINKEIEQRQKP | |
| | ATETSMNTASHSLCEQTMVNSENTYSSGSSFPVPKSLQEANAEKVTQEIVTERS | |
| | VSSRQAQKVATPLPDPMASRNVIATETSYVTGSTMPPTTVILGPSQPQSLIVTE | |
| | RVYAPASTLVDQPYANEGTVVVTERVIQPHGGGSNPLEGTQHLQDVPYVMVR | |
| | ERESFLAPSSGVQPTLAMPNIAVGQNVTVTERVLAPASTLQSSYQIPTENSMTA | |
| | RNTTVSGAGVPGPLPDFGLEESGHSNSTITTSSTRVTKHSTVQHSYS | |
| Human | atggaggtgatgaacctgatggagcagcctatcaaggtgactgagtggcagcagacatacacctacgactcgggtatccac | 351 |
| JUP | tcgggcgccaacacctgcgtgccctccgtcagcagcaagggcatcatggaggaggatgaggcctgcgggcgccagtac | |
| DNA | acgctcaagaaaaccaccacttacacccagggggtgccccccagccaaggtgatctggagtaccagatgtccacaacagc | |
| | cagggccaaacgggtgcgggaggccatgtgccctggtgtgtcaggcgaggacagctcgcttctgctggccacccaggtg | |
| | gaggggcaggccaccaacctgcagcgactggccgagccgtcccagctgctcaagtcggccattgtgcatctcatcaacta | |
| | ccaggacgatgccgagctggccactcgcgccctgcccgagctcaccaaactgctcaacgacgaggacccggtggtggtg | |
| | accaaggcggccatgattgtgaaccagctgtcgaagaaggaggcgtcgcggcgggccctgatgggctcgccccagctg | |
| | gtggccgctgtcgtgcgtaccatgcagaataccagcgacctggacacagcccgctgcaccaccagcatcctgcacaacct | |
| | ctcccaccaccgggaggggctgctcgccatcttcaagtcgggtggcatccctgctctggtccgcatgctcagctcccctgtg | |
| | gagtcggtcctgttctatgccatcaccacgctgcacaacctgctcctgtaccaggagggcgccaagatggccgtgcgcctg | |
| | gccgacgggctgcaaaagatggtgcccctgctcaacaagaacaaccccaagttcctggccatcaccaccgactgcctgca | |
| | gctcctggcctacggcaaccaggagagcaagctgatcatcctggccaatggtgggccccaggccctcgtgcagatcatgc | |
| | gtaactacagttatgaaaagctgctctggaccaccagtcgtgtgctcaaggtgctatccgtgtgtcccagcaataagcctgc | |
| | cattgtggaggctggtgggatgcaggccctgggcaagcacctgaccagcaacagcccccgcctggtgcagaactgcctgt | |
| | ggaccctgcgcaacctctcagatgtggccaccaagcaggagggcctggagagtgtgctgaagattctggtgaatcagctg | |
| | agtgtggatgacgtcaacgtcctcacctgtgccacgggcacactctccaacctgacatgcaacaacagcaagaacaagac | |
| | gctggtgacacagaacagcggtgtggaggctctcatccatgccatcctgcgtgctggtgacaaggacgacatcacggagc | |
| | ctgccgtctgcgctctgcgccacctcactagccgccaccctgaggccgagatggcccagaactctgtgcgtctcaactatgg | |
| | catcccagccatcgtgaagctgctcaaccagcccaaccagtggccactggtcaaggcaaccatcggcttgatcaggaatct | |
| | ggccctgtgcccagccaaccatgccccgctgcaggaggcagcggtcatcccccgcctcgtccaactgctggtgaaggcc | |
| | caccaggatgcccagcgccacgtagctgcaggcacacagcagccctacacggatggtgtgaggatggaggagattgtgg | |
| | agggctgcaccggagcactgcacatcctcgcccgggaccccatgaaccgcatggagatcttccggctcaacaccattccc | |
| | ctgtttgtgcagctcctgtactcgtcggtggagaacatccagcgcgtggctgccggggtgctgtgtgagctggcccaggac | |
| | aaggaggcggccgacgccattgatgcagagggggcctcggccccactcatggagttgctgcactcccgcaacgagggc | |
| | actgccacctacgctgctgccgtcctgttccgcatctccgaggacaagaacccagactaccggaagcgcgtgtccgtggag | |
| | ctcaccaactccctcttcaagcatgacccggctgcctgggaggctgcccagagcatgattcccatcaatgagccctatggag | |
| | atgacatggatgccacctaccgccccatgtactccagcgatgtgccccttgacccgctggagatgcacatggacatggatg | |
| | gagactaccccatcgacacctacagcgacggcctcaggcccccgtaccccactgcagaccacatgctggcctag | |
| Human | MEVMNLMEQPIKVTEWQQTYTYDSGIHSGANTCVPSVSSKGIMEEDEACGRQ | 352 |
| JUP | YTLKKTTTYTQGVPPSQGDLEYQMSTTARAKRVREAMCPGVSGEDSSLLLAT | |
| protein | QVEGQATNLQRLAEPSQLLKSAIVHLINYQDDAELATRALPELTKLLNDEDPV | |
| | VVTKAAMIVNQLSKKEASRRALMGSPQLVAAVVRTMQNTSDLDTARCTTSIL | |
| | HNLSHHREGLLAIFKSGGIPALVRMLSSPVESVLFYAITTLHNLLLYQEGAKMA | |
| | VRLADGLQKMVPLLNKNNPKFLAITTDCLQLLAYGNQESKLIILANGGPQALV | |
| | QIMRNYSYEKLLWTTSRVLKVLSVCPSNKPAIVEAGGMQALGKHLTSNSPRL | |
| | VQNCLWTLRNLSDVATKQEGLESVLKILVNQLSVDDVNVLTCATGTLSNLTC | |
| | NNSKNKTLVTQNSGVEALIHAILRAGDKDDITEPAVCALRHLTSRHPEAEMAQ | |
| | NSVRLNYGIPAIVKLLNQPNQWPLVKATIGLIRNLALCPANHAPLQEAAVIPRL | |
| | VQLLVKAHQDAQRHVAAGTQQPYTDGVRMEEIVEGCTGALHILARDPMNRM | |
| | EIFRLNTIPLFVQLLYSSVENIQRVAAGVLCELAQDKEAADAIDAEGASAPLME | |
| | LLHSRNEGTATYAAAVLFRISEDKNPDYRKRVSVELTNSLFKHDPAAWEAAQ | |
| | SMIPINEPYGDDMDATYRPMYSSDVPLDPLEMHMDMDGDYPIDTYSDGLRPP | |
| | YPTADHMLA | |
| Human | atggctccggccgcctggctccgcagcgcggccgcgcgcgccctcctgcccccgatgctgctgctgctgctccagccgcc | 353 |
| MMP11 | gccgctgctggcccgggctctgccgccggacgcccaccacctccatgccgagaggagggggccacagccctggcatgc | |
| DNA | agccctgcccagtagcccggcacctgcccctgccacgcaggaagccccccggcctgccagcagcctcaggcctccccg | |
| | ctgtggcgtgcccgacccatctgatgggctgagtgcccgcaaccgacagaagaggttcgtgctttctggcgggcgctggg | |
| | agaagacggacctcacctacaggatccttcggttcccatggcagttggtgcaggagcaggtgcggcagacgatggcaga | |
| | ggccctaaaggtatggagcgatgtgacgccactcacctttactgaggtgcacgagggccgtgctgacatcatgatcgacttc | |
| | gccaggtactggcatggggacgacctgccgtttgatgggcctgggggcatcctggcccatgccttcttccccaagactcac | |
| | cgagaaggggatgtccacttcgactatgatgagacctggactatcggggatgaccagggcacagacctgctgcaggtggc | |
| | agcccatgaatttggccacgtgctggggctgcagcacacaacagcagccaaggccctgatgtccgccttctacacctttcgc | |
| | tacccactgagtctcagcccagatgactgcaggggcgttcaacacctatatggccagccctggcccactgtcacctccagg | |
| | accccagccctgggcccccaggctgggatagacaccaatgagattgcaccgctggagccagacgccccgccagatgcct | |
| | gtgaggcctcctttgacgcggtctccaccatccgaggcgagctctttttcttcaaagcgggctttgtgtggcgcctccgtgg | |
| | gggccagctgcagcccggctacccagcattggcctctcgccactggcagggactgcccagccctgtggacgctgccttcga | |
| | ggatgcccagggccacatttggttcttccaaggtgctcagtactgggtgtacgacggtgaaaagccagtcctgggccccgc | |
| | acccctcaccgagctgggcctggtgaggttcccggtccatgctgccttggtctggggtcccgagaagaacaagatctacttc | |
| | ttccgaggcagggactactggcgtttccaccccagcacccggcgtgtagacagtcccgtgccccgcagggccactgactg | |
| | gagaggggtgccctctgagatcgacgctgccttccaggatgctgatggctatgcctacttcctgcgcggccgcctctactgg | |
| | aagtttgaccctgtgaaggtgaaggctctggaaggcttcccccgtctcgtgggtcctgacttctttggctgtgccgagcctg | |
| Human | MAPAAWLRSAAARALLPPMLLLLLQPPPLLARALPPDAHHLHAERRGPQPWH | 354 |
| MMP11 | AALPSSPAPAPATQEAPRPASSLRPPRCGVPDPSDGLSARNRQKRFVLSGGRW | |
| protein | EKTDLTYRILRFPWQLVQEQVRQTMAEALKVWSDVTPLTFTEVHEGRADIMI | |
| | DFARYWHGDDLPFDGPGGILAHAFFPKTHREGDVHFDYDETWTIGDDQGTDL | |
| | LQVAAHEFGHVLGLQHTTAAKALMSAFYTFRYPLSLSPDDCRGVQHLYGQP | |
| | WPTVTSRTPALGPQAGIDTNEIAPLEPDAPPDACEASFDAVSTIRGELFFFKAGF | |
| | VWRLRGGQLQPGYPALASRHWQGLPSPVDAAFEDAQGHIWFFQGAQYWVY | |
| | DGEKPVLGPAPLTELGLVRFPVHAALVWGPEKNKIYFFRGRDYWRFHPSTRR | |
| | VDSPVPRRATDWRGVPSEIDAAFQDADGYAYFLRGRLYWKFDPVKVKALEG | |
| | FPRLVGPDFFGCAEPANTFL | |
| Human | atgggtgctgaggaggaggtgctggtcacactatcagggggagccccctggggcttccgacttcatgggggggccgagc | 355 |
| SYNPO2 | agaggaaaccgttacaggtgtctaagattcgaagacggagccaggctggcagagcaggactccgagagagggaccagc | |
| LA DNA | tcttggcaatcaatggggtctcttgcaccaacctctcccatgccagtgccatgagcctcatcgatgcctcaggaaatcagct | |
| | tgtcctcactgtgcagcggttagcagacgagggtcctgtgcaatctccatctccccatgagcttcaggtgctgtcaccctta | |
| | tctccactaagtcctgagccccctggtgctccagttcctcagcctcttcagcctgggagccttcgttcacctcctgatagtg | |
| | aggcttactacggagagactgacagtgatgctgatggccctgccacccaggagaagccccgtcgacctcgccgccgaggccc | |
| | cacaaggcccacccctccgggtgccccacctgatgaggtctacctgtctgacagccctgcagagccagcacctactatccct | |
| | ggccctcccagccagggtgacagcgtgtgagctccccgtcttgggaggatggggcagcccttcagccacccccagctgagg | |
| | ctctgctgttaccccatggccccctccgacctggtcctcatctcatccctatggtggggcctgttccccacccagtggcaga | |
| | agatcttactaccacctacacccagaaggccaagcaagccaaactgcaacgtgcagagagcctccaagagaagagcataaa | |
| | agaggccaagaccaaatgcaggacaattgcatccctgctcactgcagcccccaacccccactccaaaggggtacttatgttt | |
| | aagaaacggcggcagagagccaagaagtacaccctggtgagcttcggggctgctgctgggacaggcgctgaggagga | |
| | ggacggcgttccccccacgagtgagtccgagctggacgaagaagccttctctgacgcccgcagcctcaccaatcaatctg | |
| | actgggacagtccctatctggacatggagcttgccagggcgggctcaagagcatcagagggccagggctctgggctggg | |
| | agggcagctgagtgaggtctctgggcgaggggtgcagctctttgaacagcagcgccagcgcgcagactccagcacccag | |
| | gaactggcacgggtcgaaccagcagccatgctcaacggggaaggcctgcagtcaccacctcgggcccagagtgctcccc | |
| | cagaggcagctgtgctcccacccagccccttgccggcgcctgtagccagccccagacccttccaaccaggtggtggagcc | |
| | ccgaccccagctccaagcatctttaaccggtcagccaggccctttaccccgggcctacaagggcagcggccaactaccac | |
| | ctcggttattttccggcctttagcccccaaaagggcgaacgacagcctggggggcctcagccccgccccaccccccttcttg | |
| | tcttcgcaggggcccacccctctgcccagcttcacttcaggggttcccagccacgcgccagtctctggttcccccagcaccc | |
| | cacgctcctcgggccctgtgacagccaccagctccctgtacatcccagcccctagtcggcctgtcaccccaggtggagctc | |
| | cagagccccccgctcctcctagcgcagctgccatgacctccaccgcttctatcttcctatctgcgcctttgcgaccctctgc | |
| | gcgcccagaggcgcctgccccaggcccaggggctcctgagccccccagcgctcgcgagcagcgcatctctgtgccagctg | |
| | cccgcacgggtatcctgcaggaggcccggcgccgggggacccggaagcagatgttccggccgggaaaggaggagac | |
| | gaagaactcgcccaaccccgagctgctatcgctggtacagaacctggatgaaaagcctcgggccgggggtgcagaatctg | |
| | gtcctgaagaagatgctctgagcctcggggctgaagcctgcaacttcatgcagccagtaggggccaggagttacaagacc | |
| | ctgcctcacgtgacacctaagaccccccctccaatggctcccaagaccccgccccctatgactcctaagactccaccccca | |
| | gtggctcctaagcccccatctcgagggctccttgatgggctcgtgaatggggcagcctcttcggctggaatccctgagccac | |
| | caaggctgcagggcaggggtggggagctgtttgctaagcggcagagccgtgcggacaggtatgtggtggaaggtacacc | |
| | tggtcctggtcttggccctcggcctagaagtccttctcctaccccgtctctgcccccttcctggaaatattcacccaacatc | |
| | cgtgccccgcctcctattgcttacaacccactgctctctccctttttcccccaggcggcccgaactctccctaaggcccaat | |
| | cccaggggcctcgggcaacacccaagcagggcatcaaggctctagattttatgcggcatcagccctatcaacttaaaactgc | |
| | catgttctgttttgatgaggttcccccgactcctggccctatcgcctcagggtcccccaaaactgcccgagtccaggagat | |
| | tcgccggttttccactccggcaccccagcccactgcagaacccctggctcccactgtgcttgccccccgagcagccactac | |
| | actggatgagcccatctggagaacagaactggcctcagcccctgttcctagcccagcccctcctccagaggctcccagggg | |
| | ccttggggcttctcccagctcctgcggtttccaggtagccaggccccgattttcagccaccagaacaggattgcaagctca | |
| | tgtgtggaggcctggggcagggcaccag | |
| Human | MGAEEEVLVTLSGGAPWGFRLHGGAEQRKPLQVSKIRRRSQAGRAGLRERDQ | 356 |
| SYNPO2 | LLAINGVSCTNLSHASAMSLIDASGNQLVLTVQRLADEGPVQSPSPHELQVLSP | |
| LA | LSPLSPEPPGAPVPQPLQPGSLRSPPDSEAYYGETDSDADGPATQEKPRRPRRR | |
| protein | GPTRPTPPGAPPDEVYLSDSPAEPAPTIPGPPSQGDSRVSSPSWEDGAALQPPPA | |
| | EALLLPHGPLRPGPHLIPMVGPVPHPVAEDLTTTYTQKAKQAKLQRAESLQEK | |
| | SIKEAKTKCRTIASLLTAAPNPHSKGVLMFKKRRQRAKKYTLVSFGAAAGTG | |
| | AEEEDGVPPTSESELDEEAFSDARSLTNQSDWDSPYLDMELARAGSRASEGQG | |
| | SGLGGQLSEVSGRGVQLFEQQRQRADSSTQELARVEPAAMLNGEGLQSPPRA | |
| | QSAPPEAAVLPPSPLPAPVASPRPFQPGGGAPTPAPSIFNRSARPFTPGLQGQRP | |
| | TTTSVIFRPLAPKRANDSLGGLSPAPPPFLSSQGPTPLPSFTSGVPSHAPVSGSPS | |
| | TPRSSGPVTATSSLYIPAPSRPVTPGGAPEPPAPPSAAAMTSTASIFLSAPLRPSA | |
| | RPEAPAPGPGAPEPPSAREQRISVPAARTGILQEARRRGTRKQMFRPGKEETKN | |
| | SPNPELLSLVQNLDEKPRAGGAESGPEEDALSLGAEACNFMQPVGARSYKTLP | |
| | HVTPKTPPPMAPKTPPPMTPKTPPPVAPKPPSRGLLDGLVNGAASSAGIPEPPR | |
| | LQGRGGELFAKRQSRADRYVVEGTPGPGLGPRPRSPSPTPSLPPSWKYSPNIRA | |
| | PPPIAYNPLLSPFFPQAARTLPKAQSQGPRATPKQGIKALDFMRHQPYQLKTA | |
| | MFCFDEVPPTPGPIASGSPKTARVQEIRRFSTPAPQPTAEPLAPTVLAPRAATTL | |
| | DEPIWRTELASAPVPSPAPPPEAPRGLGASPSSCGFQVARPRFSATRTGLQAHV | |
| | WRPGAGHQ | |
| Human | atggagacctttgagcccatcagccaagagcccctcagccaagccagctatgacaaagccccagacccagttcctgagctc | 357 |
| SYNPO2 | caagactcgttctatgcagaactgcaacgtgcagagagcctccaagagaagagcataaaagaggccaagaccaaatgca | |
| LB DNA | ggacaattgcatccctgctcactgcagcccccaacccccactccaaaggggtacttatgtttaagaaacggcggcagagag | |
| | ccaagaagtacaccctggtgagcttcggggctgctgctgggacaggcgctgaggaggaggacggcgttccccccacga | |
| | gtgagtccgagctggacgaagaagccttctctgacgcccgcagcctcaccaatcaatctgactgggacagtccctatctgga | |
| | catggagcttgccagggcgggctcaagagcatcagagggccagggctctgggctgggagggcagctgagtgaggtctct | |
| | gggcgaggggtgcagctctttgaacagcagcgccagcgcgcagactccagcacccaggaactggcacgggtcgaacca | |
| | gcagccatgctcaacggggaaggcctgcagtcaccacctcgggcccagagtgctcccccagaggcagctgtgctcccac | |
| | ccagccccttgccggcgcctgtagccagccccagacccttccaaccaggtggtggagccccgaccccagctccaagcatc | |
| | tttaaccggtcagccaggccctttaccccgggcctacaagggcagcggccaactaccacctcggttattttccggcctttag | |
| | cccccaaaagggcgaacgacagcctggggggcctcagccccgccccaccccccttcttgtcttcgcaggggcccacccct | |
| | ctgcccagcttcacttcaggggttcccagccacgcgccagtctctggttcccccagcaccccacgctcctcgggccctgtga | |
| | cagccaccagctccctgtacatcccagcccctagtcggcctgtcaccccaggtggagctccagagccccccgctcctccta | |
| | gcgcagctgccatgacctccaccgcttctatcttcctatctgcgcctttgcgaccctctgcgcgcccagaggcgcctgcccc | |
| | aggcccaggggctcctgagccccccagcgctcgcgagcagcgcatctctgtgccagctgcccgcacgggtatcctgcagg | |
| | aggcccggcgccgggggacccggaagcagatgttccggccgggaaaggaggagacgaagaactcgcccaaccccga | |
| | gctgctatcgctggtacagaacctggatgaaaagcctcgggccgggggtgcagaatctggtcctgaagaagatgctctgag | |
| | cctcggggctgaagcctgcaacttcatgcagccagtaggggccaggagttacaagaccctgcctcacgtgacacctaaga | |
| | ccccccctccaatggctcccaagaccccgccccctatgactcctaagactccacccccagtggctcctaagcccccatctcg | |
| | agggctccttgatgggctcgtgaatggggcagcctcttcggctggaatccctgagccaccaaggctgcagggcaggggtg | |
| | gggagctgtttgctaagcggcagagccgtgcggacaggtatgtggtggaaggtacacctggtcctggtcttggccctcggc | |
| | ctagaagtccttctcctaccccgtctctgcccccttcctggaaatattcacccaacatccgtgccccgcctcctattgctta | |
| | caacccactgctctctccctttttcccccaggcggcccgaactctccctaaggcccaatcccaggggcctcgggcaacacc | |
| | caagcagggcatcaaggctctagattttatgcggcatcagccctatcaacttaaaactgccatgttctgttttgatgaggtt | |
| | cccccgactcctggccctatcgcctcagggtcccccaaaactgcccgagtccaggagattcgccggttttccactccggca | |
| | ccccagcccactgcagaacccctggctcccactgtgcttgccccccgagcagccactacactggatgagcccatctggagaa | |
| | cagaactggcctcagcccctgttcctagcccagcccctcctccagaggctcccaggggccttggggcttctcccagctcct | |
| | gcggtttccaggtagccaggccccgattttcagccaccagaacaggattgcaagctcatgtgtggaggcctggggcagggc | |
| | accag | |
| Human | METFEPISQEPLSQASYDKAPDPVPELQDSFYAELQRAESLQEKSIKEAKTKCR | 358 |
| SYNPO2 | TIASLLTAAPNPHSKGVLMFKKRRQRAKKYTLVSFGAAAGTGAEEEDGVPPTS | |
| LB | ESELDEEAFSDARSLTNQSDWDSPYLDMELARAGSRASEGQGSGLGGQLSEV | |
| protein | SGRGVQLFEQQRQRADSSTQELARVEPAAMLNGEGLQSPPRAQSAPPEAAVL | |
| | PPSPLPAPVASPRPFQPGGGAPTPAPSIFNRSARPFTPGLQGQRPTTTSVIFRPLA | |
| | PKRANDSLGGLSPAPPPFLSSQGPTPLPSFTSGVPSHAPVSGSPSTPRSSGPVTAT | |
| | SSLYIPAPSRPVTPGGAPEPPAPPSAAAMTSTASIFLSAPLRPSARPEAPAPGPGA | |
| | PEPPSAREQRISVPAARTGILQEARRRGTRKQMFRPGKEETKNSPNPELLSLVQ | |
| | NLDEKPRAGGAESGPEEDALSLGAEACNFMQPVGARSYKTLPHVTPKTPPPM | |
| | APKTPPPMTPKTPPPVAPKPPSRGLLDGLVNGAASSAGIPEPPRLQGRGGELFA | |
| | KRQSRADRYVVEGTPGPGLGPRPRSPSPTPSLPPSWKYSPNIRAPPPIAYNPLLS | |
| | PFFPQAARTLPKAQSQGPRATPKQGIKALDFMRHQPYQLKTAMFCFDEVPPTP | |
| | GPIASGSPKTARVQEIRRFSTPAPQPTAEPLAPTVLAPRAATTLDEPIWRTELAS | |
| | APVPSPAPPPEAPRGLGASPSSCGFQVARPRFSATRTGLQAHVWRPGAGHQ | |
| Human | agagcgagcgccggggccgggcgcgcaggagtgaaaaggaggcggcggccgcagctgcgagcaacagatccggac | 359 |
| MTSS1 | gccgcgagctgacccgctctgctgttgggcgatttttttttaattgcagaaaaatttattaaattggaaaatcttgcgttt | |
| mRNA | ttcaatggcgctggccccgggtcagcgggcgattttctctgcatcaagatgggctttgccgtttccgtagtgggcaccagt | |
| | ggtggcctgattgtcagtcttctcccggcatttttaaggccaggagccgagcgctgcttgtaggcgaataccctacagagc | |
| | ggtttggctttttaaattactgttattattttgggcagagaacagtcggtctggtgcaccccgtcctcgctgcagaagaggc | |
| | tgcgagtccgaggtgggtctctcggaaggtgaaattccttctggggtgagcgagccccggccccgcgcgcagtccagcggcc | |
| | ccgcgtgtgtgccctcgccctgccggagccgggaaaatggaggctgtgattgagaaggaatgcagcgcgctcggaggcctct | |
| | tccagaccatcatcagcgacatgaaggggagctatccagtttgggaagatttcataaacaaagcaggaaagctgcagtccca | |
| | gcttcggacaacagtagtagcagcagctgccttcttggacgcctttcagaaagtggctgacatggccaccaacacacgtggt | |
| | gggaccagggagattggatctgctctcaccaggatgtgcatgaggcacagaagcattgaagccaagctgaggcagttttcg | |
| | agcgctttaattgattgtctgataaacccacttcaagaacagatggaagaatggaagaaagtggccaaccagctggataaa | |
| | gaccacgcaaaagaatataagaaagcccgccaagagataaaaaagaagtcctcggatacgctgaaactgcagaagaaagca | |
| | aaaaaagggagaggtgatatccagcctcagttggacagtgctctccaagatgtcaatgataagtatctcttattggaagaaa | |
| | aagcaggctgtccggaaggctttgattgaagaacgtggccgattctgtaccttcatctctatgctgcggccagtgattgaa | |
| | gacagaaagaaatctcaatgctaggggaaataacccaccttcagaccatctcggaagatctaaaaagcctgaccatggaccc | |
| | tcacaaactgccctcctcaagtgaacaggtgattctggacttgaaaggttctgattacagctggtcgtatcagacgccacc | |
| | ctcttcccccagcaccaccatgtccagaaagtccagtgtctgcagcagcctgaacagtgtcaacagcagtgactcccggtc | |
| | cagcggctcccactcgcattcccccagctcacattaccgctaccgcagctccaacctggcccagcaggctcctgtgaggctg | |
| | tccagcgtgtcctcccatgactcaggattcatatcccaggatgccttccagtccaagtcaccatcccccatgccgccagagg | |
| | cccccaaccagttgtctaacgggttttctcactatagtttatcaagtgagtcccacgtggggcccacgggtgcaggccttt | |
| | tccctcattgcctgcctgcctcccgcctgctccctcgggtcacctctgtccaccttccagactacgctcattattacacca | |
| | ttgggcccggcatgttcccgtcatctcagatccctagctggaaggactgggctaagcctgggccctatgaccagcctctgg | |
| | tgaacaccctgcagcgccgcaaagagaagcgagaaccggaccccaacgggggaggacccactaccgccagcggcccacctgc | |
| | agcagctgaggaggctcagagaccacggagcatgactgtatcggctgccaccaggcctggtgaggagatggaggcttgtgag | |
| | gagctggccctggccctgtctcggggcctgcagctggacacccagaggagcagccgggactcgcttcagtgctccagcggct | |
| | acagcacccagacaaccaccccctgctgctctgaggacaccatcccttcccaagtttcagattatgattatttctctgtaa | |
| | gtggtgaccaggaggcagatcagcaggagttcgacaagtcctccaccattccaagaaacagcgacatcagccagtcctaccg | |
| | acggatgttccaagccaagcgtccagcctcaactgctggcctccccaccaccctgggacctgctatggtcactccaggggtt | |
| | gcaactatccgacggaccccttccaccaagccttctgtccgccggggaaccattggagctggtcccatccccatcaagacac | |
| | ccgtgatccctgtcaagaccccaaccgtcccagacctcccaggggtgttgccagcccctccagatgggccagaagagcgggg | |
| | ggagcacagccctgagtcgccatctgtgggtgagggcccccaaggtgtcaccagcatgccctcctcaatgtggagcggccaa | |
| | gcttccgttaaccctccacttccaggcccgaagcccagtatccctgaggagcacagacaggcaattccagaaagtgaagctg | |
| | aagaccaggaacgggaacccccaagtgccactgtctccccaggccagattccagagagtgaccctgcagacctgagccca | |
| | agggatactccacaaggagaagacatgctgaacgccatccgaaggggcgtgaaactgaagaagaccacgacaaacgat | |
| | cgctcagcccctcgcttttcttaggttcacaagaaatgcgccggtggggaatgaactgtttcattaataaaacctaatttgt | |
| | cttgatccattccactctataataaaacaaaagattttgtaggcaactcggaatatagctcttttgaaagtactcgacacct | |
| | ttagataagaattaaaaccaacctatgtaactgacataatcttgatcttttaatttgtaaatattgacaattttctttctgc | |
| | acattttaatcttagtttcccttttgatttttctgaaggtgccaaattccatttaacttttttacaagtctttgtaaaattt | |
| | taaatgcataaagggggttggggcaggggaaccacgaagtagttaattttagaaaaggatttactatacttcactcttcttt | |
| | ttttttccccacaagcttttgtagatgcattgtagtagtctagcttagaagcaaatgcaagttattttaatgtacaaactaa | |
| | atgggtaagaggtaaaatcttcatttaaatatactatgttctggatgaaaagagcaggagtaacaattgatgagcaatattc | |
| | agagtgaagtaaatctggaaatggtagactgtgttgggattggggggagggccatgggaggggtacatcgtcaacatagcc | |
| | gatcctgttacatttaagagtagcctcgtaggttgaatttcttctggtagcttcatggtaaatgcatccgaataagccata | |
| | cctggattgcagtgtttgtttctgtagggtgtttaaggacttgacttctttctcccatgattcctctggactgcacacagc | |
| | acccacaaccagccccatgcatgctgctgcctctgggcagtcgtagaatctcccacttcagtttctcgttgattgtactca | |
| | cctttatggaatccaaatacatccaaaagggtaaggcagttttaaaaatgtgaaaacatttaaaaatgataatagcaggga | |
| | attcttagattatagtaaatgccttttacttaactgtgcccagcaggctgggtgcgttaaaaagcccaagtattttgaaaaa | |
| | actcgaacagatttgacaagggtagccagcttggagtctagcaacttgccaatgtgtttaccaatctgggggcttgttttt | |
| | cttttcttctttcaaataaatggcagttaactggctttacagtaaacattgaagagaggaggatttgtttattgtcactgg | |
| | gaatctgaccactatactgtcctttttttgtattctgggtaaatgttttttggaaaagatttgtcttttctaagtggaagt | |
| | taaatttgttatactgcccatcccctaaagccaacagagatttgtagatttaaagggatcacatttgaagacaatagtgtt | |
| | taagaaagcaagcaagtcccttagcagtcaggtcataacagggcacatttctgaccgaaccctctcaaggcagaggaggag | |
| | tttggtgggtttcatacaccctgcagattcctgttggctctaaccctcaattacctaatcttatgctttaacacataactg | |
| | cattggatgtgagagtaacgtaccgtatggtcattgttctatatattaacattgaacactgctgcgattgctcaaggacat | |
| | tttatgttacggctttaaagcaaaggcatgattattagaaactatttaagcttttttctttgaaaaacaagctccttttac | |
| | agaatataaacaacagtagtgcctgtggtttagcccaccaatcttgatgactaaaagtagctgatgcattgtgcatatgatg | |
| | cttgagatggtttttgcaaaagcagaaatcgctgcaaggtaatcacaatagataaaagtggtattttaaacctttgaaataa | |
| | atggatgtaactgtaccttggtacagcttttcacttgtttagtttttaaacgttagtataatctgaataaataaaatgttg | |
| | ccaaattcaatgtagaaagaatgtgacaacacaccttgggtagttctgcttgtgtttttgcatattgtaaaagcagtgtca | |
| | cagctaaaaagaaagaaatcgtttctaacagtaaattattgtgctttagttgctagtttgtactgagagttgacctctccc | |
| | tgtgcagttttttgttctaaacttgtataaataacaattgtgtaatgtgtctccctcctacattgtaacaattgcttcagc | |
| | ctacgttataaataaagaaccactagattaaaaaa | |
| saCas9 | atggccccaaagaagaagcggaaggtcggtatccacggagtcccagcagccaagcggaactacatcctgggcctggaca | 360 |
| DNA | tcggcatcaccagcgtgggctacggcatcatcgactacgagacacgggacgtgatcgatgccggcgtgcggctgttcaaa | |
| | gaggccaacgtggaaaacaacgagggcaggcggagcaagagaggcgccagaaggctgaagcggcggaggcggcat | |
| | agaatccagagagtgaagaagctgctgttcgactacaacctgctgaccgaccacagcgagctgagcggcatcaaccccta | |
| | cgaggccagagtgaagggcctgagccagaagctgagcgaggaagagttctctgccgccctgctgcacctggccaagag | |
| | aagaggcgtgcacaacgtgaacgaggtggaagaggacaccggcaacgagctgtccaccaaagagcagatcagccgga | |
| | acagcaaggccctggaagagaaatacgtggccgaactgcagctggaacggctgaagaaagacggcgaagtgcggggc | |
| | agcatcaacagattcaagaccagcgactacgtgaaagaagccaaacagctgctgaaggtgcagaaggcctaccaccagct | |
| | ggaccagagcttcatcgacacctacatcgacctgctggaaacccggcggacctactatgagggacctggcgagggcagc | |
| | cccttcggctggaaggacatcaaagaatggtacgagatgctgatgggccactgcacctacttccccgaggaactgcggag | |
| | cgtgaagtacgcctacaacgccgacctgtacaacgccctgaacgacctgaacaatctcgtgatcaccagggacgagaacg | |
| | agaagctggaatattacgagaagttccagatcatcgagaacgtgttcaagcagaagaagaagcccaccctgaagcagatc | |
| | gccaaagaaatcctcgtgaacgaagaggatattaagggctacagagtgaccagcaccggcaagcccgagttcaccaacct | |
| | gaaggtgtaccacgacatcaaggacattaccgcccggaaagagattattgagaacgccgagctgctggatcagattgccaa | |
| | gatcctgaccatctaccagagcagcgaggacatccaggaagaactgaccaatctgaactccgagctgacccaggaagag | |
| | atcgagcagatctctaatctgaagggctataccggcacccacaacctgagcctgaaggccatcaacctgatcctggacgag | |
| | ctgtggcacaccaacgacaaccagatcgctatcttcaaccggctgaagctggtgcccaagaaggtggacctgtcccagca | |
| | gaaagagatccccaccaccctggtggacgacttcatcctgagccccgtcgtgaagagaagcttcatccagagcatcaaagt | |
| | gatcaacgccatcatcaagaagtacggcctgcccaacgacatcattatcgagctggcccgcgagaagaactccaaggacg | |
| | cccagaaaatgatcaacgagatgcagaagcggaaccggcagaccaacgagcggatcgaggaaatcatccggaccaccg | |
| | gcaaagagaacgccaagtacctgatcgagaagatcaagctgcacgacatgcaggaaggcaagtgcctgtacagcctgga | |
| | agccatccctctggaagatctgctgaacaaccccttcaactatgaggtggaccacatcatccccagaagcgtgtccttcgac | |
| | aacagcttcaacaacaaggtgctcgtgaagcaggaagaaaacagcaagaagggcaaccggaccccattccagtacctgag | |
| | cagcagcgacagcaagatcagctacgaaaccttcaagaagcacatcctgaatctggccaagggcaagggcagaatcagc | |
| | aagaccaagaaagagtatctgctggaagaacgggacatcaacaggttctccgtgcagaaagacttcatcaaccggaacctg | |
| | gtggataccagatacgccaccagaggcctgatgaacctgctgcggagctacttcagagtgaacaacctggacgtgaaagt | |
| | gaagtccatcaatggcggcttcaccagctttctgcggcggaagtggaagtttaagaaagagcggaacaaggggtacaagc | |
| | accacgccgaggacgccctgatcattgccaacgccgatttcatcttcaaagagtggaagaaactggacaaggccaaaaaa | |
| | gtgatggaaaaccagatgttcgaggaaaagcaggccgagagcatgcccgagatcgaaaccgagcaggagtacaaagag | |
| | atcttcatcaccccccaccagatcaagcacattaaggacttcaaggactacaagtacagccaccgggtggacaagaagcct | |
| | aatagagagctgattaacgacaccctgtactccacccggaaggacgacaagggcaacaccctgatcgtgaacaatctgaa | |
| | cggcctgtacgacaaggacaatgacaagctgaaaaagctgatcaacaagagccccgaaaagctgctgatgtaccaccacg | |
| | acccccagacctaccagaaactgaagctgattatggaacagtacggcgacgagaagaatcccctgtacaagtactacgag | |
| | gaaaccgggaactacctgaccaagtactccaaaaaggacaacggccccgtgatcaagaagattaagtattacggcaacaa | |
| | actgaacgcccatctggacatcaccgacgactaccccaacagcagaaacaaggtcgtgaagctgtccctgaagccctaca | |
| | gattcgacgtgtacctggacaatggcgtgtacaagttcgtgaccgtgaagaatctggatgtgatcaaaaaagaaaactacta | |
| | cgaagtgaatagcaagtgctatgaggaagctaagaagctgaagaagatcagcaaccaggccgagtttatcgcctccttctac | |
| | aacaacgatctgatcaagatcaacggcgagctgtatagagtgatcggcgtgaacaacgacctgctgaaccggatcgaagtga | |
| | acatgatcgacatcacctaccgcgagtacctggaaaacatgaacgacaagaggccccccaggatcattaagacaatcgcct | |
| | ccaagacccagagcattaagaagtacagcacagacattctgggcaacctgtatgaagtgaaatctaagaagcaccctcaga | |
| | tcatcaaaaagggcaaaaggccggcggccacgaaaaaggccggccaggcaaaaaagaaaaagtaa | |
| saCas9 | MAPKKKRKVGIHGVPAAKRNYILGLDIGITSVGYGIIDYETRDVIDAGVRLFKE | 361 |
| protein | ANVENNEGRRSKRGARRLKRRRRHRIQRVKKLLFDYNLLTDHSELSGINPYEA | |
| | RVKGLSQKLSEEEFSAALLHLAKRRGVHNVNEVEEDTGNELSTKEQISRNSKA | |
| | LEEKYVAELQLERLKKDGEVRGSINRFKTSDYVKEAKQLLKVQKAYHQLDQS | |
| | FIDTYIDLLETRRTYYEGPGEGSPFGWKDIKEWYEMLMGHCTYFPEELRSVKY | |
| | AYNADLYNALNDLNNLVITRDENEKLEYYEKFQIIENVFKQKKKPTLKQIAKE | |
| | ILVNEEDIKGYRVTSTGKPEFTNLKVYHDIKDITARKEIIENAELLDQIAKILTIY | |
| | QSSEDIQEELTNLNSELTQEEIEQISNLKGYTGTHNLSLKAINLILDELWHTNDN | |
| | QIAIFNRLKLVPKKVDLSQQKEIPTTLVDDFILSPVVKRSFIQSIKVINAIIKKYGL | |
| | PNDIIIELAREKNSKDAQKMINEMQKRNRQTNERIEEIIRTTGKENAKYLIEKIK | |
| | LHDMQEGKCLYSLEAIPLEDLLNNPFNYEVDHIIPRSVSFDNSFNNKVLVKQEE | |
| | NSKKGNRTPFQYLSSSDSKISYETFKKHILNLAKGKGRISKTKKEYLLEERDIN | |
| | RFSVQKDFINRNLVDTRYATRGLMNLLRSYFRVNNLDVKVKSINGGFTSFLRR | |
| | KWKFKKERNKGYKHHAEDALIIANADFIFKEWKKLDKAKKVMENQMFEEKQ | |
| | AESMPEIETEQEYKEIFITPHQIKHIKDFKDYKYSHRVDKKPNRELINDTLYSTR | |
| | KDDKGNTLIVNNLNGLYDKDNDKLKKLINKSPEKLLMYHHDPQTYQKLKLIM | |
| | EQYGDEKNPLYKYYEETGNYLTKYSKKDNGPVIKKIKYYGNKLNAHLDITDD | |
| | YPNSRNKVVKLSLKPYRFDVYLDNGVYKFVTVKNLDVIKKENYYEVNSKCY | |
| | EEAKKLKKISNQAEFIASFYNNDLIKINGELYRVIGVNNDLLNRIEVNMIDITYR | |
| | EYLENMNDKRPPRIIKTIASKTQSIKKYSTDILGNLYEVKSKKHPQIIKKGKRPA | |
| | ATKKAGQAKKKK | |
| Split | GACAAGAAGTACAGCATCGGCCTGGACATCGGCACCAACTCTGTGGGCTG | 362 |
| spCas9 | GGCCGTGATCACCGACGAGTACAAGGTGCCCAGCAAGAAATTCAAGGTGC | |
| H840A | TGGGCAACACCGACCGGCACAGCATCAAGAAGAACCTGATCGGAGCCCTG | |
| N- | CTGTTCGACAGCGGCGAAACAGCCGAGGCCACCCGGCTGAAGAGAACCGC | |
| term- | CAGAAGAAGATACACCAGACGGAAGAACCGGATCTGCTATCTGCAAGAGA | |
| inal | TCTTCAGCAACGAGATGGCCAAGGTGGACGACAGCTTCTTCCACAGACTG | |
| frag- | GAAGAGTCCTTCCTGGTGGAAGAGGATAAGAAGCACGAGCGGCACCCCAT | |
| ment | CTTCGGCAACATCGTGGACGAGGTGGCCTACCACGAGAAGTACCCCACCA | |
| DNA | TCTACCACCTGAGAAAGAAACTGGTGGACAGCACCGACAAGGCCGACCTG | |
| | CGGCTGATCTATCTGGCCCTGGCCCACATGATCAAGTTCCGGGGCCACTTC | |
| | CTGATCGAGGGCGACCTGAACCCCGACAACAGCGACGTGGACAAGCTGTT | |
| | CATCCAGCTGGTGCAGACCTACAACCAGCTGTTCGAGGAAAACCCCATCA | |
| | ACGCCAGCGGCGTGGACGCCAAGGCCATCCTGTCTGCCAGACTGAGCAAG | |
| | AGCAGAAAGCTGGAAAATCTGATCGCCCAGCTGCCCGGCGAGAAGAAGAA | |
| | TGGCCTGTTCGGAAACCTGATTGCCCTGAGCCTGGGCCTGACCCCCAACTT | |
| | CAAGAGCAACTTCGACCTGGCCGAGGATGCCAAACTGCAGCTGAGCAAGG | |
| | ACACCTACGACGACGACCTGGACAACCTGCTGGCCCAGATCGGCGACCAG | |
| | TACGCCGACCTGTTTCTGGCCGCCAAGAACCTGTCCGACGCCATCCTGCTG | |
| | AGCGACATCCTGAGAGTGAACACCGAGATCACCAAGGCCCCCCTGAGCGC | |
| | CTCTATGATCAAGAGATACGACGAGCACCACCAGGACCTGACCCTGCTGA | |
| | AAGCTCTCGTGCGGCAGCAGCTGCCTGAGAAGTACAAAGAGATTTTCTTCG | |
| | ACCAGAGCAAGAACGGCTACGCCGGCTACATTGACGGCGGAGCCAGCCAG | |
| | GAAGAGTTCTACAAGTTCATCAAGCCCATCCTGGAAAAGATGGACGGCAC | |
| | CGAGGAACTGCTCGTGAAGCTGAAGAGAGAGGACCTGCTGCGGAAGCAGC | |
| | GGACCTTCGACAACGGCAGCATCCCCCACCAGATCCACCTGGGAGAGCTG | |
| | CACGCCATTCTGCGGCGGCAGGAAGATTTTTACCCATTCCTGAAGGACAAC | |
| | CGGGAAAAGATCGAGAAGATCCTGACCTTCCGCATCCCCTACTACGTGGG | |
| | CCCTCTGGCCAGGGGAAACAGCAGATTCGCCTGGATGACCAGAAAGAGCG | |
| | AGGAAACCATCACCCCCTGGAACTTCGAGGAAGTGGTGGACAAGGGCGCT | |
| | TCCGCCCAGAGCTTCATCGAGCGGATGACCAACTTCGATAAGAACCTGCCC | |
| | AACGAGAAGGTGCTGCCCAAGCACAGCCTGCTGTACGAGTACTTCACCGT | |
| | GTATAACGAGCTGACCAAAGTGAAATACGTGACCGAGGGAATGAGAAAGC | |
| | CCGCCTTCCTGAGCGGCGAGCAGAAAAAGGCCATCGTGGACCTGCTGTTC | |
| | AAGACCAACCGGAAAGTGACCGTGAAGCAGCTGAAAGAGGACTACTTCAA | |
| | GAAAATCGAGTGCTTCGACTCCGTGGAAATCTCCGGCGTGGAAGATCGGTT | |
| | CAACGCCTCCCTGGGCACATACCACGATCTGCTGAAAATTATCAAGGACA | |
| | AGGACTTCCTGGACAATGAGGAAAACGAGGACATTCTGGAAGATATCGTG | |
| | CTGACCCTGACACTGTTTGAGGACAGAGAGATGATCGAGGAACGGCTGAA | |
| | AACCTATGCCCACCTGTTCGACGACAAAGTGATGAAGCAGCTGAAGCGGC | |
| | GGAGATACACCGGCTGGGGCAGGCTGAGCCGGAAGCTGATCAACGGCATC | |
| | CGGGACAAGCAGTCCGGCAAGACAATCCTGGATTTCCTGAAGTCCGACGG | |
| | CTTCGCCAACAGAAACTTCATGCAGCTGATCCACGACGACAGCCTGACCTT | |
| | TAAAGAGGACATCCAGAAAGCCCAGGTGTCCGGCCAGGGCGATAGCCTGC | |
| | ACGAGCACATTGCCAATCTGGCCGGCAGCCCCGCCATTAAGAAGGGCATC | |
| | CTGCAGACAGTGAAGGTGGTGGACGAGCTCGTGAAAGTGATGGGCCGGCA | |
| | CAAGCCCGAGAACATCGTGATCGAAATGGCCAGAGAGAACCAGACCACCC | |
| | AGAAGGGACAGAAGAACAGCCGCGAGAGAATGAAGCGGATCGAAGAGGG | |
| | CATCAAAGAGCTGGGCAGCCAGATCCTGAAAGAACACCCCGTGGAAAACA | |
| | CCCAGCTGCAGAACGAGAAGCTGTACCTGTACTACCTGCAGAATGGGCGG | |
| | GATATGTACGTGGACCAGGAACTGGACATCAACCGGCTGTCCGACTACGA | |
| | TGTGGACGCTATCGTGCCTCAGAGCTTTCTGAAGGACGACTCCATCGACAA | |
| | CAAGGTGCTGACCAGAAGCGACAAGAACCGGGGCAAGAGCGACAACGTG | |
| | CCCTCCGAAGAGGTCGTGAAGAAGATGAAGAACTACTGGCGGCAGCTGCT | |
| | GAACGCCAAGCTGATTACCCAGAGAAAGTTCGACAATCTGACCAAGGCCG | |
| | AGAGAGGCGGCCTGAGCGAACTGGATAAGGCCGGCTTCATCAAGAGACAG | |
| | CTGGTGGAAACCCGGCAGATCACAAAGCACGTGGCACAGATCCTGGACTC | |
| | CCGGATGAACACTAAGTACGACGAGAATGACAAGCTGATCCGGGAAGTGA | |
| | AAGTGATCACCCTGAAGTCCAAGCTGGTGTCCGATTTCCGGAAGGATTTCC | |
| | AGTTTTACAAAGTGCGCGAGATCAACAACTACCACCACGCCCACGACGCC | |
| | TACCTGAACGCCGTCGTGGGAACCGCCCTGATCAAAAAGTACCCTAAGCT | |
| | GGAAAGCGAGTTCGTGTACGGCGACTACAAGGTGTACGACGTGCGGAAGA | |
| | TGATCGCCAAG | |
| Split | DKKYSIGLDIGTNSVGWAVITDEYKVPSKKFKVLGNTDRHSIKKNLIGALLFDS | 363 |
| spCas9 | GETAEATRLKRTARRRYTRRKNRICYLQEIFSNEMAKVDDSFFHRLEESFLVEE | |
| H840A | DKKHERHPIFGNIVDEVAYHEKYPTIYHLRKKLVDSTDKADLRLIYLALAHMI | |
| N- | KFRGHFLIEGDLNPDNSDVDKLFIQLVQTYNQLFEENPINASGVDAKAILSARL | |
| term- | SKSRKLENLIAQLPGEKKNGLFGNLIALSLGLTPNFKSNFDLAEDAKLQLSKDT | |
| inal | YDDDLDNLLAQIGDQYADLFLAAKNLSDAILLSDILRVNTEITKAPLSASMIKR | |
| frag- | YDEHHQDLTLLKALVRQQLPEKYKEIFFDQSKNGYAGYIDGGASQEEFYKFIK | |
| ment | PILEKMDGTEELLVKLKREDLLRKQRTFDNGSIPHQIHLGELHAILRRQEDFYP | |
| protein | FLKDNREKIEKILTFRIPYYVGPLARGNSRFAWMTRKSEETITPWNFEEVVDKG | |
| | ASAQSFIERMTNFDKNLPNEKVLPKHSLLYEYFTVYNELTKVKYVTEGMRKP | |
| | AFLSGEQKKAIVDLLFKTNRKVTVKQLKEDYFKKIECFDSVEISGVEDRFNASL | |
| | GTYHDLLKIIKDKDFLDNEENEDILEDIVLTLTLFEDREMIEERLKTYAHLFDD | |
| | KVMKQLKRRRYTGWGRLSRKLINGIRDKQSGKTILDFLKSDGFANRNFMQLI | |
| | HDDSLTFKEDIQKAQVSGQGDSLHEHIANLAGSPAIKKGILQTVKVVDELVKV | |
| | MGRHKPENIVIEMARENQTTQKGQKNSRERMKRIEEGIKELGSQILKEHPVEN | |
| | TQLQNEKLYLYYLQNGRDMYVDQELDINRLSDYDVDAIVPQSFLKDDSIDNK | |
| | VLTRSDKNRGKSDNVPSEEVVKKMKNYWRQLLNAKLITQRKFDNLTKAERG | |
| | GLSELDKAGFIKRQLVETRQITKHVAQILDSRMNTKYDENDKLIREVKVITLKS | |
| | KLVSDFRKDFQFYKVREINNYHHAHDAYLNAVVGTALIKKYPKLESEFVYGD | |
| | YKVYDVRKMIAK | |
| Split | GAAATCGGCAAGGCTACCGCCAAGTACTTCTTCTACAGCAACATCATGAAC | 364 |
| spCas9 | TTTTTCAAGACCGAGATTACCCTGGCCAACGGCGAGATCCGGAAGCGGCCT | |
| H840A | CTGATCGAGACAAACGGCGAAACCGGGGAGATCGTGTGGGATAAGGGCCG | |
| C- | GGATTTTGCCACCGTGCGGAAAGTGCTGAGCATGCCCCAAGTGAATATCGT | |
| term- | GAAAAAGACCGAGGTGCAGACAGGCGGCTTCAGCAAAGAGTCTATCCTGC | |
| inal | CCAAGAGGAACAGCGATAAGCTGATCGCCAGAAAGAAGGACTGGGACCCT | |
| frag- | AAGAAGTACGGCGGCTTCGACAGCCCCACCGTGGCCTATTCTGTGCTGGTG | |
| ment | GTGGCCAAAGTGGAAAAGGGCAAGTCCAAGAAACTGAAGAGTGTGAAAG | |
| DNA | AGCTGCTGGGGATCACCATCATGGAAAGAAGCAGCTTCGAGAAGAATCCC | |
| | ATCGACTTTCTGGAAGCCAAGGGCTACAAAGAAGTGAAAAAGGACCTGAT | |
| | CATCAAGCTGCCTAAGTACTCCCTGTTCGAGCTGGAAAACGGCCGGAAGA | |
| | GAATGCTGGCCTCTGCCGGCGAACTGCAGAAGGGAAACGAACTGGCCCTG | |
| | CCCTCCAAATATGTGAACTTCCTGTACCTGGCCAGCCACTATGAGAAGCTG | |
| | AAGGGCTCCCCCGAGGATAATGAGCAGAAACAGCTGTTTGTGGAACAGCA | |
| | CAAGCACTACCTGGACGAGATCATCGAGCAGATCAGCGAGTTCTCCAAGA | |
| | GAGTGATCCTGGCCGACGCTAATCTGGACAAAGTGCTGTCCGCCTACAACA | |
| | AGCACCGGGATAAGCCCATCAGAGAGCAGGCCGAGAATATCATCCACCTG | |
| | TTTACCCTGACCAATCTGGGAGCCCCTGCCGCCTTCAAGTACTTTGACACC | |
| | ACCATCGACCGGAAGAGGTACACCAGCACCAAAGAGGTGCTGGACGCCAC | |
| | CCTGATCCACCAGAGCATCACCGGCCTGTACGAGACACGGATCGACCTGTC | |
| | TCAGCTGGGAGGTGAC | |
| Split | EIGKATAKYFFYSNIMNFFKTEITLANGEIRKRPLIETNGETGEIVWDKGRDFAT | 365 |
| spCas9 | VRKVLSMPQVNIVKKTEVQTGGFSKESILPKRNSDKLIARKKDWDPKKYGGF | |
| H840A | DSPTVAYSVLVVAKVEKGKSKKLKSVKELLGITIMERSSFEKNPIDFLEAKGY | |
| C- | KEVKKDLIIKLPKYSLFELENGRKRMLASAGELQKGNELALPSKYVNFLYLAS | |
| term- | HYEKLKGSPEDNEQKQLFVEQHKHYLDEIIEQISEFSKRVILADANLDKVLSAY | |
| inal | NKHRDKPIREQAENIIHLFTLTNLGAPAAFKYFDTTIDRKRYTSTKEVLDATLIH | |
| frag- | QSITGLYETRIDLSQLGGD | |
| ment | | |
| protein | | |

In some embodiments, the transgene comprises a polynucleotide sequence that encodes one or more gene products selected from MYBPC3, KCNH2, TRPM4, DSG2, ATP2A2, CACNA1C, DMD, DMPK, EPG5, EVC, EVC2, FBN1, NF1, SCN5A, SOS1, NPR1, ERBB4, VIP, MYH6, MYH7, or a mutant, variant, or fragment thereof. In some embodiments, the transgene comprises a polynucleotide sequence that encodes one or more gene products selected from TGFBR2, TGFBR1, EMD, KCNQ1, TAZ, COL3A1, JUP, CASQ2, MLRP44, DNAJC19, LMNA, TNNI3, DSP, DSG2, RAF1, SOS1, FBN1, LAMP2, FXN, RAF1, BAG3, KCNQ1, MYLK3, CRYAB, ALPK3 and ACTN2. In some embodiments the transgene comprises a polynucleotide sequence that encodes one or more gene products selected from MYBPC3, DWORF, JPH2, BAG3, CRYAB, Lamin A isoform of LMNA, Lamin C isoform of LMNA, TNNI3, PLN, LAMP2a, LAMP2b, LAMP2c, DPI isoform of DSP, DPII isoform of DSP, DSG2, MYH6, MYH7, RBM20, and JUP.

In some embodiments, the transgene comprises a polynucleotide sequence that encodes one or more gene products selected from ASCL1, MYOCD, MEF2C, and TBX5. In some embodiments, the transgene comprises a polynucleotide sequence that encodes one or more gene products selected from ASCL1, MYOCD, MEF2C, AND TBX5, CCNB1, CCND1, CDK1, CDK4, AURKB, OCT4, BAF60C, ESRRG, GATA4, GATA6, HAND2, IRX4, ISLL, MESP1, MESP2, NKX2.5, SRF, TBX20, ZFPM2, and MIR-133.

In some embodiments, the transgene comprises a polynucleotide sequence that encodes one or more gene products selected from MYBPC3, DWORF, KCNH2, TRPM4, DSG2, and ATP2A2.

In some embodiments, the transgene comprises a polynucleotide sequence that encodes one or more gene products selected from TGFBR2, TGFBR1, EMD, KCNQ1, TAZ, COL3A1, JUP, CASQ2, MLRP44, DNAJC19, LMNA, TNNI3, DSP, DSG2, RAF1, SOS1, FBN1, LAMP2, FXN, RAF1, BAG3, KCNQ1, MYLK3, CRYAB, ALPK3 and ACTN2.

In some embodiments, the transgene comprises a polynucleotide sequence that encodes one or more gene products selected from CACNA1C, DMD, DMPK, EPG5, EVC, EVC2, FBN1, NF1, SCN5A, SOS1, NPR1, ERBB4, VIP, MYH6, MYH7, and split Cas9. In some embodiments, the transgene comprises a polynucleotide sequence that encodes saCas9. In some embodiments, the transgene comprises a first polynucleotide sequence that encodes an N-terminal spCas9 fragment polypeptide and a second polynucleotide sequence that encodes a C-terminal spCas9 fragment polypeptide.

In some embodiments, the transgene comprises a polynucleotide sequence that encodes one or more gene products selected from MYOCD, ASCL1, GATA4, MEF2C, TBX5, miR-133, and MESP1.

In some embodiments, the transgene comprises a polynucleotide sequence that encodes one or more gene products selected from MMP11, SYNPO2L (e.g., SYNPO2LA or SYNPO2LA), and an inhibitory oligonucleotide targeting MTSS1.

In some embodiments, the transgene comprises a polynucleotide sequence that encodes any of the above-identified gene products.

In some embodiments, the engineer capsid protein described herein improves heart transduction efficiency of any of the transgenes described herein (and encoding, and resulting in the expression of, any of the gene products described herein).

### Additional Description of Capsids, Transgenes, and Virions

Efforts to identify capsid variants with properties useful for gene therapy have included shuffling the DNA of AAV2 and AAV5 cap genes as described in U.S. Pat. No. 9,233,131; as well as directed evolution as described in Int'l Pat. Appl. Nos. WO2012/145601A2 and WO2018/222503A1. The disclosures of these documents are incorporated here for all purposes, and particularly for the methods of making and using AAV virions and for the polynucleotide sequences and gene products therein disclosed, as well as for the combinations of transcription factors useful in treating cardiac diseases or disorders.

The AAV capsid is encoded by the cap gene of AAV, which is also termed the right open-reading frame (ORF) (in contrast to the left ORF, rep). The structures of representative AAV capsids are described in various publications including Xie et al. (2002) Proc. Natl. Acad. Sci USA 99:10405-1040 (AAV2); Govindasamy et al. (2006) J. Virol. 80:11556-11570 (AAV4); Nam et a. (2007) J. Virol. 81:12260-12271 (AAV8) and Govindasamy et al. (2013) J. Virol. 87:11187-11199 (AAV5).

The AAV capsid contain 60 copies (in total) of three viral proteins (VPs), VP1, VP2, and VP3, in a predicted ratio of 1:1:10, arranged with T=1 icosahedral symmetry. The three VPs are encoded by the same gene, with VP1 containing a unique N-terminal domain in addition to the entire VP2 sequence at its C-terminal region. VP2 contains an extra N-terminal sequence in addition to VP3 at its C terminus. Cryo-electron microscopy and image reconstruction data suggest that in intact AAV capsids, the N-terminal regions of the VP1 and VP2 proteins are located inside the capsid and are inaccessible for receptor and antibody binding.

In some embodiments, each of the VP1, VP2 and/or VP3 capsid protein comprises the VR-VIII substitution(s) or substitution motif described herein, and optionally any of the additional modifications described herein. In some embodiments, each of the VP1, VP2 and VP3 capsid proteins comprise the VR-VIII substitution(s) or substitution motif described herein, and optionally any of the additional modifications described herein. In some embodiments, an rAAV described herein comprises VP1, VP2 and VP3 capsid proteins, wherein each of the VP1, VP2 and VP3 capsid proteins comprise the VR-VIII substitution(s) or substitution motif described herein, and optionally any of the additional modifications described herein. In some embodiments, an rAAV described herein comprises a mixture of wild-type and engineered VP1, VP2 and VP3 capsid proteins, wherein the engineered VP1, VP2 and/or VP3 capsid protein comprises the VR-VIII substitution(s) or substitution motif described herein, and optionally any of the additional modifications described herein.

For the GH loop/loop IV of AAV capsid, see, e.g., van Vliet et al. (2006) Mol. Ther. 14:809; Padron et al. (2005) Virol. 79:5047; and Shen et al. (2007) Mol. Ther. 15: 1955. In some embodiments, a “parental” AAV capsid protein is a wild-type AAV9 capsid protein. In some embodiments, a “parental” AAV capsid protein is a wild-type AAV5 capsid protein. In some embodiments, a “parental” AAV capsid protein is a wild-type AAVrh.10 capsid protein. In some embodiments, a “parental” AAV capsid protein is a wild-type AAVrh.74 capsid protein. In some embodiments, a “parental” AAV capsid protein is a chimeric AAV capsid protein. Amino acid sequences of various AAV capsid proteins are known in the art. See, e.g., GenBank Accession No. NP_049542 for AAV1; GenBank Accession No. NP_044927 for AAV4; GenBank Accession No. AAD13756 for AAV5; GenBank Accession No. AAB95450 for AAV6; GenBank Accession No. YP_077178 for AAV7; GenBank Accession No. YP_077180 for AAV 8; GenBank Accession No. AAS99264 for AAV9 and GenBank Accession No. AAT46337 for AAV10. See, e.g., Santiago-Ortiz et al. (2015) Gene Ther. 22:934 for a predicted ancestral AAV capsid.

Adeno-associated virus (AAV) is a replication-deficient parvovirus, the single-stranded DNA genome of which is about 4.7 kb in length including two 145 nucleotide inverted terminal repeat (ITRs). There are multiple serotypes of AAV. The nucleotide sequences of the genomes of the AAV serotypes are known. For example, the AAV5 genome is provided in GenBank Accession No. AF085716. The life cycle and genetics of AAV are reviewed in Muzyczka, Current Topics in Microbiology and Immunology, 158: 97-129 (1992). Production of pseudotyped rAAV is disclosed in, for example, WO 01/83692. Other types of rAAV variants, for example rAAV with capsid mutations, are also contemplated. See, for example, Marsic et al., Molecular Therapy, 22(11): 1900-1909 (2014). Illustrative AAV vectors are provided in U.S. Pat. No. 7,105,345; U.S. Ser. No. 15/782,980; U.S. Pat. Nos. 7,259,151; 6,962,815; 7,718,424; 6,984,517; 7,718,424; 6,156,303; 8,524,446; 7,790,449; 7,906,111; 9,737,618; U.S. application Ser. No. 15/433,322; U.S. Pat. No. 7,198,951, each of which is incorporated by reference in its entirety for all purposes.

The rAAV virions of the disclosure comprise a heterologous nucleic acid comprising a nucleotide sequence encoding one or more gene product. The gene product(s) may be either a polypeptide or an RNA, or both. When the gene product is a polypeptide, the nucleotide sequence encodes a messenger RNA, optionally with one or more introns, which is translated into the gene product polypeptide. The nucleotide sequence may encode one, two, three, or more gene products (though the number is limited by the packaging capacity of the rAAV virion, typically about 5.2 kb). The gene products may be operatively linked to one promoter (for a single transcriptional unit) or more than one. Multiple gene products may also be produced using internal ribosome entry signal (IRES) or a self-cleaving peptide (e.g., a 2A peptide).

In some embodiments, the gene product is a polypeptide. In some embodiments, the polypeptide gene product is a polypeptide that induces reprogramming of a cardiac fibroblast, to generate an induced cardiomyocyte-like cell (iCM). In some embodiments, the polypeptide gene product is a polypeptide that enhances the function of a cardiac cell. In some embodiments, the polypeptide gene product is a polypeptide that provides a function that is missing or defective in the cardiac cell. In some embodiments, the polypeptide gene product is a genome-editing endonuclease.

In some embodiments, the gene product comprises a fusion protein that is fused to a heterologous polypeptide. In some embodiments, the gene product comprises a genome editing nuclease fused to an amino acid sequence that provides for subcellular localization, i.e., the fusion partner is a subcellular localization sequence (e.g., one or more nuclear localization signals (NLSs) for targeting to the nucleus, two or more NLSs, three or more NLSs, etc.).

In general, a viral vector is produced by introducing a viral DNA or RNA construct into a “producer cell” or “packaging cell” line. Packaging cell lines include but are not limited to any easily-transfectable cell line. Packaging cell lines can be based on HEK291, 293T cells, NIH3T3, COS, HeLa or Sf9 cell lines. Examples of packaging cell lines include but are not limited to: Sf9 (ATCC® CRL-1711™). Exemplary packing cell lines and methods for generating rAAV virions are provided by Int'l Pat. Pub. Nos. WO2017075627, WO2015/031686, WO2013/063379, WO2011/020710, WO2009/104964, WO2008/024998, WO2003/042361, and WO1995/013392; U.S. Pat. Nos. 9,441,206B2, 8,679,837, and 7,091,029B2.

In some embodiments, the gene product is a functional cardiac protein. In some embodiments, the gene product is a genome-editing endonuclease (optionally with a guide RNA, single-guide RNA, and/or repair template) that replaces or repairs a non-functional cardiac protein into a functional cardiac protein. Functional cardiac proteins include, but are not limited to cardiac troponin T; a cardiac sarcomeric protein; β-myosin heavy chain; myosin ventricular essential light chain 1; myosin ventricular regulatory light chain 2; cardiac a-actin; a-tropomyosin; cardiac troponin I; cardiac myosin binding protein C; four-and-a-half LIM protein 1; titin; 5′-AMP-activated protein kinase subunit gamma-2; troponin I type 3, myosin light chain 2, actin alpha cardiac muscle 1; cardiac LIM protein; caveolin 3 (CAV3); galactosidase alpha (GLA); lysosomal-associated membrane protein 2 (LAMP2); mitochondrial transfer RNA glycine (MTTG); mitochondrial transfer RNA isoleucine (MTTI); mitochondrial transfer RNA lysine (MTTK); mitochondrial transfer RNA glutamine (MTTQ); myosin light chain 3 (MYL3); troponin C (TNNC1); transthyretin (TTR); sarcoendoplasmic reticulum calcium-ATPase 2a (SERCA2a); stromal-derived factor-1 (SDF-1); adenylate cyclase-6 (AC6); beta-ARKct (β-adrenergic receptor kinase C terminus); fibroblast growth factor (FGF); platelet-derived growth factor (PDGF); vascular endothelial growth factor (VEGF); hepatocyte growth factor; hypoxia inducible growth factor; thymosin beta 4 (TMSB4X); nitric oxide synthase-3 (NOS3); unocartin 3 (UCN3); melusin; apoplipoprotein-E (ApoE); superoxide dismutase (SOD); and S100A1 (a small calcium binding protein; see, e.g., Ritterhoff and Most (2012) Gene Ther. 19:613; Kraus et al. (2009) Mol. Cell. Cardiol. 47:445).

In some embodiments, the gene product is a gene product whose expression complements a defect in a gene responsible for a genetic disorder. The disclosure provides rAAV virions comprising a polynucleotide encoding one or more of the following—e.g., for use, without limitation, in the disorder indicated in parentheses, or for other disorders caused by each: TAZ (Barth syndrome); FXN (Freidrich's Ataxia); CASQ2 (CPVT); FBN1 (Marfan); RAF1 and SOSis (Noonan); SCN5A (Brugada); KCNQ1 and KCNH2s (Long QT Syndrome); DMPK (Myotonic Dystrophy 1); LMNA (Limb Girdle Dystrophy Type 1B); JUP (Naxos); TGFBR2 (Loeys-Dietz); EMD (X-Linked EDMD); and ELN (SV Aortic Stenosis). In some embodiments, the rAAV virion comprises a polynucleotide encoding one or more of cardiac troponin T (TNNT2); BAG family molecular chaperone regulator 3 (BAG3); myosin heavy chain (MYH7); tropomyosin 1 (TPM1); myosin binding protein C (MYBPC3); 5′-AMP-activated protein kinase subunit gamma-2 (PRKAG2); troponin I type 3 (TNNI3); titin (TTN); myosin, light chain 2 (MYL2); actin, alpha cardiac muscle 1 (ACTC1); potassium voltage-gated channel, KQT-like subfamily, member 1 (KCNQ1); myocyte enhancer factor 2c (MEF2C); and cardiac LIM protein (CSRP3).

In some embodiments, the gene products of the disclosure are polypeptide reprogramming factors. Reprogramming factors are desirable as means to convert one cell type into another. Non-cardiomyocytes cells can be differentiated into cardiomyocytes cells in vitro or in vivo using any method available to one of skill in the art. For example, see methods described in Ieda et al. (2010) Cell 142:375-386; Christoforou et al. (2013) PLoS ONE 8:e63577; Addis et al. (2013) J. Mol. Cell Cardiol. 60:97-106; Jayawardena et al. (2012) Circ. Res. 110: 1465-1473; Nam Y et al. (2003) PNAS USA 110:5588-5593; Wada R et al. (2013) PNAS USA 110: 12667-12672; and Fu J et al. (2013) Stem Cell Reports 1:235-247.

In cardiac context, the reprogramming factors may be capable of converting a cardiac fibroblast to a cardiac myocyte either directly or through an intermediate cell type. In particular, direct reprogramming is possible, or reprogramming by first converting the fibroblast to a pluripotent or totipotent stem cell. Such a pluripotent stem cell is termed an induced pluripotent stem (iPS) cell. An iPS cell that is subsequently converted to a cardiac myocyte (CM) cell is termed an iPS-CM cell. In the examples, iPS-CM derived in vitro from cardiac fibroblasts are used in vivo to select capsid proteins of interest. The disclosure also envisions using the capsid proteins disclosure to in turn generate iPS-CM cells in vitro but, particular, in vivo, as part of a therapeutic gene therapy regimen. Induced cardiomyocyte-like (iCM) cells refer to cells directly reprogrammed into cardiomyocytes.

Induced cardiomyocytes express one or more cardiomyocyte-specific markers, where cardiomyocyte-specific markers include, but are not limited to, cardiac troponin I, cardiac troponin-C, tropomyosin, caveolin-3, myosin heavy chain, myosin light chain-2a, myosin light chain-2v, ryanodine receptor, sarcomeric a-actinin, Nkx2.5, connexin 43, and atrial natriuretic factor. Induced cardiomyocytes can also exhibit sarcomeric structures. Induced cardiomyocytes exhibit increased expression of cardiomyocyte-specific genes ACTC1 (cardiac a-actin), ACTN2 (actinin a2), MYH6 (a-myosin heavy chain), RYR2 (ryanodine receptor 2), MYL2 (myosin regulatory light chain 2, ventricular isoform), MYL7 (myosin regulatory light chain, atrial isoform), TNNT2 (troponin T type 2, cardiac), and NPPA (natriuretic peptide precursor type A), PLN (phospholamban). Expression of fibroblasts markers such as Colla2 (collagen 1a2) is downregulated in induced cardiomyocytes, compared to fibroblasts from which the iCM is derived.

Reprogramming methods involving polypeptide reprogramming factors (in some cases supplemented by small-molecule reprogramming factors supplied in conjunction with the rAAV) include those described in US2018/0112282A1, WO2018/005546, WO2017/173137, US2016/0186141, US2016/0251624, US2014/0301991, and US2013/0216503A1, which are incorporated in their entirety, particularly for the reprogramming methods and factors disclosed.

In some embodiments, cardiac cells are reprogrammed into induced cardiomyocyte-like (iCM) cells using one or more reprogramming factors that modulate the expression of one or more polynucleotides or proteins of interest, such as Achaete-scute homolog 1 (ASCL1), Myocardin (MYOCD), myocyte-specific enhancer factor 2C (MEF2C), and/or T-box transcription factor 5 (TBX5). In some embodiments, the one or more reprogramming factors are provided as a polynucleotide (e.g., an RNA, an mRNA, or a DNA polynucleotide) that encode one or more polynucleotides or proteins of interest. In some embodiments, the one or more reprogramming factors are provided as a protein.

In some embodiments, the reprogramming factors are microRNAs or microRNA antagonists, siRNAs, or small molecules that are capable of increasing the expression of one or more polynucleotides or proteins of interest. In some embodiments, expression of a polynucleotides or proteins of interest is increased by expression of a microRNA or a microRNA antagonist. For example, endogenous expression of an Oct polypeptide can be increased by introduction of microRNA-302 (miR-302), or by increased expression of miR-302. See, e.g., Hu et al., Stem Cells 31(2): 259-68 (2013), which is incorporated herein by reference in its entirety. Hence, miRNA-302 can be an inducer of endogenous Oct polypeptide expression. The miRNA-302 can be introduced alone or with a nucleic acid that encodes the Oct polypeptide. In some embodiments, a suitable nucleic acid gene product is a microRNA. Suitable microRNAs include, e.g., mir-1, mir-133, mir-208, mir-143, mir-145, and mir-499.

In some embodiments, the methods of the disclosure comprise administering an rAAV virion of the disclosure before, during, or after administration of the small-molecule reprogramming factor. In some embodiments, the small-molecule reprogramming factor is a small molecule selected from the group consisting of SB431542, LDN-193189, dexamethasone, LY364947, D4476, myricetin, IWR1, XAV939, docosahexaenoic acid (DHA), S-Nitroso-TV-acetylpenicillamine (SNAP), Hh-Ag1.5, alprostadil, cromakalim, MNITMT, A769662, retinoic acid p-hydoxyanlide, decamethonium dibromide, nifedipine, piroxicam, bacitracin, aztreonam, harmalol hydrochloride, amide-C2 (A7), Ph-C12 (CIO), mCF3-C-7 (J5), G856-7272 (A473), 5475707, or any combination thereof.

In some embodiments, the gene products comprise reprogramming factors that modulate the expression of one or more proteins of interest selected from ASCL1, MYOCD, MEF2C, and TBX5. In some embodiments, the gene products comprise one or more reprogramming factors selected from ASCL1, MYOCD, MEF2C, AND TBX5, CCNB1, CCND1, CDK1, CDK4, AURKB, OCT4, BAF60C, ESRRG, GATA4, GATA6, HAND2, IRX4, ISLL, MESP1, MESP2, NKX2.5, SRF, TBX20, ZFPM2, and miR-133.

In some embodiments, the gene products comprise GATA4, MEF2C, and TBX5 (i.e., GMT). In some embodiments, the gene products comprise MYOCD, MEF2C, and TBX5 (i.e., MyMT). In some embodiments, the gene products comprise MYOCD, ASCL1, MEF2C, and TBX5 (i.e., MyAMT). In some embodiments, the gene products comprise MYOCD and ASCL1 (i.e., MyA). In some embodiments, the gene products comprise GATA4, MEF2C, TBX5, and MYOCD (i.e., 4F). In other embodiments, the gene products comprise GATA4, MEF2C, TBX5, ESSRG, MYOCD, ZFPM2, and MESP1 (i.e., 7F). In some embodiments, the gene products comprise one or more of ASCL1, MEF2C, GATA4, TBX5, MYOCD, ESRRG, AND MESPL.

In some embodiments, the rAAV virions generate cardiac myocytes in vitro or in vivo. Cardiomyocytes or cardiac myocytes are the muscle cells that make up the cardiac muscle. Each myocardial cell contains myofibrils, which are long chains of sarcomeres, the contractile units of muscle cells. Cardiomyocytes show striations similar to those on skeletal muscle cells, but unlike multinucleated skeletal cells, they contain only one nucleus. Cardiomyocytes have a high mitochondrial density, which allows them to produce ATP quickly, making them highly resistant to fatigue. Mature cardiomyocytes can express one or more of the following cardiac markers: α-Actinin, MLC2v, MY20, cMHC, NKX2-5, GATA4, cTNT, cTNI, MEF2C, MLC2a, or any combination thereof. In some embodiments, the mature cardiomyocytes express NKX2-5, MEF2C or a combination thereof. In some embodiments, cardiac progenitor cells express early stage cardiac progenitor markers such as GATA4, ISL1 or a combination thereof.

In some embodiments, the gene product is a polynucleotide. In some embodiments, as described below, the gene product is a guide RNA capable of binding to an RNA-guided endonuclease. In some embodiments, the gene product is an inhibitory nucleic acid capable of reducing the level of an mRNA and/or a polypeptide gene product, e.g., in a cardiac cell. For example, in some embodiments, the polynucleotide gene product is an interfering RNA capable of selectively inactivating a transcript encoded by an allele that causes a cardiac disease or disorder. As an example, the allele is a myosin heavy chain 7, cardiac muscle, beta (MYH7) allele that comprises a hypertrophic cardiomyopathy-causing mutation. Other examples include, e.g., interfering RNAs that selectively inactivate a transcript encoded by an allele that causes hypertrophic cardiomyopathy (HCM), dilated cardiomyopathy (DCM) or Left Ventricular Non-Compaction (LVNC), where the allele is a MYL3 (myosin light chain 3, alkali, ventricular, skeletal slow), MYH7, TNNI3 (troponin I type 3 (cardiac)), TNNT2 (troponin T type 2 (cardiac)), TPM1 (tropomyosin 1 (alpha)) or ACTC1 allele comprising an HCM-causing, a DCM-causing or a LVNC-causing mutation. See, e.g., U.S. Pat. Pub. No. 2016/0237430 for examples of cardiac disease-causing mutations.

In some embodiments, the gene product is a polypeptide-encoding RNA. In some embodiments, the gene product is an interfering RNA. In some embodiments, the gene product is an aptamer. In some embodiments, the gene product is a polypeptide. In some embodiments, the gene product is a therapeutic polypeptide, e.g., a polypeptide that provides clinical benefit. In some embodiments, the gene product is a site-specific nuclease that provide for site-specific knock-down of gene function. In some embodiments, the gene product is an RNA-guided endonuclease that provides for modification of a target nucleic acid. In some embodiments, the gene products are: i) an RNA-guided endonuclease that provides for modification of a target nucleic acid; and ii) a guide RNA that comprises a first segment that binds to a target sequence in a target nucleic acid and a second segment that binds to the RNA-guided endonuclease. In some embodiments, the gene products are: i) an RNA-guided endonuclease that provides for modification of a target nucleic acid; ii) a first guide RNA that comprises a first segment that binds to a first target sequence in a target nucleic acid and a second segment that binds to the RNA-guided endonuclease; and iii) a first guide RNA that comprises a first segment that binds to a second target sequence in the target nucleic acid and a second segment that binds to the RNA-guided endonuclease.

A nucleotide sequence encoding a heterologous gene product in an rAAV virion of the present disclosure can be operably linked to a promoter. For example, a nucleotide sequence encoding a heterologous gene product in an rAAV virion of the present disclosure can be operably linked to a constitutive promoter, a regulatable promoter, or a cardiac cell-specific promoter. Suitable constitutive promoters include a human elongation factor 1α subunit (EF1α) promoter, a β-actin promoter, an α-actin promoter, a β-glucuronidase promoter, CAG promoter, super core promoter, and a ubiquitin promoter. In some embodiments, a nucleotide sequence encoding a heterologous gene product in an rAAV virion of the present disclosure is operably linked to a cardiac-specific transcriptional regulator element (TRE), where cardiac-specific TREs include promoters and enhancers. Suitable cardiac-specific TREs include, but are not limited to, TREs derived from the following genes: myosin light chain-2 (MLC-2), a-myosin heavy chain (a-MHC), desmin, AE3, cardiac troponin C (cTnC), and cardiac actin. Franz et al. (1997) Cardiovasc. Res. 35:560-566; Robbins et al. (1995) Ann. NY. Acad. Sci. 752:492-505; Linn et al. (1995) Circ. Res. 76:584-591; Parmacek et al. (1994) Mol. Cell. Biol. 14:1870-1885; Hunter et al. (1993) Hypertension 22:608-617; and Sartorelli et al. (1992) Proc. Natl. Acad. Sci. USA 89:4047-4051. See also, Pacak et al. (2008) Genet Vaccines Ther. 6:13. In some embodiments, the promoter is an α-MHC promoter, an MLC-2 promoter, or cTnT promoter.

The polynucleotide encoding a gene product is operably linked to a promoter and/or enhancer to facilitate expression of the gene product. Depending on the host/vector system utilized, any of a number of suitable transcription and translation control elements, including constitutive and inducible promoters, transcription enhancer elements, transcription terminators, etc. may be used in the rAAV virion (e.g., Bitter et al. (1987) Methods in Enzymology, 153:516-544).

Separate promoters and/or enhancers can be employed for each of the polynucleotides. In some embodiments, the same promoter and/or enhance is used for two or more polynucleotides in a single open reading frame. Vectors employing this configuration of genetic elements are termed “polycistronic.” An illustrative example of a polycistronic vector comprises an enhancer and a promoter operatively linked to a single open-reading frame comprising two or more polynucleotides linked by 2A region(s), whereby expression of the open-reading frame result in multiple polypeptides being generated co-translationally. The 2A region is believed to mediate generation of multiple polypeptide sequences through codon skipping; however, the present disclosure relates also to polycistronic vectors that employ post-translational cleavage to generate two or more proteins of interest from the same polynucleotide. Illustrative 2A sequences, vectors, and associated methods are provided in US20040265955A1, which is incorporated herein by reference.

Non-limiting examples of suitable eukaryotic promoters (promoters functional in a eukaryotic cell) include CMV, CMV immediate early, HSV thymidine kinase, early and late SV40, long terminal repeats (LTRs) from retrovirus, and mouse metallothionein-I. In some embodiments, promoters that are capable of conferring cardiac specific expression will be used. Non-limiting examples of suitable cardiac specific promoters include desmin (Des), alpha-myosin heavy chain (a-MHC), myosin light chain 2 (MLC-2), cardiac troponin T (cTnT) and cardiac troponin C (cTnC). Non-limiting examples of suitable neuron specific promoters include synapsin I (SYN), calcium/calmodulin-dependent protein kinase II, tubulin alpha I, neuron-specific enolase and platelet-derived growth factor beta chain promoters and hybrid promoters by fusing cytomegalovirus enhancer (E) to those neuron-specific promoters.

Examples of suitable promoters for driving expression reprogramming factors include, but are not limited to, retroviral long terminal repeat (LTR) elements; constitutive promoters such as CMV, HSV1-TK, SV40, EF-1a, β-actin, phosphoglycerol kinase (PGK); inducible promoters, such as those containing Tet-operator elements; cardiac specific promoters, such as desmin (DES), alpha-myosin heavy chain (a-MHC), myosin light chain 2 (MLC-2), cardiac troponin T (cTnT) and cardiac troponin C (cTnC); neural specific promoters, such as nestin, neuronal nuclei (NeuN), microtubule-associate protein 2 (MAP2), beta III tubulin, neuron specific enolase (NSE), oligodendrocyte lineage (Olig1/2), and glial fibrillary acidic protein (GFAP); and pancreatic specific promoters, such as Pax4, Nkx2.2, Ngn3, insulin, glucagon, and somatostatin.

In some embodiments, a polynucleotide is operably linked to a cell type-specific transcriptional regulator element (TRE), where TREs include promoters and enhancers. Suitable TREs include, but are not limited to, TREs derived from the following genes: myosin light chain-2, a-myosin heavy chain, AE3, cardiac troponin C, and cardiac actin. Franz et al. (1997) Cardiovasc. Res. 35:560-566; Robbins et al. (1995) Ann. N. Y. Acad. Sci. 752:492-505; Linn et al. (1995) Circ. Res. 76:584-591; Parmacek et al. (1994) Cell. Biol. 14:1870-1885; Hunter et al. (1993) Hypertension 22:608-617; and Sartorelli et al. (1992) PNAS USA 89:4047-4051.

The promoter can be one naturally associated with a gene or nucleic acid segment. Similarly, for RNAs (e.g., microRNAs), the promoter can be one naturally associated with a microRNA gene (e.g., an miRNA-302 gene). Such a naturally associated promoter can be referred to as the “natural promoter” and may be obtained by isolating the 5′ non-coding sequences located upstream of the coding segment and/or exon. Similarly, an enhancer may be one naturally associated with a nucleic acid sequence. However, the enhancer can be located either downstream or upstream of that sequence.

Alternatively, certain advantages will be gained by positioning the coding nucleic acid segment under the control of a recombinant or heterologous promoter, which refers to a promoter that is not normally associated with a nucleic acid in its natural environment. A recombinant or heterologous enhancer refers also to an enhancer not normally associated with a nucleic acid sequence in its natural environment. Such promoters or enhancers can include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202, 5,928,906, each incorporated herein by reference).

The promoters employed may be constitutive, inducible, developmentally-specific, tissue-specific, and/or useful under the appropriate conditions to direct high level expression of the nucleic acid segment. For example, the promoter can be a constitutive promoter such as, a CMV promoter, a CMV cytomegalovirus immediate early promoter, a CAG promoter, an EF-1α promoter, a HSV1-TK promoter, an SV40 promoter, a R-actin promoter, a PGK promoter, or a combination thereof. Examples of eukaryotic promoters that can be used include, but are not limited to, constitutive promoters, e.g., viral promoters such as CMV, SV40 and RSV promoters, as well as regulatable promoters, e.g., an inducible or repressible promoter such as the tet promoter, the hsp70 promoter and a synthetic promoter regulated by CRE. In certain embodiments, cell type-specific promoters are used to drive expression of reprogramming factors in specific cell types. Examples of suitable cell type-specific promoters useful for the methods described herein include, but are not limited to, the synthetic macrophage-specific promoter described in He et al (2006), Human Gene Therapy 17:949-959; the granulocyte and macrophage-specific lysozyme M promoter (see, e.g., Faust et al (2000), Blood 96(2):719-726); and the myeloid-specific CD11b promoter (see, e.g., Dziennis et al (1995), Blood 85(2):319-329). Other examples of promoters that can be employed include a human EF1α elongation factor promoter, a CMV cytomegalovirus immediate early promoter, a CAG chicken albumin promoter, a viral promoter associated with any of the viral vectors described herein, or a promoter that is homologous to any of the promoters described herein (e.g., from another species). Examples of prokaryotic promoters that can be used include, but are not limited to, SP6, T7, T5, tac, bla, trp, gal, lac, or maltose promoters.

In some embodiments, an internal ribosome entry sites (IRES) element can be used to create multigene, or polycistronic, messages. IRES elements are able to bypass the ribosome scanning model of 5′-methylated Cap dependent translation and begin translation at internal sites (Pelletier and Sonenberg, Nature 334(6180):320-325 (1988)). IRES elements from two members of the picornavirus family (polio and encephalomyocarditis) have been described (Pelletier and Sonenberg, Nature 334(6180):320-325 (1988)), as well an IRES from a mammalian message (Macejak & Samow, Nature 353:90-94 (1991)). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

In some embodiments, a nucleotide sequence is operably linked to a polyadenylation sequence. Suitable polyadenylation sequences include bovine growth hormone polyA signal (bGHpolyA) and short poly A signal. Optionally the rAAV vectors of the disclosure comprise the Woodchuck Post-transcriptional Regulatory Element (WPRE). In some embodiments, the polynucleotide encoding gene products are join by sequences include so-called self-cleaving peptide, e.g. P2A peptides.

In some embodiments, the gene product comprises a site-specific endonuclease that provides for site-specific knock-down of gene function, e.g., where the endonuclease knocks out an allele associated with a cardiac disease or disorder. For example, where a dominant allele encodes a defective copy of a gene that, when wild-type, is a cardiac structural protein and/or provides for normal cardiac function, a site-specific endonuclease can be targeted to the defective allele and knock out the defective allele. In some embodiments, a site-specific endonuclease is an RNA-guided endonuclease.

In addition to knocking out a defective allele, a site-specific nuclease can also be used to stimulate homologous recombination with a donor DNA that encodes a functional copy of the protein encoded by the defective allele. For example, a subject rAAV virion can be used to deliver both a site-specific endonuclease that knocks out a defective allele a functional copy of the defective allele (or fragment thereof), resulting in repair of the defective allele, thereby providing for production of a functional cardiac protein (e.g., functional troponin, etc.). In some embodiments, a subject rAAV virion comprises a heterologous nucleotide sequence that encodes a site-specific endonuclease and a heterologous nucleotide sequence that encodes a functional copy of a defective allele, where the functional copy encodes a functional cardiac protein. Functional cardiac proteins include, e.g., troponin, a chloride ion channel, and the like.

Site-specific endonucleases that are suitable for use include, e.g., zinc finger nucleases (ZFNs); meganucleases; and transcription activator-like effector nucleases (TALENs), where such site-specific endonucleases are non-naturally occurring and are modified to target a specific gene. Such site-specific nucleases can be engineered to cut specific locations within a genome, and non-homologous end joining can then repair the break while inserting or deleting several nucleotides. Such site-specific endonucleases (also referred to as “INDELs”) then throw the protein out of frame and effectively knock out the gene. See, e.g., U.S. Pat. Pub. No. 2011/0301073. Suitable site-specific endonucleases include engineered meganuclease re-engineered homing endonucleases. Suitable endonucleases include an I-Tevl nuclease. Suitable meganucleases include I-Scel (see, e.g., Bellaiche et al. (1999) Genetics 152: 1037); and I-Crel (see, e.g., Heath et al. (1997) Nature Structural Biology 4:468). Site-specific endonucleases that are suitable for use include CRISPRi systems and the Cas9-based SAM system.

In some embodiments, the gene product is an RNA-guided endonuclease. In some embodiments, the gene product comprises an RNA comprising a nucleotide sequence encoding an RNA-guided endonuclease. In some embodiments, the gene product is a guide RNA, e.g., a single-guide RNA. In some embodiments, the gene products are: 1) a guide RNA; and 2) an RNA-guided endonuclease. The guide RNA can comprise: a) a protein-binding region that binds to the RNA-guided endonuclease; and b) a region that binds to a target nucleic acid. An RNA-guided endonuclease is also referred to herein as a “genome editing nuclease.”

Examples of suitable genome editing nucleases are CRISPR/Cas endonucleases (e.g., class 2 CRISPR/Cas endonucleases such as a type II, type V, or type VI CRISPR/Cas endonucleases). A suitable genome editing nuclease is a CRISPR/Cas endonuclease (e.g., a class 2 CRISPR/Cas endonuclease such as a type II, type V, or type VI CRISPR/Cas endonuclease). In some embodiments, the gene product comprises a class 2 CRISPR/Cas endonuclease. In some embodiments, the gene product comprises a class 2 type II CRISPR/Cas endonuclease (e.g., a Cas9 protein such as saCas9 or spCas9). In some embodiments, the gene product comprises a class 2 type V CRISPR/Cas endonuclease (e.g., a Cpf1 protein, a C2c1 protein, or a C2c3 protein). In some embodiments, the gene product comprises a class 2 type VI CRISPR/Cas endonuclease (e.g., a C2c2 protein; also referred to as a “Cas13a” protein). In some embodiments, the gene product comprises a CasX protein. In some embodiments, the gene product comprises a CasY protein. In some embodiments, the gene product comprises spCas9 H840A. In some embodiments, the gene product comprises split spCas9 H840A.

Nucleic Acids, Vectors, Cells, and Generation of rAAV Virions

In some embodiments, provided are nucleic acids or polynucleotides encoding an engineered capsid protein according to various embodiments described herein.

The polynucleotide encoding the capsid protein can comprise a sequence comprising either the native codons of the capsid protein, or alternative codons selected to encode the same protein. The codon usage of the insertion can be varied. It is within the skill of those in the art to select appropriate nucleotide sequences and to derive alternative nucleotide sequences to encode any capsid protein of the disclosure. Reverse translation of the protein sequence can be performed using the codon usage table of the host organism, i.e. eukaryotic codon usage for humans.

In some embodiments, provided is a polynucleotide encoding an AAV9 derived engineered capsid protein comprising a sequence at least 80%, 85%, 90%, 95%, 99%, or 100% identical to any one of SEQ ID NOs: 243-310.

In some embodiments, provided is a vector or a plasmid comprising a nucleic acid or polynucleotide encoding an engineered capsid protein according to various embodiments described herein. In some embodiments, the vector or plasmid further comprises a promoter operably linked to the nucleic acid encoding the engineered capsid protein. In some embodiments, the promoter is any promoter active in a cell to be used for expressing the engineered capsid protein (e.g., a producer or host cell). In some embodiments, the promoter is P40 promoter. In some embodiments, the promoter is a polyhedrin promoter.

In some embodiments, the vector or plasmid comprising a nucleic acid encoding an engineered capsid protein further comprises a nucleic acid encoding a replication (Rep) protein. In some embodiments, the Rep protein is a Rep protein from the same serotype of AAV as the inverted terminal repeats (ITRs) used to flank the transgene (to be packaged into virions using any of the AAV capsid proteins described herein). In some embodiments, the Rep protein is an AAV2 Rep protein. In some embodiments, the Rep protein is an AAV8 Rep protein. In some embodiments, the vector or plasmid comprising a nucleic acid encoding any AAV capsid protein described herein does not further comprise a nucleic acid encoding a Rep protein.

In some embodiments, provided is a cell comprising a nucleic acid or polynucleotide encoding an engineered capsid protein according to various embodiments described herein. In some embodiments, provided is a cell comprising a vector or a plasmid comprising a nucleic acid encoding an engineered capsid protein according to various embodiments described herein. In some embodiments, the cell further comprises a vector or plasmid comprising a nucleic acid encoding a Rep protein, wherein the Rep protein may be expressed by the same or different vector or plasmid as the AAV capsid protein described herein.

In some embodiments, provided is a host cell comprising a nucleic acid encoding an engineered capsid protein according to various embodiments described herein. In some embodiments, provided is a host cell comprising a vector or a plasmid comprising a nucleic acid encoding an engineered capsid protein according to various embodiments described herein.

In some embodiments, a host cell comprising a nucleic acid encoding an engineered capsid protein is for producing an rAAV virion described herein (such as an rAAV virion comprising an engineered capsid protein as described herein). In some embodiments, the nucleic acid encoding an engineered capsid protein is transiently transfected into a cell. In some embodiments, the nucleic acid encoding an engineered capsid protein is stably inserted into the cell genome.

In some embodiments, the host cell is a mammalian cell. In some embodiments, the host cell is selected from the group consisting of: are HEK293, HEK293T, HeLa, Vero, MDCK, MRC-5, PER.C6, BHK21 and CHO. In some embodiments, the host cell is HEK293 cell.

In some embodiments, the host cell is an insect cell. In some embodiments, the host cell is Sf9 insect cell. In some embodiments where the insect cells are used as host cells, the vectors or plasmids described herein are first introduced into a recombinant baculovirus and then carried into insect cells by baculovirus infection.

In some embodiments, the host cells are further transfected with one or more vectors or plasmids comprising helper functions and/or viral structural proteins necessary for replication and/or encapsidation of the vector(s) carrying the transgene.

In some embodiments, the host cells are further transfected with a viral vector carrying a transgene (such as any transgene described herein). In some embodiments, the transgene is flanked by inverted terminal repeats (ITRs). In some embodiments, the ITRs are of the same serotype as the Rep protein expressed in the host cells. In some embodiments, the ITRs are AAV2 ITRs. In some embodiments, the ITRs are AAV8 ITRs. Any combinations of Rep proteins and ITRs known in the art can be used in the cells and methods described herein.

In some embodiments, a host cell (e.g., a mammalian or an insect cell) further comprises a helper plasmid expression Adenovirus helper genes.

In some embodiments, a host cell comprises one or more packaging factors stably integrated into cell genome. In some embodiments, the host cell comprises a nucleic acid encoding any of the AAV capsid proteins described herein stably integrated into its genome. In some embodiments, the host cell comprises a nucleic acid encoding a Rep protein stably integrated into its genome. In some embodiments, the host cell comprises an Adenovirus helper gene stably integrated into its genome. In some embodiments, the host cell comprises a nucleic acid encoding an AAV capsid protein described herein, a nucleic acid encoding a Rep protein, and an Adenovirus helper gene(s) stably integrated into its genome.

Methods for production of rAAV virions are known in the art. In some embodiments, an rAAV virion can be generated using the host cells as described herein.

In some embodiments, the method of producing an rAAV virion in cell comprises:
i. introducing (e.g., by transient transfection or stable integration techniques) a nucleic acid encoding any of the AAV capsid proteins described herein, a nucleic acid encoding a Rep protein (such as any AAV Rep protein known in the art or described herein), an Adenovirus helper gene(s) (such as any Adenovirus helper genes known in the art), and/or a transgene cassette comprising a transgene flanked by ITRs (e.g., wherein the transgene expresses a therapeutic protein) into the cell (e.g., via DNA transfection, viral infection, and/or stable integration), wherein each of the introduced nucleic acids or genes is operably linked to a promoter active in the cell;
ii culturing the cell (e.g., using a suspension cell culture or an adherent cell culture) under conditions suitable for production of an rAAV virion (e.g., suitable for packaging protein expression and/or suitable for viral packaging), and
iii. collecting the produced rAAV virion (e.g., from media supernatant and/or from cell lysate following cell lysis), and
iv. optionally further purifying the rAAV virion, e.g., by density gradient ultracentrifugation and/or chromatography-based methods.

In some embodiments, the vectors, promoters, packaging factors, packaging systems, host cells, and/or methods of rAAV virion production are any of those known in the art.

In some embodiments, provided are methods of identifying AAV capsid proteins (wild-type, modified, or engineered) that confer on rAAV virions increased transduction efficiency in target cells. The methods comprise providing a population of rAAV virions whose rAAV genomes comprise a library of cap polynucleotides encoding variant AAV capsid proteins; optionally contacting the population with non-target cells for a time sufficient to permit attachment of undesired rAAV virions to the non-target cells; contacting the population with target cells for a time sufficient to permit transduction of the cap polynucleotide into the target cells by the rAAV virions; and sequencing the cap polynucleotides from the target cells, thereby identifying AAV capsid proteins that confer increased transduction efficiency in the target cells. In some embodiments, the method further comprises depleting the population of rAAV virions by contacting the population with non-target cells for time sufficient to permit attachment of the rAAV virions to the non-target cells.

In some embodiments, provided are methods for generating cardiomyocytes and/or cardiomyocyte-like cells in vitro using an rAAV virion. Selected starting cells are transduced with an rAAV and optionally exposed to small-molecule reprogramming factors (before, during, or after transduction) for a time and under conditions sufficient to convert the starting cells across lineage and/or differentiation boundaries to form cardiac progenitor cells and/or cardiomyocytes. In some embodiments, the starting cells are fibroblast cells. In some embodiments, the starting cells express one or more markers indicative of a differentiated phenotype. The time for conversion of starting cells into cardiac progenitor and cardiomyocyte cells can vary. For example, the starting cells can be incubated after treatment with one or more polynucleotides or proteins of interest until cardiac or cardiomyocyte cell markers are expressed. Such cardiac or cardiomyocyte cell markers can include any of the following markers: α-GATA4, TNNT2, MYH6, RYR2, NKX2-5, MEF2C, ANP, Actinin, MLC2v, MY20, cMHC, ISL1, cTNT, cTNI, and MLC2a, or any combination thereof. In some embodiments, the induced cardiomycocyte cells are negative for one or more neuronal cells markers. Such neuronal cell markers can include any of the following markers: DCX, TUBB3, MAP2, and ENO2.

Incubation can proceed until cardiac progenitor markers are expressed by the starting cells. Such cardiac progenitor markers include GATA4, TNNT2, MYH6, RYR2, or a combination thereof. The cardiac progenitor markers such as GATA4, TNNT2, MYH6, RYR2, or a combination thereof can be expressed by about 8 days, or by about 9 days, or by about 10 days, or by about 11 days, or by about 12 days, or by about 14 days, or by about 15 days, or by about 16 days, or by about 17 days, or by about 18 days, or by about 19 days, or by about 20 days after starting incubation of cells in the compositions described herein. Further incubation of the cells can be performed until expression of late stage cardiac progenitor markers such as NKX2-5, MEF2C or a combination thereof occurs.

Reprogramming efficiency may be measured as a function of cardiomyocyte markers. Such pluripotency markers include, but are not limited to, the expression of cardiomyocyte marker proteins and mRNA, cardiomyocyte morphology and electrophysiological phenotype. Non-limiting examples of cardiomyocyte markers include, a-sarcoglycan, atrial natriuretic peptide (ANP), bone morphogenetic protein 4 (BMP4), connexin 37, connexin 40, crypto, desmin, GATA4, GATA6, MEF2C, MYH6, myosin heavy chain, NKX2.5, TBX5, and Troponin T.

The expression of various markers specific to cardiomyocytes may be detected by conventional biochemical or immunochemical methods (e.g., enzyme-linked immunosorbent assay, immunohistochemical assay, and the like). Alternatively, expression of a nucleic acid encoding a cardiomyocyte-specific marker can be assessed. Expression of cardiomyocyte-specific marker-encoding nucleic acids in a cell can be confirmed by reverse transcriptase polymerase chain reaction (RT-PCR) or hybridization analysis, molecular biological methods which have been commonly used in the past for amplifying, detecting and analyzing mRNA coding for any marker proteins. Nucleic acid sequences coding for markers specific to cardiomyocytes are known and are available through public databases such as GenBank. Thus, marker-specific sequences needed for use as primers or probes are easily determined.

Cardiomyocytes exhibit some cardiac-specific electrophysiological properties. One electrical characteristic is an action potential, which is a short-lasting event in which the difference of potential between the interior and the exterior of each cardiac cell rises and falls following a consistent trajectory. Another electrophysiological characteristic of cardiomyocytes is the cyclic variations in the cytosolic-free Ca²⁺ concentration, named as Ca²⁺ transients, which are employed in the regulation of the contraction and relaxation of cardiomyocytes. These characteristics can be detected and evaluated to assess whether a population of cells has been reprogrammed into cardiomyocytes.

In some embodiments, provided are methods of delivering a gene product to a cardiac cell, e.g., a cardiac fibroblast. The methods generally involve infecting a cardiac cell (e.g., a cardiac fibroblast) with an rAAV virion, where the gene product(s) encoded by the heterologous nucleic acid present in the rAAV virion is/are produced in the cardiac cell (e.g., cardiac fibroblast). Delivery of gene product(s) to a cardiac cell (e.g., cardiac fibroblast) can provide for treatment of a cardiac disease or disorder. Delivery of gene product(s) to a cardiac cell (e.g., cardiac fibroblast) can provide for generation of an induced cardiomyocyte-like (iCM) cell from the cardiac fibroblast. Delivery of gene product(s) to a cardiac cell (e.g., cardiac fibroblast) can provide for editing of the genome of the cardiac cell (e.g., cardiac fibroblast).

In some embodiments, infecting or transducing a cardiac cell (e.g., cardiac fibroblast) is carried out in vitro. In some embodiments, infecting or transducing a cardiac cell (e.g., cardiac fibroblast) is carried out in vitro; and the infected/transduced cardiac cell (e.g., cardiac fibroblast) is introduced into (e.g., transfused into or implanted into) an individual in need thereof, e.g., directly into cardiac tissue of an individual in need thereof. For in vitro transduction, an effective amount of rAAV virions to be delivered to cells is from about 10⁵ to about 10¹³ of the rAAV virions. Other effective dosages can be readily established by one of ordinary skill in the art through routine trials establishing dose response curves.

In some embodiments, infecting a cardiac cell (e.g., cardiac fibroblast) is carried out in vivo. For example, in some embodiments, an effective amount of an rAAV virion of the present disclosure is administered directly into cardiac tissue of an individual in need thereof. An “effective amount” will fall in a relatively broad range that can be determined through experimentation and/or clinical trials. For example, for in vivo injection, i.e., injection directly into cardiac tissue, a therapeutically effective dose will be on the order of from about 10⁶ to about 10¹⁵ of the rAAV virions, e.g., from about 10⁵ to 10¹² rAAV virions, of the present disclosure. In some embodiments, an effective amount of an rAAV virion of the present disclosure is administered via intramyocardial injection through the epicardium. In some embodiments, an effective amount of an rAAV virion of the present disclosure is administered via vascular delivery through the coronary artery. In some embodiments, an effective amount of an rAAV virion of the present disclosure is administered via systemic delivery through the superior vena cava. In some embodiments, an effective amount of an rAAV virion of the present disclosure is administered via systemic delivery through a peripheral vein.

For example, from about 10⁴ to about 10⁵, from about 10⁵ to about 10⁶, from about 10⁶ to about 10⁷, from about 10⁶ to about 10⁷, from about 10⁷ to about 10⁸, from about 10⁸ to about 10⁹, from about 10⁹ to about 10¹⁰, from about 10¹⁰ to about 10¹¹, to about 10¹, from about 10¹¹ to about 10¹², from about 10¹² to about 10¹³, from about 10¹³ to about 10¹⁴, from about 10¹⁴ to about 10¹⁵ genome copies, or more than 10¹⁵ genome copies, of an rAAV virion of the present disclosure are administered to an individual, e.g., are administered directly into cardiac tissue in the individual, or are administered via another route. The number of rAAV virions administered to an individual can be expressed in viral genomes (vg) per kilogram (kg) body weight of the individual. In some embodiments, and effective amount of an rAAV virion of the present disclosure is from about 10² vg/kg to 10⁴ vg/kg, from about 10⁴ vg/kg to about 10⁶ vg/kg, from about 10⁶ vg/kg to about 10⁸ vg/kg, from about 10⁸ vg/kg to about 10¹⁰ vg/kg, from about 10¹⁰ vg/kg to about 10¹² vg/kg, from about 10¹² vg/kg to about 10¹⁴ vg/kg, from about 10¹⁴ vg/kg to about 10¹⁶ vg/kg, from about 10¹⁶ vg/kg to about 10¹⁸ vg/kg, or more than 10¹⁸ vg/kg. In some embodiments, the rAAV viron is administered at, at least at, or at no more than, 10² vg/kg, 10³ vg/kg, 10⁴ vg/kg, 10⁵ vg/kg, 10⁶ vg/kg, 10⁸ vg/kg, 10⁹ vg/kg, 10¹⁰ vg/kg, 10¹¹ vg/kg, 10¹² vg/kg, 10¹³ vg/kg, 2×10¹³ vg/kg, 3×10¹³ vg/kg, 4×10¹³ vg/kg, 5×10¹³ vg/kg, 6×10¹³ vg/kg, 7×10¹³ vg/kg, 8×10¹³ vg/kg, 9×10¹³ vg/kg, 10¹⁴ vg/kg, 2×10¹⁴ vg/kg, 3×10¹⁴ vg/kg, 4×10¹⁴ vg/kg, 5×10¹⁴ vg/kg, 6×10¹⁴ vg/kg, 7×10¹⁴ vg/kg, 8×10¹⁴ vg/kg, 9×10¹⁴ vg/kg, 10¹⁵ vg/kg, 10¹⁶ vg/kg, 10¹⁷ vg/kg, or 10¹⁸ vg/kg (or at any range of amounts in between these values). In some embodiments, the rAAV virion is administered at 2×10¹³ vg/kg. In some embodiments, the rAAV virion is administered at 1.43×10¹³ vg/kg. In some embodiments, the rAAV virion is administered at 1.2×10¹⁴ vg/kg.

In some embodiments, an effective amount of an rAAV virion of the present disclosure is administered locally to the heart. In some embodiments, an effective amount of an rAAV virion of the present disclosure is administered via intramyocardial injection through the epicardium. In some embodiments, an effective amount of an rAAV virion of the present disclosure is administered by intracardiac catheterization. In some embodiments, an effective amount of an rAAV virion of the present disclosure is administered via vascular delivery through the coronary artery. In some embodiments, an effective amount of an rAAV virion of the present disclosure is administered via systemic delivery, e.g., intravenously. In some embodiments, an effective amount of an rAAV virion of the present disclosure is administered via systemic delivery through the superior vena cava. In some embodiments, an effective amount of an rAAV virion of the present disclosure is administered via systemic delivery through a peripheral vein.

In some embodiments, a single administration may be used to achieve the desired level of gene expression. In some embodiments, more than one administration (e.g., two, three, four or more administrations) may be employed to achieve the desired level of gene expression. In some embodiments, the more than one administration is administered at various intervals, e.g., daily, weekly, twice monthly, monthly, every 3 months, every 6 months, yearly, etc. In some embodiments, multiple administrations are administered over a period of time of from 1 month to 2 months, from 2 months to 4 months, from 4 months to 8 months, from 8 months to 12 months, from 1 year to 2 years, from 2 years to 5 years, or more than 5 years.

The present disclosure provides a method of reprogramming a cardiac fibroblast to generate an induced cardiomyocyte-like cell (iCM). The method generally involves infecting a cardiac fibroblast with an rAAV virion of the present disclosure, wherein the rAAV virion comprises a heterologous nucleic acid comprising a nucleotide sequence encoding one or more reprogramming factors.

The expression of various markers specific to cardiomyocytes is detected by conventional biochemical or immunochemical methods (e.g., enzyme-linked immunosorbent assay; immunohistochemical assay; and the like). Alternatively, expression of nucleic acid encoding a cardiomyocyte-specific marker can be assessed. Expression of cardiomyocyte-specific marker-encoding nucleic acids in a cell can be confirmed by reverse transcriptase polymerase chain reaction (RT-PCR) or hybridization analysis, molecular biological methods which have been commonly used in the past for amplifying, detecting and analyzing mRNA coding for any marker proteins. Nucleic acid sequences coding for markers specific to cardiomyocytes are known and are available through public data bases such as GenBank; thus, marker-specific sequences needed for use as primers or probes is easily determined.

Induced cardiomyocytes can also exhibit spontaneous contraction. Whether an induced cardiomyocyte exhibits spontaneous contraction can be determined using standard electrophysiological methods (e.g., patch clamp).

In some embodiments, induced cardiomyocytes can exhibit spontaneous Ca²⁺ oscillations. Ca²⁺ oscillations can be detected using standard methods, e.g., using any of a variety of calcium-sensitive dyes, intracellular Ca²⁺ ion-detecting dyes include, but are not limited to, fura-2, bis-fura 2, indo-1, Quin-2, Quin-2 AM, Benzothiaza-1, Benzothiaza-2, indo-5F, Fura-FF, BTC, Mag-Fura-2, Mag-Fura-5, Mag-Indo-1, fluo-3, rhod-2, rhod-3, fura-4F, fura-5F, fura-6F, fluo-4, fluo-5F, fluo-5N, Oregon Green 488 BAPTA, Calcium Green, Calcein, Fura-C18, Calcium Green-C18, Calcium Orange, Calcium Crimson, Calcium Green-5N, Magnesium Green, Oregon Green 488 BAPTA-1, Oregon Green 488 BAPTA-2, X-rhod-1, Fura Red, Rhod-5F, Rhod-5N, X-Rhod-5N, Mag-Rhod-2, Mag-X-Rhod-1, Fluo-5N, Fluo-5F, Fluo-4FF, Mag-Fluo-4, Aequorin, dextran conjugates or any other derivatives of any of these dyes, and others (see, e.g., the catalog or Internet site for Molecular Probes, Eugene, see, also, Nuccitelli, ed., Methods in Cell Biology, Volume 40: A Practical Guide to the Study of Calcium in Living Cells, Academic Press (1994); Lambert, ed., Calcium Signaling Protocols (Methods in Molecular Biology Volume 114), Humana Press (1999); W. T. Mason, ed., Fluorescent and Luminescent Probes for Biological Activity. A Practical Guide to Technology for Quantitative Real-Time Analysis, Second Ed, Academic Press (1999); Calcium Signaling Protocols (Methods in Molecular Biology), 2005, D. G. Lamber, ed., Humana Press.).

In some embodiments, an iCM is generated in vitro; and the iCM is introduced into an individual, e.g., the iCM is implanted into a cardiac tissue of an individual in need thereof. A method of the present disclosure can comprise infecting a population of cardiac fibroblasts in vitro, to generate a population of iCMs; and the population of iCMs is implanted into a cardiac tissue of an individual in need thereof.

In some embodiments, an iCM is generated in vivo. For example, in some embodiments, an rAAV virion of the present disclosure that comprises a heterologous nucleic acid comprising a nucleotide sequence encoding one or more reprogramming factors is administered to an individual. In some embodiments, the rAAV virion is administered directly into cardiac tissue of an individual in need thereof. In some embodiments, from about 10⁶ to about 10⁵, from about 10⁵ to about 10⁹, from about 10⁹ to about 10¹⁰, from about 10¹⁰ to about 10¹¹, from about 10¹¹ to about 10¹², from about 10¹² to about 10¹³, from about 10¹³ to about 10¹⁴, from about 10¹⁴ to about 10¹⁵ genome copies, or more than 10¹⁵ genome copies, of an rAAV virion of the present disclosure that comprises a heterologous nucleic acid comprising a nucleotide sequence encoding one or more reprogramming factors are administered to an individual, e.g., are administered directly into cardiac tissue in the individual or via another route of administration. The number of rAAV virions administered to an individual can be expressed in viral genomes (vg) per kilogram (kg) body weight of the individual. In some embodiments, and effective amount of an rAAV virion of the present disclosure is from about 10² vg/kg to 10⁴ vg/kg, from about 10⁴ vg/kg to about 10⁶ vg/kg, from about 10⁶ vg/kg to about 10⁸ vg/kg, from about 10⁸ vg/kg to about 10¹⁰ vg/kg, from about 10¹⁰ vg/kg to about 10¹² vg/kg, from about 10¹² vg/kg to about 10¹⁴ vg/kg, from about 10¹⁴ vg/kg to about 10¹⁴ vg/kg, from about 10¹⁴ vg/kg to about 10¹⁶ vg/kg, or more than 10¹⁶ vg/kg. In some embodiments, an effective amount of an rAAV virion of the present disclosure is administered via intramyocardial injection through the epicardium. In some embodiments, an effective amount of an rAAV virion of the present disclosure is administered via vascular delivery through the coronary artery. In some embodiments, an effective amount of an rAAV virion of the present disclosure is administered via systemic delivery through the superior vena cava. In some embodiments, an effective amount of an rAAV virion of the present disclosure is administered via systemic delivery through a peripheral vein.

In some embodiments, provided are methods of modifying (“editing”) the genome of a cardiac cell. The present disclosure provides a method of modifying (“editing”) the genome of a cardiac fibroblast. The present disclosure provides a method of modifying (“editing”) the genome of a cardiomyocyte. The methods generally involve infecting a cardiac cell (e.g., a cardiac fibroblast or a cardiomyocyte) with an rAAV virion of the present disclosure, wherein the rAAV virion comprises a heterologous nucleic acid comprising a nucleotide sequence encoding a genome-editing endonuclease. In some embodiments, the method comprises infecting a cardiac fibroblast or a cardiomyocyte with an rAAV virion of the present disclosure, wherein the rAAV virion comprises a heterologous nucleic acid comprising a nucleotide sequence encoding an RNA-guided genome-editing endonuclease. In some embodiments, the method comprises infecting a cardiac fibroblast or a cardiomyocyte with an rAAV virion of the present disclosure, wherein the rAAV virion comprises a heterologous nucleic acid comprising a nucleotide sequence encoding: i) an RNA-guided genome-editing endonuclease; and ii) one or more guide RNAs. In some embodiments, the method comprises infecting a cardiac fibroblast or a cardiomyocyte with an rAAV virion of the present disclosure, wherein the rAAV virion comprises a heterologous nucleic acid comprising a nucleotide sequence encoding: i) an RNA-guided genome-editing endonuclease; ii) a guide RNAs; and iii) a donor template DNA. Suitable RNA-guided genome-editing endonucleases are described above.

In some embodiments, infecting a cardiac cell (e.g., cardiac fibroblast; a cardiomyocyte) is carried out in vitro. In some embodiments, infecting a cardiac cell (e.g., cardiac fibroblast; a cardiomyocyte) is carried out in vitro; and the infected cardiac cell (e.g., cardiac fibroblast) is introduced into (e.g., implanted into) an individual in need thereof, e.g., directly into cardiac tissue of an individual in need thereof. For in vitro transduction, an effective amount of rAAV virions to be delivered to cells will be on the order of from about 10^{s} to about 10¹³ of the rAAV virions. Other effective dosages can be readily established by one of ordinary skill in the art through routine trials establishing dose response curves.

In some embodiments, infecting a cardiac cell (e.g., cardiac fibroblast; a cardiomyocyte) is carried out in vivo. For example, in some embodiments, an effective amount of an rAAV virion of the present disclosure is administered directly into cardiac tissue of an individual in need thereof. An “effective amount” will fall in a relatively broad range that can be determined through experimentation and/or clinical trials. For example, for in vivo injection, i.e., injection directly into cardiac tissue, a therapeutically effective dose will be on the order of from about 10⁶ to about 10¹⁵ of the rAAV virions, e.g., from about 10¹¹ to 10¹² rAAV virions, of the present disclosure. In some embodiments, an effective amount of an rAAV virion of the present disclosure is administered via intramyocardial injection through the epicardium. In some embodiments, an effective amount of an rAAV virion of the present disclosure is administered via vascular delivery through the coronary artery. In some embodiments, an effective amount of an rAAV virion of the present disclosure is administered via systemic delivery through the superior vena cava. In some embodiments, an effective amount of an rAAV virion of the present disclosure is administered via systemic delivery through a peripheral vein.

For example, from about 10⁶ to about 10⁷, from about 10⁷ to about 10⁸, from about 10⁸ to about 10⁹, from about 10⁹ to about 10¹⁰, from about 10¹⁰ to about 10¹¹, from about 10¹¹ to about 10¹², from about 10¹² to about 10¹³, from about 10¹³ to about 10¹⁴, from about 10¹⁴ to about 10¹⁵ genome copies, or more than 10¹⁵ genome copies, of an rAAV virion of the present disclosure are administered to an individual, e.g., are administered directly into cardiac tissue in the individual. The number of rAAV virions administered to an individual can be expressed in viral genomes (vg) per kilogram (kg) body weight of the individual. In some embodiments, and effective amount of an rAAV virion of the present disclosure is from about 10² vg/kg to 10⁴ vg/kg, from about 10⁴ vg/kg to about 10⁶ vg/kg, from about 10⁶ vg/kg to about 10⁸ vg/kg, from about 10⁸ vg/kg to about 10¹⁰ vg/kg, from about 10¹⁰ vg/kg to about 10¹² vg/kg, from about 10¹² vg/kg to about 10¹⁴ vg/kg, from about 10¹⁴ vg/kg to about 10¹⁶ vg/kg, from about 10¹⁶ vg/kg to about 10¹⁸ vg/kg, or more than 10¹⁸ vg/kg. In some embodiments, an effective amount of an rAAV virion of the present disclosure is administered via intramyocardial injection through the epicardium. In some embodiments, an effective amount of an rAAV virion of the present disclosure is administered via vascular delivery through the coronary artery. In some embodiments, an effective amount of an rAAV virion of the present disclosure is administered via systemic delivery through the superior vena cava. In some embodiments, an effective amount of an rAAV virion of the present disclosure is administered via systemic delivery through a peripheral vein.

In some embodiments, the genome editing comprises homology-directed repair (HDR). In some embodiments, the HDR corrects a defect in an endogenous target nucleic acid in the cardiac fibroblast or the cardiomyocyte, wherein the defect is associated with, or leads to, a defect in structure and/or function of the cardiac fibroblast or the cardiomyocyte, or a component of the cardiac fibroblast or the cardiomyocyte.

In some embodiments, the genome editing comprises non-homologous end joining (NHEJ). In some embodiments, the NHEJ deletes a defect in an endogenous target nucleic acid in the cardiac fibroblast or the cardiomyocyte, wherein the defect is associated with, or leads to, a defect in structure and/or function of the cardiac fibroblast or the cardiomyocyte, or a component of the cardiac fibroblast or the cardiomyocyte.

A method of the present disclosure for editing the genome of a cardiac cell can be used to correct any of a variety of genetic defects that give rise to a cardiac disease or disorder. Mutations of interest include mutations in one or more of the following genes: cardiac troponin T (TNNT2); myosin heavy chain (MYH7); tropomyosin 1 (TPM1); myosin binding protein C (MYBPC3); 5′-AMP-activated protein kinase subunit gamma-2 (PRKAG2); troponin I type 3 (TNNI3); titin (TTN); myosin, light chain 2 (MYL2); actin, alpha cardiac muscle 1 (ACTC1); potassium voltage-gated channel, KQT-like subfamily, member 1 (KCNQ1); myocyte enhancer factor 2c (MEF2C); and cardiac LIM protein (CSRP3). Specific mutations of interest include, without limitation, MYH7 R663H mutation; TNNT2 R173W; and KCNQ1 G269S missense mutation. Mutations of interest include mutations in one or more of the following genes: MYH6, ACTN2, SERCA2, GATA4, TBX5, MYOCD, NKX2-5, NOTCHI, MEF2C, HAND2, and HAND1. In some embodiments, the mutations of interest include mutations in the following genes: MEF2C, TBX5, and MYOCD. Cardiac diseases and disorders that can be treated with a method of the present disclosure include coronary heart disease, cardiomyopathy, endocarditis, congenital cardiovascular defects, and congestive heart failure. Cardiac diseases and disorders that can be treated with a method of the present disclosure include hypertrophic cardiomyopathy; a valvular heart disease; myocardial infarction; congestive heart failure; long QT syndrome; atrial arrhythmia; ventricular arrhythmia; diastolic heart failure; systolic heart failure; cardiac valve disease; cardiac valve calcification; left ventricular non-compaction; ventricular septal defect; and ischemia.

In some embodiments, provided are methods of transducing a cardiac cell. In some embodiments, the disclosure provides a method of transducing a cardiac cell, comprising contacting the cardiac cell with an rAAV virion described herein, wherein the rAAV virion transduces the cardiac cell. In some embodiments, the cardiac cell is a cardiomyocyte.

In some embodiments, provided are methods of transducing a cardiac cell, comprising contacting the cardiac cell with an rAAV virion, wherein the rAAV virion comprises a capsid protein, wherein the capsid protein is any capsid protein described herein.

In some embodiments, provided are methods of delivering one or more gene products to a cardiac cell. In some embodiments, the method of delivering one or more gene products to a cardiac cell comprises contacting the cardiac cell with an rAAV virion described herein. In some embodiments, the cardiac cell is a cardiomyocyte.

In some embodiments, provided are methods of delivering one or more gene products to a cardiac cell with an rAAV virion comprising a capsid protein, wherein the capsid protein is any capsid protein described herein.

### Methods of Treatment

In some embodiments, provided are methods of treating a cardiac pathology in a subject in need thereof, comprising administering a therapeutically effective amount of a pharmaceutical composition comprising an rAAV virion to the subject, wherein the rAAV virion comprises an engineered capsid protein according to various embodiments disclosed herein.

Subjects in need of treatment using compositions and methods of the present disclosure include, but are not limited to, individuals having a congenital heart defect, individuals suffering from a degenerative muscle disease, individuals suffering from a condition that results in ischemic heart tissue (e.g., individuals with coronary artery disease), and the like. In some examples, a method is useful to treat a degenerative muscle disease or condition (e.g., familial cardiomyopathy, dilated cardiomyopathy, hypertrophic cardiomyopathy, restrictive cardiomyopathy, or coronary artery disease with resultant ischemic cardiomyopathy). In some examples, a subject method is useful to treat individuals having a cardiac or cardiovascular disease or disorder, for example, cardiovascular disease, aneurysm, angina, arrhythmia, atherosclerosis, cerebrovascular accident (stroke), cerebrovascular disease, congenital heart disease, congestive heart failure, myocarditis, valve disease coronary, artery disease dilated, diastolic dysfunction, endocarditis, high blood pressure (hypertension), cardiomyopathy, hypertrophic cardiomyopathy, restrictive cardiomyopathy, coronary artery disease with resultant ischemic cardiomyopathy, mitral valve prolapse, myocardial infarction (heart attack), or venous thromboembolism.

Subjects suitable for treatment using the compositions, cells and methods of the present disclosure include individuals (e.g., mammalian subjects, such as humans, non-human primates, domestic mammals, experimental non-human mammalian subjects such as mice, rats, etc.) having a cardiac condition including but limited to a condition that results in ischemic heart tissue (e.g., individuals with coronary artery disease) and the like.

In some examples, an individual suitable for treatment suffers from a cardiac or cardiovascular disease or condition, e.g., cardiovascular disease, aneurysm, angina, arrhythmia, atherosclerosis, cerebrovascular accident (stroke), cerebrovascular disease, congenital heart disease, congestive heart failure, myocarditis, valve disease coronary, artery disease dilated, diastolic dysfunction, endocarditis, high blood pressure (hypertension), cardiomyopathy, hypertrophic cardiomyopathy, restrictive cardiomyopathy, coronary artery disease with resultant ischemic cardiomyopathy, mitral valve prolapse, myocardial infarction (heart attack), or venous thromboembolism. In some examples, individuals suitable for treatment with a subject method include individuals who have a degenerative muscle disease, e.g., familial cardiomyopathy, dilated cardiomyopathy, hypertrophic cardiomyopathy, restrictive cardiomyopathy, or coronary artery disease with resultant ischemic cardiomyopathy.

For example, the cardiac pathology can be selected from the group consisting of congestive heart failure, myocardial infarction, cardiac ischemia, myocarditis and arrhythmia. In some embodiments, the subject is diabetic. In some embodiments, the subject is non-diabetic. In some embodiments, the subject suffers from diabetic cardiomyopathy.

For therapy, the rAAV virions of the disclosure and/or pharmaceutical compositions thereof can be administered locally or systemically. An rAAV virion can be introduced by injection, catheter, implantable device, or the like. An rAAV virion can be administered in any physiologically acceptable excipient or carrier that does not adversely affect the cells. For example, rAAV virions of the disclosure and/or pharmaceutical compositions thereof can be administered intravenously or through an intracardiac route (e.g., epicardially or intramyocardially). Methods of administering rAAV virions of the disclosure and/or pharmaceutical compositions thereof to subjects, particularly human subjects include injection or infusion of the pharmaceutical compositions (e.g., compositions comprising rAAV virions). Injection may include direct muscle injection and infusion may include intravascular infusion. The rAAV virions or pharmaceutical compositions can be inserted into a delivery device which facilitates introduction by injection into the subjects. Such delivery devices include tubes, e.g., catheters, for injecting cells and fluids into the body of a recipient subject. The tubes can additionally include a needle, e.g., a syringe, through which the cells of the invention can be introduced into the subject at a desired location.

In some embodiments, the rAAV virion is administered by subcutaneous, intravenous, intramuscular, intraperitoneal, or intracardiac injection or by intracardiac catheterization. In some embodiments, the rAAV virion is administered by direct intramyocardial injection or transvascular administration. In some embodiments, the rAAV virion is administered by direct intramyocardial injection, antegrade intracoronary injection, retrograde injection, transendomyocardial injection, or molecular cardiac surgery with recirculating delivery (MCARD).

The rAAV virions can be inserted into such a delivery device, e.g., a syringe, in different forms. The rAAV virion can be supplied in the form of a pharmaceutical composition. Such a composition can include an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. The choice of the excipient and any accompanying constituents of the composition can be adapted to optimize administration by the route and/or device employed.

Recombinant AAV may be administered locally or systemically. Recombinant AAV may be engineered to target specific cell types by selecting the appropriate capsid protein of the disclosure. To determine the suitability of various therapeutic administration regimens and dosages of AAV virion compositions, the rAAV virions can first be tested in a suitable animal model. At one level, recombinant AAV are assessed for their ability to infect target cells in vivo. Recombinant AAV can also be assessed to ascertain whether it migrates to target tissues, whether they induce an immune response in the host, or to determine an appropriate number, or dosage, of rAAV virions to be administered. It may be desirable or undesirable for the recombinant AAV to generate an immune response, depending on the disease to be treated. Generally, if repeated administration of a virion is required, it will be advantageous if the virion is not immunogenic. For testing purposes, rAAV virion compositions can be administered to immunodeficient animals (such as nude mice, or animals rendered immunodeficient chemically or by irradiation). Target tissues or cells can be harvested after a period of infection and assessed to determine if the tissues or cells have been infected and if the desired phenotype (e.g. induced cardiomyocyte) has been induced in the target tissue or cells.

Recombinant AAV virions can be administered by various routes, including without limitation direct injection into the heart or cardiac catheterization. Alternatively, the rAAV virions can be administered systemically such as by intravenous infusion. When direct injection is used, it may be performed either by open-heart surgery or by minimally invasive surgery. In some embodiments, the recombinant viruses are delivered to the pericardial space by injection or infusion. Injected or infused recombinant viruses can be traced by a variety of methods. For example, recombinant AAV labeled with or expressing a detectable label (such as green fluorescent protein, or beta-galactosidase) can readily be detected. The recombinant AAV may be engineered to cause the target cell to express a marker protein, such as a surface-expressed protein or a fluorescent protein. Alternatively, the infection of target cells with recombinant AAV can be detected by their expression of a cell marker that is not expressed by the animal employed for testing (for example, a human-specific antigen when injecting cells into an experimental animal). The presence and phenotype of the target cells can be assessed by fluorescence microscopy (e.g., for green fluorescent protein, or beta-galactosidase), by immunohistochemistry (e.g., using an antibody against a human antigen), by ELISA (using an antibody against a human antigen), or by RT-PCR analysis using primers and hybridization conditions that cause amplification to be specific for RNA indicative of a cardiac phenotype.

In some embodiments, provided are methods of treating a cardiac pathology in a subject in need thereof, comprising administering a therapeutically effective amount of an rAAV virion comprising an engineered capsid protein according to various embodiments described herein.

In some embodiments, provided are methods of treating a cardiac pathology in a subject in need thereof, comprising administering a therapeutically effective amount of an rAAV virion comprising an engineered capsid protein according to various embodiments described herein.

### Pharmaceutical Compositions

In some embodiments, provided are pharmaceutical compositions comprising an rAAV virion, wherein the rAAV virion comprises an engineered capsid protein according to various embodiments disclosed herein.

The pharmaceutical composition may include one or more of a pharmaceutically acceptable carrier, diluent, excipient, and buffer. In some embodiments, the pharmaceutically acceptable carrier, diluent, excipient, or buffer is suitable for use in a human. Such excipients, carriers, diluents, and buffers include any pharmaceutical agent that can be administered without undue toxicity. Pharmaceutically acceptable excipients include, but are not limited to, liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as pH buffering substances may be present in such vehicles. A wide variety of pharmaceutically acceptable excipients are known in the art and need not be discussed in detail herein. Pharmaceutically acceptable excipients have been amply described in a variety of publications, including, for example, A. Gennaro (2000) Remington: The Science and Practice of Pharmacy, 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

To prepare the composition, the rAAV virion is generated and purified as necessary or desired. The rAAV can be mixed with or suspended in a pharmaceutically acceptable carrier. These rAAV can be adjusted to an appropriate concentration, and optionally combined with other agents. The concentration of rAAV virion and/or other agent included in a unit dose can vary widely. The dose and the number of administrations can be optimized by those skilled in the art. For example, about 10²-10¹⁰ vector genomes (vg) may be administered. In some embodiments, the dose is at least about 10² vg, about 10³ vg, about 10⁴ vg, about 10⁵ vg, about 10⁶ vg, about 10⁷ vg, about 10⁸ vg, about 10⁹ vg, about 10¹⁰ vg, or more vector genomes. Daily doses of the compounds can vary as well. Such daily doses can range, for example, from at least about 10² vg/day, about 10³ vg/day, about 10⁴ vg/day, to about 10⁵ vg/day, about 10⁶ vg/day, about 10⁷ vg/day, about 10⁸ vg/day, about 10⁹ vg/day, about 10¹⁰ vg/day, or more vector genomes per day.

In certain embodiments, the pharmaceutical composition further comprises, and/or the method of treatment is enhanced by the administration of, one or more anti-inflammatory agents, e.g., an anti-inflammatory steroid or a nonsteroidal anti-inflammatory drug (NSAID).

Anti-inflammatory steroids for use in the invention include the corticosteroids, and in particular those with glucocorticoid activity, e.g., dexamethasone and prednisone. Nonsteroidal anti-inflammatory drugs (NSAIDs) for use in the invention generally act by blocking the production of prostaglandins that cause inflammation and pain, cyclooxygenase-1 (COX-1) and/or cyclooxygenase-2 (COX-2). Traditional NSAIDs work by blocking both COX-1 and COX-2. The COX-2 selective inhibitors block only the COX-2 enzyme. In certain embodiment, the NSAID is a COX-2 selective inhibitor, e.g., celecoxib (Celebrex©), rofecoxib (Vioxx), and valdecoxib (B extra). In certain embodiments, the anti-inflammatory is an NSAID prostaglandin inhibitor, e.g., Piroxicam.

The amount of rAAV virion for use in treatment will vary not only with the particular carrier selected but also with the route of administration, the nature of the condition being treated and the age and condition of the patient. Ultimately, the attendant health care provider may determine proper dosage. A pharmaceutical composition may be formulated with the appropriate ratio of each compound in a single unit dosage form for administration with or without cells. Cells or vectors can be separately provided and either mixed with a liquid solution of the compound composition, or administered separately.

Recombinant AAV can be formulated for parenteral administration (e.g., by injection, for example, bolus injection or continuous infusion) and may be presented in unit dosage form in ampoules, prefilled syringes, small volume infusion containers or multi-dose containers with an added preservative. The pharmaceutical compositions can take the form of suspensions, solutions, or emulsions in oily or aqueous vehicles, and can contain formulatory agents such as suspending, stabilizing and/or dispersing agents. Suitable carriers include saline solution, phosphate buffered saline, and other materials commonly used in the art.

The pharmaceutical compositions can also contain other ingredients such as agents useful for treatment of cardiac diseases, conditions and injuries, such as, for example, an anticoagulant (e.g., dalteparin (fragmin), danaparoid (orgaran), enoxaparin (lovenox), heparin, tinzaparin (innohep), and/or warfarin (coumadin)), an antiplatelet agent (e.g., aspirin, ticlopidine, clopidogrel, or dipyridamole), an angiotensin-converting enzyme inhibitor (e.g., Benazepril (Lotensin), Captopril (Capoten), Enalapril (Vasotec), Fosinopril (Monopril), Lisinopril (Prinivil, Zestril), Moexipril (Univasc), Perindopril (Aceon), Quinapril (Accupril), Ramipril (Altace), and/or Trandolapril (Mavik)), angiotensin II receptor blockers (e.g., Candesartan (Atacand), Eprosartan (Teveten), Irbesartan (Avapro), Losartan (Cozaar), Telmisartan (Micardis), and/or Valsartan (Diovan)), a beta blocker (e.g., Acebutolol (Sectral), Atenolol (Tenormin), Betaxolol (Kerlone), Bisoprolol/hydrochlorothiazide (Ziac), Bisoprolol (Zebeta), Carteolol (Cartrol), Metoprolol (Lopressor, Toprol XL), Nadolol (Corgard), Propranolol (Inderal), Sotalol (Betapace), and/or Timolol (Blocadren)), Calcium Channel Blockers (e.g., Amlodipine (Norvasc, Lotrel), Bepridil (Vascor), Diltiazem (Cardizem, Tiazac), Felodipine (Plendil), Nifedipine (Adalat, Procardia), Nimodipine (Nimotop), Nisoldipine (Sular), Verapamil (Calan, Isoptin, Verelan), diuretics (e.g, Amiloride (Midamor), Bumetanide (Bumex), Chlorothiazide (Diuril), Chlorthalidone (Hygroton), Furosemide (Lasix), Hydro-chlorothiazide (Esidrix, Hydrodiuril), Indapamide (Lozol) and/or Spironolactone (Aldactone)), vasodilators (e.g., Isosorbide dinitrate (Isordil), Nesiritide (Natrecor), Hydralazine (Apresoline), Nitrates and/or Minoxidil), statins, nicotinic acid, gemfibrozil, clofibrate, Digoxin, Digitoxin, Lanoxin, or any combination thereof.

Additional agents can also be included such as antibacterial agents, antimicrobial agents, anti-viral agents, biological response modifiers, growth factors; immune modulators, monoclonal antibodies and/or preservatives. The compositions of the invention may also be used in conjunction with other forms of therapy.

The pharmaceutical compositions comprising the rAAV virions described herein can be administered to a subject to treat a disease or disorder. Such a composition may be in a single dose, in multiple doses, in a continuous or intermittent manner, depending, for example, upon the recipient's physiological condition, whether the purpose of the administration is in response to traumatic injury or for more sustained therapeutic purposes, and other factors known to skilled practitioners. The administration of the compounds and compositions of the invention may be essentially continuous over a preselected period of time or may be in a series of spaced doses. Both local and systemic administration is contemplated. In some embodiments, localized delivery of rAAV virion is achieved. In some embodiments, localized delivery of rAAV virions is used to generate a population of cells within the heart. In some embodiments, such a localized population operates as “pacemaker cells” for the heart. In some embodiments, the rAAV virions are used to generate, regenerate, repair, replace, and/or rejuvenate one or more of a sinoatrial (SA) node, an atrioventricular (AV) node, a bindle of His, and/or Purkinje fibers.

To control tonicity, an aqueous pharmaceutical composition can comprise a physiological salt, such as a sodium salt. Sodium chloride (NaCl) is preferred, which may be present at between 1 and 20 mg/ml. Other salts that may be present include potassium chloride, potassium dihydrogen phosphate, disodium phosphate dehydrate, magnesium chloride and calcium chloride.

Compositions may include one or more buffers. Typical buffers include: a phosphate buffer; a Tris buffer; a borate buffer; a succinate buffer; a histidine buffer; or a citrate buffer. Buffers will typically be included at a concentration in the 5-20 mM range. The pH of a composition will generally be between 5 and 8, and more typically between 6 and 8, e.g. between 6.5 and 7.5, or between 7.0 and 7.8.

The composition is preferably sterile. The composition is preferably gluten free. The composition is preferably non-pyrogenic.

In some embodiments, a composition comprising cells may include a cryoprotectant agent. Non-limiting examples of cryoprotectant agents include a glycol (e.g., ethylene glycol, propylene glycol, and glycerol), dimethyl sulfoxide (DMSO), formamide, sucrose, trehalose, dextrose, and any combinations thereof.

One or more of the following types of compounds can also be present in the composition with the rAAV virions: a WNT agonist, a GSK3 inhibitor, a TGF-beta signaling inhibitor, an epigenetic modifier, LSD1 inhibitor, an adenylyl cyclase agonist, or any combination thereof.

### Kits

In some embodiments, provided are kits that include any composition (e.g. an rAAV virion) according to various embodiments disclosed herein. In some embodiments, provided are kits that include any composition (e.g. rAAV virions) comprising an engineered capsid protein according to various embodiments disclosed herein.

The kit can include any of compositions described herein, either mixed together or individually packaged, and in dry or hydrated form. The rAAV virions and/or other agents described herein can be packaged separately into discrete vials, bottles or other containers. Alternatively, any of the rAAV virions and/or agents described herein can be packaged together as a single composition, or as two or more compositions that can be used together or separately. The compounds and/or agents described herein can be packaged in appropriate ratios and/or amounts to facilitate conversion of selected cells across differentiation boundaries to form cardiac progenitor cells and/or cardiomyocytes.

The kit can include instructions for administering those compositions, compounds and/or agents. Such instructions can provide the information described throughout this application. The rAAV virion or pharmaceutical composition can be provided within any of the kits in the form of a delivery device. Alternatively a delivery device can be separately included in the kits, and the instructions can describe how to assemble the delivery device prior to administration to a subject.

Any of the kits can also include syringes, catheters, scalpels, sterile containers for sample or cell collection, diluents, pharmaceutically acceptable carriers, and the like. The kits can provide other factors such as any of the supplementary factors or drugs described herein for the compositions in the preceding section or other parts of the application.

### EXAMPLES

### Example 1. Reconstitution of Sequence Seeds at AAV9 VR-VIII Site

This example discloses design and identification of AAV capsids with improved properties for cardiac gene delivery. Sequence variants of the AAV9 VR-VIII region were designed by reconstituting sequences from various combinations of short sequence seeds. Short sequence seeds were categorized according to position in the sequence, based on VP1 numbering. Some sequence seeds contain insertion(s).

A schematic of an exemplary process used to generate these “reconstitution variants” is shown in FIG. 1. Each final reconstitution variant sequence comprises one sequence from each category:
(1) amino acid positions 581-585, consisting of one short sequence seed;
(2) amino acid positions 586-590 (including the insertion sequence), consisting of three short sequence seeds; and
(3) amino acid positions 591-595, consisting of one short sequence seed.

The final reconstituted sequences were 22 amino acids long and were used to replace the wild-type 15 amino acid sequence (581 to 595, based on VP1 numbering) in the AAV9 capsid protein to create new variants. Non-limiting examples of sequence seeds and reconstituted final sequences are shown.

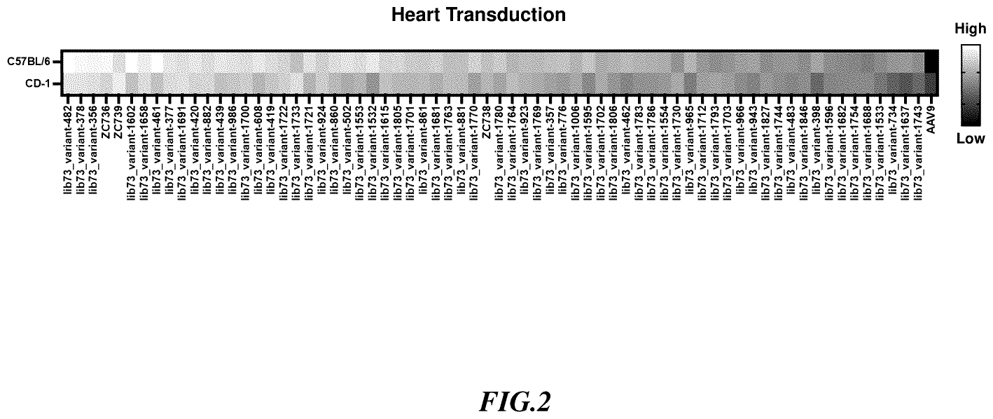

Reconstituted variants were synthesized, and the heart transduction efficiency of selected reconstitution variants was tested in a pooled study in C57BL/6 and CD-1 mice. Briefly, the variant virus pool was injected to mice at 3E+13 vg/kg dose through retro-orbital administration. At 3-week post-injection, animals were sacrificed and heart samples were collected. Viral RNA transcripts were amplified from heart samples and sequenced by next-generation sequencing (NGS). Heart transduction efficiencies were measured by NGS-based viral RNA expression quantification and are shown in FIG. 2. All candidates showed superior heart transduction efficiency relative to wild-type AAV9.

### INCORPORATION BY REFERENCE

Various references such as patents, patent applications, and publications are cited herein, the disclosures of which are hereby incorporated herein by reference in their entireties. Also, all references mentioned herein are specifically incorporated by reference to disclose and describe the methods and/or materials in connection with which the publications are cited.

1. An engineered adeno-associated virus (AAV) capsid protein, wherein the capsid protein comprises an amino acid substitution at least one, two, three, four, or five of the following positions relative to a wild-type AAV9 capsid protein sequence: S586, A587, Q588, A589, and Q590, wherein the amino acid numbering is according to the AAV9 VP1 sequence of SEQ ID NO:1.

2. The engineered capsid protein of claim 1, wherein the amino acid substitutions are selected from S586E, S586A, A587S, A587N, Q588V, Q588R, Q588T, A589T, A589N, A589S, Q590G, Q590L, and Q590R.

3. The engineered capsid protein of claim 1 or 2 comprising at least four amino acid substitutions relative to a wild-type or a parental AAV capsid protein, wherein the amino acid substitutions are selected from S586E, S586A, A587N, Q588R, and Q588T.

4. The engineered capsid protein of any one of claims 1-3, comprising amino acid substitutions selected from:
a) S586E, A587N, Q588R, and A589T;
b) S586A, A587S, Q588T, and Q590G
c) S586E, A587N, Q588R, A589T, and Q590L
d) S586A, A587S, Q588T, A589T, and Q590L;
e) S586E, A587N, Q588R, A589N, Q590R;
f) S586A, A587S, Q588T, and A589T; and
g) S586A, A587S, Q588T, A589S, and Q590G.

5. The engineered capsid protein of any one of claims 1-3, comprising amino acid substitutions selected from:
a) A587S and Q588V;
b) S586E, A587N, Q588R, and A589T;
c) S586A, A587S, Q588T, and Q590G
d) S586E, A587N, Q588R, A589T, and Q590L
e) S586E, A587N, and Q588R;
f) S586A, A587S, and Q588T;
g) S586A, A587S, Q588T, A589T, and Q590L;
h) S586E, A587N, Q588R, A589N, Q590R;
i) S586A, A587S, Q588T, and A589T; and
j) S586A, A587S, Q588T, A589S, and Q590G.

6. The engineered capsid protein of claim 4 or claim 5, comprising amino acid substitutions S586E, A587N, Q588R, and A589T.

7. The engineered capsid protein of claim 4 or claim 5, comprising amino acid substitutions S586E, A587N, and Q588R.

8. The engineered capsid protein of claim 4 or claim 5, comprising amino acid substitutions A587S, and Q588V.

9. The engineered capsid protein of claims 1-8, further comprising a polypeptide sequence inserted between positions 588 and 589, wherein the polypeptide sequence comprises an amino acid sequence RX₁DX₂X₃X₄X₅, wherein:
X₁ is Glycine (G) or Threonine (T);
X₂ is Histidine (H), Serine (S), Alanine (A), Leucine (L), Threonine (T), Glycine (G), Valine (V), or Phenylalanine (F);
X₃ is Glycine (G), Alanine (A), Valine (V), Lysine (K), Asparagine (N), or Threonine (T);
X₄ is Valine (V), Serine (S), Glycine (G), Asparagine (N), or Arginine (R); and
X₅ is Leucine (L), Tryptophan (W), Threonine (T), Glycine (G), or Arginine (R).

10. The engineered capsid protein of claim 9, wherein the polypeptide sequence is selected from SEQ ID NOs: 215-242.

11. The engineered capsid protein of claim 9 or claim 10, wherein the polypeptide sequence is SEQ ID NO: 234.

12. The engineered capsid protein of claim 9 or claim 10, wherein the polypeptide sequence is SEQ ID NO: 218.

13. The engineered capsid protein of claim 9 or claim 10, wherein the polypeptide sequence is SEQ ID NO: 241.

14. An engineered adeno-associated virus (AAV) capsid protein, comprising a non-naturally occurring amino acid motif comprising an amino acid sequence of X₁X₂X₃RX₄DX₅X₆X₇X₈ X₉X₁₀ in the VR-VIII site, wherein:
X₁ is Serine (S), Glutamic acid (E), or Alanine (A);
X₂ is Alanine (A), Serine (S), or Asparagine (N);
X₃ is Glutamine (Q), Valine (V), Arginine (R), or Threonine (T);
X₄ is Glycine (G) or Threonine (T);
X₅ is Histidine (H), Serine (S), Alanine (A), Leucine (L), Threonine (T), Glycine (G), Valine (V), or Phenylalanine (F);
X₆ is Glycine (G), Alanine (A), Valine (V), Lysine (K), Asparagine (N), or Threonine (T);
X₇ is Valine (V), Serine (S), Glycine (G), Asparagine (N), or Arginine (R);
X₈ is Leucine (L), Tryptophan (W), Threonine (T), Glycine (G), or Arginine (R);
X₉ is Alanine (A), Threonine (T), Asparagine (N), or Serine (S); and
X₁₀ is Glutamine (Q), Glycine (G), Leucine (L), or Arginine (R).

15. The engineered capsid protein of claim 14, wherein the non-naturally occurring amino acid motif comprises:
(a) an amino acid sequence selected from any one of SEQ ID NOs: 78-145, or
(b) an amino acid sequence having no more than 1 or 2 amino acid substitutions in the amino acid sequence selected from any one of SEQ ID NOs: 78-145.

16. The engineered capsid protein of claim 15, wherein the non-naturally occurring amino acid motif comprises SEQ ID NO: 81.

17. The engineered capsid protein of claim 15, wherein the non-naturally occurring amino acid motif comprises SEQ ID NO: 119.

18. The engineered capsid protein of claim 15, wherein the non-naturally occurring amino acid motif comprises SEQ ID NO: 135.

19. The engineered capsid protein of claim 15, wherein the 1 or 2 amino acid substitutions are conservative amino acid substitutions.

20. The engineered capsid protein of any one of claims 14-19, wherein the engineered AAV capsid protein is a variant of an AAV5, AAV9, AAVrh.74, or AAVrh.10 capsid protein.

21. The engineered capsid protein of any one of claims 14-20, wherein the non-naturally occurring amino acid motif comprises an amino acid insertion.

22. The engineered capsid protein of any one of claims 14-21, wherein the non-naturally occurring amino acid motif comprises an amino acid substitution, wherein the amino acid substitution is generated by one, two, three, four, five, or more amino acid substitutions in the amino acid sequence of the wild-type or parental AAV capsid protein.

23. An engineered adeno-associated virus (AAV) capsid protein, wherein the engineered capsid protein:
(i) comprises at least 80% amino acid sequence identity to SEQ ID NO: 3, and comprises an amino acid sequence of any one of SEQ ID NOs: 78-145 replacing the natural amino acid sequence at amino acid positions 586 to 590, wherein the amino acid numbering is according to SEQ ID NO: 1;
(ii) comprises at least 80% amino acid sequence identity to SEQ ID NO: 12, and comprises an amino acid sequence of any one of SEQ ID NOs: 78-145 replacing the natural amino acid sequence at amino acid positions 575 to 579, wherein the amino acid numbering is according to SEQ ID NO: 10;
(iii) comprises at least 80% amino acid sequence identity to SEQ ID NO: 21 and comprises an amino acid sequence of any one of SEQ ID NOs: 78-145 replacing the natural amino acid sequence at amino acid positions 588 to 592, wherein the amino acid numbering is according to SEQ ID NO: 19; or
(iv) comprises at least 80% amino acid sequence identity to SEQ ID NO: 30, and comprises an amino acid sequence of any one of SEQ ID NOs: 78-145 replacing the natural amino acid sequence at amino acid positions 588 to 592, wherein the amino acid numbering is according to SEQ ID NO: 28.

24. An engineered adeno-associated virus (AAV) capsid protein, wherein the engineered capsid protein:
(i) comprises at least 80% amino acid sequence identity to SEQ ID NO: 3, and comprises SEQ ID NO: 81 replacing the natural amino acid sequence at amino acid positions 586 to 590, wherein the amino acid numbering is according to SEQ ID NO: 1;
(ii) comprises at least 80% amino acid sequence identity to SEQ ID NO: 12, and comprises SEQ ID NO: 81 replacing the natural amino acid sequence at amino acid positions 575 to 579, wherein the amino acid numbering is according to SEQ ID NO: 10;
(iii) comprises at least 80% amino acid sequence identity to SEQ ID NO: 21, and comprises SEQ ID NO: 81 replacing the natural amino acid sequence at amino acid positions 588 to 592, wherein the amino acid numbering is according to SEQ ID NO: 19; or
(iv) comprises at least 80% amino acid sequence identity to SEQ ID NO: 30, and comprises SEQ ID NO: 81 replacing the natural amino acid sequence at amino acid positions 588 to 592, wherein the amino acid numbering is according to SEQ ID NO: 28.

25. An engineered adeno-associated virus (AAV) capsid protein, wherein the engineered capsid protein:
(i) comprises at least 80% amino acid sequence identity to SEQ ID NO: 3, and comprises SEQ ID NO: 119 replacing the natural amino acid sequence at amino acid positions 586 to 590, wherein the amino acid numbering is according to SEQ ID NO: 1;
(ii) comprises at least 80% amino acid sequence identity to SEQ ID NO: 12, and comprises SEQ ID NO: 119 replacing the natural amino acid sequence at amino acid positions 575 to 579, wherein the amino acid numbering is according to SEQ ID NO: 10;
(iii) comprises at least 80% amino acid sequence identity to SEQ ID NO: 21, and comprises SEQ ID NO: 119 replacing the natural amino acid sequence at amino acid positions 588 to 592, wherein the amino acid numbering is according to SEQ ID NO: 19; or
(iv) comprises at least 80% amino acid sequence identity to SEQ ID NO: 30, and comprises SEQ ID NO: 119 replacing the natural amino acid sequence at amino acid positions 588 to 592, wherein the amino acid numbering is according to SEQ ID NO: 28.

26. An engineered adeno-associated virus (AAV) capsid protein, wherein the engineered capsid protein:
(i) comprises at least 80% amino acid sequence identity to SEQ ID NO: 3, and comprises SEQ ID NO: 135 replacing the natural amino acid sequence at amino acid positions 586 to 590, wherein the amino acid numbering is according to SEQ ID NO: 1;
(ii) comprises at least 80% amino acid sequence identity to SEQ ID NO: 12, and comprises SEQ ID NO: 135 replacing the natural amino acid sequence at amino acid positions 575 to 579, wherein the amino acid numbering is according to SEQ ID NO: 10;
(iii) comprises at least 80% amino acid sequence identity to SEQ ID NO: 21, and comprises SEQ ID NO: 135 replacing the natural amino acid sequence at amino acid positions 588 to 592, wherein the amino acid numbering is according to SEQ ID NO: 19; or
(iv) comprises at least 80% amino acid sequence identity to SEQ ID NO: 30, and comprises SEQ ID NO: 135 replacing the natural amino acid sequence at amino acid positions 588 to 592, wherein the amino acid numbering is according to SEQ ID NO: 28.

27. An engineered adeno-associated virus (AAV) capsid protein, comprising a non-naturally occurring amino acid motif comprising an amino acid sequence RX₁DX₂X₃X₄X₅ in the VR-VIII site, wherein:
X₁ is Glycine (G) or Threonine (T);
X₂=Histidine (H), Serine (S), Alanine (A), Leucine (L), Threonine (T), Glycine (G), Valine (V), or Phenylalanine (F);
X₃=Glycine (G), Alanine (A), Valine (V), Lysine (K), Asparagine (N), or Threonine (T);
X₄=Valine (V), Serine (S), Glycine (G), Asparagine (N), or Arginine (R); and
X₅=Leucine (L), Tryptophan (W), Threonine (T), Glycine (G), or Arginine (R).

28. The engineered capsid protein of claim 27, wherein the non-naturally occurring amino acid motif comprises an amino acid sequence RX₁DX₂X₃X₄X₅ in the VR-VIII site, wherein:
X₁ is Glycine (G) or Threonine (T);
X₂=Serine (S), Alanine (A), Leucine (L), Threonine (T), Glycine (G), or Valine (V);
X₃=Glycine (G), Alanine (A), or Asparagine (N);
X₄=Valine (V), Serine (S), or Asparagine (N); and
X₅=Leucine (L), Tryptophan (W), or Threonine (T).

29. The engineered capsid protein of claim 28, wherein the non-naturally occurring amino acid motif comprises an amino acid sequence selected from any one of SEQ ID NOs: 215-227.

30. The engineered capsid protein of claim 28 or claim 29, wherein the non-naturally occurring amino acid motif comprises SEQ ID NO: 234.

31. The engineered capsid protein of claim 28 or claim 29, wherein the non-naturally occurring amino acid motif comprises SEQ ID NO: 218.

32. The engineered capsid protein of claim 28 or claim 29, wherein the non-naturally occurring amino acid motif comprises SEQ ID NO: 241.

33. An engineered adeno-associated virus (AAV) capsid protein, wherein the engineered capsid protein comprises at least 90% or at least 95% amino acid sequence identity to AAV9 VP3 SEQ ID NO: 3, and comprises the amino acid sequence of any one of SEQ ID NOs: 147-214 replacing the natural amino acid sequence at amino acid positions 581 to 595, wherein the amino acid numbering is according to AAV9 VP1 SEQ ID NO: 1.

34. The engineered AAV capsid protein of claim 33, wherein the engineered capsid protein comprises at least 90% or at least 95% amino acid sequence identity to AAV9 VP3 SEQ ID NO: 3, and comprises the amino acid sequence of any one of SEQ ID NO: 150 replacing the natural amino acid sequence at amino acid positions 581 to 595, wherein the amino acid numbering is according to AAV9 VP1 SEQ ID NO: 1.

35. The engineered AAV capsid protein of claim 33, wherein the engineered capsid protein comprises at least 90% or at least 95% amino acid sequence identity to AAV9 VP3 SEQ ID NO: 3, and comprises the amino acid sequence of any one of SEQ ID NO: 188 replacing the natural amino acid sequence at amino acid positions 581 to 595, wherein the amino acid numbering is according to AAV9 VP1 SEQ ID NO: 1.

36. The engineered AAV capsid protein of claim 33, wherein the engineered capsid protein comprises at least 90% or at least 95% amino acid sequence identity to AAV9 VP3 SEQ ID NO: 3, and comprises the amino acid sequence of any one of SEQ ID NO: 204 replacing the natural amino acid sequence at amino acid positions 581 to 595, wherein the amino acid numbering is according to AAV9 VP1 SEQ ID NO: 1.

37. An engineered adeno-associated virus (AAV) capsid protein, comprising or consisting of an amino acid sequence that shares at least 80%, at least 85%, at least 90%, at least 95%, at least 96% at least 97%, at least 98%, at least 99%, or 100% identity to any one of SEQ ID NOs: 243-310.

38. The engineered AAV capsid protein of claim 37, comprising or consisting of an amino acid sequence that shares at least 80%, at least 85%, at least 90%, at least 95%, at least 96% at least 97%, at least 98%, at least 99%, or 100% identity to SEQ ID NO: 246.

39. The engineered AAV capsid protein of claim 37, comprising or consisting of an amino acid sequence that shares at least 80%, at least 85%, at least 90%, at least 95%, at least 96% at least 97%, at least 98%, at least 99%, or 100% identity to SEQ ID NO: 284.

40. The engineered AAV capsid protein of claim 37, comprising or consisting of an amino acid sequence that shares at least 80%, at least 85%, at least 90%, at least 95%, at least 96% at least 97%, at least 98%, at least 99%, or 100% identity to SEQ ID NO: 300.

41. A recombinant adeno-associated virus (rAAV) virion, comprising the engineered capsid protein according to any one of claims 1-40 and a vector genome comprising an expression cassette flanked by inverted terminal repeats (ITRs).

42. The rAAV virion of claim 41, wherein the rAAV virion transduces heart cells.

43. The rAAV virion of claim 41 or claim 42, wherein the rAAV virion transduces cardiomyocytes.

44. The rAAV virion of any one of claims 41-43, wherein the rAAV virion traffics to at least one organ other than the liver.

45. The rAAV virion of any one of claims 41-44, wherein the rAAV virion traffics to the heart.

46. The rAAV virion of any one of claims 41-45, wherein the rAAV virion exhibits a higher heart transduction efficiency than an rAAV virion having an AAV9 VP1 capsid protein according to SEQ ID NO: 1.

47. The rAAV virion of any one of claims 41-46, wherein the polynucleotide cassette comprises a polynucleotide sequence encoding MYBPC3, DWORF, PKP2, KCNH2, TRPM4, DSG2, TGFBR2, TGFBR1, EMD, KCNQ1, TAZ, COL3A1, JUP, CASQ2, MLRP44, DNAJC19, LMNA, TNNI3, DSP, DSG2, RAF1, SOS1, FBN1, LAMP2, FXN, RAF1, BAG3, KCNQ1, MYLK3, CRYAB, ALPK3, ACTN2, JPH2, PLN, ATP2A2, CACNA1C, DMD, DMPK, EPG5, EVC, EVC2, FBN1, NF1, SCN5A, SOS1, NPR1, ERBB4, VIP, MYH6, MYH7, Cas9, split Cas9, RBM20, MYOCD, ASCL1, GATA4, MEF2C, TBX5, miR-133, or MESP1, or SYNPO2L.

48. The rAAV virion of any one of claims 41-47, wherein the polynucleotide cassette comprises a polynucleotide sequence which encodes a protein selected from the group consisting of: MYBPC3, DWORF, PKP2, LMNA, LAMP2, BAG3, CRYAB, JPH2, PLN, TTNI3, MYOCD, ASCL1, DSP, JUP, DSP, MYH6, MYH7, RBM20, Cas9, and splitCas9.

49. A pharmaceutical composition comprising an rAAV virion according to any one of claims 41-48 and a pharmaceutically acceptable carrier.

50. A polynucleotide encoding the capsid protein of any one of claims 1-40.

51. A method of transducing a cardiac cell, comprising contacting the cardiac cell with an rAAV virion according to any one of claims 41-48, wherein the rAAV virion transduces the cardiac cell.

52. The method of claim 51, wherein the cardiac cell is a cardiomyocyte.

53. The method of claim 51 or claim 52, wherein the rAAV virion exhibits higher transduction efficiency in the cell than an rAAV virion having an AAV9 VP1 capsid protein according to SEQ ID NO: 1.

54. A method of delivering one or more gene products to a cardiac cell, comprising contacting the cardiac cell with an rAAV virion according to any one of claims 20-27.

55. The method of claim 54, wherein the cardiac cell is a cardiomyocyte.

56. A method of treating a cardiac pathology in a subject in need thereof, comprising administering a therapeutically effective amount of an rAAV virion according to any one of claims 20-27 to the subject, wherein the rAAV virion transduces cardiac tissue.

57. A method of treating a heart disease or condition in a subject in need thereof, comprising administering a therapeutically effective amount of an rAAV virion according to any one of claims 41-48 to the subject.

58. A kit comprising a pharmaceutical composition according to claim 49 and instructions for use.


SEQUENCE LISTING (365 sequences)

SEQ ID NO: 1 - protein, 736 aa, Adeno-associated dependoparvovirus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 2 - protein, 599 aa, Adeno-associated dependoparvovirus
TAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 3 - protein, 534 aa, Adeno-associated dependoparvovirus
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 4 - protein, 8 aa, Adeno-associated dependoparvovirus
NSTSGGSS

SEQ ID NO: 5 - protein, 5 aa, Adeno-associated dependoparvovirus
NNGVK

SEQ ID NO: 6 - protein, 15 aa, Adeno-associated dependoparvovirus
SKTINGSGQNQQTLK

SEQ ID NO: 7 - protein, 14 aa, Adeno-associated dependoparvovirus
TTVTQNNNSEFAWP

SEQ ID NO: 8 - protein, 11 aa, Adeno-associated dependoparvovirus
GTGRDNVDADK

SEQ ID NO: 9 - protein, 15 aa, Adeno-associated dependoparvovirus
ATNHQSAQAQAQTGW

SEQ ID NO: 10 - protein, 724 aa, Adeno-associated dependoparvovirus
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 11 - protein, 588 aa, Adeno-associated dependoparvovirus
TAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 12 - protein, 532 aa, Adeno-associated dependoparvovirus
MSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 13 - protein, 5 aa, Adeno-associated dependoparvovirus
SGSVD

SEQ ID NO: 14 - protein, 5 aa, Adeno-associated dependoparvovirus
QDSTT

SEQ ID NO: 15 - protein, 25 aa, Adeno-associated dependoparvovirus
RFVSTNNTGGVQFNKNLAGRYANTY

SEQ ID NO: 16 - protein, 14 aa, Adeno-associated dependoparvovirus
LGSGVNRASVSAFA

SEQ ID NO: 17 - protein, 14 aa, Adeno-associated dependoparvovirus
PANPGTTATYLEGN

SEQ ID NO: 18 - protein, 15 aa, Adeno-associated dependoparvovirus
ATNNQSSTTAPATGT

SEQ ID NO: 19 - protein, 738 aa, Adeno-associated dependoparvovirus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 20 - protein, 601 aa, Adeno-associated dependoparvovirus
TAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 21 - protein, 535 aa, Adeno-associated dependoparvovirus
MAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 22 - protein, 5 aa, Adeno-associated dependoparvovirus
NGTSG

SEQ ID NO: 23 - protein, 5 aa, Adeno-associated dependoparvovirus
NEGTK

SEQ ID NO: 24 - protein, 16 aa, Adeno-associated dependoparvovirus
SRTQSTGGTAGTQQLL

SEQ ID NO: 25 - protein, 14 aa, Adeno-associated dependoparvovirus
TTLSQNNNSNFAWT

SEQ ID NO: 26 - protein, 11 aa, Adeno-associated dependoparvovirus
GAGKDNVDYSS

SEQ ID NO: 27 - protein, 15 aa, Adeno-associated dependoparvovirus
ADNLQQQNAAPIVGA

SEQ ID NO: 28 - protein, 738 aa, Adeno-associated dependoparvovirus
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 29 - protein, 601 aa, Adeno-associated dependoparvovirus
TAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 30 - protein, 535 aa, Adeno-associated dependoparvovirus
MAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 31 - protein, 50 aa, synthetic construct
MAADGYLPDWLEDNLSEGIREWWALKPGAPQPKANQQHQDNARGLVLPGY

SEQ ID NO: 32 - protein, 49 aa, synthetic construct
MSFVDHPPDWLEEVGEGLREFLGLEAGPPKPKPNQQHQDQARGLVLPGY

SEQ ID NO: 33 - protein, 34 aa, synthetic construct
KYLGPGNGLDKGEPVNAADAAALEHDKAYDQQLK

SEQ ID NO: 34 - protein, 34 aa, synthetic construct
NYLGPGNGLDRGEPVNRADEVAREHDISYNEQLE

SEQ ID NO: 35 - protein, 44 aa, synthetic construct
AGDNPYLKYNHADAEFQERLKEDTSFGGNLGRAVFQAKKRLLEP

SEQ ID NO: 36 - protein, 44 aa, synthetic construct
AGDNPYLKYNHADAEFQEKLADDTSFGGNLGKAVFQAKKRVLEP

SEQ ID NO: 37 - protein, 84 aa, synthetic construct
LGLVEEAAKTAPGKKRPVEQSPQEPDSSAGIGKSGAQPAKKRLNFGQTGDTESVPDPQPIGEPPAAPSGVGSLTMASGGGAPVA

SEQ ID NO: 38 - protein, 75 aa, synthetic construct
FGLVEEGAKTAPTGKRIDDHFPKRKKARTEEDSKPSTSSDAEAGPSGSQQLQIPAQPASSLGADTMSAGGGGPLG

SEQ ID NO: 39 - protein, 524 aa, synthetic construct
DNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 40 - protein, 524 aa, synthetic construct
DNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELKKENSKRWNPEIQYTSNYYKSNNVEFAVNTEGVYSEPRPIGTRYLTRNL

SEQ ID NO: 41 - protein, 726 aa, synthetic construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTNNYNDPQFVDFAPDSTGEYRTTRPIGTRYLTRPL

SEQ ID NO: 42 - protein, 725 aa, synthetic construct
MAADGYLPDWLEDNLSEGIREWWALKPGAPQPKANQQHQDNARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTSNYYKSNNVEFAVNTEGVYSEPRPIGTRYLTRNL

SEQ ID NO: 43 - protein, 725 aa, synthetic construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEAAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 44 - protein, 727 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 45 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVTVEMEWELKKENSKRWNPEIQYTNNYNDPQFVDFAPDSTGEYRTTRPIGTRYLTRPL

SEQ ID NO: 46 - protein, 726 aa, synthetic construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSFAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 47 - protein, 726 aa, synthetic construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 48 - protein, 726 aa, synthetic construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTNNYNDPQFVDFAPDSTGEYRTTRPIGTRYLTRPL

SEQ ID NO: 49 - protein, 727 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVTVEMEWELKKENSKRWNPEIQYTSNYYKSNNVEFAVNTEGVYSEPRPIGTRYLTRNL

SEQ ID NO: 50 - protein, 726 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 51 - protein, 726 aa, synthetic construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 52 - protein, 736 aa, synthetic construct
MAADGYLPDWLEDNLSEGIREWWALKPGAPQPKANQQHQDNARGLVLPGYNYLGPGNGLDRGEPVNRADEVAREHDISYNEQLEAGDNPYLKYNHADAEFQERLKEDTSFGGNLGKAVFQAKKRVLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGNASGDWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSKMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVTVEMEWELKKENSKRWNPEIQYTNNYNDPQFVDFAPDSTGEYRTTRPIGTRYLTRNL

SEQ ID NO: 53 - protein, 735 aa, synthetic construct
MSFVDHPPDWLEEVGEGLREFLGLEAGPPKPKPNQQHQDQARGLVLPGYNYLGPGNGLDRGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTNNYNDPQFVDFAPDSTGEYRTTRPIGTRYLTRPL

SEQ ID NO: 54 - protein, 735 aa, synthetic construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELKKENSKRWNPEIQYTSNYYKSNNVEFAVNTEGVYSEPRPIGTRYLTRNL

SEQ ID NO: 55 - protein, 726 aa, synthetic construct; identical to SEQ ID NO: 51

SEQ ID NO: 56 - protein, 726 aa, synthetic construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 57 - protein, 726 aa, synthetic construct
MSFVDHPPDWLEEVGEGLREFLGLEAGPPKPKPNQQHQDQARGLVLPGYKYLGPGNGLDKGEPVNAADAAALEHDKAYDQQLKAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 58 - protein, 727 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPFGLVEEGAKTAPTGKRIDDHFPKRKKARTEEDSKPSTSSDAEAGPSGSQQLQIPAQPASSLGADTMSAGGGGPLGDNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 59 - protein, 727 aa, synthetic construct
MAADGYLPDWLEDNLSEGIREWWALKPGAPQPKANQQHQDNARGLVLPGYKYLGPGNGLDKGEPVNAADAAALEHDKAYDQQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 60 - protein, 735 aa, synthetic construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 61 - protein, 736 aa, synthetic construct
MAADGYLPDWLEDNLSEGIREWWALKPGAPQPKANQQHQDNARGLVLPGYNYLGPGNGLDRGEPVNRADEVAREHDISYNEQLEAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 62 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQEKLADDTSFGGNLGKAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 63 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQEKLADDTSFGGNLGKAVFQAKKRVLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 64 - protein, 735 aa, synthetic construct; identical to SEQ ID NO: 53

SEQ ID NO: 65 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRGDLGLSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 66 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGASTNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRGDLLLSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 67 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRGDLLLSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 68 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGASTNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 69 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGVSLGGGQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 70 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEIAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYCKSNNVEFAVNTEGVYSEPRPIGTRYLTRNL

SEQ ID NO: 71 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQENRRGDFNNTAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 72 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRGDHASWAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 73 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSNSRGDYNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 74 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQASTRGDHGVLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 75 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLANKTTNKDARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSINVDFTVDTNGVYSEPRPIGTRYLTRNL

SEQ ID NO: 76 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLANKIQRTDARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSINVDFTVDTNGVYSEPRPIGTRYLTRNL

SEQ ID NO: 77 - protein, 5 aa, synthetic construct
SAQAQ

SEQ ID NO: 78 - protein, 12 aa, synthetic construct
SSVRGDHGVLAQ

SEQ ID NO: 79 - protein, 12 aa, synthetic construct
SSVRGDSASWAQ

SEQ ID NO: 80 - protein, 12 aa, synthetic construct
SSVRGDAGVLAQ

SEQ ID NO: 81 - protein, 12 aa, synthetic construct
ENRRGDLVSTTQ

SEQ ID NO: 82 - protein, 12 aa, synthetic construct
ASTRGDTKGLAG

SEQ ID NO: 83 - protein, 12 aa, synthetic construct
ENRRGDLVSTTL

SEQ ID NO: 84 - protein, 12 aa, synthetic construct
ENRRGDHGVLAQ

SEQ ID NO: 85 - protein, 12 aa, synthetic construct
SSVRGDTGVLAQ

SEQ ID NO: 86 - protein, 12 aa, synthetic construct
SSVRGDSGVLAQ

SEQ ID NO: 87 - protein, 12 aa, synthetic construct
ASTRGDAKGLAG

SEQ ID NO: 88 - protein, 12 aa, synthetic construct
SSVRGDGASWAQ

SEQ ID NO: 89 - protein, 12 aa, synthetic construct
SAQRGDSASWAQ

SEQ ID NO: 90 - protein, 12 aa, synthetic construct
SSVRGDLNNTAQ

SEQ ID NO: 91 - protein, 12 aa, synthetic construct
SAQRGDHGVLAQ

SEQ ID NO: 92 - protein, 12 aa, synthetic construct
ASTRGDAGVLAQ

SEQ ID NO: 93 - protein, 12 aa, synthetic construct
SSVRTDLGVLAQ

SEQ ID NO: 94 - protein, 12 aa, synthetic construct
SSVRGDGGVLAQ

SEQ ID NO: 95 - protein, 12 aa, synthetic construct
ASTRGDSASWAQ

SEQ ID NO: 96 - protein, 12 aa, synthetic construct
ASTRGDVKGLAG

SEQ ID NO: 97 - protein, 12 aa, synthetic construct
ASTRGDSGVLAQ

SEQ ID NO: 98 - protein, 12 aa, synthetic construct
SAQRGDGASWAQ

SEQ ID NO: 99 - protein, 12 aa, synthetic construct
SAQRGDAGVLAQ

SEQ ID NO: 100 - protein, 12 aa, synthetic construct
SSVRGDFNNTAQ

SEQ ID NO: 101 - protein, 12 aa, synthetic construct
ENRRGDSGVLAQ

SEQ ID NO: 102 - protein, 12 aa, synthetic construct
ENRRGDAGVLAQ

SEQ ID NO: 103 - protein, 12 aa, synthetic construct
ENRRGDLNNTAQ

SEQ ID NO: 104 - protein, 12 aa, synthetic construct
ASTRGDTGVLAQ

SEQ ID NO: 105 - protein, 12 aa, synthetic construct
ASTRGDAASWAQ

SEQ ID NO: 106 - protein, 12 aa, synthetic construct
SAQRGDAASWAQ

SEQ ID NO: 107 - protein, 12 aa, synthetic construct
ASTRGDAARLAG

SEQ ID NO: 108 - protein, 12 aa, synthetic construct
ASTRGDGGVLAQ

SEQ ID NO: 109 - protein, 12 aa, synthetic construct
SAQRGDSGVLAQ

SEQ ID NO: 110 - protein, 12 aa, synthetic construct
ASTRGDLVSTTL

SEQ ID NO: 111 - protein, 12 aa, synthetic construct
ENRRGDLGSGNR

SEQ ID NO: 112 - protein, 12 aa, synthetic construct
ASTRGDLVSTTQ

SEQ ID NO: 113 - protein, 12 aa, synthetic construct
ASTRGDGASWAQ

SEQ ID NO: 114 - protein, 12 aa, synthetic construct
SAQRGDGGVLAQ

SEQ ID NO: 115 - protein, 12 aa, synthetic construct
ASTRGDLTGRSG

SEQ ID NO: 116 - protein, 12 aa, synthetic construct
SSVRGDAASWAQ

SEQ ID NO: 117 - protein, 12 aa, synthetic construct
SAQRTDLGVLAQ

SEQ ID NO: 118 - protein, 12 aa, synthetic construct
SAQRGDFNNTAQ

SEQ ID NO: 119 - protein, 12 aa, synthetic construct
ENRRGDGGVLAQ

SEQ ID NO: 120 - protein, 12 aa, synthetic construct
ASTRGDSARLAG

SEQ ID NO: 121 - protein, 12 aa, synthetic construct
ASTRGDTASWAQ

SEQ ID NO: 122 - protein, 12 aa, synthetic construct
SSVRGDTASWAQ

SEQ ID NO: 123 - protein, 12 aa, synthetic construct
ASTRGDLNNTAQ

SEQ ID NO: 124 - protein, 12 aa, synthetic construct
ASTRGDTARLAG

SEQ ID NO: 125 - protein, 12 aa, synthetic construct
ENRRGDSASWAQ

SEQ ID NO: 126 - protein, 12 aa, synthetic construct
ASTRGDVKGLAQ

SEQ ID NO: 127 - protein, 12 aa, synthetic construct
SAQRGDTGVLAQ

SEQ ID NO: 128 - protein, 12 aa, synthetic construct
ASTRGDSKGLAG

SEQ ID NO: 129 - protein, 12 aa, synthetic construct
ASTRGDTKGLAQ

SEQ ID NO: 130 - protein, 12 aa, synthetic construct
ASTRGDSARLAQ

SEQ ID NO: 131 - protein, 12 aa, synthetic construct
SAQRGDTASWAQ

SEQ ID NO: 132 - protein, 12 aa, synthetic construct
SAQRGDLNNTAQ

SEQ ID NO: 133 - protein, 12 aa, synthetic construct
ASTRGDHASWAQ

SEQ ID NO: 134 - protein, 12 aa, synthetic construct
ASTRGDGARLAG

SEQ ID NO: 135 - protein, 12 aa, synthetic construct
SSVRGDHASWAQ

SEQ ID NO: 136 - protein, 12 aa, synthetic construct
ASTRGDFNNTAQ

SEQ ID NO: 137 - protein, 12 aa, synthetic construct
SSVRGDVGVLAQ

SEQ ID NO: 138 - protein, 12 aa, synthetic construct
ENRRGDGASWAQ

SEQ ID NO: 139 - protein, 12 aa, synthetic construct
ASTRGDAARLAQ

SEQ ID NO: 140 - protein, 12 aa, synthetic construct
ASTRGDGKGLAG

SEQ ID NO: 141 - protein, 12 aa, synthetic construct
ASTRGDAKGLAQ

SEQ ID NO: 142 - protein, 12 aa, synthetic construct
ENRRGDAASWAQ

SEQ ID NO: 143 - protein, 12 aa, synthetic construct
SAQRTDVGVLAQ

SEQ ID NO: 144 - protein, 12 aa, synthetic construct
ENRRGDTGVLAQ

SEQ ID NO: 145 - protein, 12 aa, synthetic construct
ASTRGDVASWAQ

SEQ ID NO: 146 - protein, 15 aa, synthetic construct
ATNHQSAQAQAQTGW

SEQ ID NO: 147 - protein, 22 aa, synthetic construct
ATNHQSSVRGDHGVLAQAQTGW

SEQ ID NO: 148 - protein, 22 aa, synthetic construct
ATNHQSSVRGDSASWAQAQTGW

SEQ ID NO: 149 - protein, 22 aa, synthetic construct
ATNHQSSVRGDAGVLAQAQTGW

SEQ ID NO: 150 - protein, 22 aa, synthetic construct
ATNHQENRRGDLVSTTQAQTGW

SEQ ID NO: 151 - protein, 22 aa, synthetic construct
ATNHQASTRGDTKGLAGAQTGW

SEQ ID NO: 152 - protein, 22 aa, synthetic construct
ATNHQENRRGDLVSTTLAQTGW

SEQ ID NO: 153 - protein, 22 aa, synthetic construct
ATNHQENRRGDHGVLAQAQTGW

SEQ ID NO: 154 - protein, 22 aa, synthetic construct
ATNHQSSVRGDTGVLAQAQTGW

SEQ ID NO: 155 - protein, 22 aa, synthetic construct
ATNHQSSVRGDSGVLAQAQTGW

SEQ ID NO: 156 - protein, 22 aa, synthetic construct
ATNHQASTRGDAKGLAGAQTGW

SEQ ID NO: 157 - protein, 22 aa, synthetic construct
ATNHQSSVRGDGASWAQAQTGW

SEQ ID NO: 158 - protein, 22 aa, synthetic construct
ATNHQSAQRGDSASWAQAQTGW

SEQ ID NO: 159 - protein, 22 aa, synthetic construct
ATNHQSSVRGDLNNTAQAQTGW

SEQ ID NO: 160 - protein, 22 aa, synthetic construct
ATNHQSAQRGDHGVLAQAQTGW

SEQ ID NO: 161 - protein, 22 aa, synthetic construct
ATNHQASTRGDAGVLAQAQTGW

SEQ ID NO: 162 - protein, 22 aa, synthetic construct
ATNHQSSVRTDLGVLAQAQTGW

SEQ ID NO: 163 - protein, 22 aa, synthetic construct
ATNHQSSVRGDGGVLAQAQTGW

SEQ ID NO: 164 - protein, 22 aa, synthetic construct
ATNHQASTRGDSASWAQAQTGW

SEQ ID NO: 165 - protein, 22 aa, synthetic construct
ATNHQASTRGDVKGLAGAQTGW

SEQ ID NO: 166 - protein, 22 aa, synthetic construct
ATNHQASTRGDSGVLAQAQTGW

SEQ ID NO: 167 - protein, 22 aa, synthetic construct
ATNHQSAQRGDGASWAQAQTGW

SEQ ID NO: 168 - protein, 22 aa, synthetic construct
ATNHQSAQRGDAGVLAQAQTGW

SEQ ID NO: 169 - protein, 22 aa, synthetic construct
ATNHQSSVRGDFNNTAQAQTGW

SEQ ID NO: 170 - protein, 22 aa, synthetic construct
ATNHQENRRGDSGVLAQAQTGW

SEQ ID NO: 171 - protein, 22 aa, synthetic construct
ATNHQENRRGDAGVLAQAQTGW

SEQ ID NO: 172 - protein, 22 aa, synthetic construct
ATNHQENRRGDLNNTAQAQTGW

SEQ ID NO: 173 - protein, 22 aa, synthetic construct
ATNHQASTRGDTGVLAQAQTGW

SEQ ID NO: 174 - protein, 22 aa, synthetic construct
ATNHQASTRGDAASWAQAQTGW

SEQ ID NO: 175 - protein, 22 aa, synthetic construct
ATNHQSAQRGDAASWAQAQTGW

SEQ ID NO: 176 - protein, 22 aa, synthetic construct
ATNHQASTRGDAARLAGAQTGW

SEQ ID NO: 177 - protein, 22 aa, synthetic construct
ATNHQASTRGDGGVLAQAQTGW

SEQ ID NO: 178 - protein, 22 aa, synthetic construct
ATNHQSAQRGDSGVLAQAQTGW

SEQ ID NO: 179 - protein, 22 aa, synthetic construct
ATNHQASTRGDLVSTTLAQTGW

SEQ ID NO: 180 - protein, 22 aa, synthetic construct
ATNHQENRRGDLGSGNRAQTGW

SEQ ID NO: 181 - protein, 22 aa, synthetic construct
ATNHQASTRGDLVSTTQAQTGW

SEQ ID NO: 182 - protein, 22 aa, synthetic construct
ATNHQASTRGDGASWAQAQTGW

SEQ ID NO: 183 - protein, 22 aa, synthetic construct
ATNHQSAQRGDGGVLAQAQTGW

SEQ ID NO: 184 - protein, 22 aa, synthetic construct
ATNHQASTRGDLTGRSGAQTGW

SEQ ID NO: 185 - protein, 22 aa, synthetic construct
ATNHQSSVRGDAASWAQAQTGW

SEQ ID NO: 186 - protein, 22 aa, synthetic construct
ATNHQSAQRTDLGVLAQAQTGW

SEQ ID NO: 187 - protein, 22 aa, synthetic construct
ATNHQSAQRGDFNNTAQAQTGW

SEQ ID NO: 188 - protein, 22 aa, synthetic construct
ATNHQENRRGDGGVLAQAQTGW

SEQ ID NO: 189 - protein, 22 aa, synthetic construct
ATNHQASTRGDSARLAGAQTGW

SEQ ID NO: 190 - protein, 22 aa, synthetic construct
ATNHQASTRGDTASWAQAQTGW

SEQ ID NO: 191 - protein, 22 aa, synthetic construct
ATNHQSSVRGDTASWAQAQTGW

SEQ ID NO: 192 - protein, 22 aa, synthetic construct
ATNHQASTRGDLNNTAQAQTGW

SEQ ID NO: 193 - protein, 22 aa, synthetic construct
ATNHQASTRGDTARLAGAQTGW

SEQ ID NO: 194 - protein, 22 aa, synthetic construct
ATNHQENRRGDSASWAQAQTGW

SEQ ID NO: 195 - protein, 22 aa, synthetic construct
ATNHQASTRGDVKGLAQAQTGW

SEQ ID NO: 196 - protein, 22 aa, synthetic construct
ATNHQSAQRGDTGVLAQAQTGW

SEQ ID NO: 197 - protein, 22 aa, synthetic construct
ATNHQASTRGDSKGLAGAQTGW

SEQ ID NO: 198 - protein, 22 aa, synthetic construct
ATNHQASTRGDTKGLAQAQTGW

SEQ ID NO: 199 - protein, 22 aa, synthetic construct
ATNHQASTRGDSARLAQAQTGW

SEQ ID NO: 200 - protein, 22 aa, synthetic construct
ATNHQSAQRGDTASWAQAQTGW

SEQ ID NO: 201 - protein, 22 aa, synthetic construct
ATNHQSAQRGDLNNTAQAQTGW

SEQ ID NO: 202 - protein, 22 aa, synthetic construct
ATNHQASTRGDHASWAQAQTGW

SEQ ID NO: 203 - protein, 22 aa, synthetic construct
ATNHQASTRGDGARLAGAQTGW

SEQ ID NO: 204 - protein, 22 aa, synthetic construct
ATNHQSSVRGDHASWAQAQTGW

SEQ ID NO: 205 - protein, 22 aa, synthetic construct
ATNHQASTRGDFNNTAQAQTGW

SEQ ID NO: 206 - protein, 22 aa, synthetic construct
ATNHQSSVRGDVGVLAQAQTGW

SEQ ID NO: 207 - protein, 22 aa, synthetic construct
ATNHQENRRGDGASWAQAQTGW

SEQ ID NO: 208 - protein, 22 aa, synthetic construct
ATNHQASTRGDAARLAQAQTGW

SEQ ID NO: 209 - protein, 22 aa, synthetic construct
ATNHQASTRGDGKGLAGAQTGW

SEQ ID NO: 210 - protein, 22 aa, synthetic construct
ATNHQASTRGDAKGLAQAQTGW

SEQ ID NO: 211 - protein, 22 aa, synthetic construct
ATNHQENRRGDAASWAQAQTGW

SEQ ID NO: 212 - protein, 22 aa, synthetic construct
ATNHQSAQRTDVGVLAQAQTGW

SEQ ID NO: 213 - protein, 22 aa, synthetic construct
ATNHQENRRGDTGVLAQAQTGW

SEQ ID NO: 214 - protein, 22 aa, synthetic construct
ATNHQASTRGDVASWAQAQTGW

SEQ ID NO: 215 - protein, 7 aa, synthetic construct
RGDAASW

SEQ ID NO: 216 - protein, 7 aa, synthetic construct
RGDAGVL

SEQ ID NO: 217 - protein, 7 aa, synthetic construct
RGDGASW

SEQ ID NO: 218 - protein, 7 aa, synthetic construct
RGDGGVL

SEQ ID NO: 219 - protein, 7 aa, synthetic construct
RGDLNNT

SEQ ID NO: 220 - protein, 7 aa, synthetic construct
RGDSASW

SEQ ID NO: 221 - protein, 7 aa, synthetic construct
RGDSGVL

SEQ ID NO: 222 - protein, 7 aa, synthetic construct
RGDTASW

SEQ ID NO: 223 - protein, 7 aa, synthetic construct
RGDTGVL

SEQ ID NO: 224 - protein, 7 aa, synthetic construct
RGDVASW

SEQ ID NO: 225 - protein, 7 aa, synthetic construct
RGDVGVL

SEQ ID NO: 226 - protein, 7 aa, synthetic construct
RTDLGVL

SEQ ID NO: 227 - protein, 7 aa, synthetic construct
RTDVGVL

SEQ ID NO: 228 - protein, 7 aa, synthetic construct
RGDAARL

SEQ ID NO: 229 - protein, 7 aa, synthetic construct
RGDAKGL

SEQ ID NO: 230 - protein, 7 aa, synthetic construct
RGDGARL

SEQ ID NO: 231 - protein, 7 aa, synthetic construct
RGDGKGL

SEQ ID NO: 232 - protein, 7 aa, synthetic construct
RGDLGSG

SEQ ID NO: 233 - protein, 7 aa, synthetic construct
RGDLTGR

SEQ ID NO: 234 - protein, 7 aa, synthetic construct
RGDLVST

SEQ ID NO: 235 - protein, 7 aa, synthetic construct
RGDSARL

SEQ ID NO: 236 - protein, 7 aa, synthetic construct
RGDSKGL

SEQ ID NO: 237 - protein, 7 aa, synthetic construct
RGDTARL

SEQ ID NO: 238 - protein, 7 aa, synthetic construct
RGDTKGL

SEQ ID NO: 239 - protein, 7 aa, synthetic construct
RGDVKGL

SEQ ID NO: 240 - protein, 7 aa, synthetic construct
RGDFNNT

SEQ ID NO: 241 - protein, 7 aa, synthetic construct
RGDHASW

SEQ ID NO: 242 - protein, 7 aa, synthetic construct
RGDHGVL

SEQ ID NO: 243 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSSVRGDHGVLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 244 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSSVRGDSASWAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 245 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSSVRGDAGVLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 246 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQENRRGDLVSTTQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 247 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQASTRGDTKGLAGAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 248 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQENRRGDLVSTTLAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 249 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQENRRGDHGVLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 250 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSSVRGDTGVLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 251 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSSVRGDSGVLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 252 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQASTRGDAKGLAGAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 253 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSSVRGDGASWAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 254 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRGDSASWAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 255 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSSVRGDLNNTAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 256 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRGDHGVLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 257 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQASTRGDAGVLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 258 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSSVRTDLGVLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 259 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSSVRGDGGVLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 260 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQASTRGDSASWAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 261 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQASTRGDVKGLAGAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 262 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQASTRGDSGVLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 263 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRGDGASWAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 264 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRGDAGVLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 265 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSSVRGDFNNTAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 266 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQENRRGDSGVLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 267 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQENRRGDAGVLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 268 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQENRRGDLNNTAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 269 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQASTRGDTGVLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 270 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQASTRGDAASWAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 271 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRGDAASWAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 272 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQASTRGDAARLAGAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 273 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQASTRGDGGVLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 274 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRGDSGVLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 275 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQASTRGDLVSTTLAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 276 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQENRRGDLGSGNRAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 277 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQASTRGDLVSTTQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 278 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQASTRGDGASWAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 279 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRGDGGVLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 280 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQASTRGDLTGRSGAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 281 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSSVRGDAASWAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 282 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRTDLGVLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 283 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRGDFNNTAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 284 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQENRRGDGGVLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 285 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQASTRGDSARLAGAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 286 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQASTRGDTASWAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 287 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSSVRGDTASWAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 288 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQASTRGDLNNTAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 289 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQASTRGDTARLAGAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 290 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQENRRGDSASWAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 291 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQASTRGDVKGLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 292 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRGDTGVLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 293 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQASTRGDSKGLAGAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 294 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQASTRGDTKGLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 295 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQASTRGDSARLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 296 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRGDTASWAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 297 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRGDLNNTAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 298 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQASTRGDHASWAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 299 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQASTRGDGARLAGAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 300 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSSVRGDHASWAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 301 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQASTRGDFNNTAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 302 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSSVRGDVGVLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 303 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQENRRGDGASWAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 304 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQASTRGDAARLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 305 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQASTRGDGKGLAGAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 306 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQASTRGDAKGLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 307 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQENRRGDAASWAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 308 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRTDVGVLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 309 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQENRRGDTGVLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 310 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQASTRGDVASWAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 311 - DNA, 3825 bp, Homo sapiens
  source 1..3825: mol_type = genomic DNA
atgcctgagccggggaagaagccagtctcagcttttagcaagaagccacggtcagtggaagtggccgcaggcagccctgccgtgttcgaggccgagacagagcgggcaggagtgaaggtgcgctggcagcgcggaggcagtgacatcagcgccagcaacaagtacggcctggccacagagggcacacggcatacgctgacagtgcgggaagtgggccctgccgaccagggatcttacgcagtcattgctggctcctccaaggtcaagttcgacctcaaggtcatagaggcagagaaggcagagcccatgctggcccctgcccctgcccctgctgaggccactggagcccctggagaagccccggccccagccgctgagctgggagaaagtgccccaagtcccaaagggtcaagctcagcagctctcaatggtcctacccctggagcccccgatgaccccattggcctcttcgtgatgcggccacaggatggcgaggtgaccgtgggtggcagcatcaccttctcagcccgcgtggccggcgccagcctcctgaagccgcctgtggtcaagtggttcaagggcaaatgggtggacctgagcagcaaggtgggccagcacctgcagctgcacgacagctacgaccgcgccagcaaggtctatctgttcgagctgcacatcaccgatgcccagcctgccttcactggcagctaccgctgtgaggtgtccaccaaggacaaatttgactgctccaacttcaatctcactgtccacgaggccatgggcaccggagacctggacctcctatcagccttccgccgcacgagcctggctggaggtggtcggcggatcagtgatagccatgaggacactgggattctggacttcagctcactgctgaaaaagagagacagtttccggaccccgagggactcgaagctggaggcaccagcagaggaggacgtgtgggagatcctacggcaggcacccccatctgagtacgagcgcatcgccttccagtacggcgtcactgacctgcgcggcatgctaaagaggctcaagggcatgaggcgcgatgagaagaagagcacagcctttcagaagaagctggagccggcctaccaggtgagcaaaggccacaagatccggctgaccgtggaactggctgaccatgacgctgaggtcaaatggctcaagaatggccaggagatccagatgagcggcagcaagtacatctttgagtccatcggtgccaagcgtaccctgaccatcagccagtgctcattggcggacgacgcagcctaccagtgcgtggtgggtggcgagaagtgtagcacggagctctttgtgaaagagccccctgtgctcatcacgcgccccttggaggaccagctggtgatggtggggcagcgggtggagtttgagtgtgaagtatcggaggagggggcgcaagtcaaatggctgaaggacggggtggagctgacccgggaggagaccttcaaataccggttcaagaaggacgggcagagacaccacctgatcatcaacgaggccatgctggaggacgcggggcactatgcactgtgcactagcgggggccaggcgctggctgagctcattgtgcaggaaaagaagctggaggtgtaccagagcatcgcagacctgatggtgggcgcaaaggaccaggcggtgttcaaatgtgaggtctcagatgagaatgttcggggtgtgtggctgaagaatgggaaggagctggtgcccgacagccgcataaaggtgtcccacatcgggcgggtccacaaactgaccattgacgacgtcacacctgccgacgaggctgactacagctttgtgcccgagggcttcgcctgcaacctgtcagccaagctccacttcatggaggtcaagattgacttcgtacccaggcaggaacctcccaagatccacctggactgcccaggccgcataccagacaccattgtggttgtagctggaaataagctacgtctggacgtccctatctctggggaccccgctcccactgtgatctggcagaaggctatcacgcaggggaataaggccccagccaggccagccccagatgccccagaggacacaggtgacagcgatgagtgggtgtttgacaagaagctgctgtgtgagaccgagggccgggtccgcgtggagaccaccaaggaccgcagcatcttcacggtcgagggggcagagaaggaagatgagggcgtctacacggtcacagtgaagaaccctgtgggcgaggaccaggtcaacctcacagtcaaggtcatcgacgtgccagacgcacctgcggcccccaagatcagcaacgtgggagaggactcctgcacagtacagtgggagccgcctgcctacgatggcgggcagcccatcctgggctacatcctggagcgcaagaagaagaagagctaccggtggatgcggctgaacttcgacctgattcaggagctgagtcatgaagcgcggcgcatgatcgagggcgtggtgtacgagatgcgcgtctacgcggtcaacgccatcggcatgtccaggcccagccctgcctcccagcccttcatgcctatcggtccccccagcgaacccacccacctggcagtagaggacgtctctgacaccacggtctccctcaagtggcggcccccagagcgcgtgggagcaggaggcctggatggctacagcgtggagtactgcccagagggctgctcagagtgggtggctgccctgcaggggctgacagagcacacatcgatactggtgaaggacctgcccacgggggcccggctgcttttccgagtgcgggcacacaatatggcagggcctggagcccctgttaccaccacggagccggtgacagtgcaggagatcctgcaacggccacggcttcagctgcccaggcacctgcgccagaccattcagaagaaggtcggggagcctgtgaaccttctcatccctttccagggcaagccccggcctcaggtgacctggaccaaagaggggcagcccctggcaggcgaggaggtgagcatccgcaacagccccacagacaccatcctgttcatccgggccgctcgccgcgtgcattcaggcacttaccaggtgacggtgcgcattgagaacatggaggacaaggccacgctggtgctgcaggttgttgacaagccaagtcctccccaggatctccgggtgactgacgcctggggtcttaatgtggctctggagtggaagccaccccaggatgtcggcaacacggaactctgggggtacacagtgcagaaagccgacaagaagaccatggagtggttcaccgtcttggagcattaccgccgcacccactgcgtggtgccagagctcatcattggcaatggctactacttccgcgtcttcagccagaatatggttggctttagtgacagagcggccaccaccaaggagcccgtctttatccccagaccaggcatcacctatgagccacccaactataaggccctggacttctccgaggccccaagcttcacccagcccctggtgaaccgctcggtcatcgcgggctacactgctatgctctgctgtgctgtccggggtagccccaagcccaagatttcctggttcaagaatggcctggacctgggagaagacgcccgcttccgcatgttcagcaagcagggagtgttgactctggagattagaaagccctgcccctttgacgggggcatctatgtctgcagggccaccaacttacagggcgaggcacggtgtgagtgccgcctggaggtgcgagtgcctcagtaa

SEQ ID NO: 312 - protein, 1274 aa, Homo sapiens
MPEPGKKPVSAFSKKPRSVEVAAGSPAVFEAETERAGVKVRWQRGGSDISASNKYGLATEGTRHTLTVREVGPADQGSYAVIAGSSKVKFDLKVIEAEKAEPMLAPAPAPAEATGAPGEAPAPAAELGESAPSPKGSSSAALNGPTPGAPDDPIGLFVMRPQDGEVTVGGSITFSARVAGASLLKPPVVKWFKGKWVDLSSKVGQHLQLHDSYDRASKVYLFELHITDAQPAFTGSYRCEVSTKDKFDCSNFNLTVHEAMGTGDLDLLSAFRRTSLAGGGRRISDSHEDTGILDFSSLLKKRDSFRTPRDSKLEAPAEEDVWEILRQAPPSEYERIAFQYGVTDLRGMLKRLKGMRRDEKKSTAFQKKLEPAYQVSKGHKIRLTVELADHDAEVKWLKNGQEIQMSGSKYIFESIGAKRTLTISQCSLADDAAYQCVVGGEKCSTELFVKEPPVLITRPLEDQLVMVGQRVEFECEVSEEGAQVKWLKDGVELTREETFKYRFKKDGQRHHLIINEAMLEDAGHYALCTSGGQALAELIVQEKKLEVYQSIADLMVGAKDQAVFKCEVSDENVRGVWLKNGKELVPDSRIKVSHIGRVHKLTIDDVTPADEADYSFVPEGFACNLSAKLHFMEVKIDFVPRQEPPKIHLDCPGRIPDTIVVVAGNKLRLDVPISGDPAPTVIWQKAITQGNKAPARPAPDAPEDTGDSDEWVFDKKLLCETEGRVRVETTKDRSIFTVEGAEKEDEGVYTVTVKNPVGEDQVNLTVKVIDVPDAPAAPKISNVGEDSCTVQWEPPAYDGGQPILGYILERKKKKSYRWMRLNFDLIQELSHEARRMIEGVVYEMRVYAVNAIGMSRPSPASQPFMPIGPPSEPTHLAVEDVSDTTVSLKWRPPERVGAGGLDGYSVEYCPEGCSEWVAALQGLTEHTSILVKDLPTGARLLFRVRAHNMAGPGAPVTTTEPVTVQEILQRPRLQLPRHLRQTIQKKVGEPVNLLIPFQGKPRPQVTWTKEGQPLAGEEVSIRNSPTDTILFIRAARRVHSGTYQVTVRIENMEDKATLVLQVVDKPSPPQDLRVTDAWGLNVALEWKPPQDVGNTELWGYTVQKADKKTMEWFTVLEHYRRTHCVVPELIIGNGYYFRVFSQNMVGFSDRAATTKEPVFIPRPGITYEPPNYKALDFSEAPSFTQPLVNRSVIAGYTAMLCCAVRGSPKPKISWFKNGLDLGEDARFRMFSKQGVLTLEIRKPCPFDGGIYVCRATNLQGEARCECRLEVRVPQ

SEQ ID NO: 313 - DNA, 3546 bp, synthetic construct
  source 1..3546: mol_type = other DNA
atgcctgagccggggaagaagccagtctcagcttttagcaagaagccacggtcagtggaagtggccgcaggcagccctgccgtgttcgaggccgagacagagcgggcaggagtgaaggtgcgctggcagcgcggaggcagtgacatcagcgccagcaacaagtacggcctggccacagagggcacacggcatacgctgacagtgcgggaagtgggccctgccgaccagggatcttacgcagtcattgctggctcctccaaggtcaagttcgacctcaaggtcatagaggcagagaaggcagagcccatgctggcccctgcccctgcccctgctgaggccactggagcccctggagaagccccggccccagccgctgagctgggagaaagtgccccaagtcccaaagggtcaagctcagcagctctcaatggtcctacccctggagcccccgatgaccccattggcctcttcgtgatgcggccacaggatggcgaggtgaccgtgggtggcagcatcaccttctcagcccgcgtggccggcgccagcctcctgaagccgcctgtggtcaagtggttcaagggcaaatgggtggacctgagcagcaaggtgggccagcacctgcagctgcacgacagctacgaccgcgccagcaaggtctatctgttcgagctgcacatcaccgatgcccagcctgccttcactggcagctaccgctgtgaggtgtccaccaaggacaaatttgactgctccaacttcaatctcactgtccacgaggccatgggcaccggagacctggacctcctatcagccttccgccgcacgagcctggctggaggtggtcggcggatcagtgatagccatgaggacactgggattctggacttcagctcactgctgaaaaagagagacagtttccggaccccgagggactcgaagctggaggcaccagcagaggaggacgtgtgggagatcctacggcaggcacccccatctgagtacgagcgcatcgccttccagtacggcgtcactgacctgcgcggcatgctaaagaggctcaagggcatgaggcgcgatgagaagaagagcacagcctttcagaagaagctggagccggcctaccaggtgagcaaaggccacaagatccggctgaccgtggaactggctgaccatgacgctgaggtcaaatggctcaagaatggccaggagatccagatgagcggcagcaagtacatctttgagtccatcggtgccaagcgtaccctgaccatcagccagtgctcattggcggacgacgcagcctaccagtgcgtggtgggtggcgagaagtgtagcacggagctctttgtgaaagagccccctgtgtaccagagcatcgcagacctgatggtgggcgcaaaggaccaggcggtgttcaaatgtgaggtctcagatgagaatgttcggggtgtgtggctgaagaatgggaaggagctggtgcccgacagccgcataaaggtgtcccacatcgggcgggtccacaaactgaccattgacgacgtcacacctgccgacgaggctgactacagctttgtgcccgagggcttcgcctgcaacctgtcagccaagctccacttcatggaggtcaagattgacttcgtacccaggcaggaacctcccaagatccacctggactgcccaggccgcataccagacaccattgtggttgtagctggaaataagctacgtctggacgtccctatctctggggaccccgctcccactgtgatctggcagaaggctatcacgcaggggaataaggccccagccaggccagccccagatgccccagaggacacaggtgacagcgatgagtgggtgtttgacaagaagctgctgtgtgagaccgagggccgggtccgcgtggagaccaccaaggaccgcagcatcttcacggtcgagggggcagagaaggaagatgagggcgtctacacggtcacagtgaagaaccctgtgggcgaggaccaggtcaacctcacagtcaaggtcatcgacgtgccagacgcacctgcggcccccaagatcagcaacgtgggagaggactcctgcacagtacagtgggagccgcctgcctacgatggcgggcagcccatcctgggctacatcctggagcgcaagaagaagaagagctaccggtggatgcggctgaacttcgacctgattcaggagctgagtcatgaagcgcggcgcatgatcgagggcgtggtgtacgagatgcgcgtctacgcggtcaacgccatcggcatgtccaggcccagccctgcctcccagcccttcatgcctatcggtccccccagcgaacccacccacctggcagtagaggacgtctctgacaccacggtctccctcaagtggcggcccccagagcgcgtgggagcaggaggcctggatggctacagcgtggagtactgcccagagggctgctcagagtgggtggctgccctgcaggggctgacagagcacacatcgatactggtgaaggacctgcccacgggggcccggctgcttttccgagtgcgggcacacaatatggcagggcctggagcccctgttaccaccacggagccggtgacagtgcaggagatcctgcaacggccacggcttcagctgcccaggcacctgcgccagaccattcagaagaaggtcggggagcctgtgaaccttctcatccctttccagggcaagccccggcctcaggtgacctggaccaaagaggggcagcccctggcaggcgaggaggtgagcatccgcaacagccccacagacaccatcctgttcatccgggccgctcgccgcgtgcattcaggcacttaccaggtgacggtgcgcattgagaacatggaggacaaggccacgctggtgctgcaggttgttgacaagccaagtcctccccaggatctccgggtgactgacgcctggggtcttaatgtggctctggagtggaagccaccccaggatgtcggcaacacggaactctgggggtacacagtgcagaaagccgacaagaagaccatggagtggttcaccgtcttggagcattaccgccgcacccactgcgtggtgccagagctcatcattggcaatggctactacttccgcgtcttcagccagaatatggttggctttagtgacagagcggccaccaccaaggagcccgtctttatccccagaccaggcatcacctatgagccacccaactataaggccctggacttctccgaggccccaagcttcacccagcccctggtgaaccgctcggtcatcgcgggctacactgctatgctctgctgtgctgtccggggtagccccaagcccaagatttcctggttcaagaatggcctggacctgggagaagacgcccgcttccgcatgttcagcaagcagggagtgttgactctggagattagaaagccctgcccctttgacgggggcatctatgtctgcagggccaccaacttacagggcgaggcacggtgtgagtgccgcctggaggtgcgagtgcctcagtaa

SEQ ID NO: 314 - protein, 1181 aa, synthetic construct
MPEPGKKPVSAFSKKPRSVEVAAGSPAVFEAETERAGVKVRWQRGGSDISASNKYGLATEGTRHTLTVREVGPADQGSYAVIAGSSKVKFDLKVIEAEKAEPMLAPAPAPAEATGAPGEAPAPAAELGESAPSPKGSSSAALNGPTPGAPDDPIGLFVMRPQDGEVTVGGSITFSARVAGASLLKPPVVKWFKGKWVDLSSKVGQHLQLHDSYDRASKVYLFELHITDAQPAFTGSYRCEVSTKDKFDCSNFNLTVHEAMGTGDLDLLSAFRRTSLAGGGRRISDSHEDTGILDFSSLLKKRDSFRTPRDSKLEAPAEEDVWEILRQAPPSEYERIAFQYGVTDLRGMLKRLKGMRRDEKKSTAFQKKLEPAYQVSKGHKIRLTVELADHDAEVKWLKNGQEIQMSGSKYIFESIGAKRTLTISQCSLADDAAYQCVVGGEKCSTELFVKEPPVYQSIADLMVGAKDQAVFKCEVSDENVRGVWLKNGKELVPDSRIKVSHIGRVHKLTIDDVTPADEADYSFVPEGFACNLSAKLHFMEVKIDFVPRQEPPKIHLDCPGRIPDTIVVVAGNKLRLDVPISGDPAPTVIWQKAITQGNKAPARPAPDAPEDTGDSDEWVFDKKLLCETEGRVRVETTKDRSIFTVEGAEKEDEGVYTVTVKNPVGEDQVNLTVKVIDVPDAPAAPKISNVGEDSCTVQWEPPAYDGGQPILGYILERKKKKSYRWMRLNFDLIQELSHEARRMIEGVVYEMRVYAVNAIGMSRPSPASQPFMPIGPPSEPTHLAVEDVSDTTVSLKWRPPERVGAGGLDGYSVEYCPEGCSEWVAALQGLTEHTSILVKDLPTGARLLFRVRAHNMAGPGAPVTTTEPVTVQEILQRPRLQLPRHLRQTIQKKVGEPVNLLIPFQGKPRPQVTWTKEGQPLAGEEVSIRNSPTDTILFIRAARRVHSGTYQVTVRIENMEDKATLVLQVVDKPSPPQDLRVTDAWGLNVALEWKPPQDVGNTELWGYTVQKADKKTMEWFTVLEHYRRTHCVVPELIIGNGYYFRVFSQNMVGFSDRAATTKEPVFIPRPGITYEPPNYKALDFSEAPSFTQPLVNRSVIAGYTAMLCCAVRGSPKPKISWFKNGLDLGEDARFRMFSKQGVLTLEIRKPCPFDGGIYVCRATNLQGEARCECRLEVRVPQ

SEQ ID NO: 315 - DNA, 3534 bp, synthetic construct
  source 1..3534: mol_type = other DNA
atgcctgagccggggaagaagccagtctcagcttttagcaagaagccacggtcagtggaagtggccgcaggcagccctgccgtgttcgaggccgagacagagcgggcaggagtgaaggtgcgctggcagcgcggaggcagtgacatcagcgccagcaacaagtacggcctggccacagagggcacacggcatacgctgacagtgcgggaagtgggccctgccgaccagggatcttacgcagtcattgctggctcctccaaggtcaagttcgacctcaaggtcatagaggcagagaaggcagagcccatgctggcccctgcccctgcccctgctgaggccactggagcccctggagaagccccggccccagccgctgagctgggagaaagtgccccaagtcccaaagggtcaagctcagcagctctcaatggtcctacccctggagcccccgatgaccccattggcctcttcgtgatgcggccacaggatggcgaggtgaccgtgggtggcagcatcaccttctcagcccgcgtggccggcgccagcctcctgaagccgcctgtggtcaagtggttcaagggcaaatgggtggacctgagcagcaaggtgggccagcacctgcagctgcacgacagctacgaccgcgccagcaaggtctatctgttcgagctgcacatcaccgatgcccagcctgccttcactggcagctaccgctgtgaggtgtccaccaaggacaaatttgactgctccaacttcaatctcactgtccacgaggccatgggcaccggagacctggacctcctatcagccttccgccgcacgagcctggctggaggtggtcggcggatcagtgatagccatgaggacactgggattctggacttcagctcactgctgaaaaagagagacagtttccggaccccgagggactcgaagctggaggcaccagcagaggaggacgtgtgggagatcctacggcaggcacccccatctgagtacgagcgcatcgccttccagtacggcgtcactgacctgcgcggcatgctaaagaggctcaagggcatgaggcgcgatgagaagaagagcacagcctttcagaagaagctggagccggcctaccaggtgagcaaaggccacaagatccggctgaccgtggaactggctgaccatgacgctgaggtcaaatggctcaagaatggccaggagatccagatgagcggcagcaagtacatctttgagtccatcggtgccaagcgtaccctgaccatcagccagtgctcattggcggacgacgcagcctaccagtgcgtggtgggtggcgagaagtgtagcacggagctctttgtgaaagagccccctgtgctcatcacgcgccccttggaggaccagctggtgatggtggggcagcgggtggagtttgagtgtgaagtatcggaggagggggcgcaagtcaaatggctgaaggacggggtggagctgacccgggaggagaccttcaaataccggttcaagaaggacgggcagagacaccacctgatcatcaacgaggccatgctggaggacgcggggcactatgcactgtgcactagcgggggccaggcgctggctgagctcattgtgcaggaaaagaagctggagcctcccaagatccacctggactgcccaggccgcataccagacaccattgtggttgtagctggaaataagctacgtctggacgtccctatctctggggaccccgctcccactgtgatctggcagaaggctatcacgcaggggaataaggccccagccaggccagccccagatgccccagaggacacaggtgacagcgatgagtgggtgtttgacaagaagctgctgtgtgagaccgagggccgggtccgcgtggagaccaccaaggaccgcagcatcttcacggtcgagggggcagagaaggaagatgagggcgtctacacggtcacagtgaagaaccctgtgggcgaggaccaggtcaacctcacagtcaaggtcatcgacgtgccagacgcacctgcggcccccaagatcagcaacgtgggagaggactcctgcacagtacagtgggagccgcctgcctacgatggcgggcagcccatcctgggctacatcctggagcgcaagaagaagaagagctaccggtggatgcggctgaacttcgacctgattcaggagctgagtcatgaagcgcggcgcatgatcgagggcgtggtgtacgagatgcgcgtctacgcggtcaacgccatcggcatgtccaggcccagccctgcctcccagcccttcatgcctatcggtccccccagcgaacccacccacctggcagtagaggacgtctctgacaccacggtctccctcaagtggcggcccccagagcgcgtgggagcaggaggcctggatggctacagcgtggagtactgcccagagggctgctcagagtgggtggctgccctgcaggggctgacagagcacacatcgatactggtgaaggacctgcccacgggggcccggctgcttttccgagtgcgggcacacaatatggcagggcctggagcccctgttaccaccacggagccggtgacagtgcaggagatcctgcaacggccacggcttcagctgcccaggcacctgcgccagaccattcagaagaaggtcggggagcctgtgaaccttctcatccctttccagggcaagccccggcctcaggtgacctggaccaaagaggggcagcccctggcaggcgaggaggtgagcatccgcaacagccccacagacaccatcctgttcatccgggccgctcgccgcgtgcattcaggcacttaccaggtgacggtgcgcattgagaacatggaggacaaggccacgctggtgctgcaggttgttgacaagccaagtcctccccaggatctccgggtgactgacgcctggggtcttaatgtggctctggagtggaagccaccccaggatgtcggcaacacggaactctgggggtacacagtgcagaaagccgacaagaagaccatggagtggttcaccgtcttggagcattaccgccgcacccactgcgtggtgccagagctcatcattggcaatggctactacttccgcgtcttcagccagaatatggttggctttagtgacagagcggccaccaccaaggagcccgtctttatccccagaccaggcatcacctatgagccacccaactataaggccctggacttctccgaggccccaagcttcacccagcccctggtgaaccgctcggtcatcgcgggctacactgctatgctctgctgtgctgtccggggtagccccaagcccaagatttcctggttcaagaatggcctggacctgggagaagacgcccgcttccgcatgttcagcaagcagggagtgttgactctggagattagaaagccctgcccctttgacgggggcatctatgtctgcagggccaccaacttacagggcgaggcacggtgtgagtgccgcctggaggtgcgagtgcctcagtaa

SEQ ID NO: 316 - protein, 1177 aa, synthetic construct
MPEPGKKPVSAFSKKPRSVEVAAGSPAVFEAETERAGVKVRWQRGGSDISASNKYGLATEGTRHTLTVREVGPADQGSYAVIAGSSKVKFDLKVIEAEKAEPMLAPAPAPAEATGAPGEAPAPAAELGESAPSPKGSSSAALNGPTPGAPDDPIGLFVMRPQDGEVTVGGSITFSARVAGASLLKPPVVKWFKGKWVDLSSKVGQHLQLHDSYDRASKVYLFELHITDAQPAFTGSYRCEVSTKDKFDCSNFNLTVHEAMGTGDLDLLSAFRRTSLAGGGRRISDSHEDTGILDFSSLLKKRDSFRTPRDSKLEAPAEEDVWEILRQAPPSEYERIAFQYGVTDLRGMLKRLKGMRRDEKKSTAFQKKLEPAYQVSKGHKIRLTVELADHDAEVKWLKNGQEIQMSGSKYIFESIGAKRTLTISQCSLADDAAYQCVVGGEKCSTELFVKEPPVLITRPLEDQLVMVGQRVEFECEVSEEGAQVKWLKDGVELTREETFKYRFKKDGQRHHLIINEAMLEDAGHYALCTSGGQALAELIVQEKKLEPPKIHLDCPGRIPDTIVVVAGNKLRLDVPISGDPAPTVIWQKAITQGNKAPARPAPDAPEDTGDSDEWVFDKKLLCETEGRVRVETTKDRSIFTVEGAEKEDEGVYTVTVKNPVGEDQVNLTVKVIDVPDAPAAPKISNVGEDSCTVQWEPPAYDGGQPILGYILERKKKKSYRWMRLNFDLIQELSHEARRMIEGVVYEMRVYAVNAIGMSRPSPASQPFMPIGPPSEPTHLAVEDVSDTTVSLKWRPPERVGAGGLDGYSVEYCPEGCSEWVAALQGLTEHTSILVKDLPTGARLLFRVRAHNMAGPGAPVTTTEPVTVQEILQRPRLQLPRHLRQTIQKKVGEPVNLLIPFQGKPRPQVTWTKEGQPLAGEEVSIRNSPTDTILFIRAARRVHSGTYQVTVRIENMEDKATLVLQVVDKPSPPQDLRVTDAWGLNVALEWKPPQDVGNTELWGYTVQKADKKTMEWFTVLEHYRRTHCVVPELIIGNGYYFRVFSQNMVGFSDRAATTKEPVFIPRPGITYEPPNYKALDFSEAPSFTQPLVNRSVIAGYTAMLCCAVRGSPKPKISWFKNGLDLGEDARFRMFSKQGVLTLEIRKPCPFDGGIYVCRATNLQGEARCECRLEVRVPQ

SEQ ID NO: 317 - DNA, 3546 bp, synthetic construct
  source 1..3546: mol_type = other DNA
atgcctgagccggggaagaagccagtctcagcttttagcaagaagccacggtcagtggaagtggccgcaggcagccctgccgtgttcgaggccgagacagagcgggcaggagtgaaggtgcgctggcagcgcggaggcagtgacatcagcgccagcaacaagtacggcctggccacagagggcacacggcatacgctgacagtgcgggaagtgggccctgccgaccagggatcttacgcagtcattgctggctcctccaaggtcaagttcgacctcaaggtcatagaggcagagaaggcagagcccatgctggcccctgcccctgcccctgctgaggccactggagcccctggagaagccccggccccagccgctgagctgggagaaagtgccccaagtcccaaagggtcaagctcagcagctctcaatggtcctacccctggagcccccgatgaccccattggcctcttcgtgatgcggccacaggatggcgaggtgaccgtgggtggcagcatcaccttctcagcccgcgtggccggcgccagcctcctgaagccgcctgtggtcaagtggttcaagggcaaatgggtggacctgagcagcaaggtgggccagcacctgcagctgcacgacagctacgaccgcgccagcaaggtctatctgttcgagctgcacatcaccgatgcccagcctgccttcactggcagctaccgctgtgaggtgtccaccaaggacaaatttgactgctccaacttcaatctcactgtccacgaggccatgggcaccggagacctggacctcctatcagccttccgccgcacgagcctggctggaggtggtcggcggatcagtgatagccatgaggacactgggattctggacttcagctcactgctgaaaaagagagacagtttccggaccccgagggactcgaagctggaggcaccagcagaggaggacgtgtgggagatcctacggcaggcacccccatctgagtacgagcgcatcgccttccagtacggcgtcactgacctgcgcggcatgctaaagaggctcaagggcatgaggcgcgatgagaagaagagcacagcctttcagaagaagctggagccggcctaccaggtgagcaaaggccacaagatccggctgaccgtggaactggctgaccatgacgctgaggtcaaatggctcaagaatggccaggagatccagatgagcggcagcaagtacatctttgagtccatcggtgccaagcgtaccctgaccatcagccagtgctcattggcggacgacgcagcctaccagtgcgtggtgggtggcgagaagtgtagcacggagctctttgtgaaagagccccctgtgctcatcacgcgccccttggaggaccagctggtgatggtggggcagcgggtggagtttgagtgtgaagtatcggaggagggggcgcaagtcaaatggctgaaggacggggtggagctgacccgggaggagaccttcaaataccggttcaagaaggacgggcagagacaccacctgatcatcaacgaggccatgctggaggacgcggggcactatgcactgtgcactagcgggggccaggcgctggctgagctcattgtgcaggaaaagaagctggagcccaggcaggaacctcccaagatccacctggactgcccaggccgcataccagacaccattgtggttgtagctggaaataagctacgtctggacgtccctatctctggggaccccgctcccactgtgatctggcagaaggctatcacgcaggggaataaggccccagccaggccagccccagatgccccagaggacacaggtgacagcgatgagtgggtgtttgacaagaagctgctgtgtgagaccgagggccgggtccgcgtggagaccaccaaggaccgcagcatcttcacggtcgagggggcagagaaggaagatgagggcgtctacacggtcacagtgaagaaccctgtgggcgaggaccaggtcaacctcacagtcaaggtcatcgacgtgccagacgcacctgcggcccccaagatcagcaacgtgggagaggactcctgcacagtacagtgggagccgcctgcctacgatggcgggcagcccatcctgggctacatcctggagcgcaagaagaagaagagctaccggtggatgcggctgaacttcgacctgattcaggagctgagtcatgaagcgcggcgcatgatcgagggcgtggtgtacgagatgcgcgtctacgcggtcaacgccatcggcatgtccaggcccagccctgcctcccagcccttcatgcctatcggtccccccagcgaacccacccacctggcagtagaggacgtctctgacaccacggtctccctcaagtggcggcccccagagcgcgtgggagcaggaggcctggatggctacagcgtggagtactgcccagagggctgctcagagtgggtggctgccctgcaggggctgacagagcacacatcgatactggtgaaggacctgcccacgggggcccggctgcttttccgagtgcgggcacacaatatggcagggcctggagcccctgttaccaccacggagccggtgacagtgcaggagatcctgcaacggccacggcttcagctgcccaggcacctgcgccagaccattcagaagaaggtcggggagcctgtgaaccttctcatccctttccagggcaagccccggcctcaggtgacctggaccaaagaggggcagcccctggcaggcgaggaggtgagcatccgcaacagccccacagacaccatcctgttcatccgggccgctcgccgcgtgcattcaggcacttaccaggtgacggtgcgcattgagaacatggaggacaaggccacgctggtgctgcaggttgttgacaagccaagtcctccccaggatctccgggtgactgacgcctggggtcttaatgtggctctggagtggaagccaccccaggatgtcggcaacacggaactctgggggtacacagtgcagaaagccgacaagaagaccatggagtggttcaccgtcttggagcattaccgccgcacccactgcgtggtgccagagctcatcattggcaatggctactacttccgcgtcttcagccagaatatggttggctttagtgacagagcggccaccaccaaggagcccgtctttatccccagaccaggcatcacctatgagccacccaactataaggccctggacttctccgaggccccaagcttcacccagcccctggtgaaccgctcggtcatcgcgggctacactgctatgctctgctgtgctgtccggggtagccccaagcccaagatttcctggttcaagaatggcctggacctgggagaagacgcccgcttccgcatgttcagcaagcagggagtgttgactctggagattagaaagccctgcccctttgacgggggcatctatgtctgcagggccaccaacttacagggcgaggcacggtgtgagtgccgcctggaggtgcgagtgcctcagtaa

SEQ ID NO: 318 - protein, 1181 aa, synthetic construct
MPEPGKKPVSAFSKKPRSVEVAAGSPAVFEAETERAGVKVRWQRGGSDISASNKYGLATEGTRHTLTVREVGPADQGSYAVIAGSSKVKFDLKVIEAEKAEPMLAPAPAPAEATGAPGEAPAPAAELGESAPSPKGSSSAALNGPTPGAPDDPIGLFVMRPQDGEVTVGGSITFSARVAGASLLKPPVVKWFKGKWVDLSSKVGQHLQLHDSYDRASKVYLFELHITDAQPAFTGSYRCEVSTKDKFDCSNFNLTVHEAMGTGDLDLLSAFRRTSLAGGGRRISDSHEDTGILDFSSLLKKRDSFRTPRDSKLEAPAEEDVWEILRQAPPSEYERIAFQYGVTDLRGMLKRLKGMRRDEKKSTAFQKKLEPAYQVSKGHKIRLTVELADHDAEVKWLKNGQEIQMSGSKYIFESIGAKRTLTISQCSLADDAAYQCVVGGEKCSTELFVKEPPVLITRPLEDQLVMVGQRVEFECEVSEEGAQVKWLKDGVELTREETFKYRFKKDGQRHHLIINEAMLEDAGHYALCTSGGQALAELIVQEKKLEPRQEPPKIHLDCPGRIPDTIVVVAGNKLRLDVPISGDPAPTVIWQKAITQGNKAPARPAPDAPEDTGDSDEWVFDKKLLCETEGRVRVETTKDRSIFTVEGAEKEDEGVYTVTVKNPVGEDQVNLTVKVIDVPDAPAAPKISNVGEDSCTVQWEPPAYDGGQPILGYILERKKKKSYRWMRLNFDLIQELSHEARRMIEGVVYEMRVYAVNAIGMSRPSPASQPFMPIGPPSEPTHLAVEDVSDTTVSLKWRPPERVGAGGLDGYSVEYCPEGCSEWVAALQGLTEHTSILVKDLPTGARLLFRVRAHNMAGPGAPVTTTEPVTVQEILQRPRLQLPRHLRQTIQKKVGEPVNLLIPFQGKPRPQVTWTKEGQPLAGEEVSIRNSPTDTILFIRAARRVHSGTYQVTVRIENMEDKATLVLQVVDKPSPPQDLRVTDAWGLNVALEWKPPQDVGNTELWGYTVQKADKKTMEWFTVLEHYRRTHCVVPELIIGNGYYFRVFSQNMVGFSDRAATTKEPVFIPRPGITYEPPNYKALDFSEAPSFTQPLVNRSVIAGYTAMLCCAVRGSPKPKISWFKNGLDLGEDARFRMFSKQGVLTLEIRKPCPFDGGIYVCRATNLQGEARCECRLEVRVPQ

SEQ ID NO: 319 - DNA, 108 bp, Homo sapiens
  source 1..108: mol_type = genomic DNA
atggctgaaaaagcggggtctacattttcacaccttctggttcctattcttctcctgattggctggattgtgggctgcatcataatgatttatgttgtcttctcttag

SEQ ID NO: 320 - DNA, 108 bp, synthetic construct
  source 1..108: mol_type = other DNA
atggccgagaaggccggatctaccttcagccacctgctggtccctattctgctgctgatcggctggatcgtgggctgcatcatcatgatctacgtggtgttcagctga

SEQ ID NO: 321 - protein, 35 aa, Homo sapiens
MAEKAGSTFSHLLVPILLLIGWIVGCIIMIYVVFS

SEQ ID NO: 322 - DNA, 2091 bp, Homo sapiens
  source 1..2091: mol_type = genomic DNA
atgagtgggggccgcttcgactttgatgatggaggggcgtactgcgggggctgggaggggggaaaggcccatgggcatggactgtgcacaggccccaagggccagggcgaatactctggctcctggaactttggctttgaggtggcaggtgtctacacctggcccagcggaaacacctttgagggatactggagccagggcaaacggcatgggctgggcatagagaccaaggggcgctggctctacaagggcgagtggacacatggcttcaagggacgctacggaatccggcagagctcaagcagcggtgccaagtatgagggcacctggaacaatggcctgcaagacggctatggcaccgagacctatgctgatggagggacgtaccaaggccagttcaccaacggcatgcgccatggctacggagtacgccagagcgtgccctacgggatggccgtggtggtgcgctcgccgctgcgcacgtcgctgtcgtccctgcgcagcgagcacagcaacggcacggtggccccggactctcccgcctcgccggcctccgacggccccgcgctgccctcgcccgccatcccgcgtggcggcttcgcgctcagcctcctggccaatgccgaggcggccgcgcgggcgcccaagggcggcggcctcttccagcggggcgcgctgctgggcaagctgcggcgcgcagagtcgcgcacgtccgtgggtagccagcgcagccgtgtcagcttccttaagagcgacctcagctcgggcgccagcgacgccgcgtccaccgccagcctgggagaggccgccgagggcgccgacgaggccgcacccttcgaggccgatatcgacgccaccaccaccgagacctacatgggcgagtggaagaacgacaaacgctcgggcttcggcgtgagcgaacgctccagtggcctccgctacgagggcgagtggctggacaacctgcgccacggctatggctgcaccacgctgcccgacggccaccgcgaggagggcaagtaccgccacaacgtgctggtcaaggacaccaagcgccgcatgctgcagctcaagagcaacaaggtccgccagaaagtggagcacagtgtggagggtgcccagcgcgccgctgctatcgcgcgccagaaggccgagattgccgcctccaggacaagccacgccaaggccaaagctgaggcagcggaacaggccgccctggctgccaaccaggagtccaacattgctcgcactttggccagggagctggctccggacttctaccagccaggtccggaatatcagaagcgccggctgctgcaggagatcctggagaactcggagagcctgctggagccccccgaccggggcgccggcgcagcgggcctcccacagccgccccgcgagagcccgcagctgcacgagcgtgagacccctcggcccgagggtggctccccgtcaccggccgggacgcccccgcagcccaagcggcccaggcccggggtgtccaaggacggcctgctgagcccaggcgcctggaacggcgagcccagcggtgagggcagccggtcagtcactccgtccgagggcgcgggccgccgcagccccgcgcgtccagccaccgagcgcatggccatcgaggctctgcaggcaccgcctgcgccgtcgcgggagccggaggtggcgctttaccagggctaccacagctatgctgtgcgcaccacgccgcccgagcccccaccctttgaggaccagcccgagcccgaggtctccgggtccgagtccgcgccctcgtccccggccaccgccccgctgcaggcccccacgctccgaggccccgagcctgcacgcgagacccccgccaagctggagcccaagcccatcatccccaaagccgagcccagggccaaggcccgcaagactgaggctcgagggctgaccaaggcgggggccaagaagaaggcgcggaaggaggccgcactggcggcagaggcggaggtggaggtggaagaggtccccaacaccatcctcatctgcatggtgatcctgctgaacatcggcctggccatcctctttgttcacctcctgacctga

SEQ ID NO: 323 - protein, 696 aa, Homo sapiens
MSGGRFDFDDGGAYCGGWEGGKAHGHGLCTGPKGQGEYSGSWNFGFEVAGVYTWPSGNTFEGYWSQGKRHGLGIETKGRWLYKGEWTHGFKGRYGIRQSSSSGAKYEGTWNNGLQDGYGTETYADGGTYQGQFTNGMRHGYGVRQSVPYGMAVVVRSPLRTSLSSLRSEHSNGTVAPDSPASPASDGPALPSPAIPRGGFALSLLANAEAAARAPKGGGLFQRGALLGKLRRAESRTSVGSQRSRVSFLKSDLSSGASDAASTASLGEAAEGADEAAPFEADIDATTTETYMGEWKNDKRSGFGVSERSSGLRYEGEWLDNLRHGYGCTTLPDGHREEGKYRHNVLVKDTKRRMLQLKSNKVRQKVEHSVEGAQRAAAIARQKAEIAASRTSHAKAKAEAAEQAALAANQESNIARTLARELAPDFYQPGPEYQKRRLLQEILENSESLLEPPDRGAGAAGLPQPPRESPQLHERETPRPEGGSPSPAGTPPQPKRPRPGVSKDGLLSPGAWNGEPSGEGSRSVTPSEGAGRRSPARPATERMAIEALQAPPAPSREPEVALYQGYHSYAVRTTPPEPPPFEDQPEPEVSGSESAPSSPATAPLQAPTLRGPEPARETPAKLEPKPIIPKAEPRAKARKTEARGLTKAGAKKKARKEAALAAEAEVEVEEVPNTILICMVILLNIGLAILFVHLLT

SEQ ID NO: 324 - DNA, 1716 bp, synthetic construct
  source 1..1716: mol_type = other DNA
atgagtgggggccgcttcgactttgatgatggaggggcgtactgcgggggctgggaggggggaaaggcccatgggcatggactgtgcacaggccccaagggccagggcgaatactctggctcctggaactttggctttgaggtggcaggtgtctacacctggcccagcggaaacacctttgagggatactggagccagggcaaacggcatgggctgggcatagagaccaaggggcgctggctctacaagggcgagtggacacatggcttcaagggacgctacggaatccggcagagctcaagcagcggtgccaagtatgagggcacctggaacaatggcctgcaagacggctatggcaccgagacctatgctgatggagggacgtaccaaggccagttcaccaacggcatgcgccatggctacggagtacgccagagcgtgccctacgggatggccgtggtggtgcgctcgccgctgcgcacgtcgctgtcgtccctgcgcagcgagcacagcaacggcacggtggccccggactctcccgcctcgccggcctccgacggccccgcgctgccctcgcccgccatcccgcgtggcggcttcgcgctcagcctcctggccaatgccgaggcggccgcgcgggcgcccaagggcggcggcctcttccagcggggcgcgctgctgggcaagctgcggcgcgcagagtcgcgcacgtccgtgggtagccagcgcagccgtgtcagcttccttaagagcgacctcagctcgggcgccagcgacgccgcgtccaccgccagcctgggagaggccgccgagggcgccgacgaggccgcacccttcgaggccgatatcgacgccaccaccaccgagacctacatgggcgagtggaagaacgacaaacgctcgggcttcggcgtgagcgaacgctccagtggcctccgctacgagggcgagtggctggacaacctgcgccacggctatggctgcaccacgctgcccgacggccaccgcgaggagggcaagtaccgccacaacgtgctggtcaaggacaccaagcgccgcatgctgcagctcaagagcaacaaggtccgccagaaagtggagcacagtgtggagggtgcccagcgcgccgctgctatcgcgcgccagaaggccgagattgccgcctccaggacaagccacgccaaggccaaagctgaggcagcggaacaggccgccctggctgccaaccaggagtccaacattgctcgcactttggccagggagctggctccggacttctaccagccaggtccggaatatcagaagcgccggctgctgcaggagatcctggagaactcggagagcctgctggagccccccgaccggggcgccggcgcagcgggcctcccacagccgccccgcgagagcccgcagctgcacgagcgtgagacccctcggcccgagggtggctccccgtcaccggccgggacgcccccgcagcccaagcggcccaggcccggggtgtccaaggacggcctgctgagcccaggcgcctggaacggcgagcccagcggtgagggcagccggtcagtcactccgtccgagggcgcgggccgccgcagccccgcgcgtccagccaccgagcgcatggccatcgaggctctgcaggcaccgcctgcgccgtcgcgggagccggaggtggcgctttaccagggctaccacagctatgctgtgcgc

SEQ ID NO: 325 - protein, 572 aa, synthetic construct
MSGGRFDFDDGGAYCGGWEGGKAHGHGLCTGPKGQGEYSGSWNFGFEVAGVYTWPSGNTFEGYWSQGKRHGLGIETKGRWLYKGEWTHGFKGRYGIRQSSSSGAKYEGTWNNGLQDGYGTETYADGGTYQGQFTNGMRHGYGVRQSVPYGMAVVVRSPLRTSLSSLRSEHSNGTVAPDSPASPASDGPALPSPAIPRGGFALSLLANAEAAARAPKGGGLFQRGALLGKLRRAESRTSVGSQRSRVSFLKSDLSSGASDAASTASLGEAAEGADEAAPFEADIDATTTETYMGEWKNDKRSGFGVSERSSGLRYEGEWLDNLRHGYGCTTLPDGHREEGKYRHNVLVKDTKRRMLQLKSNKVRQKVEHSVEGAQRAAAIARQKAEIAASRTSHAKAKAEAAEQAALAANQESNIARTLARELAPDFYQPGPEYQKRRLLQEILENSESLLEPPDRGAGAAGLPQPPRESPQLHERETPRPEGGSPSPAGTPPQPKRPRPGVSKDGLLSPGAWNGEPSGEGSRSVTPSEGAGRRSPARPATERMAIEALQAPPAPSREPEVALYQGYHSYAVR

SEQ ID NO: 326 - DNA, 1728 bp, Homo sapiens
  source 1..1728: mol_type = genomic DNA
atgagcgccgccacccactcgcccatgatgcaggtggcgtccggcaacggtgaccgcgaccctttgccccccggatgggagatcaagatcgacccgcagaccggctggcccttcttcgtggaccacaacagccgcaccactacgtggaacgacccgcgcgtgccctctgagggccccaaggagactccatcctctgccaatggcccttcccgggagggctctaggctgccgcctgctagggaaggccaccctgtgtacccccagctccgaccaggctacattcccattcctgtgctccatgaaggcgctgagaaccggcaggtgcaccctttccatgtctatccccagcctgggatgcagcgattccgaactgaggcggcagcagcggctcctcagaggtcccagtcacctctgcggggcatgccagaaaccactcagccagataaacagtgtggacaggtggcagcggcggcggcagcccagcccccagcctcccacggacctgagcggtcccagtctccagctgcctctgactgctcatcctcatcctcctcggccagcctgccttcctccggcaggagcagcctgggcagtcaccagctcccgcgggggtacatctccattccggtgatacacgagcagaacgttacccggccagcagcccagccctccttccaccaagcccagaagacgcactacccagcgcagcagggggagtaccagacccaccagcctgtgtaccacaagatccagggggatgactgggagccccggcccctgcgggcggcatccccgttcaggtcatctgtccagggtgcatcgagccgggagggctcaccagccaggagcagcacgccactccactccccctcgcccatccgtgtgcacaccgtggtcgacaggcctcagcagcccatgacccatcgagaaactgcacctgtttcccagcctgaaaacaaaccagaaagtaagccaggcccagttggaccagaactccctcctggacacatcccaattcaagtgatccgcaaagaggtggattctaaacctgtttcccagaagcccccacctccctctgagaaggtagaggtgaaagttccccctgctccagttccttgtcctcctcccagccctggcccttctgctgtcccctcttcccccaagagtgtggctacagaagagagggcagcccccagcactgcccctgcagaagctacacctccaaaaccaggagaagccgaggctcccccaaaacatccaggagtgctgaaagtggaagccatcctggagaaggtacaggggctggagcaggctgtagacaactttgaaggcaagaagactgacaaaaagtacctgatgatcgaagagtatttgaccaaagagctgctggccctggattcagtggaccccgagggacgagccgatgtgcgtcaggccaggagagacggtgtcaggaaggttcagaccatcttggaaaaacttgaacagaaagccattgatgtcccaggtcaagtccaggtctatgaactccagcccagcaaccttgaagcagatcagccactgcaggcaatcatggagatgggtgccgtggcagcagacaagggcaagaaaaatgctggaaatgcagaagatccccacacagaaacccagcagccagaagccacagcagcagcgacttcaaaccccagcagcatgacagacacccctggtaacccagcagcaccgtag

SEQ ID NO: 327 - protein, 575 aa, Homo sapiens
MSAATHSPMMQVASGNGDRDPLPPGWEIKIDPQTGWPFFVDHNSRTTTWNDPRVPSEGPKETPSSANGPSREGSRLPPAREGHPVYPQLRPGYIPIPVLHEGAENRQVHPFHVYPQPGMQRFRTEAAAAAPQRSQSPLRGMPETTQPDKQCGQVAAAAAAQPPASHGPERSQSPAASDCSSSSSSASLPSSGRSSLGSHQLPRGYISIPVIHEQNVTRPAAQPSFHQAQKTHYPAQQGEYQTHQPVYHKIQGDDWEPRPLRAASPFRSSVQGASSREGSPARSSTPLHSPSPIRVHTVVDRPQQPMTHRETAPVSQPENKPESKPGPVGPELPPGHIPIQVIRKEVDSKPVSQKPPPPSEKVEVKVPPAPVPCPPPSPGPSAVPSSPKSVATEERAAPSTAPAEATPPKPGEAEAPPKHPGVLKVEAILEKVQGLEQAVDNFEGKKTDKKYLMIEEYLTKELLALDSVDPEGRADVRQARRDGVRKVQTILEKLEQKAIDVPGQVQVYELQPSNLEADQPLQAIMEMGAVAADKGKKNAGNAEDPHTETQQPEATAAATSNPSSMTDTPGNPAAP

SEQ ID NO: 328 - protein, 575 aa, Homo sapiens [sequence omitted]

SEQ ID NO: 329 - DNA, 528 bp, Homo sapiens
  source 1..528: mol_type = genomic DNA
atggacatcgccatccaccacccctggatccgccgccccttctttcctttccactcccccagccgcctctttgaccagttcttcggagagcacctgttggagtctgatcttttcccgacgtctacttccctgagtcccttctaccttcggccaccctccttcctgcgggcacccagctggtttgacactggactctcagagatgcgcctggagaaggacaggttctctgtcaacctggatgtgaagcacttctccccagaggaactcaaagttaaggtgttgggagatgtgattgaggtgcatggaaaacatgaagagcgccaggatgaacatggtttcatctccagggagttccacaggaaataccggatcccagctgatgtagaccctctcaccattacttcatccctgtcatctgatggggtcctcactgtgaatggaccaaggaaacaggtctctggccctgagcgcaccattcccatcacccgtgaagagaagcctgctgtcaccgcagcccccaagaaatag

SEQ ID NO: 330 - protein, 175 aa, Homo sapiens
MDIAIHHPWIRRPFFPFHSPSRLFDQFFGEHLLESDLFPTSTSLSPFYLRPPSFLRAPSWFDTGLSEMRLEKDRFSVNLDVKHFSPEELKVKVLGDVIEVHGKHEERQDEHGFISREFHRKYRIPADVDPLTITSSLSSDGVLTVNGPRKQVSGPERTIPITREEKPAVTAAPKK

SEQ ID NO: 331 - DNA, 1450 bp, Homo sapiens
  source 1..1450: mol_type = genomic DNA
atggagaccccgtcccagcggcgcgccacccgcagcggggcgcaggccagctccactccgctgtcgcccacccgcatcacccggctgcaggagaaggaggacctgcaggagctcaatgatcgcttggcggtctacatcgaccgtgtgcgctcgctggaaacggagaacgcagggctgcgccttcgcatcaccgagtctgaagaggtggtcagccgcgaggtgtccggcatcaaggccgcctacgaggccgagctcggggatgcccgcaagacccttgactcagtagccaaggagcgcgcccgcctgcagctggagctgagcaaagtgcgtgaggagtttaaggagctgaaagcgcgcaataccaagaaggagggtgacctgatagctgctcaggctcggctgaaggacctggaggctctgctgaactccaaggaggccgcactgagcactgctctcagtgagaagcgcacgctggagggcgagctgcatgatctgcggggccaggtggccaagcttgaggcagccctaggtgaggccaagaagcaacttcaggatgagatgctgcggcgggtggatgctgagaacaggctgcagaccatgaaggaggaactggacttccagaagaacatctacagtgaggagctgcgtgagaccaagcgccgtcatgagacccgactggtggagattgacaatgggaagcagcgtgagtttgagagccggctggcggatgcgctgcaggaactgcgggcccagcatgaggaccaggtggagcagtataagaaggagctggagaagacttattctgccaagctggacaatgccaggcagtctgctgagaggaacagcaacctggtgggggctgcccacgaggagctgcagcagtcgcgcatccgcatcgacagcctctctgcccagctcagccagctccagaagcagctggcagccaaggaggcgaagcttcgagacctggaggactcactggcccgtgagcgggacaccagccggcggctgctggcggaaaaggagcgggagatggccgagatgcgggcaaggatgcagcagcagctggacgagtaccaggagcttctggacatcaagctggccctggacatggagatccacgcctaccgcaagctcttggagggcgaggaggagaggctacgcctgtcccccagccctacctcgcagcgcagccgtggccgtgcttcctctcactcatcccagacacagggtgggggcagcgtcaccaaaaagcgcaaactggagtccactgagagccgcagcagcttctcacagcacgcacgcactagcgggcgcgtggccgtggaggaggtggatgaggagggcaagtttgtccggctgcgcaacaagtccaatgaggaccagtccatgggcaattggcagatcaagcgccagaatggagatgatcccttgctgacttaccggttcc

SEQ ID NO: 332 - protein, 664 aa, Homo sapiens
METPSQRRATRSGAQASSTPLSPTRITRLQEKEDLQELNDRLAVYIDRVRSLETENAGLRLRITESEEVVSREVSGIKAAYEAELGDARKTLDSVAKERARLQLELSKVREEFKELKARNTKKEGDLIAAQARLKDLEALLNSKEAALSTALSEKRTLEGELHDLRGQVAKLEAALGEAKKQLQDEMLRRVDAENRLQTMKEELDFQKNIYSEELRETKRRHETRLVEIDNGKQREFESRLADALQELRAQHEDQVEQYKKELEKTYSAKLDNARQSAERNSNLVGAAHEELQQSRIRIDSLSAQLSQLQKQLAAKEAKLRDLEDSLARERDTSRRLLAEKEREMAEMRARMQQQLDEYQELLDIKLALDMEIHAYRKLLEGEEERLRLSPSPTSQRSRGRASSHSSQTQGGGSVTKKRKLESTESRSSFSQHARTSGRVAVEEVDEEGKFVRLRNKSNEDQSMGNWQIKRQNGDDPLLTYRFPPKFTLKAGQVVTIWAAGAGATHSPPTDLVWKAQNTWGCGNSLRTALINSTGEEVAMRKLVRSVTVVEDDEDEDGDDLLHHHHGSHCSSSGDPAEYNLRSRTVLCGTCGQPADKASASGSGAQVGGPISSGSSASSVTVTRSYRSVGGSGGGSFGDNLVTRSYLLGNSSPRTQSPQNCSIM

SEQ ID NO: 333 - DNA, 1450 bp, Homo sapiens; identical to SEQ ID NO: 331
  source 1..1450: mol_type = genomic DNA

SEQ ID NO: 334 - protein, 572 aa, Homo sapiens
METPSQRRATRSGAQASSTPLSPTRITRLQEKEDLQELNDRLAVYIDRVRSLETENAGLRLRITESEEVVSREVSGIKAAYEAELGDARKTLDSVAKERARLQLELSKVREEFKELKARNTKKEGDLIAAQARLKDLEALLNSKEAALSTALSEKRTLEGELHDLRGQVAKLEAALGEAKKQLQDEMLRRVDAENRLQTMKEELDFQKNIYSEELRETKRRHETRLVEIDNGKQREFESRLADALQELRAQHEDQVEQYKKELEKTYSAKLDNARQSAERNSNLVGAAHEELQQSRIRIDSLSAQLSQLQKQLAAKEAKLRDLEDSLARERDTSRRLLAEKEREMAEMRARMQQQLDEYQELLDIKLALDMEIHAYRKLLEGEEERLRLSPSPTSQRSRGRASSHSSQTQGGGSVTKKRKLESTESRSSFSQHARTSGRVAVEEVDEEGKFVRLRNKSNEDQSMGNWQIKRQNGDDPLLTYRFPPKFTLKAGQVVTIWAAGAGATHSPPTDLVWKAQNTWGCGNSLRTALINSTGEEVAMRKLVRSVTVVEDDEDEDGDDLLHHHHVSGSRR

SEQ ID NO: 335 - DNA, 633 bp, Homo sapiens
  source 1..633: mol_type = genomic DNA
atggcggatgggagcagcgatgcggctagggaacctcgccctgcaccagccccaatcagacgccgctcctccaactaccgcgcttatgccacggagccgcacgccaagaaaaaatctaagatctccgcctcgagaaaattgcagctgaagactctgctgctgcagattgcaaagcaagagctggagcgagaggcggaggagcggcgcggagagaaggggcgcgctctgagcacccgctgccagccgctggagttggccgggctgggcttcgcggagctgcaggacttgtgccgacagctccacgcccgtgtggacaaggtggatgaagagagatacgacatagaggcaaaagtcaccaagaacatcacggagattgcagatctgactcagaagatctttgaccttcgaggcaagtttaagcggcccaccctgcggagagtgaggatctctgcagatgccatgatgcaggcgctgctgggggcccgggctaaggagtccctggacctgcgggcccacctcaagcaggtgaagaaggaggacaccgagaaggaaaaccgggaggtgggagactggcgcaagaacatcgatgcactgagtggaatggagggccgcaagaaaaagtttgagagctga

SEQ ID NO: 336 - protein, 210 aa, Homo sapiens
MADGSSDAAREPRPAPAPIRRRSSNYRAYATEPHAKKKSKISASRKLQLKTLLLQIAKQELEREAEERRGEKGRALSTRCQPLELAGLGFAELQDLCRQLHARVDKVDEERYDIEAKVTKNITEIADLTQKIFDLRGKFKRPTLRRVRISADAMMQALLGARAKESLDLRAHLKQVKKEDTEKENREVGDWRKNIDALSGMEGRKKKFES

SEQ ID NO: 337 - DNA, 159 bp, Homo sapiens
  source 1..159: mol_type = genomic DNA
atggagaaagtccaatacctcactcgctcagctataagaagagcctcaaccattgaaatgcctcaacaagcacgtcaaaagctacagaatctatttatcaatttctgtctcatcttaatatgtctcttgctgatctgtatcatcgtgatgcttctctga

SEQ ID NO: 338 - protein, 52 aa, Homo sapiens
MEKVQYLTRSAIRRASTIEMPQQARQKLQNLFINFCLILICLLLICIIVMLL

SEQ ID NO: 339 - DNA, 1233 bp, Homo sapiens
  source 1..1233: mol_type = genomic DNA
atggtgtgcttccgcctcttcccggttccgggctcagggctcgttctggtctgcctagtcctgggagctgtgcggtcttatgcattggaacttaatttgacagattcagaaaatgccacttgcctttatgcaaaatggcagatgaatttcacagtacgctatgaaactacaaataaaacttataaaactgtaaccatttcagaccatggcactgtgacatataatggaagcatttgtggggatgatcagaatggtcccaaaatagcagtgcagttcggacctggcttttcctggattgcgaattttaccaaggcagcatctacttattcaattgacagcgtctcattttcctacaacactggtgataacacaacatttcctgatgctgaagataaaggaattcttactgttgatgaacttttggccatcagaattccattgaatgacctttttagatgcaatagtttatcaactttggaaaagaatgatgttgtccaacactactgggatgttcttgtacaagcttttgtccaaaatggcacagtgagcacaaatgagttcctgtgtgataaagacaaaacttcaacagtggcacccaccatacacaccactgtgccatctcctactacaacacctactccaaaggaaaaaccagaagctggaacctattcagttaataatggcaatgatacttgtctgctggctaccatggggctgcagctgaacatcactcaggataaggttgcttcagttattaacatcaaccccaatacaactcactccacaggcagctgccgttctcacactgctctacttagactcaatagcagcaccattaagtatctagactttgtctttgctgtgaaaaatgaaaaccgattttatctgaaggaagtgaacatcagcatgtatttggttaatggctccgttttcagcattgcaaataacaatctcagctactgggatgcccccctgggaagttcttatatgtgcaacaaagagcagactgtttcagtgtctggagcatttcagataaatacctttgatctaagggttcagcctttcaatgtgacacaaggaaagtattctacagctcaagactgcagtgcagatgacgacaacttccttgtgcccatagcggtgggagctgccttggcaggagtacttattctagtgttgctggcttattttattggtctcaagcaccatcatgctggatatgagcaattttag

SEQ ID NO: 340 - protein, 410 aa, Homo sapiens
MVCFRLFPVPGSGLVLVCLVLGAVRSYALELNLTDSENATCLYAKWQMNFTVRYETTNKTYKTVTISDHGTVTYNGSICGDDQNGPKIAVQFGPGFSWIANFTKAASTYSIDSVSFSYNTGDNTTFPDAEDKGILTVDELLAIRIPLNDLFRCNSLSTLEKNDVVQHYWDVLVQAFVQNGTVSTNEFLCDKDKTSTVAPTIHTTVPSPTTTPTPKEKPEAGTYSVNNGNDTCLLATMGLQLNITQDKVASVININPNTTHSTGSCRSHTALLRLNSSTIKYLDFVFAVKNENRFYLKEVNISMYLVNGSVFSIANNNLSYWDAPLGSSYMCNKEQTVSVSGAFQINTFDLRVQPFNVTQGKYSTAQDCSADDDNFLVPIAVGAALAGVLILVLLAYFIGLKHHHAGYEQF

SEQ ID NO: 341 - DNA, 1233 bp, Homo sapiens
  source 1..1233: mol_type = genomic DNA
atggtgtgcttccgcctcttcccggttccgggctcagggctcgttctggtctgcctagtcctgggagctgtgcggtcttatgcattggaacttaatttgacagattcagaaaatgccacttgcctttatgcaaaatggcagatgaatttcacagtacgctatgaaactacaaataaaacttataaaactgtaaccatttcagaccatggcactgtgacatataatggaagcatttgtggggatgatcagaatggtcccaaaatagcagtgcagttcggacctggcttttcctggattgcgaattttaccaaggcagcatctacttattcaattgacagcgtctcattttcctacaacactggtgataacacaacatttcctgatgctgaagataaaggaattcttactgttgatgaacttttggccatcagaattccattgaatgacctttttagatgcaatagtttatcaactttggaaaagaatgatgttgtccaacactactgggatgttcttgtacaagcttttgtccaaaatggcacagtgagcacaaatgagttcctgtgtgataaagacaaaacttcaacagtggcacccaccatacacaccactgtgccatctcctactacaacacctactccaaaggaaaaaccagaagctggaacctattcagttaataatggcaatgatacttgtctgctggctaccatggggctgcagctgaacatcactcaggataaggttgcttcagttattaacatcaaccccaatacaactcactccacaggcagctgccgttctcacactgctctacttagactcaatagcagcaccattaagtatctagactttgtctttgctgtgaaaaatgaaaaccgattttatctgaaggaagtgaacatcagcatgtatttggttaatggctccgttttcagcattgcaaataacaatctcagctactgggatgcccccctgggaagttcttatatgtgcaacaaagagcagactgtttcagtgtctggagcatttcagataaatacctttgatctaagggttcagcctttcaatgtgacacaaggaaagtattctacagcccaagagtgttcgctggatgatgacaccattctaatcccaattatagttggtgctggtctttcaggcttgattatcgttatagtgattgcttacgtaattggcagaagaaaaagttatgctggatatcagactctgtaa

SEQ ID NO: 342 - protein, 410 aa, Homo sapiens
MVCFRLFPVPGSGLVLVCLVLGAVRSYALELNLTDSENATCLYAKWQMNFTVRYETTNKTYKTVTISDHGTVTYNGSICGDDQNGPKIAVQFGPGFSWIANFTKAASTYSIDSVSFSYNTGDNTTFPDAEDKGILTVDELLAIRIPLNDLFRCNSLSTLEKNDVVQHYWDVLVQAFVQNGTVSTNEFLCDKDKTSTVAPTIHTTVPSPTTTPTPKEKPEAGTYSVNNGNDTCLLATMGLQLNITQDKVASVININPNTTHSTGSCRSHTALLRLNSSTIKYLDFVFAVKNENRFYLKEVNISMYLVNGSVFSIANNNLSYWDAPLGSSYMCNKEQTVSVSGAFQINTFDLRVQPFNVTQGKYSTAQECSLDDDTILIPIIVGAGLSGLIIVIVIAYVIGRRKSYAGYQTL

SEQ ID NO: 343 - DNA, 1236 bp, Homo sapiens
  source 1..1236: mol_type = genomic DNA
atggtgtgcttccgcctcttcccggttccgggctcagggctcgttctggtctgcctagtcctgggagctgtgcggtcttatgcattggaacttaatttgacagattcagaaaatgccacttgcctttatgcaaaatggcagatgaatttcacagtacgctatgaaactacaaataaaacttataaaactgtaaccatttcagaccatggcactgtgacatataatggaagcatttgtggggatgatcagaatggtcccaaaatagcagtgcagttcggacctggcttttcctggattgcgaattttaccaaggcagcatctacttattcaattgacagcgtctcattttcctacaacactggtgataacacaacatttcctgatgctgaagataaaggaattcttactgttgatgaacttttggccatcagaattccattgaatgacctttttagatgcaatagtttatcaactttggaaaagaatgatgttgtccaacactactgggatgttcttgtacaagcttttgtccaaaatggcacagtgagcacaaatgagttcctgtgtgataaagacaaaacttcaacagtggcacccaccatacacaccactgtgccatctcctactacaacacctactccaaaggaaaaaccagaagctggaacctattcagttaataatggcaatgatacttgtctgctggctaccatggggctgcagctgaacatcactcaggataaggttgcttcagttattaacatcaaccccaatacaactcactccacaggcagctgccgttctcacactgctctacttagactcaatagcagcaccattaagtatctagactttgtctttgctgtgaaaaatgaaaaccgattttatctgaaggaagtgaacatcagcatgtatttggttaatggctccgttttcagcattgcaaataacaatctcagctactgggatgcccccctgggaagttcttatatgtgcaacaaagagcagactgtttcagtgtctggagcatttcagataaatacctttgatctaagggttcagcctttcaatgtgacacaaggaaagtattctacagctgaagaatgttctgctgactctgacctcaactttcttattcctgttgcagtgggtgtggccttgggcttccttataattgttgtctttatctcttatatgattggaagaaggaaaagtcgtactggttatcagtctgtgtaa

SEQ ID NO: 344 - protein, 411 aa, Homo sapiens
MVCFRLFPVPGSGLVLVCLVLGAVRSYALELNLTDSENATCLYAKWQMNFTVRYETTNKTYKTVTISDHGTVTYNGSICGDDQNGPKIAVQFGPGFSWIANFTKAASTYSIDSVSFSYNTGDNTTFPDAEDKGILTVDELLAIRIPLNDLFRCNSLSTLEKNDVVQHYWDVLVQAFVQNGTVSTNEFLCDKDKTSTVAPTIHTTVPSPTTTPTPKEKPEAGTYSVNNGNDTCLLATMGLQLNITQDKVASVININPNTTHSTGSCRSHTALLRLNSSTIKYLDFVFAVKNENRFYLKEVNISMYLVNGSVFSIANNNLSYWDAPLGSSYMCNKEQTVSVSGAFQINTFDLRVQPFNVTQGKYSTAEECSADSDLNFLIPVAVGVALGFLIIVVFISYMIGRRKSRTGYQSV

SEQ ID NO: 345 - DNA, 8616 bp, Homo sapiens
  source 1..8616: mol_type = genomic DNA
atgagctgcaacggaggctcccacccgcggatcaacactctgggccgcatgatccgcgccgagtctggcccggacctgcgctacgaggtgaccagcggcggcgggggcaccagcaggatgtactattctcggcgcggcgtgatcaccgaccagaactcggacggctactgtcaaaccggcacgatgtccaggcaccagaaccagaacaccatccaggagctgctgcagaactgctccgactgcttgatgcgagcagagctcatcgtgcagcctgaattgaagtatggagatggaatacaactgactcggagtcgagaattggatgagtgttttgcccaggccaatgaccaaatggaaatcctcgacagcttgatcagagagatgcggcagatgggccagccctgtgatgcttaccagaaaaggcttcttcagctccaagagcaaatgcgagccctttataaagccatcagtgtccctcgagtccgcagggccagctccaagggtggtggaggctacacttgtcagagtggctctggctgggatgagttcaccaaacatgtcaccagtgaatgtttggggtggatgaggcagcaaagggcggagatggacatggtggcctggggtgtggacctggcctcagtggagcagcacattaacagccaccggggcatccacaactccatcggcgactatcgctggcagctggacaaaatcaaagccgacctgcgcgagaaatctgcgatctaccagttggaggaggagtatgaaaacctgctgaaagcgtcctttgagaggatggatcacctgcgacagctgcagaacatcattcaggccacgtccagggagatcatgtggatcaatgactgcgaggaggaggagctgctgtacgactggagcgacaagaacaccaacatcgctcagaaacaggaggccttctccatacgcatgagtcaactggaagttaaagaaaaagagctcaataagctgaaacaagaaagtgaccaacttgtcctcaatcagcatccagcttcagacaaaattgaggcctatatggacactctgcagacgcagtggagttggattcttcagatcaccaagtgcattgatgttcatctgaaagaaaatgctgcctactttcagttttttgaagaggcgcagtctactgaagcatacctgaaggggctccaggactccatcaggaagaagtacccctgcgacaagaacatgcccctgcagcacctgctggaacagatcaaggagctggagaaagaacgagagaaaatccttgaatacaagcgtcaggtgcagaacttggtaaacaagtctaagaagattgtacagctgaagcctcgtaacccagactacagaagcaataaacccattattctcagagctctctgtgactacaaacaagatcagaaaatcgtgcataagggggatgagtgtatcctgaaggacaacaacgagcgcagcaagtggtacgtgacgggcccgggaggcgttgacatgcttgttccctctgtggggctgatcatccctcctccgaacccactggccgtggacctctcttgcaagattgagcagtactacgaagccatcttggctctgtggaaccagctctacatcaacatgaagagcctggtgtcctggcactactgcatgattgacatagagaagatcagggccatgacaatcgccaagctgaaaacaatgcggcaggaagattacatgaagacgatagccgaccttgagttacattaccaagagttcatcagaaatagccaaggctcagagatgtttggagatgatgacaagcggaaaatacagtctcagttcaccgatgcccagaagcattaccagaccctggtcattcagctccctggctatccccagcaccagacagtgaccacaactgaaatcactcatcatggaacctgccaagatgtcaaccataataaagtaattgaaaccaacagagaaaatgacaagcaagaaacatggatgctgatggagctgcagaagattcgcaggcagatagagcactgcgagggcaggatgactctcaaaaacctccctctagcagaccagggatcttctcaccacatcacagtgaaaattaacgagcttaagagtgtgcagaatgattcacaagcaattgctgaggttctcaaccagcttaaagatatgcttgccaacttcagaggttctgaaaagtactgctatttacagaatgaagtatttggactatttcagaaactggaaaatatcaatggtgttacagatggctacttaaatagcttatgcacagtaagggcactgctccaggctattctccaaacagaagacatgttaaaggtttatgaagccaggctcactgaggaggaaactgtctgcctggacctggataaagtggaagcttaccgctgtggactgaagaaaataaaaaatgacttgaacttgaagaagtcgttgttggccactatgaagacagaactacagaaagcccagcagatccactctcagacttcacagcagtatccactttatgatctggacttgggcaagttcggtgaaaaagtcacacagctgacagaccgctggcaaaggatagataaacagatcgactttaggttatgggacctggagaaacaaatcaagcaattgaggaattatcgtgataactatcaggctttctgcaagtggctctatgatgctaaacgccgccaggattccttagaatccatgaaatttggagattccaacacagtcatgcggtttttgaatgagcagaagaacttgcacagtgaaatatctggcaaacgagacaaatcagaggaagtacaaaaaattgctgaactttgcgccaattcaattaaggattatgagctccagctggcctcatacacctcaggactggaaactctgctgaacatacctatcaagaggaccatgattcagtccccttctggggtgattctgcaagaggctgcagatgttcatgctcggtacattgaactacttacaagatctggagactattacaggttcttaagtgagatgctgaagagtttggaagatctgaagctgaaaaataccaagatcgaagttttggaagaggagctcagactggcccgagatgccaactcggaaaactgtaataagaacaaattcctggatcagaacctgcagaaataccaggcagagtgttcccagttcaaagcgaagcttgcgagcctggaggagctgaagagacaggctgagctggatgggaagtcggctaagcaaaatctagacaagtgctacggccaaataaaagaactcaatgagaagatcacccgactgacttatgagattgaagatgaaaagagaagaagaaaatctgtggaagacagatttgaccaacagaagaatgactatgaccaactgcagaaagcaaggcaatgtgaaaaggagaaccttggttggcagaaattagagtctgagaaagccatcaaggagaaggagtacgagattgaaaggttgagggttctactgcaggaagaaggcacccggaagagagaatatgaaaatgagctggcaaaggtaagaaaccactataatgaggagatgagtaatttaaggaacaagtatgaaacagagattaacattacgaagaccaccatcaaggagatatccatgcaaaaagaggatgattccaaaaatcttagaaaccagcttgatagactttcaagggaaaatcgagatctgaaggatgaaattgtcaggctcaatgacagcatcttgcaggccactgagcagcgaaggcgagctgaagaaaacgcccttcagcaaaaggcctgtggctctgagataatgcagaagaagcagcatctggagatagaactgaagcaggtcatgcagcagcgctctgaggacaatgcccggcacaagcagtccctggaggaggctgccaagaccattcaggacaaaaataaggagatcgagagactcaaagctgagtttcaggaggaggccaagcgccgctgggaatatgaaaatgaactgagtaaggtaagaaacaattatgatgaggagatcattagcttaaaaaatcagtttgagaccgagatcaacatcaccaagaccaccatccaccagctcaccatgcagaaggaagaggataccagtggctaccgggctcagatagacaatctcacccgagaaaacaggagcttatctgaagaaataaagaggctgaagaacactctaacccagaccacagagaatctcaggagggtggaagaagacatccaacagcaaaaggccactggctctgaggtgtctcagaggaaacagcagctggaggttgagctgagacaagtcactcagatgcgaacagaggagagcgtaagatataagcaatctcttgatgatgctgccaaaaccatccaggataaaaacaaggagatagaaaggttaaaacaactgatcgacaaagaaacaaatgaccggaaatgcctggaagatgaaaacgcgagattacaaagggtccagtatgacctgcagaaagcaaacagtagtgcgacggagacaataaacaaactgaaggttcaggagcaagaactgacacgcctgaggatcgactatgaaagggtttcccaggagaggactgtgaaggaccaggatatcacgcggttccagaactctctgaaagagctgcagctgcagaagcagaaggtggaagaggagctgaatcggctgaagaggaccgcgtcagaagactcctgcaagaggaagaagctggaggaagagctggaaggcatgaggaggtcgctgaaggagcaagccatcaaaatcaccaacctgacccagcagctggagcaggcatccattgttaagaagaggagtgaggatgacctccggcagcagagggacgtgctggatggccacctgagggaaaagcagaggacccaggaagagctgaggaggctctcttctgaggtcgaggccctgaggcggcagttactccaggaacaggaaagtgtcaaacaagctcacttgaggaatgagcatttccagaaggcgatagaagataaaagcagaagcttaaatgaaagcaaaatagaaattgagaggctgcagtctctcacagagaacctgaccaaggagcacttgatgttagaagaagaactgcggaacctgaggctggagtacgatgacctgaggagaggacgaagcgaagcggacagtgataaaaatgcaaccatcttggaactaaggagccagctgcagatcagcaacaaccggaccctggaactgcaggggctgattaatgatttacagagagagagggaaaatttgagacaggaaattgagaaattccaaaagcaggctttagaggcatctaataggattcaggaatcaaagaatcagtgtactcaggtggtacaggaaagagagagccttctggtgaaaatcaaagtcctggagcaagacaaggcaaggctgcagaggctggaggatgagctgaatcgtgcaaaatcaactctagaggcagaaaccagggtgaaacagcgcctggagtgtgagaaacagcaaattcagaatgacctgaatcagtggaagactcaatattcccgcaaggaggaggctattaggaagatagaatcggaaagagaaaagagtgagagagagaagaacagtcttaggagtgagatcgaaagactccaagcagagatcaagagaattgaagagaggtgcaggcgtaagctggaggattctaccagggagacacagtcacagttagaaacagaacgctcccgatatcagagggagattgataaactcagacagcgcccatatgggtcccatcgagagacccagactgagtgtgagtggaccgttgacacctccaagctggtgtttgatgggctgaggaagaaggtgacagcaatgcagctctatgagtgtcagctgatcgacaaaacaaccttggacaaactattgaaggggaagaagtcagtggaagaagttgcttctgaaatccagccattccttcggggtgcaggatctatcgctggagcatctgcttctcctaaggaaaaatactctttggtagaggccaagagaaagaaattaatcagcccagaatccacagtcatgcttctggaggcccaggcagctacaggtggtataattgatccccatcggaatgagaagctgactgtcgacagtgccatagctcgggacctcattgacttcgatgaccgtcagcagatatatgcagcagaaaaagctatcactggttttgatgatccattttcaggcaagacagtatctgtttcagaagccatcaagaaaaatttgattgatagagaaaccggaatgcgcctgctggaagcccagattgcttcagggggtgtagtagaccctgtgaacagtgtctttttgccaaaagatgtcgccttggcccgggggctgattgatagagatttgtatcgatccctgaatgatccccgagatagtcagaaaaactttgtggatccagtcaccaaaaagaaggtcagttacgtgcagctgaaggaacggtgcagaatcgaaccacatactggtctgctcttgctttcagtacagaagagaagcatgtccttccaaggaatcagacaacctgtgaccgtcactgagctagtagattctggtatattgagaccgtccactgtcaatgaactggaatctggtcagatttcttatgacgaggttggtgagagaattaaggacttcctccagggttcaagctgcatagcaggcatatacaatgagaccacaaaacagaagcttggcatttatgaggccatgaaaattggcttagtccgacctggtactgctctggagttgctggaagcccaagcagctactggctttatagtggatcctgttagcaacttgaggttaccagtggaggaagcctacaagagaggtctggtgggcattgagttcaaagagaagctcctgtctgcagaacgagctgtcactgggtataatgatcctgaaacaggaaacatcatctctttgttccaagccatgaataaggaactcatcgaaaagggccacggtattcgcttattagaagcacagatcgcaaccggggggatcattgacccaaaggagagccatcgtttaccagttgacatagcatataagaggggctatttcaatgaggaactcagtgagattctctcagatccaagtgatgataccaaaggattttttgaccccaacactgaagaaaatcttacctatctgcaactaaaagaaagatgcattaaggatgaggaaacagggctctgtcttctgcctctgaaagaaaagaagaaacaggtgcagacatcacaaaagaataccctcaggaagcgtagagtggtcatagttgacccagaaaccaataaagaaatgtctgttcaggaggcctacaagaagggcctaattgattatgaaaccttcaaagaactgtgtgagcaggaatgtgaatgggaagaaataaccatcacgggatcagatggctccaccagggtggtcctggtagatagaaagacaggcagtcagtatgatattcaagatgctattgacaagggccttgttgacaggaagttctttgatcagtaccgatccggcagcctcagcctcactcaatttgctgacatgatctccttgaaaaatggtgtcggcaccagcagcagcatgggcagtggtgtcagcgatgatgtttttagcagctcccgacatgaatcagtaagtaagatttccaccatatccagcgtcaggaatttaaccataaggagcagctctttttcagacaccctggaagaatcgagccccattgcagccatctttgacacagaaaacctggagaaaatctccattacagaaggtatagagcggggcatcgttgacagcatcacgggtcagaggcttctggaggctcaggcctgcacaggtggcatcatccacccaaccacgggccagaagctgtcacttcaggacgcagtctcccagggtgtgattgaccaagacatggccaccaggctgaagcctgctcagaaagccttcataggcttcgagggtgtgaagggaaagaagaagatgtcagcagcagaggcagtgaaagaaaaatggctcccgtatgaggctggccagcgcttcctggagttccagtacctcacgggaggtcttgttgacccggaagtgcatgggaggataagcaccgaagaagccatccggaaggggttcatagatggccgcgccgcacagaggctgcaagacaccagcagctatgccaaaatcctgacctgccccaaaaccaaattaaaaatatcctataaggatgccataaatcgctccatggtagaagatatcactgggctgcgccttctggaagccgcctccgtgtcgtccaagggcttacccagcccttacaacatgtcttcggctccggggtcccgctccggctcccgctcgggatctcgctccggatctcgctccgggtcccgcagtgggtcccggagaggaagctttgacgccacagggaattcttcctactcttattcctactcatttagcagtagttctattgggcactag

SEQ ID NO: 346 - protein, 2871 aa, Homo sapiens
MSCNGGSHPRINTLGRMIRAESGPDLRYEVTSGGGGTSRMYYSRRGVITDQNSDGYCQTGTMSRHQNQNTIQELLQNCSDCLMRAELIVQPELKYGDGIQLTRSRELDECFAQANDQMEILDSLIREMRQMGQPCDAYQKRLLQLQEQMRALYKAISVPRVRRASSKGGGGYTCQSGSGWDEFTKHVTSECLGWMRQQRAEMDMVAWGVDLASVEQHINSHRGIHNSIGDYRWQLDKIKADLREKSAIYQLEEEYENLLKASFERMDHLRQLQNIIQATSREIMWINDCEEEELLYDWSDKNTNIAQKQEAFSIRMSQLEVKEKELNKLKQESDQLVLNQHPASDKIEAYMDTLQTQWSWILQITKCIDVHLKENAAYFQFFEEAQSTEAYLKGLQDSIRKKYPCDKNMPLQHLLEQIKELEKEREKILEYKRQVQNLVNKSKKIVQLKPRNPDYRSNKPIILRALCDYKQDQKIVHKGDECILKDNNERSKWYVTGPGGVDMLVPSVGLIIPPPNPLAVDLSCKIEQYYEAILALWNQLYINMKSLVSWHYCMIDIEKIRAMTIAKLKTMRQEDYMKTIADLELHYQEFIRNSQGSEMFGDDDKRKIQSQFTDAQKHYQTLVIQLPGYPQHQTVTTTEITHHGTCQDVNHNKVIETNRENDKQETWMLMELQKIRRQIEHCEGRMTLKNLPLADQGSSHHITVKINELKSVQNDSQAIAEVLNQLKDMLANFRGSEKYCYLQNEVFGLFQKLENINGVTDGYLNSLCTVRALLQAILQTEDMLKVYEARLTEEETVCLDLDKVEAYRCGLKKIKNDLNLKKSLLATMKTELQKAQQIHSQTSQQYPLYDLDLGKFGEKVTQLTDRWQRIDKQIDFRLWDLEKQIKQLRNYRDNYQAFCKWLYDAKRRQDSLESMKFGDSNTVMRFLNEQKNLHSEISGKRDKSEEVQKIAELCANSIKDYELQLASYTSGLETLLNIPIKRTMIQSPSGVILQEAADVHARYIELLTRSGDYYRFLSEMLKSLEDLKLKNTKIEVLEEELRLARDANSENCNKNKFLDQNLQKYQAECSQFKAKLASLEELKRQAELDGKSAKQNLDKCYGQIKELNEKITRLTYEIEDEKRRRKSVEDRFDQQKNDYDQLQKARQCEKENLGWQKLESEKAIKEKEYEIERLRVLLQEEGTRKREYENELAKVRNHYNEEMSNLRNKYETEINITKTTIKEISMQKEDDSKNLRNQLDRLSRENRDLKDEIVRLNDSILQATEQRRRAEENALQQKACGSEIMQKKQHLEIELKQVMQQRSEDNARHKQSLEEAAKTIQDKNKEIERLKAEFQEEAKRRWEYENELSKVRNNYDEEIISLKNQFETEINITKTTIHQLTMQKEEDTSGYRAQIDNLTRENRSLSEEIKRLKNTLTQTTENLRRVEEDIQQQKATGSEVSQRKQQLEVELRQVTQMRTEESVRYKQSLDDAAKTIQDKNKEIERLKQLIDKETNDRKCLEDENARLQRVQYDLQKANSSATETINKLKVQEQELTRLRIDYERVSQERTVKDQDITRFQNSLKELQLQKQKVEEELNRLKRTASEDSCKRKKLEEELEGMRRSLKEQAIKITNLTQQLEQASIVKKRSEDDLRQQRDVLDGHLREKQRTQEELRRLSSEVEALRRQLLQEQESVKQAHLRNEHFQKAIEDKSRSLNESKIEIERLQSLTENLTKEHLMLEEELRNLRLEYDDLRRGRSEADSDKNATILELRSQLQISNNRTLELQGLINDLQRERENLRQEIEKFQKQALEASNRIQESKNQCTQVVQERESLLVKIKVLEQDKARLQRLEDELNRAKSTLEAETRVKQRLECEKQQIQNDLNQWKTQYSRKEEAIRKIESEREKSEREKNSLRSEIERLQAEIKRIEERCRRKLEDSTRETQSQLETERSRYQREIDKLRQRPYGSHRETQTECEWTVDTSKLVFDGLRKKVTAMQLYECQLIDKTTLDKLLKGKKSVEEVASEIQPFLRGAGSIAGASASPKEKYSLVEAKRKKLISPESTVMLLEAQAATGGIIDPHRNEKLTVDSAIARDLIDFDDRQQIYAAEKAITGFDDPFSGKTVSVSEAIKKNLIDRETGMRLLEAQIASGGVVDPVNSVFLPKDVALARGLIDRDLYRSLNDPRDSQKNFVDPVTKKKVSYVQLKERCRIEPHTGLLLLSVQKRSMSFQGIRQPVTVTELVDSGILRPSTVNELESGQISYDEVGERIKDFLQGSSCIAGIYNETTKQKLGIYEAMKIGLVRPGTALELLEAQAATGFIVDPVSNLRLPVEEAYKRGLVGIEFKEKLLSAERAVTGYNDPETGNIISLFQAMNKELIEKGHGIRLLEAQIATGGIIDPKESHRLPVDIAYKRGYFNEELSEILSDPSDDTKGFFDPNTEENLTYLQLKERCIKDEETGLCLLPLKEKKKQVQTSQKNTLRKRRVVIVDPETNKEMSVQEAYKKGLIDYETFKELCEQECEWEEITITGSDGSTRVVLVDRKTGSQYDIQDAIDKGLVDRKFFDQYRSGSLSLTQFADMISLKNGVGTSSSMGSGVSDDVFSSSRHESVSKISTISSVRNLTIRSSSFSDTLEESSPIAAIFDTENLEKISITEGIERGIVDSITGQRLLEAQACTGGIIHPTTGQKLSLQDAVSQGVIDQDMATRLKPAQKAFIGFEGVKGKKKMSAAEAVKEKWLPYEAGQRFLEFQYLTGGLVDPEVHGRISTEEAIRKGFIDGRAAQRLQDTSSYAKILTCPKTKLKISYKDAINRSMVEDITGLRLLEAASVSSKGLPSPYNMSSAPGSRSGSRSGSRSGSRSGSRSGSRRGSFDATGNSSYSYSYSFSSSSIGH

SEQ ID NO: 347 - DNA, 6819 bp, Homo sapiens
  source 1..6819: mol_type = genomic DNA
atgagctgcaacggaggctcccacccgcggatcaacactctgggccgcatgatccgcgccgagtctggcccggacctgcgctacgaggtgaccagcggcggcgggggcaccagcaggatgtactattctcggcgcggcgtgatcaccgaccagaactcggacggctactgtcaaaccggcacgatgtccaggcaccagaaccagaacaccatccaggagctgctgcagaactgctccgactgcttgatgcgagcagagctcatcgtgcagcctgaattgaagtatggagatggaatacaactgactcggagtcgagaattggatgagtgttttgcccaggccaatgaccaaatggaaatcctcgacagcttgatcagagagatgcggcagatgggccagccctgtgatgcttaccagaaaaggcttcttcagctccaagagcaaatgcgagccctttataaagccatcagtgtccctcgagtccgcagggccagctccaagggtggtggaggctacacttgtcagagtggctctggctgggatgagttcaccaaacatgtcaccagtgaatgtttggggtggatgaggcagcaaagggcggagatggacatggtggcctggggtgtggacctggcctcagtggagcagcacattaacagccaccggggcatccacaactccatcggcgactatcgctggcagctggacaaaatcaaagccgacctgcgcgagaaatctgcgatctaccagttggaggaggagtatgaaaacctgctgaaagcgtcctttgagaggatggatcacctgcgacagctgcagaacatcattcaggccacgtccagggagatcatgtggatcaatgactgcgaggaggaggagctgctgtacgactggagcgacaagaacaccaacatcgctcagaaacaggaggccttctccatacgcatgagtcaactggaagttaaagaaaaagagctcaataagctgaaacaagaaagtgaccaacttgtcctcaatcagcatccagcttcagacaaaattgaggcctatatggacactctgcagacgcagtggagttggattcttcagatcaccaagtgcattgatgttcatctgaaagaaaatgctgcctactttcagttttttgaagaggcgcagtctactgaagcatacctgaaggggctccaggactccatcaggaagaagtacccctgcgacaagaacatgcccctgcagcacctgctggaacagatcaaggagctggagaaagaacgagagaaaatccttgaatacaagcgtcaggtgcagaacttggtaaacaagtctaagaagattgtacagctgaagcctcgtaacccagactacagaagcaataaacccattattctcagagctctctgtgactacaaacaagatcagaaaatcgtgcataagggggatgagtgtatcctgaaggacaacaacgagcgcagcaagtggtacgtgacgggcccgggaggcgttgacatgcttgttccctctgtggggctgatcatccctcctccgaacccactggccgtggacctctcttgcaagattgagcagtactacgaagccatcttggctctgtggaaccagctctacatcaacatgaagagcctggtgtcctggcactactgcatgattgacatagagaagatcagggccatgacaatcgccaagctgaaaacaatgcggcaggaagattacatgaagacgatagccgaccttgagttacattaccaagagttcatcagaaatagccaaggctcagagatgtttggagatgatgacaagcggaaaatacagtctcagttcaccgatgcccagaagcattaccagaccctggtcattcagctccctggctatccccagcaccagacagtgaccacaactgaaatcactcatcatggaacctgccaagatgtcaaccataataaagtaattgaaaccaacagagaaaatgacaagcaagaaacatggatgctgatggagctgcagaagattcgcaggcagatagagcactgcgagggcaggatgactctcaaaaacctccctctagcagaccagggatcttctcaccacatcacagtgaaaattaacgagcttaagagtgtgcagaatgattcacaagcaattgctgaggttctcaaccagcttaaagatatgcttgccaacttcagaggttctgaaaagtactgctatttacagaatgaagtatttggactatttcagaaactggaaaatatcaatggtgttacagatggctacttaaatagcttatgcacagtaagggcactgctccaggctattctccaaacagaagacatgttaaaggtttatgaagccaggctcactgaggaggaaactgtctgcctggacctggataaagtggaagcttaccgctgtggactgaagaaaataaaaaatgacttgaacttgaagaagtcgttgttggccactatgaagacagaactacagaaagcccagcagatccactctcagacttcacagcagtatccactttatgatctggacttgggcaagttcggtgaaaaagtcacacagctgacagaccgctggcaaaggatagataaacagatcgactttaggttatgggacctggagaaacaaatcaagcaattgaggaattatcgtgataactatcaggctttctgcaagtggctctatgatgctaaacgccgccaggattccttagaatccatgaaatttggagattccaacacagtcatgcggtttttgaatgagcagaagaacttgcacagtgaaatatctggcaaacgagacaaatcagaggaagtacaaaaaattgctgaactttgcgccaattcaattaaggattatgagctccagctggcctcatacacctcaggactggaaactctgctgaacatacctatcaagaggaccatgattcagtccccttctggggtgattctgcaagaggctgcagatgttcatgctcggtacattgaactacttacaagatctggagactattacaggttcttaagtgagatgctgaagagtttggaagatctgaagctgaaaaataccaagatcgaagttttggaagaggagctcagactggcccgagatgccaactcggaaaactgtaataagaacaaattcctggatcagaacctgcagaaataccaggcagagtgttcccagttcaaagcgaagcttgcgagcctggaggagctgaagagacaggctgagctggatgggaagtcggctaagcaaaatctagacaagtgctacggccaaataaaagaactcaatgagaagatcacccgactgacttatgagattgaagatgaaaagagaagaagaaaatctgtggaagacagatttgaccaacagaagaatgactatgaccaactgcagaaagcaaggcaatgtgaaaaggagaaccttggttggcagaaattagagtctgagaaagccatcaaggagaaggagtacgagattgaaaggttgagggttctactgcaggaagaaggcacccggaagagagaatatgaaaatgagctggcaaaggcatctaataggattcaggaatcaaagaatcagtgtactcaggtggtacaggaaagagagagccttctggtgaaaatcaaagtcctggagcaagacaaggcaaggctgcagaggctggaggatgagctgaatcgtgcaaaatcaactctagaggcagaaaccagggtgaaacagcgcctggagtgtgagaaacagcaaattcagaatgacctgaatcagtggaagactcaatattcccgcaaggaggaggctattaggaagatagaatcggaaagagaaaagagtgagagagagaagaacagtcttaggagtgagatcgaaagactccaagcagagatcaagagaattgaagagaggtgcaggcgtaagctggaggattctaccagggagacacagtcacagttagaaacagaacgctcccgatatcagagggagattgataaactcagacagcgcccatatgggtcccatcgagagacccagactgagtgtgagtggaccgttgacacctccaagctggtgtttgatgggctgaggaagaaggtgacagcaatgcagctctatgagtgtcagctgatcgacaaaacaaccttggacaaactattgaaggggaagaagtcagtggaagaagttgcttctgaaatccagccattccttcggggtgcaggatctatcgctggagcatctgcttctcctaaggaaaaatactctttggtagaggccaagagaaagaaattaatcagcccagaatccacagtcatgcttctggaggcccaggcagctacaggtggtataattgatccccatcggaatgagaagctgactgtcgacagtgccatagctcgggacctcattgacttcgatgaccgtcagcagatatatgcagcagaaaaagctatcactggttttgatgatccattttcaggcaagacagtatctgtttcagaagccatcaagaaaaatttgattgatagagaaaccggaatgcgcctgctggaagcccagattgcttcagggggtgtagtagaccctgtgaacagtgtctttttgccaaaagatgtcgccttggcccgggggctgattgatagagatttgtatcgatccctgaatgatccccgagatagtcagaaaaactttgtggatccagtcaccaaaaagaaggtcagttacgtgcagctgaaggaacggtgcagaatcgaaccacatactggtctgctcttgctttcagtacagaagagaagcatgtccttccaaggaatcagacaacctgtgaccgtcactgagctagtagattctggtatattgagaccgtccactgtcaatgaactggaatctggtcagatttcttatgacgaggttggtgagagaattaaggacttcctccagggttcaagctgcatagcaggcatatacaatgagaccacaaaacagaagcttggcatttatgaggccatgaaaattggcttagtccgacctggtactgctctggagttgctggaagcccaagcagctactggctttatagtggatcctgttagcaacttgaggttaccagtggaggaagcctacaagagaggtctggtgggcattgagttcaaagagaagctcctgtctgcagaacgagctgtcactgggtataatgatcctgaaacaggaaacatcatctctttgttccaagccatgaataaggaactcatcgaaaagggccacggtattcgcttattagaagcacagatcgcaaccggggggatcattgacccaaaggagagccatcgtttaccagttgacatagcatataagaggggctatttcaatgaggaactcagtgagattctctcagatccaagtgatgataccaaaggattttttgaccccaacactgaagaaaatcttacctatctgcaactaaaagaaagatgcattaaggatgaggaaacagggctctgtcttctgcctctgaaagaaaagaagaaacaggtgcagacatcacaaaagaataccctcaggaagcgtagagtggtcatagttgacccagaaaccaataaagaaatgtctgttcaggaggcctacaagaagggcctaattgattatgaaaccttcaaagaactgtgtgagcaggaatgtgaatgggaagaaataaccatcacgggatcagatggctccaccagggtggtcctggtagatagaaagacaggcagtcagtatgatattcaagatgctattgacaagggccttgttgacaggaagttctttgatcagtaccgatccggcagcctcagcctcactcaatttgctgacatgatctccttgaaaaatggtgtcggcaccagcagcagcatgggcagtggtgtcagcgatgatgtttttagcagctcccgacatgaatcagtaagtaagatttccaccatatccagcgtcaggaatttaaccataaggagcagctctttttcagacaccctggaagaatcgagccccattgcagccatctttgacacagaaaacctggagaaaatctccattacagaaggtatagagcggggcatcgttgacagcatcacgggtcagaggcttctggaggctcaggcctgcacaggtggcatcatccacccaaccacgggccagaagctgtcacttcaggacgcagtctcccagggtgtgattgaccaagacatggccaccaggctgaagcctgctcagaaagccttcataggcttcgagggtgtgaagggaaagaagaagatgtcagcagcagaggcagtgaaagaaaaatggctcccgtatgaggctggccagcgcttcctggagttccagtacctcacgggaggtcttgttgacccggaagtgcatgggaggataagcaccgaagaagccatccggaaggggttcatagatggccgcgccgcacagaggctgcaagacaccagcagctatgccaaaatcctgacctgccccaaaaccaaattaaaaatatcctataaggatgccataaatcgctccatggtagaagatatcactgggctgcgccttctggaagccgcctccgtgtcgtccaagggcttacccagcccttacaacatgtcttcggctccggggtcccgctccggctcccgctcgggatctcgctccggatctcgctccgggtcccgcagtgggtcccggagaggaagctttgacgccacagggaattcttcctactcttattcctactcatttagcagtagttctattgggcactag

SEQ ID NO: 348 - protein, 2272 aa, Homo sapiens
MSCNGGSHPRINTLGRMIRAESGPDLRYEVTSGGGGTSRMYYSRRGVITDQNSDGYCQTGTMSRHQNQNTIQELLQNCSDCLMRAELIVQPELKYGDGIQLTRSRELDECFAQANDQMEILDSLIREMRQMGQPCDAYQKRLLQLQEQMRALYKAISVPRVRRASSKGGGGYTCQSGSGWDEFTKHVTSECLGWMRQQRAEMDMVAWGVDLASVEQHINSHRGIHNSIGDYRWQLDKIKADLREKSAIYQLEEEYENLLKASFERMDHLRQLQNIIQATSREIMWINDCEEEELLYDWSDKNTNIAQKQEAFSIRMSQLEVKEKELNKLKQESDQLVLNQHPASDKIEAYMDTLQTQWSWILQITKCIDVHLKENAAYFQFFEEAQSTEAYLKGLQDSIRKKYPCDKNMPLQHLLEQIKELEKEREKILEYKRQVQNLVNKSKKIVQLKPRNPDYRSNKPIILRALCDYKQDQKIVHKGDECILKDNNERSKWYVTGPGGVDMLVPSVGLIIPPPNPLAVDLSCKIEQYYEAILALWNQLYINMKSLVSWHYCMIDIEKIRAMTIAKLKTMRQEDYMKTIADLELHYQEFIRNSQGSEMFGDDDKRKIQSQFTDAQKHYQTLVIQLPGYPQHQTVTTTEITHHGTCQDVNHNKVIETNRENDKQETWMLMELQKIRRQIEHCEGRMTLKNLPLADQGSSHHITVKINELKSVQNDSQAIAEVLNQLKDMLANFRGSEKYCYLQNEVFGLFQKLENINGVTDGYLNSLCTVRALLQAILQTEDMLKVYEARLTEEETVCLDLDKVEAYRCGLKKIKNDLNLKKSLLATMKTELQKAQQIHSQTSQQYPLYDLDLGKFGEKVTQLTDRWQRIDKQIDFRLWDLEKQIKQLRNYRDNYQAFCKWLYDAKRRQDSLESMKFGDSNTVMRFLNEQKNLHSEISGKRDKSEEVQKIAELCANSIKDYELQLASYTSGLETLLNIPIKRTMIQSPSGVILQEAADVHARYIELLTRSGDYYRFLSEMLKSLEDLKLKNTKIEVLEEELRLARDANSENCNKNKFLDQNLQKYQAECSQFKAKLASLEELKRQAELDGKSAKQNLDKCYGQIKELNEKITRLTYEIEDEKRRRKSVEDRFDQQKNDYDQLQKARQCEKENLGWQKLESEKAIKEKEYEIERLRVLLQEEGTRKREYENELAKASNRIQESKNQCTQVVQERESLLVKIKVLEQDKARLQRLEDELNRAKSTLEAETRVKQRLECEKQQIQNDLNQWKTQYSRKEEAIRKIESEREKSEREKNSLRSEIERLQAEIKRIEERCRRKLEDSTRETQSQLETERSRYQREIDKLRQRPYGSHRETQTECEWTVDTSKLVFDGLRKKVTAMQLYECQLIDKTTLDKLLKGKKSVEEVASEIQPFLRGAGSIAGASASPKEKYSLVEAKRKKLISPESTVMLLEAQAATGGIIDPHRNEKLTVDSAIARDLIDFDDRQQIYAAEKAITGFDDPFSGKTVSVSEAIKKNLIDRETGMRLLEAQIASGGVVDPVNSVFLPKDVALARGLIDRDLYRSLNDPRDSQKNFVDPVTKKKVSYVQLKERCRIEPHTGLLLLSVQKRSMSFQGIRQPVTVTELVDSGILRPSTVNELESGQISYDEVGERIKDFLQGSSCIAGIYNETTKQKLGIYEAMKIGLVRPGTALELLEAQAATGFIVDPVSNLRLPVEEAYKRGLVGIEFKEKLLSAERAVTGYNDPETGNIISLFQAMNKELIEKGHGIRLLEAQIATGGIIDPKESHRLPVDIAYKRGYFNEELSEILSDPSDDTKGFFDPNTEENLTYLQLKERCIKDEETGLCLLPLKEKKKQVQTSQKNTLRKRRVVIVDPETNKEMSVQEAYKKGLIDYETFKELCEQECEWEEITITGSDGSTRVVLVDRKTGSQYDIQDAIDKGLVDRKFFDQYRSGSLSLTQFADMISLKNGVGTSSSMGSGVSDDVFSSSRHESVSKISTISSVRNLTIRSSSFSDTLEESSPIAAIFDTENLEKISITEGIERGIVDSITGQRLLEAQACTGGIIHPTTGQKLSLQDAVSQGVIDQDMATRLKPAQKAFIGFEGVKGKKKMSAAEAVKEKWLPYEAGQRFLEFQYLTGGLVDPEVHGRISTEEAIRKGFIDGRAAQRLQDTSSYAKILTCPKTKLKISYKDAINRSMVEDITGLRLLEAASVSSKGLPSPYNMSSAPGSRSGSRSGSRSGSRSGSRSGSRRGSFDATGNSSYSYSYSFSSSSIGH

SEQ ID NO: 349 - DNA, 1900 bp, Homo sapiens
  source 1..1900: mol_type = genomic DNA
atggcgcggagcccgggacgcgcgtacgccctgctgcttctcctgatctgctttaacgttggaagtggacttcacttacaggtcttaagcacaagaaatgaaaataagctgcttcctaaacatcctcatttagtgcggcaaaagcgcgcctggatcaccgcccccgtggctcttcgggagggagaggatctgtccaagaagaatccaattgccaagatacattctgatcttgcagaagaaagaggactcaaaattacttacaaatacactggaaaagggattacagagccaccttttggtatatttgtctttaacaaagatactggagaactgaatgttaccagcattcttgatcgagaagaaacaccattttttctgctaacaggttacgctttggatgcaagaggaaacaatgtagagaaacccttagagctacgcattaaggttcttgatatcaatgacaacgaaccagtgttcacacaggatgtctttgttgggtctgttgaagagttgagtgcagcacatactcttgtgatgaaaatcaatgcaacagatgcagatgagcccaataccctgaattcgaaaatttcctatagaatcgtatctctggagcctgcttatcctccagtgttctacctaaataaagatacaggagagatttatacaaccagtgttaccttggacagagaggaacacagcagctacactttgacagtagaagcaagagatggcaatggagaagttacagacaaacctgtaaaacaagctcaagttcagattcgtattttggatgtcaatgacaatatacctgtagtagaaaataaagtgcttgaagggatggttgaagaaaatcaagtcaacgtagaagttacgcgcataaaagtgttcgatgcagatgaaataggttctgataattggctggcaaattttacatttgcatcaggaaatgaaggaggttatttccacatagaaacagatgctcaaactaacgaaggaattgtgacccttattaaggaagtagattatgaagaaatgaagaatcttgacttcagtgttattgtcgctaataaagcagcttttcacaagtcgattaggagtaaatacaagcctacacccattcccatcaaggtcaaagtgaaaaatgtgaaagaaggcattcattttaaaagcagcgtcatctcaatttatgttagcgagagcatggatagatcaagcaaaggccaaataattggaaattttcaagcttttgatgaggacactggactaccagcccatgcaagatatgtaaaattagaagatagagataattggatctctgtggattctgtcacatctgaaattaaacttgcaaaacttcctgattttgaatctagatatgttcaaaatggcacatacactgtaaagattgtggccatatcagaagattatcctagaaaaaccatcactggcacagtccttatcaatgttgaagacatcaacgacaactgtcccacactgatagagcctgtgcagacaatctgtcacgatgcagagtatgtgaatgttactgcagaggacctggatggacacccaaacagtggccctttcagtttctccgtcattgacaaaccacctggcatggcagaaaaatggaaaatagcacgccaagaaagtaccagtgtgctgctgcaacaaagtgagaaaaagcttgggagaagtgaaattcagttcctgatttcagacaatcagggttttagttgtcctgaaaagcaggtccttacactcacagtttgtgagtgtctgcatggcagcggctgcagggaagcacagcatgactcctatgtgggcctgggacccgcagcaattgcgctcatgattttggcctttctgctcctgctattggtaccacttttactgctga

SEQ ID NO: 350 - protein, 1118 aa, Homo sapiens
MARSPGRAYALLLLLICFNVGSGLHLQVLSTRNENKLLPKHPHLVRQKRAWITAPVALREGEDLSKKNPIAKIHSDLAEERGLKITYKYTGKGITEPPFGIFVFNKDTGELNVTSILDREETPFFLLTGYALDARGNNVEKPLELRIKVLDINDNEPVFTQDVFVGSVEELSAAHTLVMKINATDADEPNTLNSKISYRIVSLEPAYPPVFYLNKDTGEIYTTSVTLDREEHSSYTLTVEARDGNGEVTDKPVKQAQVQIRILDVNDNIPVVENKVLEGMVEENQVNVEVTRIKVFDADEIGSDNWLANFTFASGNEGGYFHIETDAQTNEGIVTLIKEVDYEEMKNLDFSVIVANKAAFHKSIRSKYKPTPIPIKVKVKNVKEGIHFKSSVISIYVSESMDRSSKGQIIGNFQAFDEDTGLPAHARYVKLEDRDNWISVDSVTSEIKLAKLPDFESRYVQNGTYTVKIVAISEDYPRKTITGTVLINVEDINDNCPTLIEPVQTICHDAEYVNVTAEDLDGHPNSGPFSFSVIDKPPGMAEKWKIARQESTSVLLQQSEKKLGRSEIQFLISDNQGFSCPEKQVLTLTVCECLHGSGCREAQHDSYVGLGPAAIALMILAFLLLLLVPLLLLMCHCGKGAKGFTPIPGTIEMLHPWNNEGAPPEDKVVPSFLPVDQGGSLVGRNGVGGMAKEATMKGSSSASIVKGQHEMSEMDGRWEEHRSLLSGRATQFTGATGAIMTTETTKTARATGASRDMAGAQAAAVALNEEFLRNYFTDKAASYTEEDENHTAKDCLLVYSQEETESLNASIGCCSFIEGELDDRFLDDLGLKFKTLAEVCLGQKIDINKEIEQRQKPATETSMNTASHSLCEQTMVNSENTYSSGSSFPVPKSLQEANAEKVTQEIVTERSVSSRQAQKVATPLPDPMASRNVIATETSYVTGSTMPPTTVILGPSQPQSLIVTERVYAPASTLVDQPYANEGTVVVTERVIQPHGGGSNPLEGTQHLQDVPYVMVRERESFLAPSSGVQPTLAMPNIAVGQNVTVTERVLAPASTLQSSYQIPTENSMTARNTTVSGAGVPGPLPDFGLEESGHSNSTITTSSTRVTKHSTVQHSYS

SEQ ID NO: 351 - DNA, 2238 bp, Homo sapiens
  source 1..2238: mol_type = genomic DNA
atggaggtgatgaacctgatggagcagcctatcaaggtgactgagtggcagcagacatacacctacgactcgggtatccactcgggcgccaacacctgcgtgccctccgtcagcagcaagggcatcatggaggaggatgaggcctgcgggcgccagtacacgctcaagaaaaccaccacttacacccagggggtgccccccagccaaggtgatctggagtaccagatgtccacaacagccagggccaaacgggtgcgggaggccatgtgccctggtgtgtcaggcgaggacagctcgcttctgctggccacccaggtggaggggcaggccaccaacctgcagcgactggccgagccgtcccagctgctcaagtcggccattgtgcatctcatcaactaccaggacgatgccgagctggccactcgcgccctgcccgagctcaccaaactgctcaacgacgaggacccggtggtggtgaccaaggcggccatgattgtgaaccagctgtcgaagaaggaggcgtcgcggcgggccctgatgggctcgccccagctggtggccgctgtcgtgcgtaccatgcagaataccagcgacctggacacagcccgctgcaccaccagcatcctgcacaacctctcccaccaccgggaggggctgctcgccatcttcaagtcgggtggcatccctgctctggtccgcatgctcagctcccctgtggagtcggtcctgttctatgccatcaccacgctgcacaacctgctcctgtaccaggagggcgccaagatggccgtgcgcctggccgacgggctgcaaaagatggtgcccctgctcaacaagaacaaccccaagttcctggccatcaccaccgactgcctgcagctcctggcctacggcaaccaggagagcaagctgatcatcctggccaatggtgggccccaggccctcgtgcagatcatgcgtaactacagttatgaaaagctgctctggaccaccagtcgtgtgctcaaggtgctatccgtgtgtcccagcaataagcctgccattgtggaggctggtgggatgcaggccctgggcaagcacctgaccagcaacagcccccgcctggtgcagaactgcctgtggaccctgcgcaacctctcagatgtggccaccaagcaggagggcctggagagtgtgctgaagattctggtgaatcagctgagtgtggatgacgtcaacgtcctcacctgtgccacgggcacactctccaacctgacatgcaacaacagcaagaacaagacgctggtgacacagaacagcggtgtggaggctctcatccatgccatcctgcgtgctggtgacaaggacgacatcacggagcctgccgtctgcgctctgcgccacctcactagccgccaccctgaggccgagatggcccagaactctgtgcgtctcaactatggcatcccagccatcgtgaagctgctcaaccagcccaaccagtggccactggtcaaggcaaccatcggcttgatcaggaatctggccctgtgcccagccaaccatgccccgctgcaggaggcagcggtcatcccccgcctcgtccaactgctggtgaaggcccaccaggatgcccagcgccacgtagctgcaggcacacagcagccctacacggatggtgtgaggatggaggagattgtggagggctgcaccggagcactgcacatcctcgcccgggaccccatgaaccgcatggagatcttccggctcaacaccattcccctgtttgtgcagctcctgtactcgtcggtggagaacatccagcgcgtggctgccggggtgctgtgtgagctggcccaggacaaggaggcggccgacgccattgatgcagagggggcctcggccccactcatggagttgctgcactcccgcaacgagggcactgccacctacgctgctgccgtcctgttccgcatctccgaggacaagaacccagactaccggaagcgcgtgtccgtggagctcaccaactccctcttcaagcatgacccggctgcctgggaggctgcccagagcatgattcccatcaatgagccctatggagatgacatggatgccacctaccgccccatgtactccagcgatgtgccccttgacccgctggagatgcacatggacatggatggagactaccccatcgacacctacagcgacggcctcaggcccccgtaccccactgcagaccacatgctggcctag

SEQ ID NO: 352 - protein, 745 aa, Homo sapiens
MEVMNLMEQPIKVTEWQQTYTYDSGIHSGANTCVPSVSSKGIMEEDEACGRQYTLKKTTTYTQGVPPSQGDLEYQMSTTARAKRVREAMCPGVSGEDSSLLLATQVEGQATNLQRLAEPSQLLKSAIVHLINYQDDAELATRALPELTKLLNDEDPVVVTKAAMIVNQLSKKEASRRALMGSPQLVAAVVRTMQNTSDLDTARCTTSILHNLSHHREGLLAIFKSGGIPALVRMLSSPVESVLFYAITTLHNLLLYQEGAKMAVRLADGLQKMVPLLNKNNPKFLAITTDCLQLLAYGNQESKLIILANGGPQALVQIMRNYSYEKLLWTTSRVLKVLSVCPSNKPAIVEAGGMQALGKHLTSNSPRLVQNCLWTLRNLSDVATKQEGLESVLKILVNQLSVDDVNVLTCATGTLSNLTCNNSKNKTLVTQNSGVEALIHAILRAGDKDDITEPAVCALRHLTSRHPEAEMAQNSVRLNYGIPAIVKLLNQPNQWPLVKATIGLIRNLALCPANHAPLQEAAVIPRLVQLLVKAHQDAQRHVAAGTQQPYTDGVRMEEIVEGCTGALHILARDPMNRMEIFRLNTIPLFVQLLYSSVENIQRVAAGVLCELAQDKEAADAIDAEGASAPLMELLHSRNEGTATYAAAVLFRISEDKNPDYRKRVSVELTNSLFKHDPAAWEAAQSMIPINEPYGDDMDATYRPMYSSDVPLDPLEMHMDMDGDYPIDTYSDGLRPPYPTADHMLA

SEQ ID NO: 353 - DNA, 1450 bp, Homo sapiens
  source 1..1450: mol_type = genomic DNA
atggctccggccgcctggctccgcagcgcggccgcgcgcgccctcctgcccccgatgctgctgctgctgctccagccgccgccgctgctggcccgggctctgccgccggacgcccaccacctccatgccgagaggagggggccacagccctggcatgcagccctgcccagtagcccggcacctgcccctgccacgcaggaagccccccggcctgccagcagcctcaggcctccccgctgtggcgtgcccgacccatctgatgggctgagtgcccgcaaccgacagaagaggttcgtgctttctggcgggcgctgggagaagacggacctcacctacaggatccttcggttcccatggcagttggtgcaggagcaggtgcggcagacgatggcagaggccctaaaggtatggagcgatgtgacgccactcacctttactgaggtgcacgagggccgtgctgacatcatgatcgacttcgccaggtactggcatggggacgacctgccgtttgatgggcctgggggcatcctggcccatgccttcttccccaagactcaccgagaaggggatgtccacttcgactatgatgagacctggactatcggggatgaccagggcacagacctgctgcaggtggcagcccatgaatttggccacgtgctggggctgcagcacacaacagcagccaaggccctgatgtccgccttctacacctttcgctacccactgagtctcagcccagatgactgcaggggcgttcaacacctatatggccagccctggcccactgtcacctccaggaccccagccctgggcccccaggctgggatagacaccaatgagattgcaccgctggagccagacgccccgccagatgcctgtgaggcctcctttgacgcggtctccaccatccgaggcgagctctttttcttcaaagcgggctttgtgtggcgcctccgtgggggccagctgcagcccggctacccagcattggcctctcgccactggcagggactgcccagccctgtggacgctgccttcgaggatgcccagggccacatttggttcttccaaggtgctcagtactgggtgtacgacggtgaaaagccagtcctgggccccgcacccctcaccgagctgggcctggtgaggttcccggtccatgctgccttggtctggggtcccgagaagaacaagatctacttcttccgaggcagggactactggcgtttccaccccagcacccggcgtgtagacagtcccgtgccccgcagggccactgactggagaggggtgccctctgagatcgacgctgccttccaggatgctgatggctatgcctacttcctgcgcggccgcctctactggaagtttgaccctgtgaaggtgaaggctctggaaggcttcccccgtctcgtgggtcctgacttctttggctgtgccgagcctg

SEQ ID NO: 354 - protein, 488 aa, Homo sapiens
MAPAAWLRSAAARALLPPMLLLLLQPPPLLARALPPDAHHLHAERRGPQPWHAALPSSPAPAPATQEAPRPASSLRPPRCGVPDPSDGLSARNRQKRFVLSGGRWEKTDLTYRILRFPWQLVQEQVRQTMAEALKVWSDVTPLTFTEVHEGRADIMIDFARYWHGDDLPFDGPGGILAHAFFPKTHREGDVHFDYDETWTIGDDQGTDLLQVAAHEFGHVLGLQHTTAAKALMSAFYTFRYPLSLSPDDCRGVQHLYGQPWPTVTSRTPALGPQAGIDTNEIAPLEPDAPPDACEASFDAVSTIRGELFFFKAGFVWRLRGGQLQPGYPALASRHWQGLPSPVDAAFEDAQGHIWFFQGAQYWVYDGEKPVLGPAPLTELGLVRFPVHAALVWGPEKNKIYFFRGRDYWRFHPSTRRVDSPVPRRATDWRGVPSEIDAAFQDADGYAYFLRGRLYWKFDPVKVKALEGFPRLVGPDFFGCAEPANTFL

SEQ ID NO: 355 - DNA, 2930 bp, Homo sapiens
  source 1..2930: mol_type = genomic DNA
atgggtgctgaggaggaggtgctggtcacactatcagggggagccccctggggcttccgacttcatgggggggccgagcagaggaaaccgttacaggtgtctaagattcgaagacggagccaggctggcagagcaggactccgagagagggaccagctcttggcaatcaatggggtctcttgcaccaacctctcccatgccagtgccatgagcctcatcgatgcctcaggaaatcagcttgtcctcactgtgcagcggttagcagacgagggtcctgtgcaatctccatctccccatgagcttcaggtgctgtcacccttatctccactaagtcctgagccccctggtgctccagttcctcagcctcttcagcctgggagccttcgttcacctcctgatagtgaggcttactacggagagactgacagtgatgctgatggccctgccacccaggagaagccccgtcgacctcgccgccgaggccccacaaggcccacccctccgggtgccccacctgatgaggtctacctgtctgacagccctgcagagccagcacctactatccctggccctcccagccagggtgacagcgtgtgagctccccgtcttgggaggatggggcagcccttcagccacccccagctgaggctctgctgttaccccatggccccctccgacctggtcctcatctcatccctatggtggggcctgttccccacccagtggcagaagatcttactaccacctacacccagaaggccaagcaagccaaactgcaacgtgcagagagcctccaagagaagagcataaaagaggccaagaccaaatgcaggacaattgcatccctgctcactgcagcccccaacccccactccaaaggggtacttatgtttaagaaacggcggcagagagccaagaagtacaccctggtgagcttcggggctgctgctgggacaggcgctgaggaggaggacggcgttccccccacgagtgagtccgagctggacgaagaagccttctctgacgcccgcagcctcaccaatcaatctgactgggacagtccctatctggacatggagcttgccagggcgggctcaagagcatcagagggccagggctctgggctgggagggcagctgagtgaggtctctgggcgaggggtgcagctctttgaacagcagcgccagcgcgcagactccagcacccaggaactggcacgggtcgaaccagcagccatgctcaacggggaaggcctgcagtcaccacctcgggcccagagtgctcccccagaggcagctgtgctcccacccagccccttgccggcgcctgtagccagccccagacccttccaaccaggtggtggagccccgaccccagctccaagcatctttaaccggtcagccaggccctttaccccgggcctacaagggcagcggccaactaccacctcggttattttccggcctttagcccccaaaagggcgaacgacagcctggggggcctcagccccgccccaccccccttcttgtcttcgcaggggcccacccctctgcccagcttcacttcaggggttcccagccacgcgccagtctctggttcccccagcaccccacgctcctcgggccctgtgacagccaccagctccctgtacatcccagcccctagtcggcctgtcaccccaggtggagctccagagccccccgctcctcctagcgcagctgccatgacctccaccgcttctatcttcctatctgcgcctttgcgaccctctgcgcgcccagaggcgcctgccccaggcccaggggctcctgagccccccagcgctcgcgagcagcgcatctctgtgccagctgcccgcacgggtatcctgcaggaggcccggcgccgggggacccggaagcagatgttccggccgggaaaggaggagacgaagaactcgcccaaccccgagctgctatcgctggtacagaacctggatgaaaagcctcgggccgggggtgcagaatctggtcctgaagaagatgctctgagcctcggggctgaagcctgcaacttcatgcagccagtaggggccaggagttacaagaccctgcctcacgtgacacctaagaccccccctccaatggctcccaagaccccgccccctatgactcctaagactccacccccagtggctcctaagcccccatctcgagggctccttgatgggctcgtgaatggggcagcctcttcggctggaatccctgagccaccaaggctgcagggcaggggtggggagctgtttgctaagcggcagagccgtgcggacaggtatgtggtggaaggtacacctggtcctggtcttggccctcggcctagaagtccttctcctaccccgtctctgcccccttcctggaaatattcacccaacatccgtgccccgcctcctattgcttacaacccactgctctctccctttttcccccaggcggcccgaactctccctaaggcccaatcccaggggcctcgggcaacacccaagcagggcatcaaggctctagattttatgcggcatcagccctatcaacttaaaactgccatgttctgttttgatgaggttcccccgactcctggccctatcgcctcagggtcccccaaaactgcccgagtccaggagattcgccggttttccactccggcaccccagcccactgcagaacccctggctcccactgtgcttgccccccgagcagccactacactggatgagcccatctggagaacagaactggcctcagcccctgttcctagcccagcccctcctccagaggctcccaggggccttggggcttctcccagctcctgcggtttccaggtagccaggccccgattttcagccaccagaacaggattgcaagctcatgtgtggaggcctggggcagggcaccag

SEQ ID NO: 356 - protein, 977 aa, Homo sapiens
MGAEEEVLVTLSGGAPWGFRLHGGAEQRKPLQVSKIRRRSQAGRAGLRERDQLLAINGVSCTNLSHASAMSLIDASGNQLVLTVQRLADEGPVQSPSPHELQVLSPLSPLSPEPPGAPVPQPLQPGSLRSPPDSEAYYGETDSDADGPATQEKPRRPRRRGPTRPTPPGAPPDEVYLSDSPAEPAPTIPGPPSQGDSRVSSPSWEDGAALQPPPAEALLLPHGPLRPGPHLIPMVGPVPHPVAEDLTTTYTQKAKQAKLQRAESLQEKSIKEAKTKCRTIASLLTAAPNPHSKGVLMFKKRRQRAKKYTLVSFGAAAGTGAEEEDGVPPTSESELDEEAFSDARSLTNQSDWDSPYLDMELARAGSRASEGQGSGLGGQLSEVSGRGVQLFEQQRQRADSSTQELARVEPAAMLNGEGLQSPPRAQSAPPEAAVLPPSPLPAPVASPRPFQPGGGAPTPAPSIFNRSARPFTPGLQGQRPTTTSVIFRPLAPKRANDSLGGLSPAPPPFLSSQGPTPLPSFTSGVPSHAPVSGSPSTPRSSGPVTATSSLYIPAPSRPVTPGGAPEPPAPPSAAAMTSTASIFLSAPLRPSARPEAPAPGPGAPEPPSAREQRISVPAARTGILQEARRRGTRKQMFRPGKEETKNSPNPELLSLVQNLDEKPRAGGAESGPEEDALSLGAEACNFMQPVGARSYKTLPHVTPKTPPPMAPKTPPPMTPKTPPPVAPKPPSRGLLDGLVNGAASSAGIPEPPRLQGRGGELFAKRQSRADRYVVEGTPGPGLGPRPRSPSPTPSLPPSWKYSPNIRAPPPIAYNPLLSPFFPQAARTLPKAQSQGPRATPKQGIKALDFMRHQPYQLKTAMFCFDEVPPTPGPIASGSPKTARVQEIRRFSTPAPQPTAEPLAPTVLAPRAATTLDEPIWRTELASAPVPSPAPPPEAPRGLGASPSSCGFQVARPRFSATRTGLQAHVWRPGAGHQ

SEQ ID NO: 357 - DNA, 2259 bp, Homo sapiens
  source 1..2259: mol_type = genomic DNA
atggagacctttgagcccatcagccaagagcccctcagccaagccagctatgacaaagccccagacccagttcctgagctccaagactcgttctatgcagaactgcaacgtgcagagagcctccaagagaagagcataaaagaggccaagaccaaatgcaggacaattgcatccctgctcactgcagcccccaacccccactccaaaggggtacttatgtttaagaaacggcggcagagagccaagaagtacaccctggtgagcttcggggctgctgctgggacaggcgctgaggaggaggacggcgttccccccacgagtgagtccgagctggacgaagaagccttctctgacgcccgcagcctcaccaatcaatctgactgggacagtccctatctggacatggagcttgccagggcgggctcaagagcatcagagggccagggctctgggctgggagggcagctgagtgaggtctctgggcgaggggtgcagctctttgaacagcagcgccagcgcgcagactccagcacccaggaactggcacgggtcgaaccagcagccatgctcaacggggaaggcctgcagtcaccacctcgggcccagagtgctcccccagaggcagctgtgctcccacccagccccttgccggcgcctgtagccagccccagacccttccaaccaggtggtggagccccgaccccagctccaagcatctttaaccggtcagccaggccctttaccccgggcctacaagggcagcggccaactaccacctcggttattttccggcctttagcccccaaaagggcgaacgacagcctggggggcctcagccccgccccaccccccttcttgtcttcgcaggggcccacccctctgcccagcttcacttcaggggttcccagccacgcgccagtctctggttcccccagcaccccacgctcctcgggccctgtgacagccaccagctccctgtacatcccagcccctagtcggcctgtcaccccaggtggagctccagagccccccgctcctcctagcgcagctgccatgacctccaccgcttctatcttcctatctgcgcctttgcgaccctctgcgcgcccagaggcgcctgccccaggcccaggggctcctgagccccccagcgctcgcgagcagcgcatctctgtgccagctgcccgcacgggtatcctgcaggaggcccggcgccgggggacccggaagcagatgttccggccgggaaaggaggagacgaagaactcgcccaaccccgagctgctatcgctggtacagaacctggatgaaaagcctcgggccgggggtgcagaatctggtcctgaagaagatgctctgagcctcggggctgaagcctgcaacttcatgcagccagtaggggccaggagttacaagaccctgcctcacgtgacacctaagaccccccctccaatggctcccaagaccccgccccctatgactcctaagactccacccccagtggctcctaagcccccatctcgagggctccttgatgggctcgtgaatggggcagcctcttcggctggaatccctgagccaccaaggctgcagggcaggggtggggagctgtttgctaagcggcagagccgtgcggacaggtatgtggtggaaggtacacctggtcctggtcttggccctcggcctagaagtccttctcctaccccgtctctgcccccttcctggaaatattcacccaacatccgtgccccgcctcctattgcttacaacccactgctctctccctttttcccccaggcggcccgaactctccctaaggcccaatcccaggggcctcgggcaacacccaagcagggcatcaaggctctagattttatgcggcatcagccctatcaacttaaaactgccatgttctgttttgatgaggttcccccgactcctggccctatcgcctcagggtcccccaaaactgcccgagtccaggagattcgccggttttccactccggcaccccagcccactgcagaacccctggctcccactgtgcttgccccccgagcagccactacactggatgagcccatctggagaacagaactggcctcagcccctgttcctagcccagcccctcctccagaggctcccaggggccttggggcttctcccagctcctgcggtttccaggtagccaggccccgattttcagccaccagaacaggattgcaagctcatgtgtggaggcctggggcagggcaccag

SEQ ID NO: 358 - protein, 753 aa, Homo sapiens
METFEPISQEPLSQASYDKAPDPVPELQDSFYAELQRAESLQEKSIKEAKTKCRTIASLLTAAPNPHSKGVLMFKKRRQRAKKYTLVSFGAAAGTGAEEEDGVPPTSESELDEEAFSDARSLTNQSDWDSPYLDMELARAGSRASEGQGSGLGGQLSEVSGRGVQLFEQQRQRADSSTQELARVEPAAMLNGEGLQSPPRAQSAPPEAAVLPPSPLPAPVASPRPFQPGGGAPTPAPSIFNRSARPFTPGLQGQRPTTTSVIFRPLAPKRANDSLGGLSPAPPPFLSSQGPTPLPSFTSGVPSHAPVSGSPSTPRSSGPVTATSSLYIPAPSRPVTPGGAPEPPAPPSAAAMTSTASIFLSAPLRPSARPEAPAPGPGAPEPPSAREQRISVPAARTGILQEARRRGTRKQMFRPGKEETKNSPNPELLSLVQNLDEKPRAGGAESGPEEDALSLGAEACNFMQPVGARSYKTLPHVTPKTPPPMAPKTPPPMTPKTPPPVAPKPPSRGLLDGLVNGAASSAGIPEPPRLQGRGGELFAKRQSRADRYVVEGTPGPGLGPRPRSPSPTPSLPPSWKYSPNIRAPPPIAYNPLLSPFFPQAARTLPKAQSQGPRATPKQGIKALDFMRHQPYQLKTAMFCFDEVPPTPGPIASGSPKTARVQEIRRFSTPAPQPTAEPLAPTVLAPRAATTLDEPIWRTELASAPVPSPAPPPEAPRGLGASPSSCGFQVARPRFSATRTGLQAHVWRPGAGHQ

SEQ ID NO: 359 - RNA, 4994 nt, Homo sapiens
  source 1..4994: mol_type = mRNA
agagcgagcgccggggccgggcgcgcaggagtgaaaaggaggcggcggccgcagctgcgagcaacagatccggacgccgcgagctgacccgctctgctgttgggcgatttttttttaattgcagaaaaatttattaaattggaaaatcttgcgtttttcaatggcgctggccccgggtcagcgggcgattttctctgcatcaagatgggctttgccgtttccgtagtgggcaccagtggtggcctgattgtcagtcttctcccggcatttttaaggccaggagccgagcgctgcttgtaggcgaataccctacagagcggtttggctttttaaattactgttattattttgggcagagaacagtcggtctggtgcaccccgtcctcgctgcagaagaggctgcgagtccgaggtgggtctctcggaaggtgaaattccttctggggtgagcgagccccggccccgcgcgcagtccagcggccccgcgtgtgtgccctcgccctgccggagccgggaaaatggaggctgtgattgagaaggaatgcagcgcgctcggaggcctcttccagaccatcatcagcgacatgaaggggagctatccagtttgggaagatttcataaacaaagcaggaaagctgcagtcccagcttcggacaacagtagtagcagcagctgccttcttggacgcctttcagaaagtggctgacatggccaccaacacacgtggtgggaccagggagattggatctgctctcaccaggatgtgcatgaggcacagaagcattgaagccaagctgaggcagttttcgagcgctttaattgattgtctgataaacccacttcaagaacagatggaagaatggaagaaagtggccaaccagctggataaagaccacgcaaaagaatataagaaagcccgccaagagataaaaaagaagtcctcggatacgctgaaactgcagaagaaagcaaaaaaagggagaggtgatatccagcctcagttggacagtgctctccaagatgtcaatgataagtatctcttattggaagaaacagaaaagcaggctgtccggaaggctttgattgaagaacgtggccgattctgtaccttcatctctatgctgcggccagtgattgaagaagaaatctcaatgctaggggaaataacccaccttcagaccatctcggaagatctaaaaagcctgaccatggaccctcacaaactgccctcctcaagtgaacaggtgattctggacttgaaaggttctgattacagctggtcgtatcagacgccaccctcttcccccagcaccaccatgtccagaaagtccagtgtctgcagcagcctgaacagtgtcaacagcagtgactcccggtccagcggctcccactcgcattcccccagctcacattaccgctaccgcagctccaacctggcccagcaggctcctgtgaggctgtccagcgtgtcctcccatgactcaggattcatatcccaggatgccttccagtccaagtcaccatcccccatgccgccagaggcccccaaccagttgtctaacgggttttctcactatagtttatcaagtgagtcccacgtggggcccacgggtgcaggccttttccctcattgcctgcctgcctcccgcctgctccctcgggtcacctctgtccaccttccagactacgctcattattacaccattgggcccggcatgttcccgtcatctcagatccctagctggaaggactgggctaagcctgggccctatgaccagcctctggtgaacaccctgcagcgccgcaaagagaagcgagaaccggaccccaacgggggaggacccactaccgccagcggcccacctgcagcagctgaggaggctcagagaccacggagcatgactgtatcggctgccaccaggcctggtgaggagatggaggcttgtgaggagctggccctggccctgtctcggggcctgcagctggacacccagaggagcagccgggactcgcttcagtgctccagcggctacagcacccagacaaccaccccctgctgctctgaggacaccatcccttcccaagtttcagattatgattatttctctgtaagtggtgaccaggaggcagatcagcaggagttcgacaagtcctccaccattccaagaaacagcgacatcagccagtcctaccgacggatgttccaagccaagcgtccagcctcaactgctggcctccccaccaccctgggacctgctatggtcactccaggggttgcaactatccgacggaccccttccaccaagccttctgtccgccggggaaccattggagctggtcccatccccatcaagacacccgtgatccctgtcaagaccccaaccgtcccagacctcccaggggtgttgccagcccctccagatgggccagaagagcggggggagcacagccctgagtcgccatctgtgggtgagggcccccaaggtgtcaccagcatgccctcctcaatgtggagcggccaagcttccgttaaccctccacttccaggcccgaagcccagtatccctgaggagcacagacaggcaattccagaaagtgaagctgaagaccaggaacgggaacccccaagtgccactgtctccccaggccagattccagagagtgaccctgcagacctgagcccaagggatactccacaaggagaagacatgctgaacgccatccgaaggggcgtgaaactgaagaagaccacgacaaacgatcgctcagcccctcgcttttcttaggttcacaagaaatgcgccggtggggaatgaactgtttcattaataaaacctaatttgtcttgatccattccactctataataaaacaaaagattttgtaggcaactcggaatatagctcttttgaaagtactcgacacctttagataagaattaaaaccaacctatgtaactgacataatcttgatcttttaatttgtaaatattgacaattttctttctgcacattttaatcttagtttcccttttgatttttctgaaggtgccaaattccatttaacttttttacaagtctttgtaaaattttaaatgcataaagggggttggggcaggggaaccacgaagtagttaattttagaaaaggatttactatacttcactcttctttttttttccccacaagcttttgtagatgcattgtagtagtctagcttagaagcaaatgcaagttattttaatgtacaaactaaatgggtaagaggtaaaatcttcatttaaatatactatgttctggatgaaaagagcaggagtaacaattgatgagcaatattcagagtgaagtaaatctggaaatggtagactgtgttgggattggggggagggccatgggaggggtacatcgtcaacatagccgatcctgttacatttaagagtagcctcgtaggttgaatttcttctggtagcttcatggtaaatgcatccgaataagccatactggattgcagtgtttgtttctgtagggtgtttaaggacttgacttcctttctcccatgattcctctggactgcacacagcacccacaaccagccccatgcatgctgctgcctctgggcagtcgtagaatctcccacttcagtttctcgttgattgtactcacctttatggaatccaaatacatccaaaagggtaaggcagttttaaaaatgtgaaaacatttaaaaatgataatagcagggaattcttagattatagtaaatgccttttacttaactgtgcccagcaggctgggtgcgttaaaaagcccaagtattttgaaaaaactcgaacagatttgacaagggtagccagcttggagtctagcaacttgccaatgtgtttaccaatctgggggcttgtttttcttttcttctttcaaataaatggcagttaactggctttacagtaaacattgaagagaggaggatttgtttattgtcactgggaatctgaccactatactgtcctttttttgtattctgggtaaatgttttttggaaaagatttgtcttttctaagtggaagttaaatttgttatactgcccatcccctaaagccaacagagatttgtagatttaaagggatcacatttgaagacaatagtgtttaagaaagcaagcaagtcccttagcagtcaggtcataacagggcacatttctgaccgaaccctctcaaggcagaggaggagtttggtgggtttcatacaccctgcagattcctgttggctctaaccctcaattacctaatcttatgctttaacacataactgcattggatgtgagagtaacgtaccgtatggtcattgttctatatattaacattgaacactgctgcgattgctcaaggacattttatgttacggctttaaagcaaaggcatgattattagaaactatttaagcttttttctttgaaaaacaagctccttttacagaatataaacaacagtagtgcctgtggtttagcccaccaatcttgatgactaaaagtagctgatgcattgtgcatatgatgcttgagatggtttttgcaaaagcagaaatcgctgcaaggtaatcacaatagataaaagtggtattttaaacctttgaaataaatggatgtaactgtaccttggtacagcttttcacttgtttagtttttaaacgttagtataatctgaataaataaaatgttgccaaattcaatgtagaaagaatgtgacaacacaccttgggtagttctgcttgtgtttttgcatattgtaaaagcagtgtcacagctaaaaagaaagaaatcgtttctaacagtaaattattgtgctttagttgctagtttgtactgagagttgacctctccctgtgcagttttttgttctaaacttgtataaataacaattgtgtaatgtgtctccctcctacattgtaacaattgcttcagcctacgttataaataaagaaccactagattaaaaaa

SEQ ID NO: 360 - DNA, 3258 bp, synthetic construct
  source 1..3258: mol_type = other DNA
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagtaa

SEQ ID NO: 361 - protein, 1085 aa, synthetic construct
MAPKKKRKVGIHGVPAAKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKK

SEQ ID NO: 362 - DNA, 3069 bp, synthetic construct
  source 1..3069: mol_type = other DNA
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagaaagctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaagagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaag

SEQ ID NO: 363 - protein, 1023 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAK

SEQ ID NO: 364 - DNA, 1023 bp, synthetic construct
  source 1..1023: mol_type = other DNA
gaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgac

SEQ ID NO: 365 - protein, 341 aa, synthetic construct
EIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD